SEQUENCE LISTING (206 sequences)

SEQ ID NO: 1 - DNA, 1318 bp, Rattus sp.
tgagttgggtccctggaaccacacagtggaaagagaactgtctgccacaagttgtcctctgtcctccacacgcacagtgacacatgccctcattcaccccagtgtgcacggcatcaataaatgtaattgcttttttagatgtatttacttttgatgtgtatgagtatttgcctggatgaaagtatgtgcacagactttagatgcccctggaaccagagttacagatagttgtgaaccaccatgctagtgccaggaaccaaacccaatttctgtaagagtgctcttgcttgcttagccttctcaccagccctctagtttaaattaaagcaacaaacaaacaaacaagtgatgaagaattgcatgaaggaaaagtaaacttaagaaaataatagccggttgtggtggtacacgctggtaggatttgctgaaagaggcagaggcaggaggattgtgagtttgaggccagcctaggctacacattagcctttctggtggtgacaacctccctaagagagcaggcctcgaaaaggatctctttcatccctctactgaggagaagaggaaatgcaagaaaaatcgcctggtgcagagccctaattcctactttatagatgtgaaatgcccaggatgctataaaatcaccatggtctctagccatgcacaaatggtagtcttgtgtgttggctgctccactgtcctgtcagcctacaggtgggaaagcaaggctgacagaaagacgctccttcaggaggaagcagcactgaaagcccctgactcaagatgagtgggaaccttcccaataaacacattttgaatataagaaaaaagaaaataataacaatgtctaaggattagccttgtagctcaggcctgctgtcctagcattcaggaggcaagagagtagaatggtcatgaggcttgttttgggggtacagagtaaggcttggtctcaaacaacaacagcaataacaaactggacatggtagtacgagttatgtcatagttctcaggagaagcatcaggaattcaaagtcatcttcacggatacctggtaagttcaaggctatcctgggccacatagtgagttctaagtctgcttgggacacataagaccctgtcccaaaaagcaaaatatataaagcgtatatatatttttaaaagatttatttattgtatataagtacactgtagctgtctttaaacataccagaaaagggcaccagatctcattacagatggttgtgagccatcatatggttgctgggaattgacctcaggacctctggaagagcagtcagtgctcttaaccgctgagcca

SEQ ID NO: 2 - DNA, 1378 bp, Mus musculus
tctggaaaggaaagggagatccaaatcccctggtcctgctttttgctttcctagtttaagctttccccacctgctagaggactgtaggtatctaatgcctggatcaggtgcaccgcctacggggaccccttagagtttccaccccctggaccattcgggaaccacctcacctcccgccgcatcactgggctaccctcctatcctctggtggcgagggtctcagcctttaagcagacgatctctaaggactgctcgccgggcacgcgcagagctggaagcccagaagttggaagaggggcggggacctgcgccctactggctggctgacagggggagcggcgggggcggaggccccctccggtgggtgctgggactgtagccactagaggcctggaggggaggggagagtgaccgtgagtctgtctgactgacaggctgcgaagagcagccaatatatataagaaaggctctggagcaagcaggtttcagtagcggcgctgctcgcaggctaggaacccgaggccaagagctgcagccaaagtcacttgggtgcagtgtactccctcactagcccgctcgagaccctaggatttgctccaggacacgtacttagagcagccaccgcccagtcgccctcacctggattacctaccgaggcatcgagcagcggagtttttgagaaggcgacaagggagcagcgtcccgaggggaatcagcttttcaggaactcggctggcagacgggacttgcgggagagcgacatccctaacaagcagattcggagtcccggagtggagaggacaccccaagggatgacgcctgcgtcccggagcgcctgtcgctgggcgctactgctgctggcggtactgccgcagtgggtaatgtctcacgtcctctccgccccctcccgcagcgctccgggcttgcgccccggccccggctgagcctgaccgctctcctccctccttctctcggtccctgtgcagcagcgcgctgcgggctccggcatcttccagctgcggctgcaggagttcgtcaaccagcgcggtatgctggccaatgggcagtcctgcgaaccgggctgccggactttcttccgcatctgccttaagcacttccaggcaaccttctccgagggaccctgcacctttggcaatgtctccacgccggtattgggcaccaactccttcgtcgtcagggacaagaatagcggcagtggtcgcaaccctctgcagttgcccttcaatttcacctggccggtaagcacaacttaaatgcaccgggagataaccgaagggaaagaagggagcgccgggacaccagagctcctttccaaagcgctctctggagagccccaagggctctttctcttctgccc

SEQ ID NO: 3 - DNA, 1273 bp, Mus musculus
cagtagcggcgctgctcgcaggctaggaacccgaggccaagagctgcagccaaagtcacttgggtgcagtgtactccctcactagcccgctcgagaccctaggatttgctccaggacacgtacttagagcagccaccgcccagtcgccctcacctggattacctaccgaggcatcgagcagcggagtttttgagaaggcgacaagggagcagcgtcccgaggggaatcagcttttcaggaactcggctggcagacgggacttgcgggagagcgacatccctaacaagcagattcggagtcccggagtggagaggacaccccaagggatgacgcctgcgtcccggagcgcctgtcgctgggcgctactgctgctggcggtactgtggccgcaggtaatgtctcacgtcctctccgccccctcccgcagcgctccgggcttgcgccccggccccggctgagcctgaccgctctcctccctccttctctcggtccctgtgcagcagcgcgctgcgggctccggcatcttccagctgcggctgcaggagttcgtcaaccagcgcggtatgctggccaatgggcagtcctgcgaaccgggctgccggactttcttccgcatctgccttaagcacttccaggcaaccttctccgagggaccctgcacctttggcaatgtctccacgccggtattgggcaccaactccttcgtcgtcagggacaagaatagcggcagtggtcgcaaccctctgcagttgcccttcaatttcacctggccggtaagcacaacttaaatgcaccgggagataaccgaagggaaagaagggagcgccgggacaccagagctcctttccaaagcgctctctggagagccccaagggctctttctcttctgcccccgcccccctgttctctcataggatcatcccggagaggctttggttagtctttcctcccagtttcttccctttccttctccccaattcttgggatacgaatttcattaccaaacccccaacgcggcgccgcccgcccaccccccggctctcacttacactcccgcatccctcatccctcccctgccttctcagctcgcgcgcagcgctgcgcgaacaccagttatgttgagccgagctccgtaactatatcctgcaattagattaattaaacaggctgctgcgaggcaccccctcctttccctccctgctgatatcgctatctctaatgtcccccacccccccttttgcttcccagggaaccttctcactcaacatccaag

SEQ ID NO: 4 - DNA, 1058 bp, Mus musculus
gttgcccttcaatttcacctggccggtaagcacaacttaaatgcaccgggagataaccgaagggaaagaagggagcgccgggacaccagagctcctttccaaagcgctctctggagagccccaagggctctttctcttctgcccccgcccccctgttctctcataggatcatcccggagaggctttggttagtctttcctcccagtttcttccctttccttctccccaattcttgggatacgaatttcattaccaaacccccaacgcggcgccgcccgcccaccccccggctctcacttacactcccgcatccctcatccctcccctgccttctcagctcgcgcgcagcgctgcgcgaacaccagttatgttgagccgagctccgtaactatatcctgcaattagattaattaaacaggctgctgcgaggcaccccctcctttccctccctgctgatatcgctatctctaatgtcccccacccccccttttgcttcccagggaaccttctcactcaacatccaagcttggcacacaccgggagacgacctgcggccaggtgagtatctaacttctcggccacaggggggcgacatcacacagcgccgaaagagttaaccagttataggcgggggtgggggttggggacgcaggcttggggggtgggggccaggacgcttagcttggccggagctgcgccccgcgctggacgctcggattccgctcgctgcctggactcagagcacaattgcgtttcctgcgggttatttttggcgtgggaacgcggggagcacggcggtgagaaaggccgaggctgccagcgccgctgacgggcctcttcctgtattttacaccttttgcgaattccgctcctttggaaagggaataatggctttgggatgttgttctgacacagaggaaaaggatatttcaccagcacaacaattctcactttgaaaaggaaaaagaaaaaccattacctacgtctagaacagaaccccttgctcccagttctcgaaccagaaaacttccccctttaaattttttctttttttccat

SEQ ID NO: 5 - DNA, 1264 bp, Mus musculus
cgctcctttggaaagggaataatggctttgggatgttgttctgacacagaggaaaaggatatttcaccagcacaacaattctcactttgaaaaggaaaaagaaaaaccattacctacgtctagaacagaaccccttgctcccagttctcgaaccagaaaacttccccctttaaattttttctttttttccattttgacctcttttcctctttcccctccgtatctgcctccacaaccctaggatatcttaacatccgtccattgtacccttttttgaatgctatcaagccccctgcacatgcacacacccagggagactaagtagcaagattctgggaccctctggcctgtgcttacttgcaggtagagttaatctagataattagagtgtgaactgaccaccatagtcacaactaaagagagagttggcagcagtcaactctctctgaatcaggttggctttctgaatcaggttctctgaccaaagcctctttctgcagagacttcgccaggaaactctctcatcagccaaatcatcatccaaggctctcttgctgtgggtaagatttggcgaacagacgagcaaaatgacaccctcaccagactgagctactcttaccgggtcatctgcagtgacaactactatggagagagctgttctcgcctatgcaagaagcgcgatgaccacttcggacattatgagtgccagccagatggcagcctgtcctgcctgccgggctggactgggaagtactgtgaccagcgtaagtagccaggccccctgtgagaatagaagggatgggattttcccaagaaagcactcagaatgggtctgtgctgggtctcaggaccagctggggatgctgtactgtacccttagtctcagagcctcctccgcagtgcttaagcctacagggtccttattcttcatcccatgcagtgtgatgttctcccaccccctcgtcccctggtcccctttaagataaccatggctcctcttggaggccaagagcaggaagtgagcccaggggagcaggagaggaggttgaagcttcagagtccatggtaccacaacctcatccagcccaatttcttttccttagctatatgtctttctggctgtcatgagcagaatggttactgcagcaagccagatgagtgcatgtaagtggggacaggaaacgggagtaggggggctctcccttgtgagcaggtctcccatcttacactgggctcccctcttgtcttaacagctgccgtccag

SEQ ID NO: 6 - DNA, 1061 bp, Mus musculus
actgagctactcttaccgggtcatctgcagtgacaactactatggagagagctgttctcgcctatgcaagaagcgcgatgaccacttcggacattatgagtgccagccagatggcagcctgtcctgcctgccgggctggactgggaagtactgtgaccagcgtaagtagccaggccccctgtgagaatagaagggatgggattttcccaagaaagcactcagaatgggtctgtgctgggtctcaggaccagctggggatgctgtactgtacccttagtctcagagcctcctccgcagtgcttaagcctacagggtccttattcttcatcccatgcagtgtgatgttctcccaccccctcgtcccctggtcccctttaagataaccatggctcctcttggaggccaagagcaggaagtgagcccaggggagcaggagaggaggttgaagcttcagagtccatggtaccacaacctcatccagcccaatttcttttccttagctatatgtctttctggctgtcatgagcagaatggttactgcagcaagccagatgagtgcatgtaagtggggacaggaaacgggagtaggggggctctcccttgtgagcaggtctcccatcttacactgggctcccctcttgtcttaacagctgccgtccaggttggcagggtcgcctgtgcaatgaatgtatcccccacaatggctgtcgtcatggcacctgcagcatcccctggcagtgtgcctgcgatgagggatggggaggtctgttttgtgaccaaggtgagtaagggaaggagagatggggtggcagggcctgaaactagagatggtgactggacacctttcttggtggtcagtaactgactttcctattattctattattgttcaaggacaagtctcaggacttgtctatgtgcacatgtgtgtgtgtggtgtgtgtgtgtgtgtgtgaggtattaggaatggaactcagggtctctaacatactaggcaagcactccacctgtgagctctaccccagtccactcactgcttttaaaggctctttacaactgacc

SEQ ID NO: 7 - DNA, 1131 bp, Mus musculus
ctgtgaccagcgtaagtagccaggccccctgtgagaatagaagggatgggattttcccaagaaagcactcagaatgggtctgtgctgggtctcaggaccagctggggatgctgtactgtacccttagtctcagagcctcctccgcagtgcttaagcctacagggtccttattcttcatcccatgcagtgtgatgttctcccaccccctcgtcccctggtcccctttaagataaccatggctcctcttggaggccaagagcaggaagtgagcccaggggagcaggagaggaggttgaagcttcagagtccatggtaccacaacctcatccagcccaatttcttttccttagctatatgtctttctggctgtcatgagcagaatggttactgcagcaagccagatgagtgcatgtaagtggggacaggaaacgggagtaggggggctctcccttgtgagcaggtctcccatcttacactgggctcccctcttgtcttaacagctgccgtccaggttggcagggtcgcctgtgcaatgaatgtatcccccacaatggctgtcgtcatggcacctgcagcatcccctggcagtgtgcctgcgatgagggatggggaggtctgttttgtgaccaaggtgagtaagggaaggagagatggggtggcagggcctgaaactagagatggtgactggacacctttcttggtggtcagtaactgactttcctattattctattattgttcaaggacaagtctcaggacttgtctatgtgcacatgtgtgtgtgtggtgtgtgtgtgtgtgtgtgaggtattaggaatggaactcagggtctctaacatactaggcaagcactccacctgtgagctctaccccagtccactcactgcttttaaaggctctttacaactgacctaaaatgggatggtgggacattcttctccacatcctaaagagccaggatgactaaagaggccaggggtaacagcttggcagcctgcagctcatactcataaattctagcaagactaaagagaaagggaaggttagcgctgttcctctttctgccttgtagttacctattaaccccctgagtgtttgctcaccttccaaggctctcccctaaacagctgtc

SEQ ID NO: 8 - DNA, 1170 bp, Mus musculus
tgctttgtctggctctccctccttgttcttctccaacaagaaaacaaatagctatcattcaaaatacactttcaacacctcctttagaagatgccggtcctgcctgtagtggctcttatgccccctcactctttccacctgttgaatttttactcttccttctagtccttccaggcatgcacctcctctatgaagcctttcctgacttcctcattggctaagggcctcttcttcatcttgctctggctggaaatgtttgtgagcatacccagcaggatgctggccaattgtgctttgtctcccacttttggtatacgtatacagtttagatgtagtttcaggcgagtgaatgctcaggatttcacaggcagagaaaggagggatgcctgggcctggaaaacttatgagtttaaacttctttgggccaaatacagggtgatcttgagtggaggtgggagagttcctggtcccactcagctggttttttcctgtctccacagatctcaactactgtactcaccactctccgtgcaagaatggatcaacgtgttccaacagtgggccaaagggttatacctgcacctgtctcccaggctacactggtgagcactgtgagctgggactcagcaagtgtgccagcaacccctgtcgaaatggtggcagctgtaaggtgagacccacatcagctcaggaaggcacaggtctaaccaggtagcatccagtcagtgtggttgtatatgcatgcatggatgggcactctggacaagtagaggctaggcaccaacctcatacatccttgtcccatcccccgggcccaactttagcctgtttatattctctctccaggaccaggagaatagctaccactgcctgtgtcccccaggctactatggccagcactgtgagcatagtaccttgacctgcgcggactcaccctgcttcaatgggggctcttgccgggagcgcaaccaggggtccagttatgcctgcgaatgcccccccaactttaccggctctaactgtgagaagaaagtagacaggtgtaccagcaacccgtgtgccaatggtaagctcttctgtcaccttaccaacctgctgaagtggccccggggccagagagcctgagaaaatcgtgaggagagagacctgtctgctattgtggtcaggctg

SEQ ID NO: 9 - DNA, 1220 bp, Mus musculus
tgaatgctcaggatttcacaggcagagaaaggagggatgcctgggcctggaaaacttatgagtttaaacttctttgggccaaatacagggtgatcttgagtggaggtgggagagttcctggtcccactcagctggttttttcctgtctccacagatctcaactactgtactcaccactctccgtgcaagaatggatcaacgtgttccaacagtgggccaaagggttatacctgcacctgtctcccaggctacactggtgagcactgtgagctgggactcagcaagtgtgccagcaacccctgtcgaaatggtggcagctgtaaggtgagacccacatcagctcaggaaggcacaggtctaaccaggtagcatccagtcagtgtggttgtatatgcatgcatggatgggcactctggacaagtagaggctaggcaccaacctcatacatccttgtcccatcccccgggcccaactttagcctgtttatattctctctccaggaccaggagaatagctaccactgcctgtgtcccccaggctactatggccagcactgtgagcatagtaccttgacctgcgcggactcaccctgcttcaatgggggctcttgccgggagcgcaaccaggggtccagttatgcctgcgaatgcccccccaactttaccggctctaactgtgagaagaaagtagacaggtgtaccagcaacccgtgtgccaatggtaagctcttctgtcaccttaccaacctgctgaagtggccccggggccagagagcctgagaaaatcgtgaggagagagacctgtctgctattgtggtcaggctgactgaaacaggctcctctttggtttggagggtgaagaactttatgcattatggagagtagggctttggagaagaaggtcatcgataggctgtaggtgagggtagtgcccatctgtatgacctctgttcttattccatgggtgagcctctgcctgacaaggctagcttgtgccctcctcaggaagccttgaattagaaaaaaggatgttggaggcttcacattcccttttcaagggaggctggtgatagagctctgtaccttgaagcctcatcttccccgacattcacctagaacaagtgtagcacaaagaatctggaatacccagtgctttttgatacacagaacacgttggtaggtgatggtctaagaggcaggcaggcaggcaggcaaccattc

SEQ ID NO: 10 - DNA, 1703 bp, Mus musculus
gatggtctaagaggcaggcaggcaggcaggcaaccattcaggagctgacaaggccctgggagtggcaatgtgatctattcgcactctcctgtgcgtggtctatactattctaccttgcgtcttccctactgatcctcagggctacctgtgaaaggatcaaattctccagtttagcaggtgaggaaaccaagactaagggatcaggtgaagctacaggtgtcagatcccctgccagagagatcaagccaagcatatctgtctgaccctaaattctggaagacgccagtaactattatccaatagcccactgtggcatagagaaccaggattaccccagaggccaggactttggagtctttgtacagttctctgtagagagctctctgtccacgcaggctgaacacacggtggtagaagagcttaagctcgtgggggagacctcattcccttcacccgggaatccacaggctattgctgatgcgggtcctgtgcccttacaggaggccagtgccagaacagaggtccaagccgaacctgccgctgccggcctggattcacaggcacccactgtgaactgcacatcagcgattgtgcccgaagtccctgtgcccacgggggcacttgccacgatctggagaatgggcctgtgtgcacctgccccgctggcttctctggaaggcgctgcgaggtgcggataacccacgatgcctgtgcctccggaccctgcttcaatggggccacctgctacactggcctctccccaaacaacttcgtctgcaactgtccttatggctttgtgggcagccgctgcgagtttcccgtgggcttgccacccagcttcccctgggtagctgtctcgctgggcgtggggctagtggtactgctggtgctcctggtcatggtggtagtggctgtgcggcagctgcggcttcggaggcccgatgacgagagcagggaagccatgaacaatctgtcagacttccagaaggacaacctaatccctgccgcccagctcaaaaacacaaaccagaagaaggagctggaagtggactgtggtctggacaagtccaattgtggcaaactgcagaaccacacattggactacaatctagccccgggactcctaggacggggcggcatgcctgggaagtatcctcacagtgacaagagcttaggagagaaggtgccacttcggttacacaggtaagccacacctggaagcccatagcttggtcacagacccttccatagtttgacaggatctcctaggctgagtgggaggctggcatcaggccttggcaacttttaatcaagtaagattgtagtactgacaagaagacactctagttacatttatttttttttgtggggggtggggtggggttttttgagacaaagtttctctgtgtagccctagctgtcctggaactcactttgtagaccaggctggcctccaactcagaaattcacctgcctctgcctcccgagtgctgggattaaaggcgtgcgtcaccacgccaggcttctagttacatttctatagggacccaggcacagtggcacagactttgtagccctacctacttaggctaagacaggaggattgctagtttgatgctagcctgggtaacatagcagcagatcatgtctcaaaaacattgagatggctcagagagtaaaggcacctgctgccaagcctggtg

SEQ ID NO: 11 - DNA, 1109 bp, Mus musculus
ttctgagttcgaggccagcctggtctacaaagtgaattccaggacagccagggctacacagagaaaccctgtttcaaaaaaccaaaaaaccaaaaaaaaaaaaaaaaaaaagaattctgctaggaatcttttactagggaagtccctctcattcattcattcattcattcattcattcattcattcagtcttaactcccaaataccaagtgtccacagtatatctctcgtggtttccatcttcccacagtagggcttgtctctagtggcaatactggcttttttctggtttggtcggctcctgtagctcccttagaggcctattctaaccccctgcccctgaaggccagcactgagcatcctccccgatgccctccccgaacccctccggacccctgcagcacataccctaaaggggatctctgggatctagactgacagctccttgcttgtcccctccctattccttccttcttacctccctgtcttctgttccttcagtgagaagccagagtgtcgaatatcagccatttgctctcccagggactctatgtaccaatcagtgtgtttgatatcagaagagaggaacgagtgtgtgattgccacagaggtgagttcttccctctaagcatgtcccctcctgctttgtgtggtgggaaaaaaatgtcctgttcactagcaatctcattcctgaaggggtgggtcagagatctcttctttggtgtgtagtggctcttgggtgccctgctggccccattgccacagagggagtgatactggagccaggtggtatgcctgacttggcagctgtgccagggaaagggacatagtcaaggagcaggagaaggctcaggcagagctttcaggaactatttcccacttgcctttgggtgaagacacagggttacctgtgtctgcttcctccattgaactcctcttgtgtgtcttagagcaggaggctgagggtactaactccctcagtggtgtctcctagagaggtccagagcacctctggatcattgcataccgcccccccccacccccggtataaccattttcccattttgtatgtgatccccaggtataaggcaggagcctactcagacaccc

SEQ ID NO: 12 - DNA, 2029 bp, Mus musculus
agaggaacgagtgtgtgattgccacagaggtgagttcttccctctaagcatgtcccctcctgctttgtgtggtgggaaaaaaatgtcctgttcactagcaatctcattcctgaaggggtgggtcagagatctcttctttggtgtgtagtggctcttgggtgccctgctggccccattgccacagagggagtgatactggagccaggtggtatgcctgacttggcagctgtgccagggaaagggacatagtcaaggagcaggagaaggctcaggcagagctttcaggaactatttcccacttgcctttgggtgaagacacagggttacctgtgtctgcttcctccattgaactcctcttgtgtgtcttagagcaggaggctgagggtactaactccctcagtggtgtctcctagagaggtccagagcacctctggatcattgcataccgcccccccccacccccggtataaccattttcccattttgtatgtgatccccaggtataaggcaggagcctactcagacacccagctccggcccagcagctgggccttccttctgcattgtttacattgcatcctgtatgggacatctttagtatgcacagtgctgctctgcggaggaggaggaaatggcatgaactgaacagactgtgaacccgccaagagtcgcaccggctctgcacacctccaggagtctgcctggcttcagatgggcagccccgccaagggaacagagttgaggagttagaggagcatcagttgagctgatatctaaggtgcctctcgaacttggacttgctctgccaacagtggtcatcatggagctcttgactgttctccagagagtggcagtggccctagtgggtcttggcgctgctgtagctcctgtgggcatctgtatttccaaagtgcctttgcccagactccatcctcacagctgggcccaaatgagaaagcagagaggaggcttgcaaaggataggcctcccgcaggcagaacagccttggagtttggcattaagcaggagctactctgcaggtgaggaaagcccgaggaggggacacgtgtgactcctgcctccaaccccagtaggtggagtgccacctgtagcctctaggcaagagttggtccttcccctggtcctggtgcctctgggctcatgtgaacagatgggcttagggcacgccccttttgccagccaggggtacaggcctcactggggagctcagggccttcatgctaaactcccaataagggagatggggggaagggggctgtggcctaggcccttccctccctcacacccatttctgggcccttgagcctgggctccaccagtgcccactgctgccccgagaccaaccttgaagccgatcttcaaaaatcaataatatgaggttttgttttgtagtttattttggaatctagtattttgataatttaagaatcagaagcactggcctttctacattttataacattattttgtatataatgtgtatttataatatgaaacagatgtgtacaggaatttattacttcttgggtcctatctgtgtaatagcagagtggggatctttttcctgcctccctatcccaaattcaaagccccttatcttctcactgtggagtggctctgcgggaaccccagggcctgtgtatgcttgggtttgccatgttgtgtagaagggtggtctttgggtagcctggccttggcctcctgtgcctggtgactttaagcctgggtgttcctactccactttaagttccatggggctattttcccctgccttgattttctcccattgaggaagagcctctcattacttattcaaaagtgctcttttattagatcatgagtgtcgtcccctcgggtacttcctcctgccactggggcatttgggttgtactggcacctgcacttctggtactagtgtggaactggtctgggttgaggaaggggaggggatctgccctagatttatctttttcgaagttgggcagagctgaagggatggctgaggcta

SEQ ID NO: 13 - DNA, 1368 bp, Homo sapiens
gaaaaaaaaaaaaaatccaggtcttatacccgctgggcaaggtgcttatgcaccactgagccacacttgtgacccattcagctacttcctctggaagctagggggccctggggtgaaagaagggacactcagactgttctgcctgtgcctgtgaaggtggactgaggcagcaattcgattgaggaacaggaggtagtgtgacagatggaggagcaccgccggcctttcagtgacagtctgagggagtgaagtctgagctgcggaagcctaggatccgcgcaagcctgttactggacgccgctgggaaatcccgggctgcctcctagcgccaacatgaggaactgcagcagcagggtggcccgcaagcgctcctcccccgccgcgccgagtctccgacaacgcctccttgtggaggctcaggtgcccaggactccagttaacactcgcggctgaaaaacccggaagcacgtcataagcacgcgtcacgggggcgggagtcagctgagctgcctgggcgaggttgtgattacctgggatctgctaaggggagcctataatctcttgggactggagctggggtgcaaagctgcgccatggactagttggggctcctcgatcgtctcgtccttcacagagaaagcgaaaagggccccagaatttttaaaaggttctcaggatctgtcagacgctggggaagcacaacttgcaaatccaggacgaccggggtcccagttgtgacccccagcccccagatcagagatccagaacttgattgccatggcgtccatcctggatgagtacgaggactcattgtcccgctcggccgtcttgcagactggttgccctagcgtgggcatcccccattctggtaagttagaagaggccgcctctgcccctttctgttatggtagcatttacgggggagagaagctctagagcttgcagcgtgtgtttaggatctaagccccaggcctttcttgtcttgcccagcttgtcattggtcgaagtttgaaagagccacaggaacagcctcgtgatctaggccttaactcaaaggtccttcaaaagagggaacttgggctcagataaggaagtacttgttacttttgtctataggctgcagttgggagaactgaatataggttccaggcttctgtgtcttgttctatggattttttttaaagcttttggagtctgtgtatgtgttcgttcccaccagacttcatcttgaggtagcagaggaggcagaatgagcactgaacttggtggaacgtgaatgttataagtgaaggagggtcgatccaaaagggaattttgttactgaacaggaacaatggagatgattgtcccggggtaaagctggctgca

SEQ ID NO: 14 - DNA, 1142 bp, Homo sapiens
gcagggttacccacactatgatgatattgcagcggactatcgagaaagcaactgtagcctatgaactggactgataggactgggttcccagaactctctacatacaatggagactcactgaaccatactgtgtggacagcaaggaagggactttagtttctcttggccaaagaccataagggagaatgccaaaaccatcctccctgtactgtctccccccacccccgttaccactgggaacgtgcatatcatgatacaacctcatctcaggcacagagaaccaatcccttactactgtcacaagtgtgggaagagcccaggacccttccaataaagacaaccagaaaactatttcatgttgaactctttatgaaccaagaccctgtgggtaggaatatacttgggaagcagaagtatcagctaacactggggtttctctgggtttctgggtgcctcacccccctctggacgcctttttctcttgtcttccccacctatagggtatgtaagtgcccacctggagaaggaggtgcccatcttcaccaagcagcgggttgacttcaccccctcggagcggatcactagcctcgtggtctcctgcaatcagctctgcatgagcctgggcaaggatacactgctccggtagtatctttggagacttttagctgactagggtctttttccatacttagcaaatgacacaatagcttttcaggagatgcactattccttcccaaagagaaccagtgtggtggctgaagatcagagagaactatgttcagattccacccatgctgttgaacgagtcagctaatcattctggtctttcaacagggggaaaggtgctctcagcctcccagagttagccacacagatcattgggacagtctggaatcctgaagtatgtccgctatttgccaaaagtgttgacagcgttgtttatcatttgggataagccatctggaataagacagttagacctctgtcccacaaggatgaatcccagcacgaatagttattagaacaacgggtgttgggattgtggcccagtgatagagtacctggcattcctgaggccttgggtttgattcccggcactgcaaatgaaatgctgtagataagtgaggctggagttctgttgg

SEQ ID NO: 15 - DNA, 865 bp, Homo sapiens
aaaaaaaaaggaagaaagacaagaaaaaagttagcatagtaataataaaatgctttagttgtttatctgaaccaagacgtttagtctcactaggaaagcactcccatgcgtatacatctgcatcacagatatcttgtgttgtgttttgttttggagacagggtctcctgtagctcaaggctgtgactccctaaccagccgtctaccccactcaagtgctagagctacaaatgttcaaatctagtagtaaacagcgctggcaaaggtacagggaagaggactgccacgcctggcacactgagggcctagtgcccacatggcagccaacaactccagttctagggggacccagcaccttcctctgacttccatgggttcctgcacacatgatacgcttacatgcattcatgaacaacatgtacacattaagtaaattaattaattaatattttaagaagggggacttctcttgagagtgacaactaactcttgcccttacagcattgacttgggcaaggcaagtgagcccaaccgtgtggaactggggcgcaaggacgacgccaaagtccacaagatgttcctggaccatactggtgagtggcgctggagatctgaggaggcttcctgtgagagttcactgatgatgggcgggatctgaacgggggtggggtggggatggggggactcgaggtccactcagtgctgttttccatccgccccaggctctcatctgctggttgcgctgagtagcaccgaggtcctttacatgaaccgcaatggacagaaggcccggcccctggctcgctggaagggacagctggtggagagtgtgggatggaacaaggccatgggcaacgagagcagca

SEQ ID NO: 16 - DNA, 1152 bp, Homo sapiens
  modified_base (644)..(844): a, t, c, g, other or unknown
agctacaaatgttcaaatctagtagtaaacagcgctggcaaaggtacagggaagaggactgccacgcctggcacactgagggcctagtgcccacatggcagccaacaactccagttctagggggacccagcaccttcctctgacttccatgggttcctgcacacatgatacgcttacatgcattcatgaacaacatgtacacattaagtaaattaattaattaatattttaagaagggggacttctcttgagagtgacaactaactcttgcccttacagcattgacttgggcaaggcaagtgagcccaaccgtgtggaactggggcgcaaggacgacgccaaagtccacaagatgttcctggaccatactggtgagtggcgctggagatctgaggaggcttcctgtgagagttcactgatgatgggcgggatctgaacgggggtggggtggggatggggggactcgaggtccactcagtgctgttttccatccgccccaggctctcatctgctggttgcgctgagtagcaccgaggtcctttacatgaaccgcaatggacagaaggcccggcccctggctcgctggaagggacagctggtggagagtgtgggatggaacaaggccatgggcaacgagagcagcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgagctcggtaccctggtcggcacagctcaaggacagatctttgaagcagagctctcagctagcgagggtggcctctttggccctgccccagatctctacttccgtccactgtatgtgttaaatgaagaagggggtccagcccctgtgtgctccctcgaggctgagcgtggccccgatggccgaggctttgtcattgccaccactcggcagcgcctcttccagttcataggccgagctgtggaagatactgaagcccagggcttcgcaggactctttgctgcctatacagaccacccgcccccattcc

SEQ ID NO: 17 - DNA, 2719 bp, Homo sapiens
  modified_base (291)..(490): a, t, c, g, other or unknown
gttcctggaccatactggtgagtggcgctggagatctgaggaggcttcctgtgagagttcactgatgatgggcgggatctgaacgggggtggggtggggatggggggactcgaggtccactcagtgctgttttccatccgccccaggctctcatctgctggttgcgctgagtagcaccgaggtcctttacatgaaccgcaatggacagaaggcccggcccctggctcgctggaagggacagctggtggagagtgtgggatggaacaaggccatgggcaacgagagcagcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgagctcggtaccctggtcggcacagctcaaggacagatctttgaagcagagctctcagctagcgagggtggcctctttggccctgccccagatctctacttccgtccactgtatgtgttaaatgaagaagggggtccagcccctgtgtgctccctcgaggctgagcgtggccccgatggccgaggctttgtcattgccaccactcggcagcgcctcttccagttcataggccgagctgtggaagatactgaagcccagggcttcgcaggactctttgctgcctatacagaccacccgcccccattccgtgagtttcctagcaatttggggtatagtgagttggctttctatacccctaagttacgctcggcacctcgcgcctttgcctggatgatgggagatggagtgctgtatggctcactggactgcgggcgtcctgactcgctgctgagtgaggagcgagtgtgggaatacccagcgggggttggtcctggggccaatccacccttagccatcgtcctgacccagttccatttcctactgctgctggccgaccgggtggaggctgtgtgcacgctaacagggcaggtggtgctacgggatcacttcctggagaagtttggaccactgaggcacatggtgaaggactcatccacaggccacctatgggcctacactgagcgtgcggtcttccgctaccatgtgcaacgtgaggcacgggatgtctggcgcacctacttggacatgaaccgctttgacctggccaaagagtattgtagagagcggcctgattgcctggacacggtcctggcccgagaggctgatttctgttttcgtcagcatcgctacctggagagcgcccgctgctacgcgctgacccagagctattttgaggagattgccctcaagttcttggaggcccggcaagaggaggcgctggccgagtttcttcagcggaaactggccggcttgaagccgacggagcgtacccaggccacactgctgaccacttggctgacagagctctacctgagccgcctgggtgctctgcagggtgacccagatgctctgactctctaccgggacacacgggagtgtttccgtacttttctcagtagccctcggcacaaagagtggctctttgccagccgggcctctatccacgagctgctcgccagtcacggagacacagagcacatggtgtattttgcagtgatcatgcaggactatgaacgggtggtcgcataccactgccagcatgaggcttacgaggaggccctggctgtgcttgcccgccaccgggacccccagctcttctacaaattctcccccattctcatccgccacatcccccgccagctcgtagacgcctggattgagatgggcagccggctggatgctcggcagctcatccctgccctggtgaactacagccagggcggtgaggcccagcaggtgagccaggccatccgctacatggaattctgcgtgaatgtgctcggtgagacggagcaggccattcacaactacctgctgtccctgtatgcccgtggccagccagcctcacttctggcatacctggaacaagctggggccagcccgcaccgtgtacattatgatctcaaatatgcacttcgactttgtgctgagcacggccaccaccgcgcctgcgtccatgtctataaggtgttggagctatatgaggaggctgtggacctggccctgcaggtaagtcttacctttatccagaagagggacctggagaaagagcctgagaggttgggggaggtgtggctggcgcttgctggcttctcatgaagagaagtgctgtgaggcagaggctgaaactcagactcagctgagttggtggtgcaagctcgttgccggtaatccctgcacttgggtgcttaaggcagaaagagcacagtttaggcaacaccgtgagtccccgtctttaaaaacaaaaaacaaaacaaagttagcaggaaaggaagaggggaaaaaaagaacacgaattaaataaaactttggggtttgcattctggctttgccagcttgctgtgtgatttggggctggtttctatacctctctgtgccttagattatccattaaatggtggtaatgagccagcgagatggctcggtaggtagaagcacttgtacaagcctgacaagctaagttctgtccccagagctgatggtagaaggagaaaactgactgccgcaaa

SEQ ID NO: 18 - DNA, 1916 bp, Homo sapiens
tgaacttgccagcattcctgcttccccctcctgggtgctgtgctttcagcattaccagctgatctttttttttttttaatatttatttatttattatttatttattattatatgtaagtacactgtagctgtcttcagacactccagaagggggagtcagatctcattacagatggttgtgagccaccatgtggttgctgggatttgaactctggaccttcggaagagcagtcgggtgctcttacctgctgagccatctcaccagccccccagctgatctttttatatccactaaaattgcctgccaccaaaatagccttccggtagctgtgacagggcagaccaatgcaggttcctgttgtgcctcttgctacccggagtgcggagaatcctgcttgggccctctcctggggcccagatttctgtggccatctgtctttccagtagtctgatgctgggtggagcgtggcttgcccctgacaccggtggtttccccacaggtggatgtggacctggccaagcagtgtgcagacttgccggaggaggatgaggaacttcgcaagaaactatggctcaagatcgcccggcacgtggtgcaggaggaagaagacgtccagacagccatggcctgcttggccagctgccccttgctcaagatcgaggatgtgctgcccttcttccctgactttgtcaccatcgaccacttcaaggaggcaatctgtagttccctgaaggcctacaaccaccacatccaggagctgcagcgcgagatggaagaagccacagccagtgcccagcgcatccgacgagacttgcaggagctccgaggccgctatggcaccgtggagccccaggacaaatgctccacctgtgactttcctttgctcaaccgacccttttacctgtttctctgtggccacatgtttcacgctgactgtctcctacaggctgtgcggcccggcctccctgcctacaagcaggccagacttgaggaactgcagcgcaagcttggggcggcgcctcctccgaccaaaggctccgtgaaggccaaggaggcagaagctggggctgcagcggtggggcccagtcgggagcagctcaaggctgacctggatgagctggtggctgctgagtgtgtgtactgtggggagctgatgattcggtctatcgaccggcccttcattgatccgcagcgctatgaggaggagcacctcagctggctataggaagttcccccgcgtgggtggacagacatggatccagctgccagaccctcctgcgaaagccaggccctggttggtgttcggtccttgagcttgccgggtctgcgattgtgagggggttgcagccaagcttcagccgggaggttttcaggtgcgaacgccgagctgtgcttcagacctgtcttagagctgcccgtcccaggcagcccgagctggtgagatcagccgtgatccttttaaccactacatctaacagttcctctgttctcgtcagcatgttcacctggactgagtggcccgctgtccttccttcctccgtccttccacagcctctggacctccagctcgccctcttctgcctcaactaggtgacccaggagcaagcaggcagaccctaggcacttgtggggagagtgggcatgtggagatgctaatgtgacctggggctccagtgtcgggtgccctgaggctgagggcacacctgggacaaggggaaatagggagagtattaaatcacgctacaatggtggccctggacttgatggctttgtgttgatgctgaagctaacaggatatgatcccaagaaaggatggaatgccacaagaaggctgcctgcaccaagtgtggaatagaacaggtcaggtcacggccagcctagtcgacataataagttctaggccagcaaaggctag

SEQ ID NO: 19 - DNA, 1736 bp, Mus Musculus
cacagatgaaatttgagattatgataagtgaaaaaagctaggggttttaaaggcatattgtatgactccaattgtataagtattcaaaattatagagtactgctgtgcttttagggaaggtagggagaggtttaatgggtatgatatttcagctttgtgaggtgaaaagaattctaggaatggatagtggtgattgcataatggtatgattgcactttgtgccccaaactagtcccataaggaaaattaaagccaggcacagaggtgtgcgcctaagtcccagcactcaggagataaaggcattaagattgtgaaactgagagcagcctgggctacacataaagaccctctctcgaaaaacaaagttttatgtcctttgaattttaccacaataaaaacaattagagcttctggtgtccatccgtgccgggagctgatcctgtgccacagcaatccacacccaaatactgccgggagagagcttgtttcccaggagtgctgacaagcctgtgagcacagaatcccctggtgatgttcccgaacatccattgtttatcacagtgggtgactgactggattctataaagacgggcaatgcaagagtaacttcctagcagtgggttttacaacgtttgatctgatttcaagaatgagcatagatgacattaggggagttcagagtcacacttatcagacatcagagacaaatagcatcatacaaactctgcatttacatatgatgcacatacaggagaaagggtttcatgtatgtaagtactataaacaacggcatcttgtgcctcagtatttctacgaagaaagatgttccctcctcggctcgttcttctgattctccagccttcccttcaccacccaccgtctcttgttgagactgaggctgcgcaccttacgattacgcttccaccctggacttcagtgtcatgctgcacaggtcctctctgaatagcatgcaagcaaaactctggcccacggcagatcatcactgcacgataatactcatataagcaacagcatggatgtgtaactttgtagagaaatgattgggaagtgttcctggacttaacctaaaggaatttgtccttaagtgtgtcatcttcaaagatgtatgtggagtttgatgatagataagaaggagatagcagaccattcgttcatgttttgtgatcaagtagcttccaatgtcacatacttctttttcaatagttttactcagctgtataggtttttttttgttggttttttttgtttttgttttttcagatttatttattttacttatatgagtacactgtaactgtcttcagacacaccagaagaggacatcagatcccattacagatggttgtgagccatcatgtggttgctgggaattgaattcaggacttctacttttttagaccctgcttgctccagctccgctcactctggcccaaagatttatttattattatatgtaagtataataatatactgtagctgtctttagacacaccagaagagggcgtcagatctcattacagatggttgtgagccaccatgtggttgctgggatttgaactcaggacctttggaagtgcagtcagtgcccttacatgctgagccatctctccagcccccagctctacaggttttgttgttgttttattttaaatgacttaactgttggtgcctgatgttgttacaatgatttgcagagataattaatgattccatgt

SEQ ID NO: 20 - DNA, 1081 bp, Mus Musculus
gatagataagaaggagatagcagaccattcgttcatgttttgtgatcaagtagcttccaatgtcacatacttctttttcaatagttttactcagctgtataggtttttttttgttggttttttttgtttttgttttttcagatttatttattttacttatatgagtacactgtaactgtcttcagacacaccagaagaggacatcagatcccattacagatggttgtgagccatcatgtggttgctgggaattgaattcaggacttctacttttttagaccctgcttgctccagctccgctcactctggcccaaagatttatttattattatatgtaagtataataatatactgtagctgtctttagacacaccagaagagggcgtcagatctcattacagatggttgtgagccaccatgtggttgctgggatttgaactcaggacctttggaagtgcagtcagtgcccttacatgctgagccatctctccagcccccagctctacaggttttgttgttgttttattttaaatgacttaactgttggtgcctgatgttgttacaatgatttgcagagataattaatgattccatgtactgcaattgtatccggtgtgactacagtatgaagggcattgttggggagggagatggacaaaaatactgtgcttttaaatcttcttcttcttcttttttctttaagatttatttatttactttatgtatgtgagtacactgtagctgtacagatggttgtgagccaccatgtggttgctgggacctgaactcaggactcagaagagcagtcagtgctcctacccactgagccatcttgccagcccttaaatcttctcaaacaaacaaacaaacacacaaacaaaaaactggaaaggtaagcaggtacatttgaatagaggctatattacaggagaacatgccacaaggagagagtcagagggaagaggcaaaactgagtcagatggtcaagataacctgtgcaaagggcacagaagctatggtatgagggccggccgaaggttgcccactggcagcctgcaatagtactgtcaccaaacagtta

SEQ ID NO: 21 - DNA, 1045 bp, Unknown Organism
  Description of Unknown Organism EMBL No. mmaa11531
tgattcccctcgggacgctgctcccttgtcgccttctcaaaaactccgctgctcgatgcctcggtaggtaatccaggtgagggcgactgggcggtggctgctctaagtacgtgtcctggagcaaatcctagggtctcgagcgggctagtgagggagtacactgcacccaagtgactttggctgcagctcttggcctcgggttcctagcctgcgagcagcgccgctactgaaacctgcttgctccagagcctttcttatatatattggctgctcttcgcagcctgtcagtcagacagactcacggtcactctcccctcccctccaggcctctagtggctacagtcccagcacccaccggagggggcctccgcccccgccgctccccctgtcagccagccagtagggcgcaggtccccgcccctcttccaacttctgggcttccagctctgcgcgtgcccggcgagcagtccttagagatcgtctgcttaaaggctgagaccctcgccaccagaggataggagggtagcccagtgatgcggcgggaggtgaggtggttcccgaatggtccagggggtggaaactctaaggggtccccgtaggcggtgcacctgatccaggcattagatacctacagtcctctagcaggtggggaaagcttaaactaggaaagcaaaaagcaggaccaggggatttggatctccctttcctttccagactttaggtagctcctgcctcgctggcctccagcctatgtgtgcccaccctccacgcgtccccagcaccgcagggaagccatccgacgccttaacctttccctggctattccaagcaaggcaaagggccaagcgtctaggactctggtcttctctgcacctggtaggcgtgtcacctcaagcagcagggcgtgcccagcgcgtgccttctctctttgtcccccacccccactccctcctggctcgtgctagagccaggactagaggttcgaaagagaggtggggggcgtggttctcgaagtggggggggggtcctcagctgtatggtaatg

SEQ ID NO: 22 - DNA, 1162 bp, Unknown Organism
  Description of Unknown Organism EMBL No. mmaa11531
gcagcgccgctactgaaacctgcttgctccagagcctttcttatatatattggctgctcttcgcagcctgtcagtcagacagactcacggtcactctcccctcccctccaggcctctagtggctacagtcccagcacccaccggagggggcctccgcccccgccgctccccctgtcagccagccagtagggcgcaggtccccgcccctcttccaacttctgggcttccagctctgcgcgtgcccggcgagcagtccttagagatcgtctgcttaaaggctgagaccctcgccaccagaggataggagggtagcccagtgatgcggcgggaggtgaggtggttcccgaatggtccagggggtggaaactctaaggggtccccgtaggcggtgcacctgatccaggcattagatacctacagtcctctagcaggtggggaaagcttaaactaggaaagcaaaaagcaggaccaggggatttggatctccctttcctttccagactttaggtagctcctgcctcgctggcctccagcctatgtgtgcccaccctccacgcgtccccagcaccgcagggaagccatccgacgccttaacctttccctggctattccaagcaaggcaaagggccaagcgtctaggactctggtcttctctgcacctggtaggcgtgtcacctcaagcagcagggcgtgcccagcgcgtgccttctctctttgtcccccacccccactccctcctggctcgtgctagagccaggactagaggttcgaaagagaggtggggggcgtggttctcgaagtggggggggggtcctcagctgtatggtaatgaggttctagtcttttccctcagtgcctatctcacctgggtcgcgcttggtctccatatcctcatcctggcccccacccgcgtcccctagctccgctggagtagggaggaaactttcttaattccagaatttccttgggccacttgagaggcaaggtgagagagcttgaggctgatcaggtctagatctctctgcatagggcaggatgtagggtggaactggaggcgcatagagttgtttgagtttcgggtggtttcacccatgtcacacgcgcgcctctcccgggtggcctccgagcgcaaaccctgcagctgctttcccagaacccgtcg

SEQ ID NO: 23 - DNA, 1132 bp, Unknown Organism
  Description of Unknown Organism EMBL No. mmaa11531
cagggggtggaaactctaaggggtccccgtaggcggtgcacctgatccaggcattagatacctacagtcctctagcaggtggggaaagcttaaactaggaaagcaaaaagcaggaccaggggatttggatctccctttcctttccagactttaggtagctcctgcctcgctggcctccagcctatgtgtgcccaccctccacgcgtccccagcaccgcagggaagccatccgacgccttaacctttccctggctattccaagcaaggcaaagggccaagcgtctaggactctggtcttctctgcacctggtaggcgtgtcacctcaagcagcagggcgtgcccagcgcgtgccttctctctttgtcccccacccccactccctcctggctcgtgctagagccaggactagaggttcgaaagagaggtggggggcgtggttctcgaagtggggggggggtcctcagctgtatggtaatgaggttctagtcttttccctcagtgcctatctcacctgggtcgcgcttggtctccatatcctcatcctggcccccacccgcgtcccctagctccgctggagtagggaggaaactttcttaattccagaatttccttgggccacttgagaggcaaggtgagagagcttgaggctgatcaggtctagatctctctgcatagggcaggatgtagggtggaactggaggcgcatagagttgtttgagtttcgggtggtttcacccatgtcacacgcgcgcctctcccgggtggcctccgagcgcaaaccctgcagctgctttcccagaacccgtcgtgctcccccgaggctgcagaccggggagggctggcttctcccagttttggggggtgctagatttctctctctctctctctctctctctctctctctctctctctctctctctctctctctctctctctctctctctctctctctctctctctctctctctcctacagtgagagcggtgggggccctgtggcttcagctgtcagccctcccgcgtggtgattgctggctatggtcggacgataagatcagtgcgggcgggggttgaggggagcagggacgcggctccagccccagggaggccgtggtagggggaagtggaaccc

SEQ ID NO: 24 - DNA, 1112 bp, Unknown Organism
  Description of Unknown Organism EMBL No. mmaa11531
cccgcttagcaagcagcgccccgcggcgcggcgcacttttccctactgccccttggagaaactgcgtacttggagtttttgcccggggtcagagtggattccccagagaataggcctgaggctgagggaggagagaggaagatgatgacttagcattggcctcgaggactggctggggggcttcaaggaaggaacaggatgcaggaactgatgataaaatcccagggctaggagagaagaggggcatggtgttgtaagtcacaatgggttctgaggattgaagaggaggcccgagcactgaggtggcctggggtctcaccccattctccccttagtctggtgaatgagtatgactcggatttggccaagaaacgtaaccacggctctacatctgttctttcatccctgcaaagtgataataatagcgatctgggagactgtattggactatccattttgcaagattcaaagaataatttagttcctttctttccctctaggcaacatgaagacaaccagaggccacctcgggggcggggcacaaatgaaattcacgggtagaggaaaagacagaggacacactcctaagggaagaggcaataggagcagaacggaggaggggaaagagcagagcgtgaaaagtcaaggtttcggggccttttttggtaggaggcctgtgataaggttcttgactgaacaacctggcctagagaaaaccttcccactgagacaactctcctgctggagggtagtctttcagagcagcggcctgcagaaaggaagagatgtcatccttgaatggatctaatcagagagggtgctggcctcagagagagggggggcgcgggggcgcgctcggcttttcctcatacctctgagagaccctacttcctgggttcactgtccactgtctggttgctcatgcctgatgactcaacatatgccaaataaaggatacccaagtgggcaggattactgggcacggcagagaactgcttgggtttctcctaaggcctggaaggcctcccacctcctttcccggggtcccaggcacagtgtctggagtagtttcatactgtttccttggctttccagaacacagagcctggcac

SEQ ID NO: 25 - DNA, 1188 bp, Unknown Organism
  Description of Unknown Organism EMBL No. q9ulg1
aaggtattcttcctgggagtctttcgactctggggaagtttataattttccttaatctatgatccatctgctttaaaaaaacaaaaataaaatctttaattccagttctgaggagagattttgtagaatactctgatatatatatatattctggtagataggattcttcaaacttatggatgaatgaaattgttgggaacaagcactgtcctcagcacttcacttcgttaattgtaattcattcttgttattagtggaggacaaataggaagtgagtataacgtctcagggttctttgagaaagtgctttataaatgatagtgtttctatactttgcccttgcagtaggaggcagtgtctaatgaactttctgtagtctgttattgtcacagtgtcttgtgtttgagagggcctgattcctgtttgtactgaaggcaagcatgcaggagaaggagaagaggaataatcttggatttttttttaacttatctccattttgcagctgtgcagttggaggtcctcgtgcatgaagacaaatttgttctatggcctcggagttgggtgccggggacgatggcagcagcactgagctggcaaagcccctttatcttcagtacctggagagagccttgcggttggaccactttctgcgacaaacatcagccatcttcaacaggaatatttctaggtgtgttattttatctcttgacagtgttttattacatttctgatttctgtctttaggatttaaggttaaacttgtatggtttttacttttagtattttcttcctctttttttcacataaatcaactttttcaaagctatttgttctttacgttttcatttatagtaaccttaatcacttaattattgttttccttttcttctttccttcctccctcctttccttccttccttatttcctctttcttcctttttttttttttttgagacagggtttttctgtgtatccctggctgtcctggaactcactctagaccaggctggcctagaactcagaaatccgcctgtcaccaaaaaaaaaaaaaaaaaaaaagagccgggcatggtggtgcgcctttaatcccagcacttgggaggcagaggcaggtggatttctgagtttgatgccagcctggtctacagagtgagttctaggacagccagtaccggttcgttctttctt

SEQ ID NO: 26 - DNA, 1170 bp, Unknown Organism
  Description of Unknown Organism EMBL No. q9ulg1
ctctctctctctctctctctctctctctttctttctttctttctttctttctttcattctttctttctttctttctttctttctttctttattgttttttgagacagagtttctctgtgtagctctggctgtcttggagctcattttggagaccaggctggcttcaaactcagaaatctgcctgcctctgcctcccaagtgctgggattaaaggtgtgcgccaccactgcccagcttctttctttcttttttgctcaatatgtagcccagactgtcctagaactcacagatctcctgcctctacccctggagcactgtaaaggtttaaaggtatgtgtgccatcttgcctgactccttagattatgtacacacatatatgtacataatcctcttgtctcctgtctccaccaggtttataggcatatgctatcacactcggctcaaaatgtgttttgttggataacctttaattatggacttactgttcttccatttatagtgatgacagtgaagatggactggatgacaataatcccttattgcctgagtctggggatcccttaatacaagtaaaagaagagcctccaaattcattgcttggcgaaacctctggagcaagcagttctgggttgttgaacccatattcactgaacggcgttctgcagtcaggtcagtgtggagtgggagccctttgcagctgcagttctcttgggggcagtcagcgtttcatttttttttgtttttgttttttgtttttttttttttactctttatgtttctttatccagaatcaaaatctgacaaggggaatttatataacttctctaagttgaagaaaagcagaaagtggctaaaggtaagagataagacagaacatttccagtccaaataacctactaagattacagttagaagggaagttatccaaccattctggaatattgacagttttctagacaaagttattctatactgggatttgttttatgaagaaagttcaacaaagtaattttttagtaggctttttattaaatatgggacactgagaaaagaggagataaatggatcagataagatcctcccttcattggcttatcttgtaatcaggaatgttaatttaggttttcatgagtttttgagtacttcattcaacaaatgctcagaatgca

SEQ ID NO: 27 - DNA, 1068 bp, Unknown Organism
  Description of Unknown Organism EMBL No. q9ulg1
atctcctgcctctacccctggagcactgtaaaggtttaaaggtatgtgtgccatcttgcctgactccttagattatgtacacacatatatgtacataatcctcttgtctcctgtctccaccaggtttataggcatatgctatcacactcggctcaaaatgtgttttgttggataacctttaattatggacttactgttcttccatttatagtgatgacagtgaagatggactggatgacaataatcccttattgcctgagtctggggatcccttaatacaagtaaaagaagagcctccaaattcattgcttggcgaaacctctggagcaagcagttctgggttgttgaacccatattcactgaacggcgttctgcagtcaggtcagtgtggagtgggagccctttgcagctgcagttctcttgggggcagtcagcgtttcatttttttttgtttttgttttttgtttttttttttttactctttatgtttctttatccagaatcaaaatctgacaaggggaatttatataacttctctaagttgaagaaaagcagaaagtggctaaaggtaagagataagacagaacatttccagtccaaataacctactaagattacagttagaagggaagttatccaaccattctggaatattgacagttttctagacaaagttattctatactgggatttgttttatgaagaaagttcaacaaagtaattttttagtaggctttttattaaatatgggacactgagaaaagaggagataaatggatcagataagatcctcccttcattggcttatcttgtaatcaggaatgttaatttaggttttcatgagtttttgagtacttcattcaacaaatgctcagaatgcatccatgtgccatgtactattattgtaggtagacaaggctgctctgtatcatttatattctaaatgagggaggtagtgagtaagagtgtaaatacttgtttaaatacaacttgaaaatagcatttttgtacagtgttctcagtgatcagagtgagccatgtgaatgccaagggtaagagcctttgagc

SEQ ID NO: 28 - DNA, 1162 bp, Unknown Organism
  Description of Unknown Organism EMBL No. q9ulg1
cctgctggctactaatcttatttcctctccattaatttgcccattctggacatttcatataaattaaatcgtagtatgtgggctttcgtggctggctttctcacttggtatactgctttgaggtctcttatggtgtggtatatattagtacttcatttcttcttggggctagtcatctgcattttatttatccagtcatgagctactggatatttgaggttttttctcccactttggccatcacaccttgttttgtgcttgttcccttatatgctatattctttttcctattgatatactctaattttttgtttgccttgtatatttaacatttagggttttttagttaaattaaaattagtaaagattagtgtaagaatcagaaatgactatactctgactgttttggccattgttgtagcatctgggaaaaaataattggattgtaaaagaaattgcttcacagcaaaagttatattccattttcaccccttttctagagcattcttttaagtgatgagtccagtgaggcagattctcagagtgaagacaatgatgatgaagaagaagaactcagtctcagcagagaagaacttcacaacatgcttcgattacacaaatataagaaacttcaccaaaataaatatagtaaagataaggaggtgagagtttcaagagaacactcacaaccacactattttaacttatgtgaaataataaatgttaagagctttattgtgataggattcacataacataaatccagcttttggaagtaggtaactgaattttttctgagtttattcgtcgtgcatgttttcgtcatcactgtctcagttctagaacactttcctcacttcagaaagaaacctttgtgccatggcttctgcctcattctgttctttgtgccaggcacccaccagtttactttctttcttgatacttatttttattttacgtatttattcttggtcttgtataatagggtgaccttgaactccgtgttctcccatctccatcttccaagtgctggcatgtgtcgccgcgtgcagatgtgtgccactatgtgcagctctgattgttgtttggtttttggatggtgttagagattgagtccagggcctcatgcatgtgcgtttcttataaatgtgttcattctgga

SEQ ID NO: 29 - DNA, 1121 bp, Unknown Organism
  Description of Unknown Organism EMBL No. q9ulg1
ctttgttttctgttttatttatgataaggccttatttagctcaggttggcctagaattccttacctccacttcccgaatgttgaaattacctgtaagaaatgaatttcataccaaatgtgtatctgctgtgtgtagtggtacatattttgcatttctagtatttgaagtgtggaggcagaagtatcaggagttcaagtcagcctgggctacatgaacttgtgttgaagaatatagaaaagaaaagatgtgatagaagttcagattctgaaggatttccttccaaattcctttctagcagtttgtcttgtgcatagcagttgatcctgttttacttctgagttaaaggaattatctttgatgtactaatttcttattggtctctgtgggaaactatttcagagctatggttttctctggcttagctttttttaagttaagatgacttcaaggctgcgttgtggaacgcaggcagacccatcacagtgtttgcctgcttcagttgcagcagtaccagtactacagtgcaggcctgctctctacatacgaccctttctatgagcaacagcgccacttgcttggacccaaaaagaagaaattcaaggaagacaagaagcttaaaggtaagtatatttattctttcttaatttcagaactgtgtgtggtacattttaacttttggtagaaaattacttttatctgtaaaagtatagtctctttcctaattaccgacattattgaccttatttacatttgttgtgtggtcttttatagacatagatagacttaacgaactataggttgcaagcacgtggactgtgatcctagcgcttggtaggtaggagtaggagtctgtggagcaaagttcttgttggctacatagaggctgagtctaccttgggctgcctgagaccttgttttaccgcctcctccttctcttctaacccaagaaacaaagaaaactatagctgtaggttgctacacaaaggtttaggcaaaatagtttccttccttttatcttgtacactctgccatcgatgagagtattctgaggtgagaagcagttggttaagttactccagatgagcctctgtagcttctgcagtggttaccaggatgcaga

SEQ ID NO: 30 - DNA, 1215 bp, Unknown Organism
  Description of Unknown Organism EMBL No. q9ulg1
tcaaaaaacatcttttctagccttcccttctttctccataccctccttttcttgtcttccagtctccctctcaatgtaaataatttgtgccatttggtttttttcctcaaaaattttattgacctctcaattgatgcaatttttccttttattttctttagttgccttgcatttccaccaagtggaagtaggtttccatcctgtacctgtcacttgaggccagtgactcttcagctttataaacaattctacagtgttgagttcacgtcatttgatttttaggttttagtttcagtttttcatgtcaacaaaaggaacgtcagtgtccagtgttctttttgagttagtgagatcctcctcttcgacgattcattggcctgatagaaggaagaaactctgcctgcctctagtagaatgaacagagctcctggatgtcttctcaaatgatgagggtgctcgctggttctgtacagccttgtcagcatctctttcctttgcagccaagttgaaaaaagtgaagaaaaaaagaagacgtgatgaagagttttcctctgaagaatctccacgtcaccaccatcaccagaccaaagtctttgctaagttttctcatgatgcacctccccctggcaccaagaagaaacacttatccattgagcagcttaatgcccgccgcaggaaagtgtggctgagcatcgtgaaaaaggaactaccaaaggtgagtggatgggtgggtaagggagggagggaaagggagagatgggaacgaggtagcaggaatggggacaggaccaacttgtagacattcagtgatgttgtgatgtcttttacagccacaagaagtaagccaagtcattaacactgaccagcatgagggtgaatagagatattagagatgaggagaggaaggccagatgttgtagaaacaagtcaaacatgtatttgcctatgttatgtaacaccgtcagtttttagaggtagaggccaagtgggtgtctccatgatttttatactcagagtttgtaaaaaatggaaaacaatgtattatataaaactaaagaccaaacaaactctacaggagttcaggtagagtgggtagaactccccagcaagcctgccatcctcagtttgatctatgtgtgatctgttcttagttggaatgttaagggaagaaatatgtatgccttggtgtctgaggaagtttcagatgatgccctt

SEQ ID NO: 31 - DNA, 1054 bp, Unknown Organism
  Description of Unknown Organism EMBL No. q9ulg1
caatagcataagaatctaagatctagttcccagcacccacgtaaaaagccaagcattgtggtatgtacctataaatatggtacattggaggctgagacaggaagatctgtgtagtttattggccactcagtcttgctgaatttgagttccaggtttattgagaccctgtctgaaaaaataagtagcaaagtgatagagcaaagtacccagcgtctaatgctacgctctacacatgtgcacatgtacatgtgctcatacatttaaacgtatatacacgcaggtaccatacatgtggaagtaaaactaaaataagattaatggcaccttcaacagtagcttcttgttcctggtattttagtagttttatggtatgccagacaatgttttaaaagtaatttgtcttgtatgtacttgacatttttcagtccttcaggaactaaataaaagctatgtaatcttacttagaaatgaatcttaaatattactttcaaatgttttaggccaacaagcagaagtcttcagctcgaaacttatttcttaccaatagccgaaaggtaacatttttccgttttctattttgtctattgtgtgttctctaggcttattcttactactctcttatgttgtgatctgaattggataatatgtaaagaaccatctaagtagtctcattctgtttaataagtaaaattgttcttcttcatatttctcttattttctgtgtcaaattaatctctgattctttaagattcgtgctcagtttttccacttaagaagtgatgggggtcacagggatggcttggtgggtaaaggcactttctggcaagcttgacagcctgagttcagtccctggaaccactgatggaaggaaagaactgactgcccacagttgcccttcaacatccttacgtataccttggtgcatgtgcgcctgtacacacaactctcccctcccccaacaataatggtaacaataatagaaatgtggatgtgaaaaaacttctgagagaaaaacaataacttgttataagggttggatgaggtaacagtttag

SEQ ID NO: 32 - DNA, 1204 bp, Unknown Organism
  Description of Unknown Organism EMBL No. q9ulg1
tgccattgttgcatgactttttctgtggaattgtgaagaaatatgttcatcccatttagggtaccgaggactggagcaatggggtctattgcacactgcctagggagccacatcagtgactgcctcttccctcatgcatgtgtttgctgccttgaaatctcttcctacctttcacagaggaacaggtcacaagggttcagtcttgtgagggtctcaagctaataaatatagtaaagggaatttcatgtataatgtacagaataagtatactttgtttcatctaaagtgatcgatgtggataggcatcttaattagagtattttacctcaggggtctgagtcttactcaggggcattaattaaagcaggagagagagatataatcacctctttagtagaattgtaggtactaaaaagattgagggacactggtttaaagtccaggcagaactgttcttggaattgtgtccttctctgttgctataatgatgctgtttgcagcttgcccatcaatgcatgaaggaggtgcgtcgagctgctttgcaagcacagaaaaactgtaaggagactctgcctcgagcccggcgcctaacaaaagagatgcttctgtactggaagaagtatgagaaggtggagaaggagcaccgcaagcgagcagagaaggaagctctggagcagcgcaagttggatgaggaaatgcgggaggtggggcaaaagcatacattttgttttggtctggtttggtttttcttttctttctttttttttttttttttttttttttttcctttgggttgttggagacagggcttttctgtattgtcctggctttcctggaactcactctgtagaccaggctggcctcgaactcagaaatctatttgcctcttcatcccaagtgctgggattaaaggcgtgcaccaccactgcccggttttggttttgcttttcgagaccaggtttttaatgtgtagtccagctatcctggaactcattcaatagaccaggaaagactgcctttgcctcctgagtgctgggactaaaggcatgtgccaccactgccaggcacaagcatacaaattctaaaacctctatttagtaccaaagcactaaggatttagtttgttgctttgaacctttgagatagtagctctggctttcctggaactggtgacatataccagattatcctcaaactcaaaggt

SEQ ID NO: 33 - DNA, 1196 bp, Unknown Organism
  Description of Unknown Organism EMBL No. q9ulg1
taatttagttttctgagacctatcctctttttcatctaccatttttaagcttgtagccattatattgatgaatgtgatgtctctcaactgatctaggcgttgtatgtctagtaatagagaataagtaattgtttgacattggtaaaacagttgggaagaaaggtaggttcagaaaagagagtcgggattagatgatgtggttttcagatgtgaccagcatcgttctcggtggagttttgaagcctcttcctgacacttaatcatccttttcttttctctttcactctttcttctcatccttccttctcttcccttttctttcttttgtatttatatttttatgtgtttatgtgttgtttattagcagtagttgcctttttaaaagtaaaaaggttttgaaggacttatacacaggcttttattaagatttgctaaattctgttgactacatataatttatgacttggatattcttaccttcaatttccaccctactttaggccaagaggcaacaacgaaaactcaacttcttaatcactcagacagaattgtatgctcattttatgagtcgaaaacgtgatatgggtcatgatggaatccaggaagaaatcttaaggaaactagaagatagttctacccagagacaaattgacattggtggaggagtggtggttaacatcacccaagaagactatggtaagtcctgagtcaggaagtagtggaagtaggtagccttcttttttcacatcaaggttgtgtatgttaaaattcttacaggttttgtcctaattcgtgcataatactgctcagaaatgggagatttaatgcactggatcaagaaaatacgtgtatttctgcaaaggaagcctataaaataacttaagaattttcagtagaggcctgggaagagagaatgtttgcctagctgcacgaatccctggacttgatacctagcatcacataaaccaggtggtagtgatctcacacttcagaaggttaggcaagaggatcagaagttggaggtcatcttcagttacatggttagagtattttcatagtgtagtaattaagatcgtgttgacctagaaatgcagaatggaaatatttcagagtatcttgagtattaacacatgtatagatttcaattgctttctccccacataaatataattttgaatctgcatgttggtcttgcc

SEQ ID NO: 34 - DNA, 1068 bp, Unknown Organism
  Description of Unknown Organism EMBL No. q9ulg1
caattgctttctccccacataaatataattttgaatctgcatgttggtcttgcctatcctggtaattgatatcaatttataaagaaaccataggactgagagattgtagatcttaactcccagttgggttttgtgtgctgtctttcactcttggagacaagagacagccgcagctactttatacgtctaggttctgttgtttgtttatttttgttgtagtttttttgagatagtgtctcactctggctgtcctgaaactcactgtgtagaccagagtggcgtggaacacacagaaacctacttgcctcttcctactaagtgctggcattaaagttgtgtgtcaccatgccttacttgtttctaggtttttgcccactccttgtcctttgttaatcttgggataaatacagtgggaaactgggaatccacttatgtgtgggataaatctttgaagacgtgataattctaagtgcaataatctacgtgttttctcttttcagatagtaaccattttaaagcccaggccctgaagaatgctgaaaatgcttatcatattcaccaagctcgagtaagtctttaagtcaaaaggcaggctgggcatggtgaccacacctttgatcccagcactcaggaaggtagagtcagatggagctccatgagtgtgaggctagcttggtggacaatatcagttctagaccagccaaggatacatagtgagacccgtcccaaaaaaaggggagggggaggcaaaggtatattgtagttatcctaggaaatgtgccagtttcttactttattttattctttgttcattgttttcagtttaacacactggttagtgtgtgtgtggtgtgtgtgtgtgtgtgtgtgtgtgtgtgtgtgtgtgtgtgtgtgtgtgtgtgtgtgtgtgtgtatatctatgtatgtgtatgtgtttgtgagactcagactgtatatagggtgaaggctttccttacttcagttacagatggttcttgccaccatgtgggtactgggacttgaactggaagatcagccagtgcttttatttagtgagccatccctcag

SEQ ID NO: 35 - DNA, 1210 bp, Unknown Organism
  Description of Unknown Organism EMBL No. q9ulg1
taaaagtttttatttttttatttttttattttttttttattttttttggtttttcgagacagggtttctctgtgtagccctggctgtcctggaactcacttgtagaccaggctagcctcgaactcagaaatccgcctgcctctgcctcccgagtgctgggattaaaggcgtgcgccaccatgcccggctttttggtccttttttttttttttttttttttttttttttttggtaaaagtttttattcaagtagatcatgtactttcattatggttaacatagtctcctatgtacccaaactcttatatcagaacttggaacaaatagttttaattagtgagcaacatagcttagtatggtaataaaaacattttaatataattgttacattaactcagtaagatattagaaacattattagcatagtgtaaaaatgttgataaattttatgttaatttaaaaaattcttatatatttttcttgacatttttattttccagacaaggtcatttgatgaagatgcaaaagaaagtcgggcagctgcccttagggcagcagacaagtctggctctgggtttggggagagttatagcttagcaaatccatctatccgggccggtgaggatattccgcaacccactattttcaatggcaagttgaaaggttaccagctgaaaggcatgaactggttagcaaatttatatgaacaggtaaatttagaactctattgagcctattctttttactttaacatctgtttttcttttgattaatgtaattatgaagtgagtttacctttagcttttatttatttattttttaaagatttacttgtttttattatgtctgtaagaatgtttgcctgtattgtatgtaactacaccataggcatgcagtctgcatggagatcaaaagagggcatcagattcctagaaccggagttagagctgggtgtgagccatgtgggcaatagaaactgaacttgaatcctttgcaaaagcagcggtgctcttaaccactgagcaatttctccattttcaaaacttcactatcatgttagagtcagaaaggtatcagtatttctaacttggatattctgtgtgtatccagtgtatatagtatgtcttagatggtcttggtaaactgcattgtttgcttaatatgtgctacattaggtaaagaactggccttagtcttggtctaatatatc

SEQ ID NO: 36 - DNA, 1081 bp, Unknown Organism
  Description of Unknown Organism EMBL No. q9ulg1
gcatggagatcaaaagagggcatcagattcctagaaccggagttagagctgggtgtgagccatgtgggcaatagaaactgaacttgaatcctttgcaaaagcagcggtgctcttaaccactgagcaatttctccattttcaaaacttcactatcatgttagagtcagaaaggtatcagtatttctaacttggatattctgtgtgtatccagtgtatatagtatgtcttagatggtcttggtaaactgcattgtttgcttaatatgtgctacattaggtaaagaactggccttagtcttggtctaatatatcatgaatcacacatatatctctagtgttgctaaaagaaactattgcagtagtgatttaatagtcttggtactgagtatctctccaatcaatctttgctgtcttttgagctcttttggcattaagtgaaatgaattctaatgggtgcaatcaggtgttgatcatgtgtctcttactctctctgtctctcagggtattaatggaattcttgctgatgaaatgggtcttggtaaaactgtacagagcattgccctcctggcccatctggctgaggtgagtagatggaattgtcaatcttgttttccttaacttaggcctcccctcccctttcctcccctccctctcatcccccctacttccctctcctccccacttctccctccacccttcaaaacagggtttctctgtatagccttggctatcctagacctcactgtacagatatggctggcgtccaacccagagatctgcctgccttttctcccaagtactggaattaaagacctgcatcaccgtcgacaatgagccacttaaaagcagtgttactcttggtagtatatgaaaatagccgaatttgttgctatacctgaagacttttttgttgttgttgttgttttttcgagacagggtttctctgtatagccctggctgtcctcgaactcactttgtagaccaggctggcctggaactcagaaatctgcctgcctttgcctcctgagtgctgggattaaaggcgtgtgccaccaccgcccagctcaagacgtttttaaaat

SEQ ID NO: 37 - DNA, 1096 bp, Unknown Organism
  Description of Unknown Organism EMBL No. q9ulg1
acagtgaaattgtgataacatcgtagaacaaataaaagtgaattagtatgttacacacacatttttttaaaagatttatttattttatgtatatgagtacaccgttgctatcagacactagaagaggccattggattctattacagatggttgtgcgccaccatgtgattgctgggaattgaactcaggacctcttgaagagcaatcagtgctcttaaccattgagccatttctccagctccatgtcatgcacatgttatgaatgaaattaagacccaaaagtcacgagtgtcactgcttagtactttacctccttagtgtgtcatttttatactaacattgcttttcattgaaagatatgtgtagacacttgggagataggcacagtttctcttgttacagtaatatctatttttaaacttctcttgtcatgggttgattatttaggattaaataacttcagttttcctacagcaggctaacctcattttctgttgcagagagaaaacatttggggacctttcttaataatttcacctgcttctacacttaataattggcaccaggagtttacaagatttgttcctaaatttaaggtaaagatcctcttgttctttaagataagctttctgacccttgtttactgctgtctaattgtcttagacttctgtgctgtagtaaacactgtaaccgaaagcagcacagaagacagggttcattttagttcacagctttatatttcatcatcctttaaggaattctgagcaggagctcaagcaatcgggaacctggaggcaggtattgaagcagaggccttagaactagtgtgctgcttactggtttgttctcatagtttgctcagcctgcctttttcatcagtcatctatgaaggtgtggccccgcccgctgcaggataggaccttccacatctatcacttacagacttgcttagaggctaatcttggttttgtttctttttctttttctttttttttttttttttttttgcttttttgttttgttttgtttgtttatttgtttgagacagggtctctatatgcaaccttgtctatcttgaaactctctatatagacca

SEQ ID NO: 38 - DNA, 1060 bp, Unknown Organism
  Description of Unknown Organism EMBL No. q9ulg1
caacttttttgtttgtttgtttgtttgttttttcacagggtttctctgtgtagcccgggctgtcctggaactcactctgtagaccaggctggccttgaactcagaaatctgcctgcctctgcctcccgagtgctggggttaaaggtgtgcaccaccacagcccggctccttacaacttctttatttttctcataaaatgtgatttgccaaggttttttgtttgcttttgttttgattttttttttgtttgtttgtctttgttttttcgagacagggtttctctgtatagttctggctgtcctggaacttactctgtagactaggctggcctggaactcagaaatccacctgcctttggctcccaagtgctgggattaaaggcctggacaaccactgcctggcctcttaatagtttcttactaagtaattcagaataacattattgataatgcttttagtttattttcaaaaacaaaataaggtaatgatctgttttccaggtgctaccatattggggaaatcctcatgatagaaaagttatccggagattctggagtcaggtaacttcagtttctttctctctctctctctctctctctctctctctctctctctttcgctctctctcgctctcttgctctcctgtcctcttgctctgtcattctctttggcctgagtaaatcctcatgtatacaaaatctataatatgtttctgaatatgggtcaaatatgtttttattatttgaataaaatgtttttcttctacaggcaatcatttggactttaaaaagagactttttaaaaaaagatttatttttaaaaaattgtatttgtacaacaaatatatgtagaattctttttttttttaaagatttatttattattatatgttaagtacactgtagctgttttcagacacaccagaagagggcatcacatctcgttatgtatggttgtgagccaccatgtggttgctgggattcgaactcagaacctttggaagaacagtcagtgttctccaccgctgagccatctctccagcccatatgtaggattctta

SEQ ID NO: 39 - DNA, 1143 bp, Unknown Organism
  Description of Unknown Organism EMBL No. q9ulg1
atttattattatatgttaagtacactgtagctgttttcagacacaccagaagagggcatcacatctcgttatgtatggttgtgagccaccatgtggttgctgggattcgaactcagaacctttggaagaacagtcagtgttctccaccgctgagccatctctccagcccatatgtaggattcttaagaggctagagaggagggcatcagatcctcttgagctagagttacaagtgattctgagccaccatacacactccaagtggtctaactactgagctgtctgttcagtcacaccttcaagctgagcaaggttaataattaatgtgcatttaatccctagaaccaatagtggaaggagagaaccagctctggaaaattgccctttttatcaccacatctatatgcttactcatatttacacatacatatacatcaaaaaaacgtttcttataaagtgtgacataatgtttttccacatttttcttattacttttgtagaagaccttatatactcaggatgccccctttcatgtggtcatcactagctaccagctggtggttcaggatgtcaagtacttccagcgggtcaagtggcaatacatggtactggatgaagctcaggcactgaagagcagttccaggtaatatgagtaatagaaactagccttgtgggtggaaatctatccatagaatccgcaaaggccagaaatttcagaatgtcacatagcataggctatgtatgcctcagctacttgcctgaccagaatggagaaattcttatttatacttttctttgaaattcgaaaatcatgcttagaatatattgatttttcttcaatattaatctcctcagttaatggttataatttttgttgttgttgctgttgtttcctcttcttcttccttcttctcctcttccttcttcctcctcttcctactccttcttctctttccttttcctttttaaaggtaagaacagtaggaaactgtaaggccagaaaggtctctcactttacctatttgtggattttttttcccctaagccataataccattggtattgatgattctcacagttctgtttttttttaatttctttctttttgtttaaagatttatttattattatatctaaatacac

SEQ ID NO: 40 - DNA, 1085 bp, Unknown Organism
  Description of Unknown Organism EMBL No. q9ulg1
tttttgattgtgtgcatgaaacagtatatgatttttttaaagtttatttattttatgtatgtgagtacatgtagttctcttcatacacaccagaagagggcaccagatcatattacagatggttatgagctaccatgtggttggtggaaaatgaactcgggacctctggaagagcagtcaagtgctcttggggcgggggtggggtggggtgggggtgttttgagacagggtttctctgtgtagccctggctgtcctggaacttactatgtagaccaggctggtctcaaacttagaaacccacctgcctctgcctcccaagtgctgggattaaaggcatgtgccaccactgccaggccaatcaagtgctcttaaccactctccagccccagtatatgattcttaagtattatttggaaagcaattttagaatttgggtcttagtataattacagaaagacatattgccaacttataatttgtaactcattttcttgaacagtgttcgttggaagattctcttacaattccagtgtcgaaatcgacttttgttaactggaacccccattcagaataccatggcagaggtaggcactagtcaaggtaatggagtatatcttcgtgagacacctatacctacgctggactccagtcattctgtacaaattatatttaccactgtacaagaattagattgtagcttcctaagtgtcttgtaggcagtattcttactttcttactatttattaattataatgttccttttgtttccttaatattgagtacattaggaaatgttgcccatctaataaaggtaactttttactctagaattggaaaatctctaacatcgagggcctcaatttttttgtttagaattcatcataataggggtcagaaaactttcggtaaaagaggaaatagtattttacatttcatagacagtacatttcactgttaacaactgcttgtttttgcttatgcagtatggaagcagtcataagtagcgtataaatgaatggctatggctatgttcccatagaactttattcatgaaaacatatgaccagccgaatagcgtcatggc

SEQ ID NO: 41 - DNA, 1118 bp, Unknown Organism
  Description of Unknown Organism EMBL No. q9ulg1
taaaagaggaaatagtattttacatttcatagacagtacatttcactgttaacaactgcttgtttttgcttatgcagtatggaagcagtcataagtagcgtataaatgaatggctatggctatgttcccatagaactttattcatgaaaacatatgaccagccgaatagcgtcatggctgtaattgtctagccctggctgagggctattagcagctattacactactataaactgtgttcagttgtggaaccagctgtggggttgtcatttgtaaaacttattacactacaaaagctgagcaggctttgtacttctaaatttgtagttagcaattaattgagaaatcatcattttttgttatgtattagggttcactgagcaagattgtgtttggattcatttattctttctgtctcataaacactcatttcttaatatatagtcacacagtatcattttatggcaccatatctgtttcatatttttcctttgttttaagctctgggctctgctacatttcattatgccaacattatttgattcgcatgaagaatttaatgaatggttttccaaagacattgaaagtcatgctgaaaacaaatctgctattgatgagagtaagtactgctgtggctttcctctttttgtgtagagtttaagactctgtaacccaagtctcacattgcagcctagaatggcctgggattctctatgtagtgtggcctcaaacagatatttccaggttcaatttcccaagtgctgggattataggtggctctgaattttattttcaatctattagtatgggcaactatgcacagtttttttctgtgtgaagaatttctctttcattatacctacaagagttaagggtaaaactgttataaattagttaatataatgttaataaagtagctaaattctctgagttaaaaagaatagaacactactctagacaatctaagcaagatgtataaacttaataaaatattacattttggtttgtaggcattctgtcttaggtagggtttccattgctgtgaacagacatcatgaccaaggcatctcttataaaggaaaacatttaattggggctggcttaccatttcagaagtgt

SEQ ID NO: 42 - DNA, 1086 bp, Unknown Organism
  Description of Unknown Organism EMBL No. q9ulg1
gtaattcagaaaatggagccacgtgttgtgatgtatgtttttaatctcaccacttaggaggcagaggcatgtggatctctggatttgaggccaacctgatccatagagtgagttctaggatagccagagctacatagtgagaccctgtcttgaaagaaaagaaaatattcccacagtccagtcttgatgggaaagaattcctcagttggagttctctatccccagggaatgctactttgtgtcaggttgataagaattacatagcacactctggaagtcccttttagctttgagtcgatttttatacagtctcccaaataggctctgaaagtgtgttctaatgtaaacagtcggtataagatgcttgccttggaacaagttcattttttagagaatccttgatctcagtggcattttcagggtgtaggagaggggaaggtgtttattttgattgtcttaagtcagtatatggtaaattatttgtggacttcctgtttcagatcaactctctcgtttacacatgatcttgaaaccatttatgctgagaagaatcaaaaaagatgtagaaaatgaattgtctgacaaggtaagaaataaaatcttaatgagtttgaaccttagtagataccaatttatggttcttcacagatatttccttgatatatattataaggataattagtttgattaggaagatctttgtacttgaatagtcataggggacctaacgaaggcaaagtaaccttctttccctttggttactaatcctcctccagttggttttgttttatctttatagggtcttaacactgtaagctgggccagcctggaactcatgtatgtaggtgaccaaactctgaacagtcttcctgcctttgagatcataggcatgagccatcatgcctgaagtgtaaaacattacaaaaatagaataatgtaataactccactcgtgcccatgatagagcataagtagttatcaacacatggccatccctaactgtacatgtattatgaaactaatagtttcatgcagatctctcgattttcctttgaaacaatgtctcactgggtagaccaggctgag

SEQ ID NO: 43 - DNA, 1168 bp, Unknown Organism
  Description of Unknown Organism EMBL No. q9ulg1
agttccagaacagacagagagagctacacaggcaaaccttgtcttagatgaggggaaaaaataaaagaagtggaaaatattattcagggagaacatctagtgtgtgattgttcacaacataggataggattatagaaatagttaattctggtaagtattgtttctttagtcataatctggtttgtatgtcttctgttttaagttttactgttttttttgtttggttggtttttttttgtttgtttttttttttttggtcttttgagacagggcttctctgtgtagccctggctgttctggaactcactctgtagaccaggctggccttgaactcagtaatcgcctgcctctccctcccaagtgctagaattaaaggcgtttgtcaccattgccccctcctcctttttttccttcacatatacaagcatataaaaaatttttagttttttacaaacatatataaaaatgtttagtttcaaatttttcttttttttttttagattgagattctaacgtactgccaattgaccagtcgacaaaaactcttatatcaggcactaaagaataaaatttccattgaggacttactgcagtcttcaatgggctccacacaacaagcacagaacaccaccagcagcctcatgaatttagtcatgcaattcaggaaggtaagacttgggtcatttgttttgatgtttttattgacctgcttcacggagaggactttaatcttgatgccagatagagcaatagagatgaaaattgggaggtaaatattcagggagcttaaccctcagcagtgatgtccacaagggacagtaatgactgaccatttcttttcttaggtgtgtaatcacccagagttattcgaacggcaagaaacgtggtctccatttcatatttctctaaagccatatgaaatttcgaagtttatttatcgtcatggacagatcagggtcttcaaccactcacgagataggtgagcaaatagtttacatttggtgatgctgtgtgcttttgtaatttggcacattgattttttagtagtgctaggtattgaactcaaggtctcatgcttattaagtgaatgatatgtgaatgtccaataaagagatgtttaaacaagtggtgaacagctattgcgggtgcttatggggggagttggagc

SEQ ID NO: 44 - DNA, 1134 bp, Unknown Organism
  Description of Unknown Organism EMBL No. q9ulg1
gcctctccctcccaagtgctagaattaaaggcgtttgtcaccattgccccctcctcctttttttccttcacatatacaagcatataaaaaatttttagttttttacaaacatatataaaaatgtttagtttcaaatttttcttttttttttttagattgagattctaacgtactgccaattgaccagtcgacaaaaactcttatatcaggcactaaagaataaaatttccattgaggacttactgcagtcttcaatgggctccacacaacaagcacagaacaccaccagcagcctcatgaatttagtcatgcaattcaggaaggtaagacttgggtcatttgttttgatgtttttattgacctgcttcacggagaggactttaatcttgatgccagatagagcaatagagatgaaaattgggaggtaaatattcagggagcttaaccctcagcagtgatgtccacaagggacagtaatgactgaccatttcttttcttaggtgtgtaatcacccagagttattcgaacggcaagaaacgtggtctccatttcatatttctctaaagccatatgaaatttcgaagtttatttatcgtcatggacagatcagggtcttcaaccactcacgagataggtgagcaaatagtttacatttggtgatgctgtgtgcttttgtaatttggcacattgattttttagtagtgctaggtattgaactcaaggtctcatgcttattaagtgaatgatatgtgaatgtccaataaagagatgtttaaacaagtggtgaacagctattgcgggtgcttatggggggagttggagcattattaaggctcccattagatggccttaagaaccttagccattttttattatctacctgttacgggtatgtatttgcaccagaagtcaacagttaaaatactaaaatgccttggtatgcttaaagtgacttagagttgcacttactgcagcactgagtctattactttttggttaaatttactctcttgatagtgtctgcatttaaaaagttgaaatagtcactaagctaatctgctagagatcatattcataaagtggtgtatggttcattgtatatctgtaagttagataattctggattccagactt

SEQ ID NO: 45 - DNA, 1068 bp, Unknown Organism
  Description of Unknown Organism EMBL No. q9ulg1
gacttagagttgcacttactgcagcactgagtctattactttttggttaaatttactctcttgatagtgtctgcatttaaaaagttgaaatagtcactaagctaatctgctagagatcatattcataaagtggtgtatggttcattgtatatctgtaagttagataattctggattccagactttctattgtgctcttaagtgaatatctgcatgccagcttttgagagctatggactttgacagggtttctttgtgtagccctggctgtcctgaaactcactctgtagaccaggctggcctcgaactcagaaatccgccggcctctacctcctgagtgctgggattaaaggcgtgcgccatcacgcccagcctggcaaagaactttcttatgtgtagaaatagtctaaaaagatttttcttattttgaaccctccagtttaattatctttccaattcttttatttcccttgagagttactgtaattacttgctttccaggtggttgaaggttcttctctctccgtttgcaccagattatatccagcagtctctcttccataggaaaggtaggcattttgatctttggaaggaagtgtatttgggatttaccagggtgttcttaattttgcctgtggacaaattttgttttgaaatagtaatggtttgggcttttatttttattaaaaataagactgttcaattagtgtctagggaggtgacttagaatggagcattagcatttgctgcactagtgtgagaactggagttcagatctctgcaaccctggtgaatgctgcctgtgtagccctcctgtcatcggaattgggtgttggaaacaggttgtgctacatgaaacttcctgcacgtggttgcagaaacaggatccctaagggtcaggattgttctttgattttccaggtattaatgaaggaagctgtttttctttccttcgatttattgatgtctcgccagcagaaatggcaaacctcatgcttcagggacttttggccaggtgagaagtttgcttactgtttgatggaagagcattgggaataggcagggacaa

SEQ ID NO: 46 - DNA, 1094 bp, Unknown Organism
  Description of Unknown Organism EMBL No. q9ulg1
ttattttgaaccctccagtttaattatctttccaattcttttatttcccttgagagttactgtaattacttgctttccaggtggttgaaggttcttctctctccgtttgcaccagattatatccagcagtctctcttccataggaaaggtaggcattttgatctttggaaggaagtgtatttgggatttaccagggtgttcttaattttgcctgtggacaaattttgttttgaaatagtaatggtttgggcttttatttttattaaaaataagactgttcaattagtgtctagggaggtgacttagaatggagcattagcatttgctgcactagtgtgagaactggagttcagatctctgcaaccctggtgaatgctgcctgtgtagccctcctgtcatcggaattgggtgttggaaacaggttgtgctacatgaaacttcctgcacgtggttgcagaaacaggatccctaagggtcaggattgttctttgattttccaggtattaatgaaggaagctgtttttctttccttcgatttattgatgtctcgccagcagaaatggcaaacctcatgcttcagggacttttggccaggtgagaagtttgcttactgtttgatggaagagcattgggaataggcagggacaacatgtaataatgtgaagtgaaatatttagtattacaccgtctggaccccccgccccccacacctccattagtaattttttttttatgacctaggaatgatacatacctgtaatcttgggaggctgaggcaggggaattgggtgttggaaacatgttatgctacatgaaacctcatcccacccaccaataaaaaaggattatgggctggagagatggcttagcaggtaagagcaatgactgctcttccaaaggtcctgagttcaattcccagcacccacatggtggcttacaagatataataggatctgatgccttcttctagtgtgtctgaagacagaaacagtgtagtcatatacattaaataaataaataataaacatcctttttatttaaagaattacttttatttttctttaataatttttttaaaaatgtatgtgagtaca

SEQ ID NO: 47 - DNA, 1172 bp, Unknown Organism
  Description of Unknown Organism EMBL No. q9ulg1
actgattttattacattattgaatttatttataactttttaaaaaatgttgattggtgttttgcctgcatgtatgtctgtgtgagggtgttatagacaggtgtgaactgtcatgaagctgcgagaaattgaacttgggtctccaaataagcagtcagtgcttactcttaactgctgagccttctctccagtccctgttacagtgtttctaatgtactagatagctcattgggaattattgtctccccgataagatggtttttatgctaagagcttttacagtagagataaatgcttttgaatatttttgtatttgtttactttttgttgtggttttatagaacagcaaaagtatgaaattgctttatctgaatgtttgaaggaattgtttccaaaatgctcaagatctacaaaggatgtatggtaatgagatagatccttgttgtgaactcccaaagttcctaggagcactgagtgtgcacatttctctgtttgtttcaggtggttagctcttttcctatctctgaaagcctcctacaggctccaccagctgcgctcttgggcagagccggatgggacaagccatcagagctatctgagaaacaaggattttcttctcggagttgatttcccactttccttccctaacctttgcagctgccctttgttaaaggtaagccactgtctgtgcagtgtctgcttactttccttctctttgcattattcaaaatgttaaatgatgtcaagtacccagtgttctcaggagctcgtactgtgtattgagttagaacatatcatgtgtaagtttaaatcctctgtatataataacttcattgtcgtgaacaactgcttggcttgctttctctaaatgtttcctttatttttatcgtctatgttagtagttttgcctgcatgtgtctggagttgatttcccactttacacccacattacaaacaacttctcccacgaaatctggatctgcctcacacatctaccagaaggaagaggaatgaaaaggagagggacaagaggactatgagttcagagtcagcttgggaaatttagtgagacccagtctacaaaataaaaataacaataggcccgcagctgttgtagtagtttgccatttgctcatgttaaatttttcttaggaattatttgcttgaggctcc

SEQ ID NO: 48 - DNA, 1141 bp, Unknown Organism
  Description of Unknown Organism EMBL No. q9ulg1
tcccagcactcagaaagcagcagcaggtggttctctgtgacttgaagactagcctgttctacataaagaattctgggccatcaaagggtacactgtctcaaaaaggatcatctttgctagaactacatgatgctgttagcacctgattttatattaagagaaaatttagatgaacatagtcttaatcatcatggcttgagtttacatctgtagttataacaaactacttagtacacgtagtattttgtctgaaatacctcttctgaaagtagaattagaaatgacatttgtttctgtatactaataaaactgcattttgaaattaatgtgggcctaaactacgtactgtatgtttggttctaatttgtggttgtttagatataatagtattggccaagaaaaaaagttctttaagttgaatagtatatgactcattaacagtaattgtccttacactaattttggtttgttttttttgttttttgttttgttttttttagtctcttgttttcagcagccactgtaaagcagtgagtggctactcagaccatgttgtccatcagcggagatcagctacctcctcacttcgttgctgcctcctcactgagctgccgtcttttttgtgtgtggccagtccacgagtaagtgccacaggagagaaggaatgaaaccaattatcctgtaagaatgagctagtactcaagggacagtttctaagctgttcagtcttagtttgtaagaagagaagtgctgggcgtggttattcacttttgatcctaagacatgggaatcttgtgagttcaaggcaagactgaattacatagtaaaaccccatgccaaagaacattaaaaatggagaatatccatgatcctgaccttcctaagacagtacttctcaccttgaaatgtgcagatttgcaaattattatatggtacatggttgctacttttgattaagccagagactgtaaactgtaatgaaacaaggggcttgttgagtacatcattccttagcaggatcagagaaaaggaaggaagccattgtcacctgctttgatgttttattaaggtcataggattctggctcaattatttgaagattctctctggccttatctcaatacagtccaggatcaggcaa

SEQ ID NO: 49 - DNA, 1230 bp, Unknown Organism
  Description of Unknown Organism EMBL No. q9ulg1
gcttcctttctgacatcactgtcataagtggccatagcacttgtaataagtgggtggtaaatgtatattgagagtagatatccagactagaatcttactttatctacatgcacctttttatatcttttgattgcaggttcatgtgattagcgattaccagtttgcccagagaagctctcaaattgagttagtctttgccttcatcttactatgctagaggatgaaatataaagccaccagtctgaggttattcacattgatctgatttcagagttgtgggcctagggatagagctcagcattcacaagcctgaaacttgctataacccccactgggtaggtgaaaggactactcaggtggcttcttgagaatttggaggccagcctgagctatatgagatcctatctcagaaggaaaaaaagttgatttccctcaaaggcttgatgtgtgtgacaaatggcagaagagccattttattgtctgcgaattttaccctgcaggtcaccgcagtgccgttggattcttactgtaatgacagaagtgcagagtatgagagaggagttctgaaggaaggagggagtctggctgccaagcagtgtttgttgaatggggctcctgagctggctacggattggctgagtcgcagatcccagttcttcccagagccagctggagggctgctgagcattagaccccagaacggctggtctttcatcagaattccaggtgagttttctgctctttggagcatgtttccagctttgacaagaggagaaagggggagaaagcccatgtgatagtttttgttgtttggtatttggagttcctgaggtttctaactctcctaacctgtaacttataggtaaaaatatgagtactttgtgtcttgagtggaaagatgatgattgaaatgtttctagatttgttcgtgagtactctcctttgataagaagagtgtaatgtcacttttttaaatgggagaaggttggggtgctcatgggtagagtgcttatctagtatgcacaaggccctcaccagatgaccccccacacacacacaccccaaaaccccacaaagtttaggaaagacttcaggtcgaatgagtgcagttgttttgtgccattgccagtggaatgaatgagaatgtcgtcactcccacagagcctagttaagtagcataaacagtgtcctgcacaaggtctagagatctaaatgatagcaagaggaaaa

SEQ ID NO: 50 - DNA, 1286 bp, Unknown Organism
  Description of Unknown Organism EMBL No. q9ulg1
taactcccagcatgcaaacagaggcagggaaacctttttgaatccaaagccaacctggtctacatagcaaattccagactagccattgctacaaagttagaaccttgtctcaagagaaatgaggagaagaggaagagcagttaacagctaagaggtattaccaggcttcgcagtgtctctgtaatctgagcacttggatgatggaagccagaggaccaaaaactcagggtcatcctcagctacatactaagttctagagtagccaaagatagaaggaagtatctcaaaagaggggagaaaggctattgggctgaaatcgtagctcagaggtagagtgcttgccaaatgtgttcacattcctaggtttggtgctctggagtaccgcaagaaaggcaaaacaaacaatatacatgtaatgttgtatagctgaaggtctgtttctataatgacatatttttctgtaatatctgttccatgtttctacttttattggttatcagtcagatattagtatggcagcaaaagcttatgtacagattacaaggtgactgccatcatttgagttttaaagaaagaacacctttgcacagtagtacaaacctcaagatagcatccactctctagccctagattgtaattgtagagattcctgtctatttccacattgaatctttccttttcctcctaacatgtcttcctcccctcttctttaggaacgacatctttgtgtttctgttaagcacgcgagctggaggactgggtatcaatctcacagctgcagacacagtaagtgatgaggtctctactataaatagttcattcgttgcacgttttacttttcccttttgttttttcattcactttgaatgattttatacattaatttaataaaatgcttactatgtgtcaagtattttcctaacagttaagatatagcatgataataagtgaacaggagatggctgctaaccaggcagtccaattggttttgtctgaggagctataactaaacaaataaaaaagagttgttttcttattagtgcctgatttcattacttgagaaggtaatggtagaagttgtggtctaaatgtcaagagctgtttggatagtgatgaataattaaataaaataaagtaaattatattcttgcaattttttgtccattcataccaagtctcctgataagactactttagcctctgaaaggaaaaagaaattttcttttgaatcttagcattattccatggattcagatgtatttagtagcagtcatagtaacatttag

SEQ ID NO: 51 - DNA, 1159 bp, Unknown Organism
  Description of Unknown Organism EMBL No. q9ulg1
aacctggtcttgatagtgatcctttcgtgtgagcttcctaagtatcagtgactttaggcacatacaagcactcctgtctaattgttaccaccaacccctcccccttaaatgtattttatttttaattgtgtatgtgcaatgccgtgaaagctagatatgtcagattccctagaactgggggtacagaagactatcagcctcctgtcccaggtactggaaataaaacccaggccctcatcgcttagccatctgtctgtctagttcatagttggatcttttcagtgaaaactggaaagacggggggcatgcctagttttccagagcaacacacatatagcttggtcttttaaggagtttctttttcatcattacaccatagaaagaaattggacatgactaacactagttttgtagacagataagtggctatactgctctcagaatttctgtcctgcactcagaacatccatttttgttgtatccttttcttgtctatgcaggtgattttctacgacagtgactggaaccccactgtagaccagcaggccatggatagggcccaccgcttggggcaaacaaaacaggtcactgtgtaccggcttatctgtaaaggcacaattgaggagcgtattctgcagagagccaaggaaaagagtgaggtgagcacggggaaggaagggtgatcgtgctggagggacagaaagcagtcagtactgtaccgaaattatttaagacttatatttcattaggcagacaaactagtgttcttggtaagcattggagcctgtaccttacttagtcttactagctcctgtgtacactgataccaggaagggtgacatttcagagttcaccttggtttgtccccaaaaaaatcttcaaattgtcctttctttgggacaaataatgtagaggagccaggtcagtgaacaagaactagtgagccaaagcctccctttggagctgatttcacagggctgctgctgctgctgctgtatgggaaagaacacatgcagatgacattttaatccactttgtttagattcagcgcatggtgatttctggtgggaacttcaaaccagataccttgaaacccaaagaggtggttagccttcttctagatgacgaagaactggaaaagaaacgtatgtactttggc

SEQ ID NO: 52 - DNA, 1103 bp, Unknown Organism
  Description of Unknown Organism EMBL No. q9ulg1
caggccatggatagggcccaccgcttggggcaaacaaaacaggtcactgtgtaccggcttatctgtaaaggcacaattgaggagcgtattctgcagagagccaaggaaaagagtgaggtgagcacggggaaggaagggtgatcgtgctggagggacagaaagcagtcagtactgtaccgaaattatttaagacttatatttcattaggcagacaaactagtgttcttggtaagcattggagcctgtaccttacttagtcttactagctcctgtgtacactgataccaggaagggtgacatttcagagttcaccttggtttgtccccaaaaaaatcttcaaattgtcctttctttgggacaaataatgtagaggagccaggtcagtgaacaagaactagtgagccaaagcctccctttggagctgatttcacagggctgctgctgctgctgctgtatgggaaagaacacatgcagatgacattttaatccactttgtttagattcagcgcatggtgatttctggtgggaacttcaaaccagataccttgaaacccaaagaggtggttagccttcttctagatgacgaagaactggaaaagaaacgtatgtactttggcaaccgaagttctgcccttgccagatttacagaatgagataagcattgcatatgccaaggctagagtgagtgtgtgtgtgcagtgtgtatgagtgtgtgtgcagtgtgtatgagtgtgtgtgtgtgtgtgtgtgtgtgtgtgtgtgtgtgtgtgtgtgtgtgtgagattgtggttaccttccactatatgagttccaggttcaaattctcattgtcaggcttggtagcaaatacctttacctagtgagtcatctcactggcccagaaaaatattttacacataatttcaacagcaagcttctataactataaagaccctttgcatagagttgtatattctgagaatattgagtttcagatttaccctgagttcctgtgagtacttcctcacctctacatctaaactaaactcttttcaagaagaaaagacaaggaagcaacataattgagtagaatccaggtttgaacctaaaagctgttgagatac

SEQ ID NO: 53 - DNA, 1089 bp, Unknown Organism
  Description of Unknown Organism EMBL No. q9ulg1
atattgagtttcagatttaccctgagttcctgtgagtacttcctcacctctacatctaaactaaactcttttcaagaagaaaagacaaggaagcaacataattgagtagaatccaggtttgaacctaaaagctgttgagataccaataactattgttccttatttaggaaaacgatattttctcttgttttggcttttgttttgttgttgttgttgttgtttgtttgtttgggttttgctttgttttgtttttttctgaaacagaatttctttatgtatccttccttagctgttctggaactagctctgtagaccaggctggccttgaacttatgaaatctgcctagctttgcctcttgatccctgggattgaaagcatatgcccagaccaaaataaaagtttttcttaaacaaaaattacaccttgggaaccaaccacaagaggatatggccctaagtttcagaggataacagttatgactcctacattttcttgatagtgagactgcggcaggaagagaaacggcagcaggaggaaagcaaccgagtgaaagaacgcaaacgcaagcgggagaagtacgcagaaaaggtatctccagatggcttgtgggggggccaggccctgggataagttttagaagcacaaaaggcctttgggtgccatacatgcatgtatgggcgggcgcctacacacatatttgttttacgtagtaaaccacagcttgcatcagtttttccttctcacgcatgctcgtgttggtcccagttggactggacaatcagtgataactgtgatagctaaggggataatgctacgtacataattagcttttatagaacttagtttatgtggtttgcgtcagggtctttctacttatccctgctgccctggaacgcagtgggtagaccaggctggcctggaactcaaaatatcctcctgtctctgactcaggagtgtctgacttctttcatagactcttttaaaaggacctgggcaagcatgatgagaaccacctttaatcccagcaactgggagacagaatcaggcagatctctgagttccagaccagtcatggctacataatgaga

SEQ ID NO: 54 - DNA, 1156 bp, Unknown Organism
  Description of Unknown Organism EMBL No. q9ulg1
gtgcagtgcatgcatatgcacacatacagtcagtcagtcagtcaatgagcgagccagactagaaatgggctgtcaggaaagggtatgccttcctcctcctctgcgagttttcacttccttcaagattcagggcttgagaagtggctcagtggtggagcactttccctgagtgtgagaggcctttagttcagtcctcagtacacacataacctatacacacgaaaaattaattgagcctgcatagtcatgctgtcaggaagatggagaaaatttgaggtcagcctaagctatagagcaagaccctgtctcaagtcagtgtagggtgtgtgctcatggcttagacaggacaatgaggccacaacaggaactgtaacaagtgccaggctagtcagagccatataccaagactctgcctataattgttttatttgttaatctcgcttttctctttggggcaaatactcatgtttttatgtattttctgacctttgtattcccagaagaaaaaggaagatgagttggatgggaagaggaggaaagagggtgtgaacctcgtgatcccgtttgttccctctgctgacaactccaacctctccgctgatggggatgattccttcatcagtgtcgactcagccatgcccagccctttcagtgaggtgagcctcttcctgcgttctacatcacagcagtgggactgcactctgaccttgtggcagggacatggtgggcagtgttcacttccagccttgtaggaagggttgtataaggacaggctgactctcagagccaaaggcagatgggagggagaaatgctacatgtcccccagagcatgttctcacttgagtgtgagggctgtagccattgaagttattagtaagctcagcccttctgcttccagatctcaatcagcagtgaactgcacactggctctattccccctgatgagagcagcagtgacatgttagtcatcgtggatgacccagcctcctcagcacctcagtcgagagccaccaactctcctgcttccataacaggctctgtgtcagacactgtgaatggtaagtgatgctactctttcatacagcgtcagcctgctccactgctcagtgggaagcattcctctgggggtaagtgagctctgcttccttttaggat

SEQ ID NO: 55 - DNA, 1160 bp, Unknown Organism
  Description of Unknown Organism EMBL No. q9ulg1
taccaagactctgcctataattgttttatttgttaatctcgcttttctctttggggcaaatactcatgtttttatgtattttctgacctttgtattcccagaagaaaaaggaagatgagttggatgggaagaggaggaaagagggtgtgaacctcgtgatcccgtttgttccctctgctgacaactccaacctctccgctgatggggatgattccttcatcagtgtcgactcagccatgcccagccctttcagtgaggtgagcctcttcctgcgttctacatcacagcagtgggactgcactctgaccttgtggcagggacatggtgggcagtgttcacttccagccttgtaggaagggttgtataaggacaggctgactctcagagccaaaggcagatgggagggagaaatgctacatgtcccccagagcatgttctcacttgagtgtgagggctgtagccattgaagttattagtaagctcagcccttctgcttccagatctcaatcagcagtgaactgcacactggctctattccccctgatgagagcagcagtgacatgttagtcatcgtggatgacccagcctcctcagcacctcagtcgagagccaccaactctcctgcttccataacaggctctgtgtcagacactgtgaatggtaagtgatgctactctttcatacagcgtcagcctgctccactgctcagtgggaagcattcctctgggggtaagtgagctctgcttccttttaggatacaggattttattgttgttgttgttgttgttgttgttgttattaacttttttgttctgttttggatgtttttgttgttggttggttggttggttggttggtttttttaagacagggtctctcacatagtgctggcatttgctatgtagaccaggttgtccttgaacttaataagagatccacctgcttctgatttccacatcctgggattaaaagcatatactaccacatctggcacatttttatttattgtgtgtgtgtgtgtgtgtgtgtgtgtgtgtgtgtgtgtgtgtgtgtgtgtatgtgtgtgtatacacaacgtggaggtcaaaggacagaatacaaggattggttctctccttccaccactgtggtcccagggattgaatcagcctctcaggctc

SEQ ID NO: 56 - DNA, 1216 bp, Unknown Organism
  Description of Unknown Organism EMBL No. q9ulg1
gcttgtctagcgtgcactaatccaagacctgggctcagtgcccagaaggttgttttgttttgttttgttttgttttgttttgttttaaatagagatagaagttggttgttgaaggcgggaacactttagtttttgaaggaagtcttggagttcatttagtgatgggctagaataaaaagtaataaccatggtggagccactccttagaagggacaacagtaggaaaatcatagctatgttaaaagacatccccaaatgtcctcagatctcagtgagtcaggcaccatatcctgtgtctatggtctcagaactcagaatatcagtctgagattggaggattacttgagatcgggggttttaaaaaggtcagcctgaataacattaggaaaccttgtctcaaacaaaagaggaccccttgagagccagtattccctatcaggggtatttgttagcattttaagagatttatttttttccctcttggctattcgttcccacaggaatttccattcaggaagtgccagctgcaggacgtggtcactcagctcgaagccgaggccgccccaagggttcaggaagcacagccaaaggagcaggcaaaggccgaagccgcaagtccactgcaggcagtgctgctgcgatggcaggagccaaagcaggggctgcagcagcttctgcagctgcttatgctgcatacgggtacaatgtgtctaaaggttggtgcagattgtgatcaaggactgtctgagaatccatttaacatagagtgtgcaggatattccaagccaaaacccggagaccatcttaggactgagcagttctgactcacccttgcactcctgacaaatagctgtctgtgaagtagaagtagctgtggtcagagtgaactgaaggaggagctgcacttgaaacatccagtttttctgcccctcccctttaattctggttggttattgtttgcttgtttgtttgtttttctgggacatttattttccagtaatccaaacaagggtagttaacatttatccagctacctgggccattgcctcaggcagtgcacacctaagagtgcataggggtagagctggtagagcatgcatgtgcccttgaatggaacaagccagaagagaagcacgttgtggagtggtttataagcagtatacagataatggaaggaaagaggcaagcttacttgtgattacaggtcaaaagctgg

SEQ ID NO: 57 - DNA, 1949 bp, Unknown Organism
  Description of Unknown Organism EMBL No. q9ulg1
tacctttaatcccagcactcaggaggcaagacagtcagatccttgtgagttcaggaccagcctggtctacagaatgaattccaggatagtcatctctatgaaaagatgatactatctcaacaaacaaacaaaaagcaactgaattcaggtgttttccagaagacaagacagcattagtgcctggtgagctacagctctcattgtgcaaccaggccttccatgtcagacatgtgccctctccagagcctgaagagtatgtcttccactctggatggggtaatataggtgggacttagtgtttgttgttctctctcaacaatgtagacatcttaccaaaaggcaattaacaggaaaaataggcaacatcagagatgttggcaaggagcaacagacagaggggttcccacccctttcctccagcccagacttagcctaatacttcagactccaaggctgacagctggtgctgagggtcattgtggttctctccatccctacaggaatctccgcaagcagtcctctgcagacatccattgtccgacctgctggccttgcagactttggaccttcaagcgcctcttctcccttaagttcccctctgaacaaaggaaataatattcctgggactcctaaaagcctccacatgaccagcagcctagcctcagactccttgatccggaaacagggcaaaggcaccaacccctctggaggacggtaaccatggtaaccatctctgccctctagcttccttcaaccaaaccaggggctacagtcctgagccataggtggtctttggatggcttgcctagtgtggcatcttgcatcctatttggagagtggaaaggaccagttgtagcaggagagaacaggcaggtggttggtgtgagcacttttatcacctgtttccaaaggaatatcctgggttcggccccacagcatcttagatgtactctgcagccgtactcttaacctctttctggggacgggcctgacacagaacccttttatagatgctcaggcacactgtctccatccccatcctactcttcagctctgaccatgtccctcatgaaagctccatgggtgctgtgaagagccttccatagtggagacttgtctaaaggttatggtttcctgcaggactagggaggaagtggttccctggcatccatagtctgggaccgctcctgggtctctaaaccctgtttgccacagtgtcatcactgaagaacagaacacagtgaatgccagagaagatggtggctgtaaggatgagaggctggagcaagcagagccacaggcagttatcccagcatttcctcctgtccagcacagttcacaccagggaagctttaggtcccaccagcaacagtagcagcagctcccactcaagggacttcccagaagccagtaaagagaccaggaaccacctcaatcagcaggacatcctcaccaccctcttgcagatgcccctttgcccatcctgaatgcacagcctccccggctttgcccttcagcccctgcttggctgtatgcactgtctgtattgcactgagaaagaagggacagcaggagtgagatggggagggagctggctcagcaccacccctcccagccctgccccactcgcctgagcttgctgccctctgagaggggaaaagggcggctgggagggctttccacctgatgtccctggtggaacaggtgcttctgggcgggccagcctctaatttgcacacctgaaatcgcggaattgagtttagatagattgattttttaaacactttttttttttttggagtaggtgtaggggaatcatttaatttaaatcataaagattcccctacccaaaccctgactcctgtgtacagatgctctttagagggaatcagaaaaatgccaagcctttttttctctttgaatgtgctgtctatttttataactgaact

SEQ ID NO: 58 - DNA, 1369 bp, Unknown Organism
  Description of Unknown Organism EMBL No. q9ulg1
tccctggcatccatagtctgggaccgctcctgggtctctaaaccctgtttgccacagtgtcatcactgaagaacagaacacagtgaatgccagagaagatggtggctgtaaggatgagaggctggagcaagcagagccacaggcagttatcccagcatttcctcctgtccagcacagttcacaccagggaagctttaggtcccaccagcaacagtagcagcagctcccactcaagggacttcccagaagccagtaaagagaccaggaaccacctcaatcagcaggacatcctcaccaccctcttgcagatgcccctttgcccatcctgaatgcacagcctccccggctttgcccttcagcccctgcttggctgtatgcactgtctgtattgcactgagaaagaagggacagcaggagtgagatggggagggagctggctcagcaccacccctcccagccctgccccactcgcctgagcttgctgccctctgagaggggaaaagggcggctgggagggctttccacctgatgtccctggtggaacaggtgcttctgggcgggccagcctctaatttgcacacctgaaatcgcggaattgagtttagatagattgattttttaaacactttttttttttttggagtaggtgtaggggaatcatttaatttaaatcataaagattcccctacccaaaccctgactcctgtgtacagatgctctttagagggaatcagaaaaatgccaagcctttttttctctttgaatgtgctgtctatttttataactgaacttgtacatatgtataaagagagacacatctttcttttactaactggataaaaaaaatcttaaataaaatggagtgagataattttatgtaaatcaaagtgtctgtggtctttgcagctgcctttgtactttgcagtacaaagttgcgcccttggagatttgacagcgtcaaaacaatccagagccctaatttctgtgaccaaagaggaccatagtgggacaaggagtgtgcggtgtgtgtgtgctcagggggacaaggagtgagctggggagggggtgctcaggtgcagtgttaatggaacttgcggatttgtctccacctttcaaaagaagattgaccaagtccctgccatgcagaatgggccctggggatgagggttccttatggttcagtcctagtagaactggcctggagcaggcaaagttagatagtggtaacctgaatacttgtaagaaacatgaaaggaaactgtgagtgagtccctaaggattccagatgttcttagttttgacagcttgactggtcggttgaggaaaggtaccagcctagactggcagtcactgttctgtgattgaactt

SEQ ID NO: 59 - DNA, 1408 bp, Unknown Organism
  Description of Unknown Organism EMBL No. q9vci9
ccagagtggaatgcggagggtgtccaaaacagcttctctgcagtgcgtgcttttaggcggggccccaaaggcaagaattcgtgccgcccaaagtattcctactggtcgtgccctgcactggccactgctttgggcggcaatgattctgccaggtgcaccggctggactgcagggatgacaccgtttagaccactgagagggccttcccgggagacggcgggtgcccgcgcccattctaaggcccgttcccggtgccagcagcctgacgcaatctgactcgcccttgctgcatgccagttctggggaaaccttccctgccgttgcatcactcctgccaggtctgccacgggaggaggagccgaaccaatcagcacgcgcgttggccctggggcgggctgctctggtacccactcggagggcggcgcggttaaccgagaagcaaaatctaaaggtgattggccagaaagagatttgccccgccccaatcatagggacagctggaggcagtctgagggccttcctggccggagggtttaaagagcgcctctggagggaccactcagctggaacgaccgatcggtgccaggccaggtgtacgcgtccgtcggtccttccgtgcccgtgccggagaccagtctcggaggccacccgggtccgtccctgcgcccggcaccatgaagcaggagtccgcatcccagagcactccgcctccttcactgtcccctgcaccatcatccgcgcagccttcctggggggatggcgacccccaagccctgtggattttcgggtacggctccctagtgtggaagccggactttgcctatagtgacagccgtgtgggcttcgttcgtggctatagccgacggttctggcagggagacaccttccataggggcagcgacaagatggtgagcatccttctgtgcccagaggagtggagtggggttggcaggagagtgggcactcgtatcctggagtggcagggaccctgggctaactaagagaaaattgtaaacctggtttgtggtttaaatttgtgtgcctggaaatcaggttggtgattgcacctgtggattcttgtgctggttactaatctgggccgctcgttgtttgtctcttccacatctgcccatcactccatgtcactcgatgaaaggtcctctcccaggcttggggcttaggctggcactctttcctcctcttccttctcttcctcttcttcctttccctcttcctcctcttcctcttctcttcctctccctcttcctccttgttcttctcttcctcttcctcctcctcctccttttcctcctcctcttctcccccctctttttccccctcctccatcttacctatttcttgctatgtggcctcaaacttgtgacaaatctctctctctggtgctgaa

SEQ ID NO: 60 - DNA, 1036 bp, Unknown Organism
  Description of Unknown Organism EMBL No. q9vci9
cccatcactccatgtcactcgatgaaaggtcctctcccaggcttggggcttaggctggcactctttcctcctcttccttctcttcctcttcttcctttccctcttcctcctcttcctcttctcttcctctccctcttcctccttgttcttctcttcctcttcctcctcctcctccttttcctcctcctcttctcccccctctttttccccctcctccatcttacctatttcttgctatgtggcctcaaacttgtgacaaatctctctctctggtgctgaagtgctggaactacaggttcaagtcacaacaggactaaaggtgtgtgtgtgtgtgtgtgtgtgtgtgtgtgtgtgtgtgtgtgtgtgtgtgttgctggagattgaatctaaggtcttgtgcatgacaggcaagccccccatgtcaataaggttcattcatagtcctttcggaagtgctaatatctaatttgtctttttcaaaacctttcttccctgtctagcctggccgagtggtgaccctccttgaagaccgtgaggtaagtgtgcagtcaagaagagaatcctggggtctgatgtatcagggtgcagagggtcagagctcttgtgtggggagagtggaagtgtagctagcttactccatgcttcttggagaatcacttgagggagggtcttattaagatggatttctgtgagttcagagttattcttggttacataatgaatcctgggtcagacaccctgtactatatgagacgtctcaaaataaacaaaactcaagggaaatagggtgaagggtattgcttctgggtggctcagcagccagtcaaacacgttagtttttttttaaagcttcatggagaaatggctcaggagtcagaagcaccttctgcttctccaaagggcctggatttgatacccagcacccacacagtaactcacaatctgtagctccagttccagggaatcagactcttctgactgaccaccaagatcacaggtcgccaggcacacacgtccatacacataaaacttcaaa

SEQ ID NO: 61 - DNA, 1450 bp, Unknown Organism
  Description of Unknown Organism EMBL No. q9vci9
ttagtttttttttaaagcttcatggagaaatggctcaggagtcagaagcaccttctgcttctccaaagggcctggatttgatacccagcacccacacagtaactcacaatctgtagctccagttccagggaatcagactcttctgactgaccaccaagatcacaggtcgccaggcacacacgtccatacacataaaacttcaaaattacatctatttgtacatgcacgcatgtgcacacgctgtaggtcagatgtggaagcaagacgacaagatggaagtcgattctttcctccgaccattcatgtcccagggattggacttaggttatctgatcatcaggcatggtagcaactacctctatcccttgagacgtctccccaccccctccacctcttttatagaacataggtgtggaatgtgtctaggggaacattctagaatgaacacaggaccagcatcaagctctgtgactgactggagtgtctctgtttttccctagggctgcacttggggtgtggcataccaagttcgaggggagcaggtgaacgaggccctgaagtacctgaatgtgagggaagccgtgcttggtggctatgacactaaggaagtcaccttttatcctcaagacacccctgaccaacccctcacagcactggcctatgtggccaccccacagaaccctggctacctgggccctgctcctgaagaggtcattgccacacagatccttgcttgccgaggcttctctggtcacaaccttgagtacttattgcgtttggcagacttcatgcagctctgtgggcctcaggcacaagatgagcacctggaagccattgtggacgccgtaggaaccctgctaccctgctcttacctacctgagcagcctctggcactgacctgaggagccaagctcctgcaagaagtgtctgagtggtactggtggacatcagtatgcagtacttgagatagacttgatggagcaagagagagttgagaaggcatgctgggtggccaggggctttgtgtcttatgccccgcctgtctgctagccttcagctcttccttcattgacccttactcactacttgaacctttatttattgcaccatgttggtgtggtgggcagggcaggtgggggacctgccctggatgtgggccctgcagtgcggtggctctgccctagtcctctggtatgtaaccagaatccccccattgctgctgccaattccacaccacccaggcctccgtagccccagggaccctccaagatcgttgctcctctgtccacccagactaccccagccgtggtagtatcacctgcccatgttccgggccttctggatgagggacagcagagaaggaggcgaagtagctcttcaagggccaagggtcagcatccctcttcattgacgggcccggggcagagccggatcctatgcacagacacag

SEQ ID NO: 62 - DNA, 1301 bp, Unknown Organism
  Description of Unknown Organism Chp, EMBL No. q9z1y0
ctttatatgctaggcaagcactctgagctacattctcagcttcaatctcagtatattaacaaaatagtttattagtaaaattactattctgatgtttagtacttgtacatatatatatatatatatatatatatatgtatatatatacatatatatatatgtgtgtgtgtgtgtgtgtgtgttactaacttagaagagatgaatgtgatgtgtttaatttgaacatgcagtattatggattctgtagctgcatcttcctactaacaataaattgtggatttaccaaaatatcatatgcccggtctttgtccttgtaccttcgtggagcctggttttcatattcctttcttttctggatgcttggttttttttctgggtgttctgcagagggaggctccaggtcggtgggcggggtccggggcactagggacatgcagatgaggtgggcgggacccaacttatataccttctgtcagcctctagcgggcgtgcccagcttggacagattgtcagcgtccggctgcggaacctgcttccccgagtgccgcggagcctggtccgggctggcccctctgctaccccaggagcggaccatgccgccgcgggagctaagcgaggccgagccaccgcctctcccggcctcgacccctcctccgcggcggcgcagcgcccctccggagctgggcatcaaatgcgtgctggtgggcgatggcgcggtgggcaagagcagcctcatcgtcagctacacctgcaatgggtaccccgcgcgctatcggcctacagcactagacaccttctccggtatgttctatagccccggggcccctgaggatgggaagggttgtgcattcagtagcctgctggcgggagggtacggaagaccccgggagcaggcccggaggtggcgctggtgaggcctgcaggagggacgaggatgctgtgcctaagtcatgctttcccctccccacctctgcagtgcaagtcctggtcgatggagcccctgtgcgaatcgagctttgggacaccgcagggcaggtgagaacctgggtgtagccttccttgggcaaggggttggagagggttggaggctgggaggatccctgcgtggagtgggaatggaataggctttgggctttagcacttttaggtctccacccccaagggagaagcagattctggttccaatgtggagggaatgatgttgctaacaactagccttgccaccccaggaggactttgaccggcttcgttctctctgctacccggacaccgatgtctttctggcttgcttcagcgtggtg

SEQ ID NO: 63 - DNA, 1059 bp, Unknown Organism
  Description of Unknown Organism Chp, EMBL No. q9z1y0
cctctagcgggcgtgcccagcttggacagattgtcagcgtccggctgcggaacctgcttccccgagtgccgcggagcctggtccgggctggcccctctgctaccccaggagcggaccatgccgccgcgggagctaagcgaggccgagccaccgcctctcccggcctcgacccctcctccgcggcggcgcagcgcccctccggagctgggcatcaaatgcgtgctggtgggcgatggcgcggtgggcaagagcagcctcatcgtcagctacacctgcaatgggtaccccgcgcgctatcggcctacagcactagacaccttctccggtatgttctatagccccggggcccctgaggatgggaagggttgtgcattcagtagcctgctggcgggagggtacggaagaccccgggagcaggcccggaggtggcgctggtgaggcctgcaggagggacgaggatgctgtgcctaagtcatgctttcccctccccacctctgcagtgcaagtcctggtcgatggagcccctgtgcgaatcgagctttgggacaccgcagggcaggtgagaacctgggtgtagccttccttgggcaaggggttggagagggttggaggctgggaggatccctgcgtggagtgggaatggaataggctttgggctttagcacttttaggtctccacccccaagggagaagcagattctggttccaatgtggagggaatgatgttgctaacaactagccttgccaccccaggaggactttgaccggcttcgttctctctgctacccggacaccgatgtctttctggcttgcttcagcgtggtgcagcccagctccttccaaaacataacagaaaaatggctgcctgagatccgcactcacaacccccaagcacccgtgttgctggtgggcactcaggctgacctaagggacgatgtcaatgtactaattcagttggaccaaggaggccgggagggcccagtaccacaaccccaagcccagggtctggctgagaagatccgcgcctgctgctaccttgagtgctcggccttgacgcag

SEQ ID NO: 64 - DNA, 1845 bp, Unknown Organism
  Description of Unknown Organism Chp, EMBL No. q9z1y0
gcctcatcgtcagctacacctgcaatgggtaccccgcgcgctatcggcctacagcactagacaccttctccggtatgttctatagccccggggcccctgaggatgggaagggttgtgcattcagtagcctgctggcgggagggtacggaagaccccgggagcaggcccggaggtggcgctggtgaggcctgcaggagggacgaggatgctgtgcctaagtcatgctttcccctccccacctctgcagtgcaagtcctggtcgatggagcccctgtgcgaatcgagctttgggacaccgcagggcaggtgagaacctgggtgtagccttccttgggcaaggggttggagagggttggaggctgggaggatccctgcgtggagtgggaatggaataggctttgggctttagcacttttaggtctccacccccaagggagaagcagattctggttccaatgtggagggaatgatgttgctaacaactagccttgccaccccaggaggactttgaccggcttcgttctctctgctacccggacaccgatgtctttctggcttgcttcagcgtggtgcagcccagctccttccaaaacataacagaaaaatggctgcctgagatccgcactcacaacccccaagcacccgtgttgctggtgggcactcaggctgacctaagggacgatgtcaatgtactaattcagttggaccaaggaggccgggagggcccagtaccacaaccccaagcccagggtctggctgagaagatccgcgcctgctgctaccttgagtgctcggccttgacgcagaagaacttgaaggaggtgttcgactcggccattctcagtgcgattgagcacaaagcccgtctggagaagaaactgaacgcaaaaggtgtgcgcacgctgtctcgctgtcgctggaagaagttcttctgctttgtgtgagcagctatggcaagagataggcgggtggcctgagacttctggaccccgagacatcgggtactggcagggcctggccaacccctggaactcagttctctattgaacacagggcacatgggcctcaaagctgtacaccctggtgagccagggtgagctctgtctgtgcaggggctgccccatttggatttctttggtcaagactcacaggaaaatcccagcactttgattttcatgggatagcgccatcagcgtcagtgctgctgagcagcttgggatgtaattctcagtttcttatcctggggccacaggtcagtttggctgaatgcaggcccctcggggtcatccccctctcctagcacagtgcgaggatcaaggagaacagtggggcatcttgcagggctggcggatggtcagcttgatttggagacgtttgagatggggtatgaagaagagggcagtgagagattgagagggaaaacagaaagtgaggcgagtggtggacaggaggcagattagaggcctgggcctggaaggaggaagacaggaagggagaagggtggacagctgtggagaaggccgacacctgggctgccctcgagccccaaggccagggaggacaggactgctccctgggaagaactgggtctcagggttccccaggcactggccaaactggctgggctagcagaggggcaggaagtgagagttcaggcccagcaaagggaggggaggagctgcaggtaggagcctgaaggaggaaggggcagggaccgtccctttccccaaggtcatagcttagaaggtgagctatacagtctgcaaataaaaccttccatttctcaagctgctgtctgtggtctcctggagtggtcagcccaatgccccttcataacctttgcca

SEQ ID NO: 65 - DNA, 1623 bp, Mus Musculus
gatttctgagttcgaggccagcctggtctacagagtgagttccaggacagccagggctatacagagaaacccttgtctcaaaaattaaaaaaattaaaaaatttgggggctgttgagatatctggctaagtgggtaagagtacccaaatgctcttccgaaggtcctgagttcaaatcccagcaaccacatggtgactcacaaccatccctaatgagatctgactccctctcttctggagtgtctgaagacagctacaactcacatataataaataaataaatcttaaaaaacaaaacaaaacaaaaaaactgaaaaaacaaacaaaaaaacaaacaaacaaaaaaacaggtgtagtagtacacgtctgtaatcccaacatttggaaggcagagacaagagaattatgatctcaaggccagcctcaactaaatgaaacctgtgtcaaaaaaaaaaaaaaaaaagacttcaaagatgcattatcttgagcgccctcgccaccgcatctcttctgcttttgtgctctcccgcctgccatggccaaccttaaggatctcaaagggaagtggtgcctgatggaaagccacggctttgaggagtacacgaaagagctaggagtaggactggctcttaggaagatggctgccatggccaagccagactgtatcattacttgtgatggcaacaacatcaccgtcaaaaccgtgagcacagtgaagacgaccctgttctcttgtaccctgggagagaagtttgatgaaactacagctgatggcagaaaaactgagacggtctgcaccttcaaagacggtgcctggtccagcaccagcaacgggaagggaaggagaacatgataatgagaaaactgaaggatgattgtggtgcactcgggtctatgagaaggtgcaatgagggcttcctcgtcatcctggacaggagttagctgtaggagtgaatatgctcaattcaattagtggtcataaacagcaaaccgtttcacttctttggttttatttttcatgactgttgagttctctttatcacaaacactttacatggaacttcatgtcaaacttggtttacccaggatcatccctttggttagtaaataaacgtgtttgtgcttaaaaaaaaaaaaaaaaaaaagcattatcttgaggctggaaatcttgttcaatgatcatagatgatccccggcatacacgtgctgactcacaaccattcataactccagttccaggggaatataacaccttcttttgacctctgagagcaccaggcacatacattcagacaaagcagtcaatacacacattttaaaaataaaccacaccgagcggtggtggcacatgcctttaatcccagcacttgggtagcagaggcaggtggattttcaaggccagcctggtctatagagtgagttccaggacagccagggctacacagagaaaccctgtctcgaaaaaccaaagtaaataaataaataaaccaaaataagaagaggggaggggaggaaacagcaagaaggaagaaagcaaaggaggaagaaaggaaggaaggaaagagaagagaaactggagaggaagaggaagaacaggaggaagaggaaaaggagagcagcagca

SEQ ID NO: 66 - DNA, 2002 bp, Unknown Organism
  Description of Unknown Organism Mga, EMBL No. q9qxj5
ttcacttttttgtaaggttcagggattgaacttggatcaactggcttgcctgatctgctgacccatctctctgacctctactaagtttaagtagggttgcttgcataagcatgggtaggtgattatttactggaacatgggcaagtttatctgtgcctgtaccactgactggctttgaacttgtgctctttctccatctcccaagtgtgtagagtacaggtctgtgccactactcctggtatacgcagggctggaaatacccagggctttatgtgtgctaggcaaatctctaccagatgagttacagttgtatttttgttagaaaaattctgagttgactaaagttctgttttttgttcgtttgtttgttttttaggatgaaaaagcatttgtaactatgaattgactacagttttcttactactgaatttctcactggaatcatggaagagaaacagcaaatcatattggccaatcaagatggagggacagtgacaggaggagcacctaccttctttgtcatcctaaagcagccaggaaatggcaaaactgatcaaggaattttagttactaatcgagatgcccgtgctttgttgagtagggagtcatcaccaggaaaatctaaagagaaaatttgccttccggctgattgtactgtgggaaaaatcacggttacccttgataacaatagtatgtggaatgagttccataatcgaagcacagagatgattctgaccaaacagggaagacgcatgtttccttattgtcgatattggataacaggcttagattcaaatttaaagtatatccttgtaatggacatatctcctgtggatagtcatcggtataagtggaatggtcgttggtgggaacccagtgggaaggctgaaccccatattttggggagagttttcattcatccagaatctccttctacaggtcattattggatgcatcaaccagtgtctttctataaactcaaacttactaacaatacactggaccaggaaggacatattatcttgcactctatgcatcgttatctgccaaggcttcatttggtgccagcagaaaaggctacagaagtgatacagttaaatggccccggtgttcatacttttactttcccccagactgaattctttgcagtaacagcttatcagaacattcagattactcagttgaaaatagattacaatccttttgccaaaggctttcgggatgatgggttgagcagtaagcctcagagagaaggaaaacagaggaatagctccgatcaagaagggaatagtgtttctagttctcctgctcatcgtgttcgtcttacagaaggtgaagggtcagagatacattcaggtgattttgatcctgtgttaaggggtcatgaagcatcaagcttaagtttagagaaggctccccataatgtaaaacaagactttcttggatttatgaatactgattcgacacatgaagttcctcagttgaaacatgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgtattgctcattctcctgatagttgaatagttgaatagtttctgtagattcctttgacaattgctgcatagttttattttgtttgataaggaaaaaataatgaggttaaaaaattgttaagatatgtgtatgctctaaaatgtgatcttttgaggaagtttcatatgtatttctgagctattaggtggaatattcacatatatgtattaaattcatttctttgtggtagtttaagtttcttaattgtttgtttggattacatttactggtgagactgcagtactttagtttttactcatcatcattg

SEQ ID NO: 67 - DNA, 1853 bp, Unknown Organism
  Description of Unknown Organism Mga, EMBL No. q9qxj5
ctttttttccctcttattagcatttgatgatgcataagttttaagactttgattttttttggttggttatagctgacttttagtttggagttctttatatgtgatcaatatctaaagacaaacacattttataaggcactctgcattttgatttaatgataagggtaaactattcttttcctgcattttatttatacaagtggggttagtttatgtagtgttagagattgaatttaggacttttgtgttctaggcaagcagtatatcagttgagctacatgcccagccctgaaacttggctcttgaattgtatttttttgtgtgtgaaacattgataaacatgtgatgttgataatttacttttgagcactttgttaagctataatttttaagtaatgactcatgaaatctgatggatatattgattttaagaactgtttttccttctctgagtcttgttgatagatagttctgtactttttattgttttctacctttctagtcgtattgtgaacagttttgaagatgattcccagatttcctcaccatcaaacccgaatggaaactttaatgtcgtcattaaagaagagcctctagatgattatgattatgaacttggtgaatgcccagaagggataacagtgaaacaagaagagacagatgaggaaacggatgtatactcaaacagcgatgacgatcctatactagagaaacaactaaagaggcacaataaagttgataatttagaagctgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnattgtacaaaaatatacttaacttaaagagcctaattggaaatacccggacatacttgacaacagtagcacgaaaagaatacatgacagttccaaaggatcaactgcagagtcattttcaggaaaagaggacttaggcaaaaagcgaacaacaatgcttaaaatggcaataccatcaaagactgtgactgctagtcatagtgcctctccaaatactcctgggaaaagaggaagaccgagaaagttgagactctctaaggcagggcgaccacctaaaaacacagggaaatctttaactgctgccaagaatattcctgtaggccctggaagcacctttcatgatgtgaaacctgatctagaagatgtggatggggtgctctttgtttcctttgaatcaaaggtatggttgtaatgatcagctttctttttaaggtctgctaatcagttttgaattgaaaattggcttaaaatttaggacctacttttattaatgtatctatttgtgagtatgtttggggctattgagtgagtgttaaacatttggtttaattaacagtaatcatctcttaagatacttgctttgaaatagactttaaagaaatgtacctttgtgtagatctcattctataatttgttcaatgtttatctgattaaaaaagatttatgtgtgtctatgtatgtgagtgcaggtgccagtggaggccagaggtgtcaggttgtcctggagctactagagttgaagctacttgtgaactataaataacatcagtgttagaaaattgagcttgagggctggtgagatggccccgttgagagcactggctgttcttccagaggtcatgagatcagttcccagcaaccacatggtgactcacaaccatttgtagggatatctgatgc

SEQ ID NO: 68 - DNA, 1079 bp, Unknown Organism
  Description of Unknown Organism Mga, EMBL No. q9qxj5
actcctattctgtgaatttggagcaattgctaaaacatggtattttatactcttactcctatattattaatttaaaaatattctttatatgagtggcagatgtatgcatatccttgggtgtcagtgtgtatacctatgtgagtgtgtgcagaggctagagattggaacttccttctttgttcactcagtctaattctttgagacagggtatcttcactgaacttggagctcactgattgcttaggctaaccagtggatttcagagagtctcctccttcatcctaaccaccaccaggagttacagaaaagtgatacgatacctagcttttacatggatactggggataaaccagcttctcatgatcagacagcatgccctgtagctactgagcatgctctccagccactcgtcattgtcatcctttttttttcttttaaaaattgaatttcctttgaaaattttagaagcaacataactgggttttatttttaatgtataggaggctcttgatattcatgcagttgatgggacaacagaagaaccttctagtcttcagaccacaaccacaaatgattcaggtattgtctaacagtataaatttagatgtatttgctgttgggcctatatagcatggaatatcctctctgatgtacattattttatttcatcactgttttgtgagtaggttgcagaacaagaatttcccagttggaaaaggaattaatagaagatttgaagtctttgaggcataagcaggtgatacatcctgctcttcaagaaggtactagtttcaaaatgtggaaaagcccatttgtcttttgtacattttgtcttccccttttctttcctacctctttttgcttatttctccctttattttctctttcaattagcagttttttattttttccacttctaaagttcttttcaattttaaaaacacatctaggtctttttttgtttgcttttcgagacagacagggtttcgacagctgtagccctggtcattctgtacaccaggctggtggcctcgaactcagaaatctgcctgcctctgcctcccacgcactgggattaa

SEQ ID NO: 69 - DNA, 1096 bp, Unknown Organism
  Description of Unknown Organism Mga, EMBL No. q9qxj5
agtctaattctttgagacagggtatcttcactgaacttggagctcactgattgcttaggctaaccagtggatttcagagagtctcctccttcatcctaaccaccaccaggagttacagaaaagtgatacgatacctagcttttacatggatactggggataaaccagcttctcatgatcagacagcatgccctgtagctactgagcatgctctccagccactcgtcattgtcatcctttttttttcttttaaaaattgaatttcctttgaaaattttagaagcaacataactgggttttatttttaatgtataggaggctcttgatattcatgcagttgatgggacaacagaagaaccttctagtcttcagaccacaaccacaaatgattcaggtattgtctaacagtataaatttagatgtatttgctgttgggcctatatagcatggaatatcctctctgatgtacattattttatttcatcactgttttgtgagtaggttgcagaacaagaatttcccagttggaaaaggaattaatagaagatttgaagtctttgaggcataagcaggtgatacatcctgctcttcaagaaggtactagtttcaaaatgtggaaaagcccatttgtcttttgtacattttgtcttccccttttctttcctacctctttttgcttatttctccctttattttctctttcaattagcagttttttattttttccacttctaaagttcttttcaattttaaaaacacatctaggtctttttttgtttgcttttcgagacagacagggtttcgacagctgtagccctggtcattctgtacaccaggctggtggcctcgaactcagaaatctgcctgcctctgcctcccacgcactgggattaaaggtgtgcaccaccaaataatatatttaaggatacacacacttttcttggggctgaagagatggcttagtagatgaagtttgcttgatgtgaagtatatgagtatcttactttgatccccagcacacataaaaatgctaggtagagcagtacatgcctgcagtcttggtgcttggaaaaaccaaattaggatgttctaggttc

SEQ ID NO: 70 - DNA, 1132 bp, Unknown Organism
  Description of Unknown Organism Mga, EMBL No. q9qxj5
ctcatgccgactttaggagatgcccgttattgtttttagtggtaacctagtacagtattactctatataacgtctaagctgtctttgctcacctccccctttaatccagtgaaatattttcccaaaatgttgtctcttaatttataagtaatttcattttaggttcattgataataactggagaatataatcaggtcagaaactccacctgtggcttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaactgagttttttgtttgttttgctttttgaagaaggaaggggggaaaatatgaattgtggtccttttttcttgtagtgggcttaaaactgaattcagtggatccaacagtgagcattgatcttaaatacttgggagtacaattgcctttggctccagccaccagctttcccctatggaatgttacaggtaccaaccctgcctcacctggtgagtatgtagatagcatttgtataagtgaacattatttttcttctttaagtttaggttatttgtagtacaaattttaaaacattacttttatgttccaatttagggtatttagagaataaatctttcatctcttggcatagaattaatacatggatgtattcatagtagagaaagagtaatttgacatatattggttggctagaactttggcaatttctttttctttttcttttatagatgctgggtttccttttgtttctaggacggggaagaccaatgactttaccaaaatcaagggatggagaggaaaatttcaaaatgcttctgcatctaggaatgaaggtagctagatgtttggtttttgattgtctttttgttttttgttttgttagtaccacagattcagagtagttcaaacagcttttccagcactttcctattcacctcaagcccaactccttttctgtttaaaatgcagtttcattgagtgtagaatg

SEQ ID NO: 71 - DNA, 1045 bp, Unknown Organism
  Description of Unknown Organism Mga, EMBL No. q9qxj5
ttttgtttgttttgctttttgaagaaggaaggggggaaaatatgaattgtggtccttttttcttgtagtgggcttaaaactgaattcagtggatccaacagtgagcattgatcttaaatacttgggagtacaattgcctttggctccagccaccagctttcccctatggaatgttacaggtaccaaccctgcctcacctggtgagtatgtagatagcatttgtataagtgaacattatttttcttctttaagtttaggttatttgtagtacaaattttaaaacattacttttatgttccaatttagggtatttagagaataaatctttcatctcttggcatagaattaatacatggatgtattcatagtagagaaagagtaatttgacatatattggttggctagaactttggcaatttctttttctttttcttttatagatgctgggtttccttttgtttctaggacggggaagaccaatgactttaccaaaatcaagggatggagaggaaaatttcaaaatgcttctgcatctaggaatgaaggtagctagatgtttggtttttgattgtctttttgttttttgttttgttagtaccacagattcagagtagttcaaacagcttttccagcactttcctattcacctcaagcccaactccttttctgtttaaaatgcagtttcattgagtgtagaatgctggccttgagtttccagttctcctgctcagccttccaagtgttggaattacaggcatatgccaccaaagcctggctggtcactagtgtttgttttgcttttaaagtctatgtatgttttgaaaaatgtattcttggcctggggtagtagcacacttgccttttatctcaacactttggaggcagaggcaggtatatttctgtgagtttgagtccattctgagtacatagcaagttccaggccagccagggttacatagtgaggtgagaccctctttgaaatcaagttagtctcattgaaacacttacccttttcatctctctaaactgaactaaaaacagtgta

SEQ ID NO: 72 - DNA, 1659 bp, Unknown Organism
  Description of Unknown Organism Mga, EMBL No. q9qxj5
ccattctagttcattcacttgggaggcaaacagagacaaataaaggagaattgtaagtctggcctagttcctacagagctgaatcgccagattcaagacatctagggctacttagtgagaacttgtttcaaaacaaaaccagataaatgaaaatccttaaaggttgtgctctcctttttatggcttcctctctttccttgtatcatgtttcagactctagggactcaagtctcttaaataaccccttggcattttgttgatttttttttttttttctttttccctggctgttagtttttgctcatcactgtttactatagttaggtgaaaaggtctgaaggcttgtatctgagacactgtctcattttgttttggctttctgttttttttcttctttttgagtaggaacagtttagagaagcttcagtaggatatagctcagttcttagagagctaagacccttcaaggtttaaatgttttgtatctccttattttctaggtggaaattcagaggcttcactgaaaaaccgttctgctttctgtagtgataagctagatgagtacttggaaaatgaaggcaaattgatggaaacaaacataggtttctcttcaaatgctcccacatctccagtagtgtaccagctacccaccaagagtaccagctatgttcgaactcttgatagtgtactaaagaagcaatctaccatttccccttctacctctcactctgtgaagcctcagtctgtaaccactgcctctcgaaaaacaaaggctcagaacaaacagacaacactcagtggccgaactaaatcatcttataagtctattttaccataccctgtttcaccaaagcagaaaaactctcatgtgagccaaggagataaaattaccaagaattccttgagttcgacctcagataatcaggtgactaacttggttgtgccatctgtagatgaaaatgcatttccaaagcagattagtttgcggcaggcccagcaacagcaccttcagcagcaaggaactcgccctccaggcttgtcgaaatctcaagtaaagcttatggacctggaagactgtgcactctgggaaggaaaaccaaggacctatattactgaagagcgagcagatgtctccttgacaactctgcttactgctcaggtaaatattgctattttctgtaaacattggtgcttagctacaaattagacattggtactttggtaagtggataagttagagtttagcatttttttaagatttatttattatatataagtacactgtagctgtctttagacataccagtagagggtgtcagatctcattacagatggttgtgagctatcttgtggttgctgggatttgaactcaggacctttggaagagcagtcaatgctcttaaccgctgagccatctctccagccccagggtttagcattttaattaactaggtaacaattaaggggatgtcttcagcaatgagctaatcttttgagtctagctttcttctgccagtaaagggagactgactgactgactgaatgaatgaatgaatgaatgaatgaatgaatgaaaaattctttactgaattgcttgctatctaagcatgggaacttgaatttagatttatagcactgacatacaaaccaggtgagatg

SEQ ID NO: 73 - DNA, 1339 bp, Unknown Organism
  Description of Unknown Organism Mga, EMBL No. q9qxj5
cctgtactgtattttctagtcattataaagattcaagcatgtatgattctcttatttttattgtttacttaggacattttgtgtaaaatagtagtaattattttgcattttgcggaccatatatgctatcagagagttattactaggatgttctattctaggcagtatgagcttgcttgtgcttcaggaaaatcgtatgagacattgtttgacctatgtaccttagactcatcagtccctacagaagaacattatatgtggtttctattgagtgaagagaatatttggtgtttttgctcttaataagactcctttgctaatgcattagagtggcagcttctttacgcagagccctcattatatgtgtttgacttttgaattctgtgttagtcttgtgttattagtactagtaatcagaaggtagatgagtatgtgactcttaagttggtggtagtattgggatgatttagtaaaattttaatgttctcctttaactttaggcatctctcaaaactaaacctatccacacaatcataaggaaacgagctcctccatgcaacaatgacttctgtcgcctgggttgtgtgtgctctagtctagctttggaaaagcgccaacctgctcactgccgtcgaccagactgcatgtttggttgcacttgtttgaaaagaaaagttgtgttggttaaagggggatctaaaacaaagcatttccataagaaggctgctaatcgagatccattattttatgatacgctgggagaggaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaattccatgctgataattggacaattcaaatgacaacagtccaattcattgggagacatgtaactcatagcaatagaagaaccgatgggaacagttgtgtatgccattaatcctggcactcagtagctaaggttgaatatctagagttcaaggccagtatgggctacatagtgagactcaaccctaccgcagaaaactataatttcaacttgctaaagaattatgctaataacaaaagcacctgtaataatcatcaggggttgcatgaaatagtaaaaaaagaactagcacgttcctgcactagtgtgggcaactggagggccacagagctcctgccagctgacctcactgggatgttcctctgcagtgat

SEQ ID NO: 74 - DNA, 1228 bp, Unknown Organism
  Description of Unknown Organism Mga, EMBL No. q9qxj5
atgtataagcaacagacagttgaaagttgaaataaaaattctcatttaaaataacatgtaaggattggtggaattgtttagccagtaaagtttgatccctggagttcacatgatagcgagaacctactcttaaaagttgtcctctgacttacatacatgttctgttatacacaggtgtgccccaccccaatttaaaatattagctaacgagatgaggcatgataaatttaacaaaaatatctaagatatatgtacgtatgtatacatgcacatactttgttttattcttagctattctcttggttggagaataggtactcaacaaatatttgattaatagatgaataaattaatgcatctgaagacaactaaaggtcttttagggatcacctgattattacaaattactctctttgtgagggaacataccttgttttagcattgctcagtgctgaacttatattgccaaaaacaccttaaatcttggccttttcttttgttaagctgtatgtgaagcagaacctgagcagccggttcgacattacccactgtgggtgaaggtagaaggtgaagtagatccagagccggtttatattccaactccttctgtcattgaacctataaaaccattggtgttgcctcagccggatttatcttctactacgaagggcaaactaacccctggaattaaaccagcacgaacatatactcccaagcccaatcctgtggtaagcctgtttgttgttgtttttattgtaagttcataagccatttgtaaaggtgataggtttcaagtagagcttttgttaatattctgtcaagattttagacagtgtatttttaagacctttcctctctgactcagtttatttgctctcactttaacaatgtgataaaagtgaagatgcagagtaataattgaagatagccaagtttttcttcaagttcatagaaagtgaactttttcataatttgtttatatttgtgtatgtgtgtttgcctctgtctatgtaaatgccctcagagttcagatgtggttgtgggatcccctggagcttgagtcagacacagttgggaggcagagacagaggtgaatctctgagttcaagaggtctagcctagcctactattgggagttccaggatagcaagggttatacagagagatcctatattgaaaaacaaaatgaaaacacatcttttgtaatagaaacaaattaaattcttcc

SEQ ID NO: 75 - DNA, 1183 bp, Unknown Organism
  Description of Unknown Organism Mga, EMBL No. q9qxj5
gagcttttgttaatattctgtcaagattttagacagtgtatttttaagacctttcctctctgactcagtttatttgctctcactttaacaatgtgataaaagtgaagatgcagagtaataattgaagatagccaagtttttcttcaagttcatagaaagtgaactttttcataatttgtttatatttgtgtatgtgtgtttgcctctgtctatgtaaatgccctcagagttcagatgtggttgtgggatcccctggagcttgagtcagacacagttgggaggcagagacagaggtgaatctctgagttcaagaggtctagcctagcctactattgggagttccaggatagcaagggttatacagagagatcctatattgaaaaacaaaatgaaaacacatcttttgtaatagaaacaaattaaattcttcctttggattccaggataagttttttccttctatcattataatggtctgttttgttatttgaaacttacagatacgagaagaggacaaagatccagtctatttgtactttgaaagtatgatgacttgtgcccgagttcgagtatatgaacgaaagaaagaggaacagagacaactgtctccacccctgtctccatcctcatcatttcagcagcagagttcctgttactctagtcctgagaaccgtgttacaaaggttaattgcttgtttcataataaccttctaatttccagttccccgtgtctggtattagaatttttattactatcaccgtaatcatctttgtgtttcctataatgtataaaaaaaaccatttctccacatatttaagtaatctatcagtgtttgttactaaaatcctgataaaatgtatcatgtaaattattttctaagtaactcagtcatttaagaacgttgaaggtaagaagactacattttatttatctacttttgggggcacagggttgtataatattaatacaactggacagatataaagttgtagataattttactaatttatgtcctctgtagataataaatacttttttaattaatttctgtattgcttccttctttaggaacttgattctgaacagaccttaaagcagctcatttgtgacttggaggatgattctgataaatcacaaggtcagaataaaaacagataataacagttgatagtagctattatg

SEQ ID NO: 76 - DNA, 1070 bp, Unknown Organism
  Description of Unknown Organism Mga, EMBL No. q9qxj5
gtatatgaacgaaagaaagaggaacagagacaactgtctccacccctgtctccatcctcatcatttcagcagcagagttcctgttactctagtcctgagaaccgtgttacaaaggttaattgcttgtttcataataaccttctaatttccagttccccgtgtctggtattagaatttttattactatcaccgtaatcatctttgtgtttcctataatgtataaaaaaaaccatttctccacatatttaagtaatctatcagtgtttgttactaaaatcctgataaaatgtatcatgtaaattattttctaagtaactcagtcatttaagaacgttgaaggtaagaagactacattttatttatctacttttgggggcacagggttgtataatattaatacaactggacagatataaagttgtagataattttactaatttatgtcctctgtagataataaatacttttttaattaatttctgtattgcttccttctttaggaacttgattctgaacagaccttaaagcagctcatttgtgacttggaggatgattctgataaatcacaaggtcagaataaaaacagataataacagttgatagtagctattatgtaattctgatttttttttttcttttttctttttttttggtttttcaagacagggtttctctgtatagccctggctgtcctggtactcacttgtagaccaggctggcctcgatctcagaaatccccctgcctctgcctcccgagtgctgggatcaaaggcctgcgccaccatgcccggcttaattctgaattatttagtggcaaaaaatttaaaccgctgatccataattgacactgtgtattttctaacctctgcaaaaaatggagcgagcagaagcattttcttatcatttgctttaatatccataggtctgcctgttttgatcaggtatttcatttcagacaacataagaatcatctcagcaaaaaagttttctcttttcaagactcaaggataattatttattaaaatttaaccttaagtcttccatagccaccacaatctccaaatttcttt

SEQ ID NO: 77 - DNA, 1518 bp, Unknown Organism
  Description of Unknown Organism Mga, EMBL No. q9qxj5
tgttgagccatctctccagcccagaacaataacttctgtcaagtaaaatttatcatagttataggtcacactttgcaaggcagtactttttaatgtttttgagagaagtactaaggaaatttcctttttatgacataatttagtctgtgggatagtgaagacaagtgtctagttctgtgaattttttttcctagaaaactctagaatgttgagtttgcacactaatttaacactgaaatgagacaatgagaaccttgggtttctttcaaagttcagtagcatgcctttaatcccagtacttgggaggcagaggtaagtggttctctgtgattttgaggccagcctgatttacatagttccaagacagccagggcttttattcagagaacccctgttttgagaaaaagaaagaaagaaaagacccccccccccatcaaagtttagtacagaaagtctgtgatctgtgatgtagtgggtctttctatttattttttaaacagaaaagagctggaaatcctcctgcaatgaaggagagtcctcttctacctcgtatgtacatcagaggtcacctggtggtcctaccaaattgatagagatcatctcagactgcaactgggaagaagatcgaaacaagatcttgagcattttatctcagcacatcaatagcaacatgccacagtcacttaaggtgggcagcttcatcattgagttggcttctcagcggaagtgtcggggtgagaagacccctcctgtttattcttctcgtgtgaaaatctctatgccatcaagtcaagaccaagatgatatggctgagaaatctggatcagagactcctgatggtccattgtcccctgggaaaatggatgatatctctcctgtgcagacagatgccctggattcagtgagggagagattacatggaggcaaaggtctacctttttatgcagggctttctccttcggggaagcttgtggcctataaacgtaaacccagttcaactacatctgggcttatccaggtgagaattatatttaatctgggcatagtacctttttatacttagctaacattgtgattttttttaattatttttattttttattaaacactcaagcccctttgttgaccttaggtcatttctttttttttttttttttttgagaactgggtaataaaaaaccatcggtaattcttattatgataaatgtaggttaggtgaatacagaaggcaaactactgttacatcaacagagatatggagccttacattagcttatggttttctttctattcttttcagaaagttcctttttataagctttactgcacgggctctagtgatcctaagtaagcaaaaataggcatttaatttgggtcttagaatatcttactagcataaaaatttcaaaacaaatatatagactactaggtgtagtagagataaattgtctttataggtcagtgcctttaatcccagcacttgagagcagaggcaggtggatcttttgagttcaggac

SEQ ID NO: 78 - DNA, 1148 bp, Unknown Organism
  Description of Unknown Organism Mga, EMBL No. q9qxj5
atggttatcctttttgctggtgattcattaataaccacagttgcttattaaagtaaaacaattagatgaattgttggatccagttatgtcttttacatatctataatgcagaaatgagtttagtttctaagattatctatttccagactataaatgaagttcaagttagttcagatatcagatgataccatccagttattgagattatcctgattagaaagtcttcacttttccccttttacatctaaagcatagactttaaaacaccttataaaaatgaaacccctgtcaagggagagaagttagcaagagaattttatctttacagaatttctctttgccacttgtttcaggttaatggtaagagttacccgcaggctaagttgctattgggacagatgggggcattgcatccagccaataggctagctgcttatatcacaggcaggttgcgaccctcagtcctggacctctcaaccctcagtactgtcatctccaaggtagcatccaatgccaaggtggctgcatccaggaaaccacgcaccctgttgccttcaacatccaattccaaaatggcatcctctggcccagcaacaaatcgctctgggaagaatctgaaggcatttgttccagcaaaaaggccaattggtaagtaagagaatgcatatggtttggaaaggcctgtaatttaatgttggccagtgaatacaatatgaaagttataaaagtggcaaaatagatttttgtgttattattaatgccctttcttaaatattctaaaaataattcttctgatgacctatgatatctgatgaattgaaattaagttttacagtcaaaagactttgagactgtattaacatattaaaaccaaggttttattttaaaatgtttcctttacttttcctatgatgattgaggtttttcataccaatcataagcatattccttgtacttaacaaatctgagacactaacaagagctagtagaatgggttagtataggttaaaaataaaaagctcttgtaaaacaaaaacttttagtatatttctgtcttgtgtatgaaaacttcttttgcctaacactttacagtctatattaaattgccttcttgaaacattgttttagttgaaagttatacagttagt

SEQ ID NO: 79 - DNA, 1624 bp, Unknown Organism
  Description of Unknown Organism Mga, EMBL No. q9qxj5
cagtttgagggcagcctggcttaccaagtgagttccaggccaaccaggtttatagagagaccctgtgtcggaaaacaaaaaccaaacaatcgaaaagtagaattttaaaactttgctttgggtggaggacagtgtttgagttattttgtgtgtgtgtgtgtgtgtgtgtgtgtgtgtgtgtgtgtgtgtgtagcaattggtacttataggtggattgtacctttaagaaattgacccaggagccgtggattaaagtgccacctttaatcctagcacttgggggtcagaggcaggcagatttctgagtttgaggccagcctggtctacagaagtgagttccaggacagccagggctatacagagaaaccctgtctcgaaccccccctcacccctcaaagaaattaacccagtgggcacaactaatgctataagcacaatcatttagtaggagggcacagtgagagaatgaagattttttacctgcaaatttttatttgcagcagcgcggccctctcctggtggtgtgttcacacagtttgtgatgagcaaagttggtgccttacagcagaagatacccggagttcgcacacctcaaccccttacagggccacagaagttcagtatcaggccttctccagtaatggtcgtcacacctgtggtttcttctgagcaagttcaggtgtgcagcactgtagctgctgctgtcactacttcccctcaagtgtttctagaaaatgttactgctgtgccatctctgactgctaattctgatatgggagctaaagaagctacatattcttctagtgcctccactgctggggttgttgagatctctgaaactaataacactacccttgtaacatctacccagtctacagccactgtgaaccttaccaaaactactgggataactactagccctgtggcttcagtttcatttgctaaaccattggtagcatctccaaccataactcttcctgttgcttccactgcctccacctccatagtcatggtgaccacagctgcatcttcctccgtggtgaccacacctacttcatcactgagttctgtacctattatactctcaggaattaatggaagtccacccgtgagccagagaccaggtaaggactagttcttttcagctgctttattataaataaagctccttaattaaattttacagttgtaataaaattaggagtttctagtttagacattgtcatctgtaaatgcattgggctgcattcatagctatcttgagatgtatgtaggccacaatttatagatatgcctgaatacattttagagtttcagccatagtatgacatgttaacattcctaaatctttaaaattctacaaaactgaaccctaaaaataacagcttatggctagttgcaagattaggaatgaccactgtctatacagttctgaagccagattccagagtacagggatcagctgtcttcaagagattatctgattgatagattaataggactaaagttgaatccaaggaatactaaaactcgaaaacaaagtggaaatgatcgctgaaggacactaaatgatgtaaataggaccagacctctgagcattagaacagccattaaagtggcatgg

SEQ ID NO: 80 - DNA, 1076 bp, Unknown Organism
  Description of Unknown Organism Mga, EMBL No. q9qxj5
cctttgattcatatacgcataagatacacacaaaaataaatgtattgtgatttgtttgtttgttgagacagggtttctctgtataggcctggctatgctaaaagttgcattgtagaccagactagcctctaaactcatagatttgcccatcttgcctcccatattctgggattaaaagtgtggatcaccattggcaaatgtgatgacaacaataatgagccaaggcattttctttttagtttgaagccaacctcttttccatagcgagttctaggatagtcagggctctataaagagatcttgtctcaaaaaataaagcaaattatagattattcttagctatgaatagtatatactataaagggaaagtgtttcattaactttataaattcaagtaagacgttacttgcagcattattaggtcttatgtcatattatttttacccatgtacttttaaagataaattctcatagtagcttctcatttttctgttttccagaaaatgcccctcaaattccagtgactactccacagatctcttctaacaacgtgaaacgtactggacctcgattgttgttgattccagtgcagcagggttctcctacgcttagaccaatccaaaacccacagcttcagggacagcggatggtcttgcaacctgttaggggtccaagtggaatgaacctattcaggcaccccaatgggcagattgtccaactcctacctttacatcagattcgaggctctaatgcccagcccagcttacagcctgtggtatttcggaacccaggtataaagttagagatctttctgatgagtttttttttttttaaagatttacatatcttgtaactacactgtagctgtctttagacactccagaagagggcgccagatttcgttatggatggttgtgagctaccatgtggttgtgggatttgaactcaggacctctggaagagcagttagtgctcttaaccgctgagccatctctccagcccctctgatgagttttgttgttaaatcacttgactatgtgatttgtttgatatgccagaatttttttttcagtctc

SEQ ID NO: 81 - DNA, 1098 bp, Unknown Organism
  Description of Unknown Organism Mga, EMBL No. q9qxj5
gcaaatgtgatgacaacaataatgagccaaggcattttctttttagtttgaagccaacctcttttccatagcgagttctaggatagtcagggctctataaagagatcttgtctcaaaaaataaagcaaattatagattattcttagctatgaatagtatatactataaagggaaagtgtttcattaactttataaattcaagtaagacgttacttgcagcattattaggtcttatgtcatattatttttacccatgtacttttaaagataaattctcatagtagcttctcatttttctgttttccagaaaatgcccctcaaattccagtgactactccacagatctcttctaacaacgtgaaacgtactggacctcgattgttgttgattccagtgcagcagggttctcctacgcttagaccaatccaaaacccacagcttcagggacagcggatggtcttgcaacctgttaggggtccaagtggaatgaacctattcaggcaccccaatgggcagattgtccaactcctacctttacatcagattcgaggctctaatgcccagcccagcttacagcctgtggtatttcggaacccaggtataaagttagagatctttctgatgagtttttttttttttaaagatttacatatcttgtaactacactgtagctgtctttagacactccagaagagggcgccagatttcgttatggatggttgtgagctaccatgtggttgtgggatttgaactcaggacctctggaagagcagttagtgctcttaaccgctgagccatctctccagcccctctgatgagttttgttgttaaatcacttgactatgtgatttgtttgatatgccagaatttttttttcagtctcctaaagtgggagacataccttgaaaagacttgatggtatgaacagtagtgatttaagctcttcctttttctttccaggatctatggtgggaatccgactaccagctccttgcaaatcttcagagactccatcatcttctgcttcatcctctgccttctctgtcatgagtcctgtgattcaggctgttgggtcttctccaacagtaaatgtcattt

SEQ ID NO: 82 - DNA, 2488 bp, Unknown Organism
  Description of Unknown Organism Mga, EMBL No. q9qxj5
tgcaacctgttaggggtccaagtggaatgaacctattcaggcaccccaatgggcagattgtccaactcctacctttacatcagattcgaggctctaatgcccagcccagcttacagcctgtggtatttcggaacccaggtataaagttagagatctttctgatgagtttttttttttttaaagatttacatatcttgtaactacactgtagctgtctttagacactccagaagagggcgccagatttcgttatggatggttgtgagctaccatgtggttgtgggatttgaactcaggacctctggaagagcagttagtgctcttaaccgctgagccatctctccagcccctctgatgagttttgttgttaaatcacttgactatgtgatttgtttgatatgccagaatttttttttcagtctcctaaagtgggagacataccttgaaaagacttgatggtatgaacagtagtgatttaagctcttcctttttctttccaggatctatggtgggaatccgactaccagctccttgcaaatcttcagagactccatcatcttctgcttcatcctctgccttctctgtcatgagtcctgtgattcaggctgttgggtcttctccaacagtaaatgtcatttctcaggcaccttcactgctttcctctggatctagttttgtctctcaggctggtacactgactctgaggatctctcctcctgaaactcaaaaccttgcaagtaaaacaggctctgaaagcaaaataacacccagcactggaggacagcctgtaggcactgctagcctcattcctctgcagtcaggtagttttgccttgttgcagctcccaggacaaaagcctatccctagctctgttcttcagcatgttgcttcccttcaaataaaaaaggaatctcagagtacagaccagaaagatgaaacaaactctatcaaaagagaggaggaaactaagaaagctctaccatcaaaagataaagctctagactctgaggctaatataatgaagcaaaactcaaggaattattgcctcagaaaatacctcgaataattcattggatgatgggggtgatcttttggatgaagaaacccttagggaagatgccagaccttatgaatactcttatagcactgggtctcatacagatgaagacaaagatggtgatgaggactctgggaacaagaatcagaacagtccaaaagaaaaacaaactgttccagaagttagagctggctctaaaaacattgatattatggcactccaaagcatcagaagtatacggcctcaaaagtgtgttaaggtgaaggttgaaccgcaggagggatcagacaatccagagaacccagatgactttctagtcctttctaaggacagtaaatttgaattatcggggaaccaagttaaggaacagcaatctaactcacaggcagaggccaagaaggattgtgaagattctctggggaaggacagtcttagagagagatggagaaaacacctaaagggccccttaactcagaagtacattggaatttcgcagagctttagcaaagaggcgaacgttcagttctttacagaaatgaagccatgtcaagagaattctgaccaggatatctctgaattacttggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaggatgggagaagcagtgctgctgacttcacagttttggatctgtaagatgaggatgaggaagatgagaaaactgatgattctcttgatgagattgtggatgttgtctctggctaccagagtgaggaggttgatgtagaaaaggtggtgagcccgttttttgttgttgactgaaaccttgtggatttaaatgattagagctagcttttctagagaatcactaaaacctaatgtatttacttgggtgcctaggtttagatcattattaaatgactaatgtttaaggaaacactttgtgtcttactcattactcttgtaaaggttgcttcatcctttggaatatagatgctgtaaagtgcctactaagttgaatttttaaggatacctacaatcatttgatcatagtggtttctgtcacattgtctgtgatgtgattgacacttaacctgatacacttaaacactgaaaatatgcttttaaaaagtagaggagaaagagtttgtctgcatttagagggaaatttttcctcttgggaaaaactgcaaaattccacctaatatttttgtttaattttacctgaaagttgctttgtatctttcagtattgtgttggaactaaaaattctatattttttctatacttca

SEQ ID NO: 83 - DNA, 1162 bp, Unknown Organism
  Description of Unknown Organism Mga, EMBL No. q9qxj5
ccagaactggagaggttaaagcaagaggatcattaactagttccaggccagccagggctacatagtgattgatacttttgaaatgattacaaccatgtatacatttctcatataatatagctaacaaactgcagagcctgaagaaaattatattgagagacatgggaaaattcagtaagtggatgcaccatattattaggttaggataatggataataaaagagccaagaataactactatatacaaactataaaagagcagaaattaaatttaaaggcagaaacaggcagatctgagtttgaggccagaacagccatgctttcacaatgacgccctgcctatgaatgaatgaatgaatgaatgaatgaatgaatgaatgaatgaatgaatgaatgaatgaatgaatgaatgaaattttaattttagtaactatttgaacactataggtaaagtgtactgtttaataaattgtgaccatctagtataccttggagaaaagaaactaatttaggaagtttgggatgatgtagtttcataatttgtgaaatgaaattgatctgatattgaatttcagtaaaatattaaaactacttttatttttaaatctaggtgtcatagccatatctctgcagatgaaaaagcttctgaaaagagtcgaaaggtatttagaatgtatctaataaaattatttaataaaattggtgacatttatgcttaaaaacaataagatctatttactagtttattaagggcttttgtttagatcattttatttgtcctctctttacaaatatactacttggttttgcgagtatgtgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaaaagaagttgcccattgaatccctgagtatgtatcagtggtaaggtgtataaatttgagttttccaaattctactcagtgagacatattcgttgactttaggattactttcagaagataatgaagtttgtttatacaaag

SEQ ID NO: 84 - DNA, 1192 bp, Unknown Organism
  Description of Unknown Organism Mga, EMBL No. q9qxj5
actctttttgtaacttatgacatgtttagttgatatccttgggaagcctgttttgttttgtttcttaaagggaaactgaggaggagtggaggtaggagggaagggtgggagcaggggaaaatgtagtcagaatgtaatgtatgtaaagagtgaaaatttaaaaaatagagaccttttttttaaaaagaaaaccaaaagattgtggatttttgttgttgttatgtcctgaaaactcattttaacaataaattagattttaaaagctattccagatctttccttcctgaagctccagatggtccctcattcgagctggaaagtaatatgattatatagtatatattagaatttatactgttggtattgttgaaataaggaaagatagactgggaagatagcatctgagaagccttgataaaagtccagacaaggtgtatctgatttcgaacagtatgatttgggatggaggaagatgctgaggaaatactctttttggacaggtttcattaatttcttcaaaattgaaagatgggtacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcacctgaaaggctgaactggaagattgccatgagttcaaggcagcttgagctatagagtaatgcagcatcacaaacaggactagtaaggaagcagtaaatattcaggggaaaaatcattaagtcaaaataactttatgttgattgttaagaacaggagagagagagagagagagagaaagagagagagggggggtgctggtgtgcaaggaagcacatgagcctatgggtacatgcttgcttctatgagtgtgtatgcaggaatcaggaggatgttgggcgttttcttttttattactttgagacaggctctctcgtgaaactggcacgttgcctttatggctaggctgggtggccatccagctcttggaatctgtctgccttattccaatgctagtattaccatactcagctttttacgtgagtgctggggattcaaactcgggccctcatgct

SEQ ID NO: 85 - DNA, 1127 bp, Unknown Organism
  Description of Unknown Organism Mga, EMBL No. q9qxj5
taattttaactctgcacacaggctaggccatcaaaggtaacatcatttcattacttttaagtagacgttggggatatacatctggcattcatatttcgctattgaaataaacaggttgtaaacattttaaaatttgattaaaatgtaaacagaacatgagtattttatatttttccttgtagtagtggaaaagctgtttaacactcatctttggaaataatcattctttacattccataaaaatataagctagaaaacaatttaataaatactgttggattggggatatagcccagagacagactcttgctttgcatgcacaaggccctgaggttctttgccagcaccagaatcagtggctcaatgtattattcaatttctgaaacatgattagttgttttataatgttctctaaaatactaccttaaaatactttcattaaagaaattgtgtaggcccctgtactttttggagtacaggtgaatgctccattgatattactatttttattctaggcattcagtgaaattcaaggactaacagaccaggcagataaattgatagggcagaaaaatctcctgagtcgaaaacgaagtattttaatacggaaggtatcgtctctttcaggtaagctctttaagcgtctctgagaaagagagagcatagtgccagcattagcacggactccaagcctttcagagtctgtggaggtaaaggattggcatctcaggcttgcaccagggtaactttgactgagcacatcatattttctgaatcataataatcacatttgcctctcagagttctagggaatatagttaaatcagttagataacagctgtttctcattgttatatggtatcatgcacagagtgatgctttactgcctatttaccttcagggaagacagaagaagtggtcctgaagaagctagagtatatttatgcaaaacaacaagcactagaagcacaaaaaagaaaaaagaagctggggtcagatgagttttgtgtgtcccccagaattggcacacagctggagggatcttctgcatcatctgtagatcttggacagatgctcatgaataacaggagggggaaacctttgattctttccagaaaaagagacca

SEQ ID NO: 86 - DNA, 1234 bp, Unknown Organism
  Description of Unknown Organism Mga, EMBL No. q9qxj5
ataatgttctctaaaatactaccttaaaatactttcattaaagaaattgtgtaggcccctgtactttttggagtacaggtgaatgctccattgatattactatttttattctaggcattcagtgaaattcaaggactaacagaccaggcagataaattgatagggcagaaaaatctcctgagtcgaaaacgaagtattttaatacggaaggtatcgtctctttcaggtaagctctttaagcgtctctgagaaagagagagcatagtgccagcattagcacggactccaagcctttcagagtctgtggaggtaaaggattggcatctcaggcttgcaccagggtaactttgactgagcacatcatattttctgaatcataataatcacatttgcctctcagagttctagggaatatagttaaatcagttagataacagctgtttctcattgttatatggtatcatgcacagagtgatgctttactgcctatttaccttcagggaagacagaagaagtggtcctgaagaagctagagtatatttatgcaaaacaacaagcactagaagcacaaaaaagaaaaaagaagctggggtcagatgagttttgtgtgtcccccagaattggcacacagctggagggatcttctgcatcatctgtagatcttggacagatgctcatgaataacaggagggggaaacctttgattctttccagaaaaagagaccaggctacaggtaggaggggcttttttttttttggtagataaacatcaatttattttagtggactgaacatcttctcttggctgtagttctaattaaatgaattgggatgatttttaagaaaaataactttaaatagtacttttcaacgtttggctttatacaataaataactttaagagcttagtattctattgtataaatcctacttctgatagcagtctacttgtaacatgtttttagtaagtacataactgctactatatgtgaatcctccattttttttaatctattatgggttgaaaaataaattggaactttttaaacttacgcttaaggctcagaattgagttcaatctaggtaacacaatgagactgttgttttcttaaaaagtttaatttctttgaactagtccatttttcattgtctttattaaggaacaaagttaattggtttagttgtttcttaccttgtttttggaagtagctacactgatttaatgttacttggt

SEQ ID NO: 87 - DNA, 1177 bp, Unknown Organism
  Description of Unknown Organism Mga, EMBL No. q9qxj5
attgagttcaatctaggtaacacaatgagactgttgttttcttaaaaagtttaatttctttgaactagtccatttttcattgtctttattaaggaacaaagttaattggtttagttgtttcttaccttgtttttggaagtagctacactgatttaatgttacttggtaaactcatgtcttagagaaaggtgtgttcttttttattcttcagacagttcagtgtatggaccagaaaaatctgacttatcagttcacgttgattctaaaacaagaattagttctaacattacagaatagaataaataactcgtactcacaagcggaatgaaactgtggtcgagatatatgaaggactttgttaggagtgtataagatagaaagcctgtgaatagtgtatgccctcatgtaaacttacattgtaccttttaagtcagcctctctttagagtacactgacattaactggacatttgttttactaatttttcactttctttgcagaaaatgcctcaccttctgacactccgcactcttctgccaacctggtaatgactccacaagggcagttgcttaccctaaaaggccccttattctcaggaccagtggtagcggtttctcctgctctcttagaaggtggtctgaagcctcaagttgcaagcagtactatgtctcagtctggtatgtggtggtattctatggtttgtaaatatatactatggttggtaaagaatggtccattgatagaggacgtctttaaatctgatctgtagttacctagagctgttcttttggcatttgccttgttaaaaaatggtccttctagctggccagtagtggcacacgcctttaatcccagcatttgggaggcagaagcaggtggatttctgagttcaaggacagccagggctacacagacaaaccctgccttggaaaaaagaaaagaaaagaaaaaaaaaaaagaaagaaatagtcctcctagcttgatttgattggtggttaacctggagttggaaactgttttgttaataactctagtgacatttctaagcaggggaagaattaaaatggtgttcaagtgaaatgatgatgtagaagggtcaatatttaagttcttggtgttgatcgttgttttgggttgtatttggaatggaggcactcccatgttaccaatccagtga

SEQ ID NO: 88 - DNA, 2275 bp, Unknown Organism
  Description of Unknown Organism Mga, EMBL No. q9qxj5
ttgtataggcaagttcctgggttcagtctcctccactgctctcccagtccaaaaaaagaaaagagttattttgtagagattttctaaaaatgtaattaagtggaactattgctaataatgaaatacctaagtgtcttgactagattttacttgacttattgctatttcagctgttattctgttattttgtcctttttttctttttccctccctcccttccttccttccttccttccttccttccttccttccttccttccttccttccttccttcctttctttctttctttctttctttcttttttttttttttccagagctgaggaccaaacccagggccttgcgcttgcaggcaagagctctaccactgagctatagccccaacccctcagctgttattctgacaaaagtggactaagatagactgggaaggagagagagaaaaagagattttcattactgcatctaatgaactctctctttaatttttgattttcagagaatgatgacttatttatgatgccacgaattgtcaatgtgacatcattggctgcagaggaagatttgggaggtatgagtggcaacaaataccgtcacgaagttcctgatggcaagccacttgaccacctgagagacattgctgggagtgaagccagctccttaaaagatacagagagaatctcttccagaggaaaccatcgagatagcagaaaggcactgggtccaacacaggtgcttttggcaaataaagattctgggtttccacatgtagctgatgtttccactatgcaggcagcacaggaatttatacctaaaaatatgtctggtgatgtgagagggcatcggtataaatggaaggagtgtgagttgagaggagagagactaaagtcaaaggagtctcagtttcataaattaaagatgaaagatctgaaggactcaagcatagagatggaactgaggaaagtagcctcggctatcgaggaagcagccctccatcccagtgagctgctgactaacatggaagatgaggatgacactgatgagactctgacttcactgctcaatgaaattgcttttcttaatcaacagctaaatgatgactctggcctagctgaactgtcaggttctatggatacagaattctcaggagatgctcagcgagcttttatcagtaaacttgctcctgggaacagatcagctttccaagttggacacttgggagcaggtgtgaaagagttgcctgatgtccaagaggagagtgaatccatcagccccctcctcttgcacttggaagatgatgacttttctgagaatgaaaaacagcttggggacacagcctctgagccagatgtccttaagattgttattgaccctgaaataaaggattctcttgtttcccataggaaatctagtgatggagggcagagtacttctggtctccccgcagagcctgaaagtgtatcttcacctcccatcttacacatgaagactggcccggagaacagcaacacagatactttgtggaggcctatgccaaagttggccccattaggtttaaaagtagctaatccccccagtgatgccgatggtcagagtcttcaaggtgatgcctgccttggcacctatagctgccaaagttgggtccattggacccaaaatgaatttagcaggaattgaccaggaaggccgggggagcaaggtgatgcctacattggcacctgttgtacctaaattgggcaactctggagctccatcaagttcatcagggaaatgagcttgttcatcctcatacaaaagccaggctgtgaggggaaatgaatttcacctcctttctctgtaggcgtctgtttgtttgtattatggaactattatccttgacttaattatgtgtgaatattgatggagaaaggaggtgtagggtgctggtcctggtgtttgaaattctgatcatgttaaaaatgtgttaccttacttgtggtgctgggtcccctcatcttctctaagaaggtgtgattccctcttgcccaaggaattcataacaagattctggctccatcaactaccttattattttcccctgagaaaatgcttgagacttggaattactatagactaaagtataagtcatagttgttggctccaaaatctgagaattatgatacagttgtgtgggaaaaaaaaaaaaaaagaacttttagttactatttcaatagtagggcatggtaggactgggcatggtagtgcacacttgtaattccagcacttagaagacta

SEQ ID NO: 89 - DNA, 1266 bp, Rattus sp.
gccgctggtgccaccatagctactgctgcggctgcggcagcagagctgaagccgtcgatcgcgatgccggagggcgctgtgagctgggccccagcgaacgggggcgctgacgagtagtagggtttctgaagtaggcagtgggtgtggggggctaaaacggagctcccaagactcgggtgcgggagggagcttctcgcatcggctcgggccagcgtgagcccggcggccagctcggtgcttcggccccttgcccagtggcttcggaggcctcctcccttggacacctcagttaccatgggaacacttgggctgtaaccggcgcccgactggtcactagttcatccgattcttcagttaaacgcctctggcaggtgtgggcctgacatttcttcactccaggggtcgtcctccccttgtaaaaaaaaatctagcaggctagggtggggatggggagtgggaaagcgcagggcaatgcccagagtaagagttggtgacaagcagaagacacctactgtgatgataaggttgcttctgccatctcaggccagtgaaagtgcatatccgaaggctgtgttgaacgagaaggcagtaccgctgttgagacaagtccaaaggctaggccagggactgtccttaggggctcctcgttatgatggctggagaagggtcaaccattactagccgtatcaagaacttgctgaggtctccatccatcaaattacgcagaagtaaagcaggaaaccggagagaggacctcagctccaaggtgagtcctgttgggagccacctcttacctactctgtcctctcctgcacttcgttccttttttttacctttctctttcttgaggggcaggaagggggtggaagaatgaaatttctctgttggctttaaaagatcatatacgcttatctttgacgacctgatttgaagttggaagaacagcaggcgtgggtgggcatgctgagacgcagccatggcttgaagaagggcataggaaggcaaactgggtttaacagggcctcagtttaaactaagtgtgtgcttgggaaacaagttgacattttatgactcagtttctcttcttagaagagtacagtgagatcacatattctgtaaaggacctagctgtgactaatgtattagtgtaacagttggggttataatgccagactgtacaacagaaagaatatactaatacacttcatctaagagagcaaaacaattttttgacaagcttagtgatgcatgcctgtaatcccagca

SEQ ID NO: 90 - DNA, 1092 bp, Rattus sp.
cttttaactggcagacacgggaggtggagtcctttgatgggacgcaaagatcaaggatgtccgagttacctaattgatacctaacgttcttctggctctcggtagagagatcacagtggcctttgaaggtttgaaggccagtgacctcttgtcaatctcatagaccccaggtcctagcctacagttagtgccttctcacatcggaaggattcttgttcctctttggtcataaacctgtactttaacccatgttttctttcccatggtaacagcaggtggagcgtgattaatgcaggtgacacttattactggcattttctcgtatggccaagaggattttgatcagaacaaagtctgaaaaagacccagactgaataaggttgtagggaaatagtgcgtcagaggctgggttgtaggagcctttagcacccacctcgaaccctactgtgactgtcatcctatggtctgtaaggagtcacttctctgtcttgctctaacaggtaaccttggagaaggtcctgggagtaacagtatctggaggaagaggacttgcttgtgacccccgatctggcttagttgcctacccagcagggtaagttaagtgcctttcagggcatcttgctacatgaaagtgtattttttatttcttactaacatgttattcattgagaatttcatacaatatactttggtcctatttactcccttcccctactattcccacatccactcccccttcctacccattttacttggtgctctctccctcttctctccccccccacctttttaaagacccatcaagtcatatttgtgttgcccaactactctcaggtgtggggcttatccttggatgtggtcaccctcccaggggggtcacaccattaaagaaagctggctttccctcttccagcagttgccaggtgccaacagcttctgggttagatgtgggacttttgcccactcaccacttctacccactccccagtccacccatgttaggattttgactggcttgagctcactgagatcctatgaatgcagtcacaactgcagtgagttcttaggtgcaattgtcatgctgtatccaga

SEQ ID NO: 91 - DNA, 1063 bp, Rattus sp.
tggaagctttgtagccttcccaagtcattcccaaggtcctcccaagctatctctgctccctccacctccagcttaggcctccttgcagttctagcccctagcatagcccgtgtgttctttcttactcagtaggtagaagcataccatctctaacagtgattaagacatgtggctccaagcctgagaaccagttgcaagctgaggcctgagctgatcaaatcacattaactcttccatcttccattcattccttgggatatgttcaaaggctgggggaaggccctgtctctgctgtcctttaggagtatgatcagagatgggactgacagtcagggtcctggtgactaggcttctggtggagagcctttctattatgggaatctgataatagacccttttgggtggagactccgacctgttccctctgctgggtgaccagaagtctcagtgggctcctgaggaagctatggttcagtgtgacatctcactcatctcctcaggtgtgtggtcgtcctcttcaatccccggaagcacaaacagcaccacatcctcaacagctccaggtgtgtgggcctggcctcaggggctgggggtgaccgtggccgggggcctgaggccctgtggccagatagggacgcgcatatacactcttgagtttgttggccttgcctttgggaaatatgcatgaaggatggaggaagggaggttggggaagggggcacagtctagggtcaaccaaacctgccccgtctcttgtaggaaaaccattactgcccttgccttctcccctgatggcaagtacttggtcactggagaggtaagtgaagaacagggtggcaatattgtagcttatggacaccctgccttcaaccaaagccaccgtcctgtgttccagctagggtgctgtcactttggcttcagggattgataccctggttcctgctttgaccagatagctgttcttaagctttagtgtgatcaggctccctgggagggcttttcaaagtgctcacagctaggctcctactccaaagttgtttgattctataaccttcttttaaaa

SEQ ID NO: 92 - DNA, 1058 bp, Rattus sp.
tgttcaaaggctgggggaaggccctgtctctgctgtcctttaggagtatgatcagagatgggactgacagtcagggtcctggtgactaggcttctggtggagagcctttctattatgggaatctgataatagacccttttgggtggagactccgacctgttccctctgctgggtgaccagaagtctcagtgggctcctgaggaagctatggttcagtgtgacatctcactcatctcctcaggtgtgtggtcgtcctcttcaatccccggaagcacaaacagcaccacatcctcaacagctccaggtgtgtgggcctggcctcaggggctgggggtgaccgtggccgggggcctgaggccctgtggccagatagggacgcgcatatacactcttgagtttgttggccttgcctttgggaaatatgcatgaaggatggaggaagggaggttggggaagggggcacagtctagggtcaaccaaacctgccccgtctcttgtaggaaaaccattactgcccttgccttctcccctgatggcaagtacttggtcactggagaggtaagtgaagaacagggtggcaatattgtagcttatggacaccctgccttcaaccaaagccaccgtcctgtgttccagctagggtgctgtcactttggcttcagggattgataccctggttcctgctttgaccagatagctgttcttaagctttagtgtgatcaggctccctgggagggcttttcaaagtgctcacagctaggctcctactccaaagttgtttgattctataaccttcttttaaaagtaaaaccaagcccgggtggtggcgcacgcctttaatcccagcacttgggaggcagagccaggtggatttctgagttcgaggccagcctggtctacaacgtgagttccaggacagccagggctacacagagaaaccctatctcaaaaaacaaaacaacaacaaaaaagtaaaaccaaaacaagaaatattaattaatttctttatgtgggggaggggtcatgaaggttatgtcaaatgctttacacgctgaagt

SEQ ID NO: 93 - DNA, 1171 bp, Rattus sp.
agagaaaccctatctcaaaaaacaaaacaacaacaaaaaagtaaaaccaaaacaagaaatattaattaatttctttatgtgggggaggggtcatgaaggttatgtcaaatgctttacacgctgaagtcatctcttgcctcctttcccccagagcttctaacaatgtggggtagttaaggacacatttatcctaaaatattcctatcttcagatgccccctgctgggcacatgaggaacatggcccttggttttacaaggaggcagtcagggtagatgcctgctgctgcttgctagaaccattcaaggagctttgaccagacagaggcaagggctaacctctgctgacactggtgtcctcagatcctttttctatgcctttatcttgctctcccttgttgaacactggagggacagtgtttaaagctggggaggtgagagactaaggaggctacacataacaccttccacagttctcacgtgcggttttgcctccctgcagagtgggcacatgcctgccgtgcgggtttgggatgtggctgaacgtagccaggtggcagagctacaggagcataagtatggtgtggcttgtgtggctttctccccaagtgccaagtacattgtgtctgtgggctaccagcatgacatgattgtcaacgtgtgggcctggaaggtgagtaggctggtgacagccttagcggcctcatgagggttcccatgggggtggagggggaactgctgacccaagcaacctcatttccttcactgtctccagctcttccttgaacagtatttgaagtacaaaatagcacctgaggagcaagggtgtttctagaggcttttcctacacatctaaggaattaaacctttgaattcctttagaaaagaggttgggtttttgttgttattgttttgttttctgaccattaaataccagaaaggaagagttttgtttttcctctctttctgttgttgttttgtttgtttgtttgtatttgtttttattttggggtttttttttgtttttgaagtagaaattattgctagctcttgtccattctcagataccttcttaccccagtaggaagagcttgtctgttctcggataccttcttatccccaacaggcagaactttgattcttcctctggcttcccctccagaaaaacattgt

SEQ ID NO: 94 - DNA, 1138 bp, Rattus sp.
gggcctggaaggtgagtaggctggtgacagccttagcggcctcatgagggttcccatgggggtggagggggaactgctgacccaagcaacctcatttccttcactgtctccagctcttccttgaacagtatttgaagtacaaaatagcacctgaggagcaagggtgtttctagaggcttttcctacacatctaaggaattaaacctttgaattcctttagaaaagaggttgggtttttgttgttattgttttgttttctgaccattaaataccagaaaggaagagttttgtttttcctctctttctgttgttgttttgtttgtttgtttgtatttgtttttattttggggtttttttttgtttttgaagtagaaattattgctagctcttgtccattctcagataccttcttaccccagtaggaagagcttgtctgttctcggataccttcttatccccaacaggcagaactttgattcttcctctggcttcccctccagaaaaacattgtagtggcctccaacaaagtatccagtcgggtaaccgcagtgtccttttctgaagactgcagctactttgtcactgcaggcaaccggcacatcaaattctggtacctggatgacagtaagacctcaaaggtgagggctgagatgaccaaccctgaacagcactccagctgttgcctttggcttgtttaacagtcacaggttcttcatggacctgacctgcctgggctgaggcgtctcaaagaacacatgcttgccctctctctgaggccctcatttccaaggctagatggctgtagtggtgtctccacaggtgaacgccactgtgcccctgctgggccgctcggggctgctgggggagctgaggaacaacctgttcactgatgtggcctgtggccgaggggaaaaggctgatagcactttctgtatcacgtcgtcggggctgctgtgcgagttcagcgatcgcaggcttctggacaaatgggtggagctaagggtaagtaactctggctccagaacagggtctggccttatctcaaatcaggggagaactggccttggggctgtagagccaggctgggcctttgtgcatgctgaaggggactgcaggagaagactcagggcgctcgctc

SEQ ID NO: 95 - DNA, 1183 bp, Rattus sp.
ttgtttgtttgtatttgtttttattttggggtttttttttgtttttgaagtagaaattattgctagctcttgtccattctcagataccttcttaccccagtaggaagagcttgtctgttctcggataccttcttatccccaacaggcagaactttgattcttcctctggcttcccctccagaaaaacattgtagtggcctccaacaaagtatccagtcgggtaaccgcagtgtccttttctgaagactgcagctactttgtcactgcaggcaaccggcacatcaaattctggtacctggatgacagtaagacctcaaaggtgagggctgagatgaccaaccctgaacagcactccagctgttgcctttggcttgtttaacagtcacaggttcttcatggacctgacctgcctgggctgaggcgtctcaaagaacacatgcttgccctctctctgaggccctcatttccaaggctagatggctgtagtggtgtctccacaggtgaacgccactgtgcccctgctgggccgctcggggctgctgggggagctgaggaacaacctgttcactgatgtggcctgtggccgaggggaaaaggctgatagcactttctgtatcacgtcgtcggggctgctgtgcgagttcagcgatcgcaggcttctggacaaatgggtggagctaagggtaagtaactctggctccagaacagggtctggccttatctcaaatcaggggagaactggccttggggctgtagagccaggctgggcctttgtgcatgctgaaggggactgcaggagaagactcagggcgctcgctcgcagtgatctgcataaccagctcctcttgctctttctcgcctctcttccctgctttcctcttctgctcagaacacagacagcttcacagtaagtgcttctaggccttctcttctcaagctgttgctttctccttgttggggtttttctaccttccaatcttggtccctctgatggccttgatttctaccccacctcccaagtctgcagggctacaggccctctgaccatatgctgctggagggagggcatttctagatgacacctgaggggttggcccgactttggcctggctctcctttgtgagcgagtgttgctgagggcttgggaaggattaagccaagctaaatgggaagatggccctta

SEQ ID NO: 96 - DNA, 1018 bp, Rattus sp.
ttcttcatggacctgacctgcctgggctgaggcgtctcaaagaacacatgcttgccctctctctgaggccctcatttccaaggctagatggctgtagtggtgtctccacaggtgaacgccactgtgcccctgctgggccgctcggggctgctgggggagctgaggaacaacctgttcactgatgtggcctgtggccgaggggaaaaggctgatagcactttctgtatcacgtcgtcggggctgctgtgcgagttcagcgatcgcaggcttctggacaaatgggtggagctaagggtaagtaactctggctccagaacagggtctggccttatctcaaatcaggggagaactggccttggggctgtagagccaggctgggcctttgtgcatgctgaaggggactgcaggagaagactcagggcgctcgctcgcagtgatctgcataaccagctcctcttgctctttctcgcctctcttccctgctttcctcttctgctcagaacacagacagcttcacagtaagtgcttctaggccttctcttctcaagctgttgctttctccttgttggggtttttctaccttccaatcttggtccctctgatggccttgatttctaccccacctcccaagtctgcagggctacaggccctctgaccatatgctgctggagggagggcatttctagatgacacctgaggggttggcccgactttggcctggctctcctttgtgagcgagtgttgctgagggcttgggaaggattaagccaagctaaatgggaagatggcccttacttactgagctgctctgcccacagaccactgtggcccactgcatctctgtcacccaagaatacatcttctgtggctgtgctgatggcacggtgcgccttttcaatccttccaacctgcacttcctcagtaccctacctagaccccatgctcttggaacagacattgccagcatcactgaggccaggtgagctatatggggcccactgtccattcaaagctttag

SEQ ID NO: 97 - DNA, 1161 bp, Rattus sp.
gggtctggccttatctcaaatcaggggagaactggccttggggctgtagagccaggctgggcctttgtgcatgctgaaggggactgcaggagaagactcagggcgctcgctcgcagtgatctgcataaccagctcctcttgctctttctcgcctctcttccctgctttcctcttctgctcagaacacagacagcttcacagtaagtgcttctaggccttctcttctcaagctgttgctttctccttgttggggtttttctaccttccaatcttggtccctctgatggccttgatttctaccccacctcccaagtctgcagggctacaggccctctgaccatatgctgctggagggagggcatttctagatgacacctgaggggttggcccgactttggcctggctctcctttgtgagcgagtgttgctgagggcttgggaaggattaagccaagctaaatgggaagatggcccttacttactgagctgctctgcccacagaccactgtggcccactgcatctctgtcacccaagaatacatcttctgtggctgtgctgatggcacggtgcgccttttcaatccttccaacctgcacttcctcagtaccctacctagaccccatgctcttggaacagacattgccagcatcactgaggccaggtgagctatatggggcccactgtccattcaaagctttagtcactacttcatggagcagaatggagagtgcgcaatgcccaaagatcccctttgtagacagaaacctgggtttcttggtagttgtcagaatcggggagattaaatagcacagaggaccatggactgtctcacctgggtgcatcttaggtgattttaagaatgactcaagggattggagtcttccttctggttggcctcttgataagctgtcagcaccagaaagaaagtatgctgtggtaaagacacagaaagacaaagtcaccaatcatgctaggaggaaaagggtgatgtgtggtcatttttatggtttggcaagtgtcctcagtaaaagatgaggagggggaagaggaggaagaagaagaagagggggaggggaggaagaggaggaagaagaggaagaagagaaagaggaggaggagattttgaggtgctggagatagtaccaagagccttgcttgtta

SEQ ID NO: 98 - DNA, 1190 bp, Rattus sp.
tggtttggcaagtgtcctcagtaaaagatgaggagggggaagaggaggaagaagaagaagagggggaggggaggaagaggaggaagaagaggaagaagagaaagaggaggaggagattttgaggtgctggagatagtaccaagagccttgcttgttagataagtgctctaccactgagctatatcacaaaccctatttttgtggtatgtagtatagtcttgttttgatctctgtgcagaaatactcatagaaccatgtagattttgttgtgctccattgtaaccacagaattgccttgtctggtattggtgatctatgaatttgttagcatttctctggaaaacactctgcccagtagctgtggttgggtgagctcctggattggatctctaggcactgcttttgaattctcagttctacttccaaatctgtaggactggagacagtgtcctgtgctccattccttcctttgtggtaacttcctgtcctctctcccacagtcgcctcttttctggaggggtcaatgcaaggtacccagacaccattgccttgaccttcgatccaactaatcagtggctatcttgtgtatacaacgaccacagcatatatgtttgggatgtgagggaccccaagaaagtggggaaggtgtactccgctctgtatcactcctcctgtgtctggagtgtggaggtacgtgggatgggtttgcagtaattccactgtgagaatagaaagcagagaacaccaagcacggccctagggtcctgcccctcggctctcagtggcttccctgctgtagctctagagcttctagagctccagtgtgaacgttggtgcccatgcacctcagccttcttgtgtgtctctgcctagcctgagtctttgccttatcctcggctcaggaatcttggataagttactaaaccagactaagcttcagatcccttaactgtaaaatgatgtatcagattttcccaattgttctaacaatgaaatgacataacgtaattgagagattatggcacacaggaggagctgggatcgcgggcttttccttactgggtgcatatataacagtaatgtctcctttccttactaaatgtcagacaatagctaagtcttagcactctgccttggatgccataaccctttccttcttcctgagtagacaaaaatgccccccaaagcgg

SEQ ID NO: 99 - DNA, 1147 bp, Rattus sp.
tgtgagaatagaaagcagagaacaccaagcacggccctagggtcctgcccctcggctctcagtggcttccctgctgtagctctagagcttctagagctccagtgtgaacgttggtgcccatgcacctcagccttcttgtgtgtctctgcctagcctgagtctttgccttatcctcggctcaggaatcttggataagttactaaaccagactaagcttcagatcccttaactgtaaaatgatgtatcagattttcccaattgttctaacaatgaaatgacataacgtaattgagagattatggcacacaggaggagctgggatcgcgggcttttccttactgggtgcatatataacagtaatgtctcctttccttactaaatgtcagacaatagctaagtcttagcactctgccttggatgccataaccctttccttcttcctgagtagacaaaaatgccccccaaagcggaagtctaattctctccctctgtcccttcaggtctaccctgagatcaaggacagtcaccaggcctgtcttccccccagttcctttattacttgctcctcagacaacaccatccgcctgtggaacacagagagctctggggtacatggctctaccctgcaccgtaacatcctcagcaatgtgagcctcccgtttcatacttatgcccccatgcttatagattttgaccttgttttcaggaactggctccccgaatcttgatcattcagcttttatttggctggactctctgagcctagggttgattttacccagcatccctctgtgcttgcagccatcctgctggtactgcctgagctgtctcctttctcctttcccaccaggatctcattaagatcatctatgtggatgggaacactcaggctttgttggacactgagctgcctggaggagacaaagctgatgggtcgctgatggacccccgagtgggcatccggtccgtgtgtattagccccaatggacagcacctggcctccggagaccgcatggggacacttaggtaaggtcaagttctgagctaggtactgactgcctctcatggtgactgtggtagttagatcctcaagagcagcgctttaggagggcttgtctccccccaaagcttagaaacaagatattta

SEQ ID NO: 100 - DNA, 1176 bp, Rattus sp.
ataacagtaatgtctcctttccttactaaatgtcagacaatagctaagtcttagcactctgccttggatgccataaccctttccttcttcctgagtagacaaaaatgccccccaaagcggaagtctaattctctccctctgtcccttcaggtctaccctgagatcaaggacagtcaccaggcctgtcttccccccagttcctttattacttgctcctcagacaacaccatccgcctgtggaacacagagagctctggggtacatggctctaccctgcaccgtaacatcctcagcaatgtgagcctcccgtttcatacttatgcccccatgcttatagattttgaccttgttttcaggaactggctccccgaatcttgatcattcagcttttatttggctggactctctgagcctagggttgattttacccagcatccctctgtgcttgcagccatcctgctggtactgcctgagctgtctcctttctcctttcccaccaggatctcattaagatcatctatgtggatgggaacactcaggctttgttggacactgagctgcctggaggagacaaagctgatgggtcgctgatggacccccgagtgggcatccggtccgtgtgtattagccccaatggacagcacctggcctccggagaccgcatggggacacttaggtaaggtcaagttctgagctaggtactgactgcctctcatggtgactgtggtagttagatcctcaagagcagcgctttaggagggcttgtctccccccaaagcttagaaacaagatatttattctgtaaaatgaaagaatgagcttacagtggtgagctcagaaaaagccatcgcagacataggcacagctatcagtcacacatccgcatgaggaagctcaagataaggggttctaagaaggacataatattgtttctgtggtgttctttaaaaaaatgcatataactcagttcacttgtgagaggctcaaatgaggtacatacaagagagtaattgtcagtgttcttcaaaagtgtcgaggacatggaaggtaaggagggatggagagactaccacagacttcaggaaactaaagagcagtgatagctaaatgccttttacattctttaagatcctgaatccaggacatatggacaaataatggcatgttaataatata

SEQ ID NO: 101 - DNA, 1092 bp, Rattus sp.
taaagagcagtgatagctaaatgccttttacattctttaagatcctgaatccaggacatatggacaaataatggcatgttaataatatacctacttgggctggtgagacggttcagcggttaagagcaccgactgctcttccaaaagtcctgagttcaaatcccagcaaccacatggtggctcacaaccatccgtaacaaaaatctgatgccctcttctggagtgtctgaagatagctacagcgtatacacatataataaataaattaattaattaaaaaaataataatatacctacttaacagtatgtaaatgcctggctctgacatgtgctgttgttatgtatgctagttaagaactgggcaaggggatatagaacaccttcagtgtttgaactttttcttaaagctttaaaatgtttgtttaaaggtggggagaggatgtaacagaagaaggaggcctttcctctcacagccaggcaatgatgtctcttatttgtaggatacatgaactgcagtccctgagtgagatgctgaaagtggaggcccacgactctgagatcttgtgcctggagtactctaagccagacacaggtaaggcctaacccatgccctagcagggtctagctctggcacagcccctcctgtcctgccagtgccaccttatacctgccttggcttctgataatcttgggtcctatattcattcagatcacttggtgtttgcaggattgtaagatctctgcccctgttctgtggttctgtggctttagggatatgaggctctgtctacttggggctctttcccaggcactgataagggccagcttaacgtgaactgataagattcttccaaccccaggtttgaaactgctagcatcggcaagccgggaccgtctgatccacgtgctggatgctggccgggaatatagtctacagcagacactggatgagcattcatcttccatcactgctgtcaagtttgcaggtggggactgggcaattgaagctattcatccctctacctaccacctacatttatgagaagagtttgctgggaaggattgggaggtacccgggagtcatgaggctag

SEQ ID NO: 102 - DNA, 1126 bp, Rattus sp.
ggcaaggggatatagaacaccttcagtgtttgaactttttcttaaagctttaaaatgtttgtttaaaggtggggagaggatgtaacagaagaaggaggcctttcctctcacagccaggcaatgatgtctcttatttgtaggatacatgaactgcagtccctgagtgagatgctgaaagtggaggcccacgactctgagatcttgtgcctggagtactctaagccagacacaggtaaggcctaacccatgccctagcagggtctagctctggcacagcccctcctgtcctgccagtgccaccttatacctgccttggcttctgataatcttgggtcctatattcattcagatcacttggtgtttgcaggattgtaagatctctgcccctgttctgtggttctgtggctttagggatatgaggctctgtctacttggggctctttcccaggcactgataagggccagcttaacgtgaactgataagattcttccaaccccaggtttgaaactgctagcatcggcaagccgggaccgtctgatccacgtgctggatgctggccgggaatatagtctacagcagacactggatgagcattcatcttccatcactgctgtcaagtttgcaggtggggactgggcaattgaagctattcatccctctacctaccacctacatttatgagaagagtttgctgggaaggattgggaggtacccgggagtcatgaggctaggtaggaaggcaggtgaaatatagactttcagtctaggggaccctgagccagactgagggctgaggagggggtgcatgggttttgtgggatgcactctgttttatttggttggtccaccatgtagagtagctccattggggaaaataagggaaggcaggagaaaggctgccttcatatggtggaagcagccaggtccctgtcccctacagccagtgatgggcaagtgcgaatgatcagctgtggtgcagacaagagcatttacttccgaactgcacagaaggtaagggcactgggtgtccccagcagggcaggggtgggaacagggtttcaggctatgggaggtgaagagagaagtgagccctgtgagctgtgatgcatttgcagtctggagaag

SEQ ID NO: 103 - DNA, 1071 bp, Rattus sp.
ttcccaggcactgataagggccagcttaacgtgaactgataagattcttccaaccccaggtttgaaactgctagcatcggcaagccgggaccgtctgatccacgtgctggatgctggccgggaatatagtctacagcagacactggatgagcattcatcttccatcactgctgtcaagtttgcaggtggggactgggcaattgaagctattcatccctctacctaccacctacatttatgagaagagtttgctgggaaggattgggaggtacccgggagtcatgaggctaggtaggaaggcaggtgaaatatagactttcagtctaggggaccctgagccagactgagggctgaggagggggtgcatgggttttgtgggatgcactctgttttatttggttggtccaccatgtagagtagctccattggggaaaataagggaaggcaggagaaaggctgccttcatatggtggaagcagccaggtccctgtcccctacagccagtgatgggcaagtgcgaatgatcagctgtggtgcagacaagagcatttacttccgaactgcacagaaggtaagggcactgggtgtccccagcagggcaggggtgggaacagggtttcaggctatgggaggtgaagagagaagtgagccctgtgagctgtgatgcatttgcagtctggagaaggagtacagtttacacgaacgcaccacgtggtacggaagacaactctctatgacatggacgtggagcccagctggaagtacacggccatcggctgccaagaccggaatattcggtgggtgggcgttcccttctcagacttctttacaaattcattttcactttgtttattttgtgggaagaggattgagcccgtgggggtcagaactactttcaggagtggtttgtctcttaccaggacatgggttctggaggtcagacttagccatcagtccaggcagcaagcgcctttaccctctgagctatttttctggcctcctcttgtcagatttttggaccacattcctttgtgcagctcagtctcaaatctgcttttttgtccaggcttt

SEQ ID NO: 104 - DNA, 1122 bp, Rattus sp.
aagtttgcaggtggggactgggcaattgaagctattcatccctctacctaccacctacatttatgagaagagtttgctgggaaggattgggaggtacccgggagtcatgaggctaggtaggaaggcaggtgaaatatagactttcagtctaggggaccctgagccagactgagggctgaggagggggtgcatgggttttgtgggatgcactctgttttatttggttggtccaccatgtagagtagctccattggggaaaataagggaaggcaggagaaaggctgccttcatatggtggaagcagccaggtccctgtcccctacagccagtgatgggcaagtgcgaatgatcagctgtggtgcagacaagagcatttacttccgaactgcacagaaggtaagggcactgggtgtccccagcagggcaggggtgggaacagggtttcaggctatgggaggtgaagagagaagtgagccctgtgagctgtgatgcatttgcagtctggagaaggagtacagtttacacgaacgcaccacgtggtacggaagacaactctctatgacatggacgtggagcccagctggaagtacacggccatcggctgccaagaccggaatattcggtgggtgggcgttcccttctcagacttctttacaaattcattttcactttgtttattttgtgggaagaggattgagcccgtgggggtcagaactactttcaggagtggtttgtctcttaccaggacatgggttctggaggtcagacttagccatcagtccaggcagcaagcgcctttaccctctgagctatttttctggcctcctcttgtcagatttttggaccacattcctttgtgcagctcagtctcaaatctgcttttttgtccaggctttcgagcctctttgaggcagcaaagtactctgcctcacctgagagagtgcgagcgagttcccaggacaaatgggaagtcccatttgctgccagcttatagcctgggctctggtcctcaggctgctgtgaacacgtgccttccttttcaggatctttaacattagcagcggaaagcagaaaaagctgtttaaagggtcacagggtgaagatggcactctcattaaggtg

SEQ ID NO: 105 - DNA, 1076 bp, Rattus sp.
ggaagacaactctctatgacatggacgtggagcccagctggaagtacacggccatcggctgccaagaccggaatattcggtgggtgggcgttcccttctcagacttctttacaaattcattttcactttgtttattttgtgggaagaggattgagcccgtgggggtcagaactactttcaggagtggtttgtctcttaccaggacatgggttctggaggtcagacttagccatcagtccaggcagcaagcgcctttaccctctgagctatttttctggcctcctcttgtcagatttttggaccacattcctttgtgcagctcagtctcaaatctgcttttttgtccaggctttcgagcctctttgaggcagcaaagtactctgcctcacctgagagagtgcgagcgagttcccaggacaaatgggaagtcccatttgctgccagcttatagcctgggctctggtcctcaggctgctgtgaacacgtgccttccttttcaggatctttaacattagcagcggaaagcagaaaaagctgtttaaagggtcacagggtgaagatggcactctcattaaggtgagcaccccagtgggactagcagagcctgctgcctgccctgccattgggcagcttggggtcggtgctaagtgaggaggggtggtgatcccctactcttgctgtgtccccaggtgcagacagacccctcagggatctacattgccactagctgttccgataagaatctctccatttttgacttctcctcaggcgagtgtgtggccaccatgtttggccactcaggtgagtgagtgagtgtagcagccacctccctagttgaaattgtactttcttcccttggctgttgccacaccacaactctgccctaccctgggaggcatcacctgactaaagcaagagtagaaagccctgtggcagaaggggacagtatggttggtacctttcccttcccccatatccagactcagagcacaaagtgcctatgtgaggatagaacccgactctccctctcctgcagccagtgcccatccctgcagctggaagaggcaggaatttga

SEQ ID NO: 106 - DNA, 1112 bp, Rattus sp.
tgtctcttaccaggacatgggttctggaggtcagacttagccatcagtccaggcagcaagcgcctttaccctctgagctatttttctggcctcctcttgtcagatttttggaccacattcctttgtgcagctcagtctcaaatctgcttttttgtccaggctttcgagcctctttgaggcagcaaagtactctgcctcacctgagagagtgcgagcgagttcccaggacaaatgggaagtcccatttgctgccagcttatagcctgggctctggtcctcaggctgctgtgaacacgtgccttccttttcaggatctttaacattagcagcggaaagcagaaaaagctgtttaaagggtcacagggtgaagatggcactctcattaaggtgagcaccccagtgggactagcagagcctgctgcctgccctgccattgggcagcttggggtcggtgctaagtgaggaggggtggtgatcccctactcttgctgtgtccccaggtgcagacagacccctcagggatctacattgccactagctgttccgataagaatctctccatttttgacttctcctcaggcgagtgtgtggccaccatgtttggccactcaggtgagtgagtgagtgtagcagccacctccctagttgaaattgtactttcttcccttggctgttgccacaccacaactctgccctaccctgggaggcatcacctgactaaagcaagagtagaaagccctgtggcagaaggggacagtatggttggtacctttcccttcccccatatccagactcagagcacaaagtgcctatgtgaggatagaacccgactctccctctcctgcagccagtgcccatccctgcagctggaagaggcaggaatttgattaggtctttgtggcagttcatagaccatatactttactttttacagagattgtcactggcatgaaatttagtaacgattgcaaacatctcatctctgtgtcaggggacaggtgagcagaagctaacttccctgagacaattcttctctgtgccacctgtccctcctctcattttctctcctggtgtctctgggaagtaggtctgaagagggacatttcttggtg

SEQ ID NO: 107 - DNA, 1064 bp, Rattus sp.
tgggcagcttggggtcggtgctaagtgaggaggggtggtgatcccctactcttgctgtgtccccaggtgcagacagacccctcagggatctacattgccactagctgttccgataagaatctctccatttttgacttctcctcaggcgagtgtgtggccaccatgtttggccactcaggtgagtgagtgagtgtagcagccacctccctagttgaaattgtactttcttcccttggctgttgccacaccacaactctgccctaccctgggaggcatcacctgactaaagcaagagtagaaagccctgtggcagaaggggacagtatggttggtacctttcccttcccccatatccagactcagagcacaaagtgcctatgtgaggatagaacccgactctccctctcctgcagccagtgcccatccctgcagctggaagaggcaggaatttgattaggtctttgtggcagttcatagaccatatactttactttttacagagattgtcactggcatgaaatttagtaacgattgcaaacatctcatctctgtgtcaggggacaggtgagcagaagctaacttccctgagacaattcttctctgtgccacctgtccctcctctcattttctctcctggtgtctctgggaagtaggtctgaagagggacatttcttggtggggcctgacctggcagtggagggtggcttggatcgccttgccaattcgtgggaagtagggataaacgatactgctacccgcagagtccttccctgcccctcactcagtgctgtggctccaccctagctgcatctttgtctggcgtctgagctctgagatgaccatcagcatgaggcagcgcttggctgagctgcggcagcgccagcgagggatcaagcagcaaggaccaacctctccccagagggcttctggagccaagcagtaagtgggccagagatgggctctgctcacggttgtgcacagggtgtgtgctctccctgcacctccttgtgtggaacgtactgttcagcatctccggtaatctcaaggccttcctttgcatgcac

SEQ ID NO: 108 - DNA, 1135 bp, Rattus sp.
ctgtggcagaaggggacagtatggttggtacctttcccttcccccatatccagactcagagcacaaagtgcctatgtgaggatagaacccgactctccctctcctgcagccagtgcccatccctgcagctggaagaggcaggaatttgattaggtctttgtggcagttcatagaccatatactttactttttacagagattgtcactggcatgaaatttagtaacgattgcaaacatctcatctctgtgtcaggggacaggtgagcagaagctaacttccctgagacaattcttctctgtgccacctgtccctcctctcattttctctcctggtgtctctgggaagtaggtctgaagagggacatttcttggtggggcctgacctggcagtggagggtggcttggatcgccttgccaattcgtgggaagtagggataaacgatactgctacccgcagagtccttccctgcccctcactcagtgctgtggctccaccctagctgcatctttgtctggcgtctgagctctgagatgaccatcagcatgaggcagcgcttggctgagctgcggcagcgccagcgagggatcaagcagcaaggaccaacctctccccagagggcttctggagccaagcagtaagtgggccagagatgggctctgctcacggttgtgcacagggtgtgtgctctccctgcacctccttgtgtggaacgtactgttcagcatctccggtaatctcaaggccttcctttgcatgcacaagcccatcagtgttgccttcttgcccagtgagtcttctcttctgaccttcttgtcttggactcaggcaccatgctccagtggtacccccttctggaccagctctttcctcagacagtgacaaggagggagaagatgagggtactgaagaagaagaactgccagctctgcccatccttagcaagagcaccaagaaagaactaggtttgtggcagttaggtgtaaagcagacaggcactagctgctgtggtgactaggttagtccatctggaccatgggataaggaggctgggccatcacagtgatcacatgggggatggggatgttctgggctcctggtcccagaggtgggcagggaggaaacacaccttctgac

SEQ ID NO: 109 - DNA, 1137 bp, Rattus sp.
ctcctggtgtctctgggaagtaggtctgaagagggacatttcttggtggggcctgacctggcagtggagggtggcttggatcgccttgccaattcgtgggaagtagggataaacgatactgctacccgcagagtccttccctgcccctcactcagtgctgtggctccaccctagctgcatctttgtctggcgtctgagctctgagatgaccatcagcatgaggcagcgcttggctgagctgcggcagcgccagcgagggatcaagcagcaaggaccaacctctccccagagggcttctggagccaagcagtaagtgggccagagatgggctctgctcacggttgtgcacagggtgtgtgctctccctgcacctccttgtgtggaacgtactgttcagcatctccggtaatctcaaggccttcctttgcatgcacaagcccatcagtgttgccttcttgcccagtgagtcttctcttctgaccttcttgtcttggactcaggcaccatgctccagtggtacccccttctggaccagctctttcctcagacagtgacaaggagggagaagatgagggtactgaagaagaagaactgccagctctgcccatccttagcaagagcaccaagaaagaactaggtttgtggcagttaggtgtaaagcagacaggcactagctgctgtggtgactaggttagtccatctggaccatgggataaggaggctgggccatcacagtgatcacatgggggatggggatgttctgggctcctggtcccagaggtgggcagggaggaaacacaccttctgactgcctatttctgtctagcctcaggctctagtccagccttgctccgaagcctgtcccactgggaaatgagtcgggtaagttgccatcactgaaatgtgcttctaagattattagacgtgagggatgaggcattgggtaaggacctgcccacatgcaccattcctgtttccaaggaaggccttatcattaaggtcaggcagcgggtctagtcggggataagggagtgagcatcagtttttccttcttgtatcagatgaggttgcctccactagaggagggcccacctcctcatctgccaggagcctgggagaagtaccattcaagagtag

SEQ ID NO: 110 - DNA, 1056 bp, Rattus sp.
gggctctgctcacggttgtgcacagggtgtgtgctctccctgcacctccttgtgtggaacgtactgttcagcatctccggtaatctcaaggccttcctttgcatgcacaagcccatcagtgttgccttcttgcccagtgagtcttctcttctgaccttcttgtcttggactcaggcaccatgctccagtggtacccccttctggaccagctctttcctcagacagtgacaaggagggagaagatgagggtactgaagaagaagaactgccagctctgcccatccttagcaagagcaccaagaaagaactaggtttgtggcagttaggtgtaaagcagacaggcactagctgctgtggtgactaggttagtccatctggaccatgggataaggaggctgggccatcacagtgatcacatgggggatggggatgttctgggctcctggtcccagaggtgggcagggaggaaacacaccttctgactgcctatttctgtctagcctcaggctctagtccagccttgctccgaagcctgtcccactgggaaatgagtcgggtaagttgccatcactgaaatgtgcttctaagattattagacgtgagggatgaggcattgggtaaggacctgcccacatgcaccattcctgtttccaaggaaggccttatcattaaggtcaggcagcgggtctagtcggggataagggagtgagcatcagtttttccttcttgtatcagatgaggttgcctccactagaggagggcccacctcctcatctgccaggagcctgggagaagtaccattcaagagtagcttggttttgttttgagtcagagtctcactgtgcagccctggctgacctggaactcagggagatccacagggccttagctctgagtgctgagaagaaaggccttgccaccacactgtgcttaatccaaactaaaccacagcagcaacaaagcagcagcgtgcagtgaggagtaatggtgtgagtcacatgttagagcaacagcccagggagagcaaggctccagcaaggagaaacctagggaact

SEQ ID NO: 111 - DNA, 1237 bp, Rattus sp.
caagttggacttttgagtctagcactctgtgttgttggtagcagccgcacagagtggcctcgtcagggtctcagtccctcattgttggtttggccttagtgctaagatgggactctgggttctgttgggtatcttcagagaacagaattatttaagagtcctagctctagactgtaagacaccagataaaggcagaaactaaagaggttcctattatgaggtctagaatgcggctccgagtatgcactctatagagctaaccttgcctgcaggccacttaaggtcccacatggacgtgggctagtcacagaaatccactgtgcttctgtttccctcgtgtgtaagacctgggataataatgctacctactctataaaatgactgtaaagataaaatgagctaatacacagagggtggcgttcatgccgacaatatgtacatatgtttgtttgctgatagtataattctaattagtactctttttttgccacaaccaccaggcacaagagaccatggagtacctggacccagctcctgtagctaacacaggacctaaaagaagagggcgctgggctcagccaggcgtggagctgagtgttcgctccatgttggacctgagacagatagagaccttagccccaagccctcgaggccccagccaggactcactggctgtgtccccagctggtcctgggaagcatggtccacagggccctgagctgtcatgtgtcagtcaggtgagctatctttctcccagctctctagaggtctcaggaagcctcagccggtcctgtgctaacagccggggcctgcgcttaggtacttctgtgcacagaagacacagagacaggaagtctcctctgaaatgcagttgtagtagaactgactgggctgtattgtgagtggggtgggccgagaggtacagggagctttggggaggattcttggtgttgagtagcaagagcctggcagtccgagtgccaaaacagctcttccagaggcttagtattccagctgagcccagcctggcctcctgcttcctgtccccagagctacaacactctgcttccgcagcactgctgttcaccagcaccttcttctcacgccagcaatcctcacaagtacccctttctttatgtgcttccagaatgaaagggcccctcggcttcagacctcccaaccctgctcctgcccccacattatccaattgttgtcacaagaggaaggagtctttgcc

SEQ ID NO: 112 - DNA, 1173 bp, Rattus sp.
cgaggccccagccaggactcactggctgtgtccccagctggtcctgggaagcatggtccacagggccctgagctgtcatgtgtcagtcaggtgagctatctttctcccagctctctagaggtctcaggaagcctcagccggtcctgtgctaacagccggggcctgcgcttaggtacttctgtgcacagaagacacagagacaggaagtctcctctgaaatgcagttgtagtagaactgactgggctgtattgtgagtggggtgggccgagaggtacagggagctttggggaggattcttggtgttgagtagcaagagcctggcagtccgagtgccaaaacagctcttccagaggcttagtattccagctgagcccagcctggcctcctgcttcctgtccccagagctacaacactctgcttccgcagcactgctgttcaccagcaccttcttctcacgccagcaatcctcacaagtacccctttctttatgtgcttccagaatgaaagggcccctcggcttcagacctcccaaccctgctcctgcccccacattatccaattgttgtcacaagaggaaggagtctttgcccaagatctggagcctgcacccattgaagatggtattgtctacccggaacccagtgacagccctaccatggataccaggcaagggttgtgccctggccagaactcatgggctctgcggcccttacacttcctttccctctgagttttcttacgtgggcagtggttggcctccaggccaccacctccctagcagtggtcacaggcaggagctgagtggagctgctgacccctgctcactgtctttccttcctttgctggcctttccggcagtgcgtttcaggtgcaggctccaaccggaggatccctaggaagaatgtacccaggcagcaggggctcagaaaagcacagtcctgacagtgcatgctctgtggattacagcagcagccggctttccagccctgaacaccctaatgaaggtaaggctgtaccatgaggaagggacaacggggtgaagcagggaaggcagacctgtgaccgataggtacagtgtgatgcctttcccctcccccccatccagactctgagagcacagagcccctaagtgtggatggcatctcctcagacctggaagagccagccgaggg

SEQ ID NO: 113 - DNA, 1146 bp, Rattus sp.
tattccagctgagcccagcctggcctcctgcttcctgtccccagagctacaacactctgcttccgcagcactgctgttcaccagcaccttcttctcacgccagcaatcctcacaagtacccctttctttatgtgcttccagaatgaaagggcccctcggcttcagacctcccaaccctgctcctgcccccacattatccaattgttgtcacaagaggaaggagtctttgcccaagatctggagcctgcacccattgaagatggtattgtctacccggaacccagtgacagccctaccatggataccaggcaagggttgtgccctggccagaactcatgggctctgcggcccttacacttcctttccctctgagttttcttacgtgggcagtggttggcctccaggccaccacctccctagcagtggtcacaggcaggagctgagtggagctgctgacccctgctcactgtctttccttcctttgctggcctttccggcagtgcgtttcaggtgcaggctccaaccggaggatccctaggaagaatgtacccaggcagcaggggctcagaaaagcacagtcctgacagtgcatgctctgtggattacagcagcagccggctttccagccctgaacaccctaatgaaggtaaggctgtaccatgaggaagggacaacggggtgaagcagggaaggcagacctgtgaccgataggtacagtgtgatgcctttcccctcccccccatccagactctgagagcacagagcccctaagtgtggatggcatctcctcagacctggaagagccagccgagggtgatgaagacgaggaagaagagggaggcactggcctctgtgggctacaagaaggcggccctcataccccagatcaggaacagtttctaaaacagcactttgagactctggccaatgggactgctccaggtgtggtctgtgggccagccttcagtcagccacatctgcccttgtcccctagctgagagggagggtccagtaggcagtgccttaggttgctttgtaggggctgcaaagacctcgggcaagaactaaacttctgactcagttcatggtttctgtgcctcctgcagggggcccagcacgggtgctagagaggacagagtctcagag

SEQ ID NO: 114 - DNA, 1195 bp, Rattus sp.
cacccattgaagatggtattgtctacccggaacccagtgacagccctaccatggataccaggcaagggttgtgccctggccagaactcatgggctctgcggcccttacacttcctttccctctgagttttcttacgtgggcagtggttggcctccaggccaccacctccctagcagtggtcacaggcaggagctgagtggagctgctgacccctgctcactgtctttccttcctttgctggcctttccggcagtgcgtttcaggtgcaggctccaaccggaggatccctaggaagaatgtacccaggcagcaggggctcagaaaagcacagtcctgacagtgcatgctctgtggattacagcagcagccggctttccagccctgaacaccctaatgaaggtaaggctgtaccatgaggaagggacaacggggtgaagcagggaaggcagacctgtgaccgataggtacagtgtgatgcctttcccctcccccccatccagactctgagagcacagagcccctaagtgtggatggcatctcctcagacctggaagagccagccgagggtgatgaagacgaggaagaagagggaggcactggcctctgtgggctacaagaaggcggccctcataccccagatcaggaacagtttctaaaacagcactttgagactctggccaatgggactgctccaggtgtggtctgtgggccagccttcagtcagccacatctgcccttgtcccctagctgagagggagggtccagtaggcagtgccttaggttgctttgtaggggctgcaaagacctcgggcaagaactaaacttctgactcagttcatggtttctgtgcctcctgcagggggcccagcacgggtgctagagaggacagagtctcagagcatctcatcacgattccttctgcaagtgcagacctccccactcaggtacggaccaccccacatggctctgctcacctggtgctcctgcacctggcacagcatgtagccttcctcatgtctctccagaaggggacgttgtactcctagatttgaagcaggtggtctcacgcagactctgtcttgctttgtgcttgtcctctcctggtgcatctctgtctgtgagggcatctggccagctcagggaggaaagcactgagccttctctgggccttcttctgaacccattctccctgtag

SEQ ID NO: 115 - DNA, 1085 bp, Rattus sp.
agcagcagccggctttccagccctgaacaccctaatgaaggtaaggctgtaccatgaggaagggacaacggggtgaagcagggaaggcagacctgtgaccgataggtacagtgtgatgcctttcccctcccccccatccagactctgagagcacagagcccctaagtgtggatggcatctcctcagacctggaagagccagccgagggtgatgaagacgaggaagaagagggaggcactggcctctgtgggctacaagaaggcggccctcataccccagatcaggaacagtttctaaaacagcactttgagactctggccaatgggactgctccaggtgtggtctgtgggccagccttcagtcagccacatctgcccttgtcccctagctgagagggagggtccagtaggcagtgccttaggttgctttgtaggggctgcaaagacctcgggcaagaactaaacttctgactcagttcatggtttctgtgcctcctgcagggggcccagcacgggtgctagagaggacagagtctcagagcatctcatcacgattccttctgcaagtgcagacctccccactcaggtacggaccaccccacatggctctgctcacctggtgctcctgcacctggcacagcatgtagccttcctcatgtctctccagaaggggacgttgtactcctagatttgaagcaggtggtctcacgcagactctgtcttgctttgtgcttgtcctctcctggtgcatctctgtctgtgagggcatctggccagctcagggaggaaagcactgagccttctctgggccttcttctgaacccattctccctgtagggaaccatccctatcctcctcaggcttggccctgacgtccagacctgaccaggtatcacaggtgtctggtgagcagctgaaaggcagtggtgccactcctccaggagcacccccagaaatggaaccctcttctggcaactctggccccaagcaggtggctcctgtgctgttgacacgacggcataacaacttggacaacagctgggcctccaagaaaatggctgcaacccggcctttagctggactcca

SEQ ID NO: 116 - DNA, 1284 bp, Rattus sp.
gtgtggtctgtgggccagccttcagtcagccacatctgcccttgtcccctagctgagagggagggtccagtaggcagtgccttaggttgctttgtaggggctgcaaagacctcgggcaagaactaaacttctgactcagttcatggtttctgtgcctcctgcagggggcccagcacgggtgctagagaggacagagtctcagagcatctcatcacgattccttctgcaagtgcagacctccccactcaggtacggaccaccccacatggctctgctcacctggtgctcctgcacctggcacagcatgtagccttcctcatgtctctccagaaggggacgttgtactcctagatttgaagcaggtggtctcacgcagactctgtcttgctttgtgcttgtcctctcctggtgcatctctgtctgtgagggcatctggccagctcagggaggaaagcactgagccttctctgggccttcttctgaacccattctccctgtagggaaccatccctatcctcctcaggcttggccctgacgtccagacctgaccaggtatcacaggtgtctggtgagcagctgaaaggcagtggtgccactcctccaggagcacccccagaaatggaaccctcttctggcaactctggccccaagcaggtggctcctgtgctgttgacacgacggcataacaacttggacaacagctgggcctccaagaaaatggctgcaacccggcctttagctggactccagaaagcccagtctgtgcatagtttggtaccacagggtaaggaaccttgatgagttcaactacaggttgtgggggatgtgagacatatatgtgggcatgctggaggaaggtgggtctagtgtggccctggcatgtcaccatgcttgccactgcatgtcccttagtgattacacacaggggagagtagcaactggggactgagccctagcatttgcacactttcctcaccacgcgacctgcatctgtccctgtccccaatgcagatgaggtgccttcatcacgtccactgctcttccaggaggcagagacccagggcagcttaggatccctgccacaagctggtggctgctcatctcagccccactcctaccagaaccacaccaccagttctatggccaagctagcgcgtagtatttctgttggcgagaatccgggcctggcaactgaacctcaagctcctgtaccgatccgaatctcaccattcaacaaactagctctgcctagcagggctcaccttgtcctggacatccccaaac

SEQ ID NO: 117 - DNA, 1597 bp, Rattus sp.
tatcctcctcaggcttggccctgacgtccagacctgaccaggtatcacaggtgtctggtgagcagctgaaaggcagtggtgccactcctccaggagcacccccagaaatggaaccctcttctggcaactctggccccaagcaggtggctcctgtgctgttgacacgacggcataacaacttggacaacagctgggcctccaagaaaatggctgcaacccggcctttagctggactccagaaagcccagtctgtgcatagtttggtaccacagggtaaggaaccttgatgagttcaactacaggttgtgggggatgtgagacatatatgtgggcatgctggaggaaggtgggtctagtgtggccctggcatgtcaccatgcttgccactgcatgtcccttagtgattacacacaggggagagtagcaactggggactgagccctagcatttgcacactttcctcaccacgcgacctgcatctgtccctgtccccaatgcagatgaggtgccttcatcacgtccactgctcttccaggaggcagagacccagggcagcttaggatccctgccacaagctggtggctgctcatctcagccccactcctaccagaaccacaccaccagttctatggccaagctagcgcgtagtatttctgttggcgagaatccgggcctggcaactgaacctcaagctcctgtaccgatccgaatctcaccattcaacaaactagctctgcctagcagggctcaccttgtcctggacatccccaaaccacttcctgaccgtcctactctgaccacattctcacctgtatccaagggcctggcccacaatgaaacagaacaatcgggccccttggtgagcctaggaaaggctcatactacagttgaaaagcactcctgtttaggggagggtactactcataaatctaggacagagtgccaggcttatcctggacccaaccacccctgtgcccagcaactgccagtcaacaaccttctccaaggccctgagagcttgcagcccctgtcccctgagaagactcgtaacccagtggaaagcagcaggccaggggtagccctgagccaggactcaggtgtgcatggctccccagtctcacctggcccatcctttctgtctctgttggctactttctggctctgtcccatttctgtgtgcctggcctttggtgtttttgtcctgtctgctcctgtcttggtccataagtcacacctctgatggtgagacactcttgccacccttcataatctccctgtgacccagtgaaccctttagtctggatactagggctggcttttccttctaaactttgtgtggtgttggggaaggtgttggaagaaggtgggacaagaggccaggattgtcggctggtagggattatagctctgctctgagagccctgggttgggatgcctgaaaaagcagcagggcctcactggtgtttgtccttcagaactggccttgagtctgcaacagtgtgaacagctcgtggcagagctccaggggaatgtacgccaggcagtggagctctaccgagcggtgagtatggggcaaccactgtaggcaacaacagag

SEQ ID NO: 118 - DNA, 1086 bp, Rattus sp.
ccagcaactgccagtcaacaaccttctccaaggccctgagagcttgcagcccctgtcccctgagaagactcgtaacccagtggaaagcagcaggccaggggtagccctgagccaggactcaggtgtgcatggctccccagtctcacctggcccatcctttctgtctctgttggctactttctggctctgtcccatttctgtgtgcctggcctttggtgtttttgtcctgtctgctcctgtcttggtccataagtcacacctctgatggtgagacactcttgccacccttcataatctccctgtgacccagtgaaccctttagtctggatactagggctggcttttccttctaaactttgtgtggtgttggggaaggtgttggaagaaggtgggacaagaggccaggattgtcggctggtagggattatagctctgctctgagagccctgggttgggatgcctgaaaaagcagcagggcctcactggtgtttgtccttcagaactggccttgagtctgcaacagtgtgaacagctcgtggcagagctccaggggaatgtacgccaggcagtggagctctaccgagcggtgagtatggggcaaccactgtaggcaacaacagagcttcttctacacactggatggagaactcaagggtattctgagcattgtaaaggggagggtcagaagccaggccaggcctctcaaccccagaatctgttggttatttctaggcaagaaacagggtcgtagcgtagaacaccatattattcaagaaaatcccaattcatggctgttatcttttttttttatatcccaaaatggtttgggcactttagatagaagaaacaatgtaactgtcacatttgaaagagttgttgggaaaagttgtcaagaagtagatggttctaatagtctggagggatcctcatctgggccacccatcagagtgactctggccatcctgatcttgtgtgtagaactcctaggcagtcccttctactgggtgggcaaggcactggcagagccccttttctatattccactgtctactgctctccagctcactttcagtgttcctgttgcag

SEQ ID NO: 119 - DNA, 1236 bp, Rattus sp.
gtgagtatggggcaaccactgtaggcaacaacagagcttcttctacacactggatggagaactcaagggtattctgagcattgtaaaggggagggtcagaagccaggccaggcctctcaaccccagaatctgttggttatttctaggcaagaaacagggtcgtagcgtagaacaccatattattcaagaaaatcccaattcatggctgttatcttttttttttatatcccaaaatggtttgggcactttagatagaagaaacaatgtaactgtcacatttgaaagagttgttgggaaaagttgtcaagaagtagatggttctaatagtctggagggatcctcatctgggccacccatcagagtgactctggccatcctgatcttgtgtgtagaactcctaggcagtcccttctactgggtgggcaaggcactggcagagccccttttctatattccactgtctactgctctccagctcactttcagtgttcctgttgcaggtgaccagctataagacaccttcggcagagcaaagtcacatcacccgtctcctgagagacaccttctcttcggtgcgacaggagctcgaggttctggctggggcggtgctgtccagcccaggtggcagccctggggctgtgggggctgagcagacgcaggccctgttggagcaatactccgagctactgctaagagctgtggagcggcgcatggagcgtagactctgagttcctgaagcctgtcccaagagaaatgctctccattccaaactgtatccctgcttccactcaagaaaaggtgggaatgtctggagtggacagcagtgatcagtgttgagaggcaaagcagccttcccagccgccctcatggagcccctgtatttattaatttatttccctgactttgcctcacttccttgggactcctgcctctaaagcccagcctggggctcacagcagggcaagtcttgagtctacatcatcttggtgctgtgaggggtaggagggcagcctgcctcccctggggacatcgggaagggctggccttctcttcttagaagccatttgaagtttctgcagggatgagctccctggggcggagcatccacagggtactctgggatagagatgaaaggtaatgtggtatttagtgtcctaaagccaactgtagacctttacctctactcccacaccagctgcactcccctgcctgccagagcaggggtagtttcccccc

SEQ ID NO: 120 - DNA, 1065 bp, Unknown Organism
  Description of Unknown Organism CPLA2-beta, EMBL No. q9ukv7
acgcatagtcaaactcctagcccaaagaaccgaagccacttgaggtgggccccttggcagtgtgggggttagaagtcggcccactgccctgacctccaccaggttttagaggtctttatgccagcctgcccttctagagaaagcgccactaccctgcatgggtggggtcttgtggcctgtggcactttgtcctcttctgcatgtctgaggccactggcaattgctgcccatgtccagatttgtcctatcccagttttgaaagccccttatttataacttttatactgtggcgcctgtctcctccccggctgtacggggtgggactgttttaacgtttcgtttgtacggatgtatgaaattgtcaataaacacaatcatttgttaacctgctagcaaattctgagaagagcgccgcttgttgcacctcctagagggcggaaacgggggcggggctgcgatcctggagcggaaataggggcgggttcttgtcccgcggccgtcatggcggaggcggctctggaggcagttcggagggcgttgcaagagttcccggcggcggctcgcggtgagtgcactctcacaggcagcagcgaccgcgcgaggacagcgcggagggagcctgggcaccctggaactcagtgtgcatgagtgtccgaccggtggccacacagttttgtgtgtacccttctttagctggcggacggctccttagctcaggatcgcacgcctggctgccttcgtggggtgcagttgtagttttcaagacccagcacagctctgacggtagtcatggtgccattctcaaagccagcggggtgctgagtccccgtgcgctgccatgctatactttcacctcaaacatctcggatgctggtgggttctgaggagagaagcaggcgacttccctcgttcattcagactccaagctttgaggattgtaggcagccgcccagttgatcttgtgtgtttcctttcttcttcctctggtcctcactaccccatcattaaacaagctggagagatggctaagtgggtaggtaaagctcttaagcacatgatgatctg

SEQ ID NO: 121 - DNA, 1154 bp, Unknown Organism
  Description of Unknown Organism CPLA2-beta, EMBL No. q9ukv7
ggcccggtgctgggtccatttcaggcgttcagtgaatatcaccattggaggctgtcttaggattttactgccgtgaagagacaccataatcaccacaacccttataaaggacaacatttaactggggctggcttacagcttcagtctggaggccagctgagggttctacatctggatcctcaagaagagactgtcactggccagacttgagcttataaaacatcaaagcccactcctagtgacacacttcctctaacaaggccacaccgcctgttagtgctactccctgttggtcaaggattcacacacatgaatctgtggggtcatccagagccagacccaccccagttagcctgtggccaccagctcagtagtgcaggcctcctaggtcggaagcacccttgcttaggatgtccgtcatagagggcttcttggtgcagtgtgatggaagttgggtaccactgatttcacacctcctccaccaccatttctttcttaagacctcaatgtacctcgtgttgtgccctacctggatgagcccccaagcccactctgcttctaccgggattgggtgtgccccaacaggccctgcattatccgaaatgctctgcagcactggccagccctccagaagtggtccctgtcctacttaaggtaaggtgttccttgggaggttgggtggaacagtgaccattagcagcagactacaaaccaagtgtctgtttcttcagagccacggtgggctccacggaggtgagtgtggctgtgactccagatggttatgcggacgcggtgcgaggggaccgctttgtgatgcctgctgaacgccgcctgcccataagccatgtactggatgtgttggaaggtcgggcccagcacccaggagtcctctatgtgcagaaacagtgttccaacctgcccactgagctgccccagcttctgtctgacattgagtcccatgtgccctgggcctctgagtcactgggtgagtggtgggggtaagaggcggggtatggacgtgaagtggggagataagagttcagctagcttgttgattctctgccttttgtgttgattctgtgggggcaggtgctacctatctgcccgctccagggctgctgtctctgccatcctgggattgtgttcccagtgtc

SEQ ID NO: 122 - DNA, 1254 bp, Unknown Organism
  Description of Unknown Organism CPLA2-beta, EMBL No. q9ukv7
actcctagtgacacacttcctctaacaaggccacaccgcctgttagtgctactccctgttggtcaaggattcacacacatgaatctgtggggtcatccagagccagacccaccccagttagcctgtggccaccagctcagtagtgcaggcctcctaggtcggaagcacccttgcttaggatgtccgtcatagagggcttcttggtgcagtgtgatggaagttgggtaccactgatttcacacctcctccaccaccatttctttcttaagacctcaatgtacctcgtgttgtgccctacctggatgagcccccaagcccactctgcttctaccgggattgggtgtgccccaacaggccctgcattatccgaaatgctctgcagcactggccagccctccagaagtggtccctgtcctacttaaggtaaggtgttccttgggaggttgggtggaacagtgaccattagcagcagactacaaaccaagtgtctgtttcttcagagccacggtgggctccacggaggtgagtgtggctgtgactccagatggttatgcggacgcggtgcgaggggaccgctttgtgatgcctgctgaacgccgcctgcccataagccatgtactggatgtgttggaaggtcgggcccagcacccaggagtcctctatgtgcagaaacagtgttccaacctgcccactgagctgccccagcttctgtctgacattgagtcccatgtgccctgggcctctgagtcactgggtgagtggtgggggtaagaggcggggtatggacgtgaagtggggagataagagttcagctagcttgttgattctctgccttttgtgttgattctgtgggggcaggtgctacctatctgcccgctccagggctgctgtctctgccatcctgggattgtgttcccagtgtcaccttcccccagactcggcactcttctctctcacccatatttctgcatagctgccccccacacgggtggttctcagcctgatattaattcactgtgtcccacagggaagatgcctgatgccgtgaacttctggctgggtgatgcatctgcagtgacatcctgtaggtgtggagactcctcaagtggggaggggcagagggaagaaaggtgggccccagaacgagtacctcagatttcttttggtattgtgggactaggagaccacagagtttctcctgatggcaggaacactggggacagggaatactgtaccttttaagtctctagaagc

SEQ ID NO: 123 - DNA, 1057 bp, Unknown Organism
  Description of Unknown Organism CPLA2-beta, EMBL No. q9ukv7
tgtggctgtgactccagatggttatgcggacgcggtgcgaggggaccgctttgtgatgcctgctgaacgccgcctgcccataagccatgtactggatgtgttggaaggtcgggcccagcacccaggagtcctctatgtgcagaaacagtgttccaacctgcccactgagctgccccagcttctgtctgacattgagtcccatgtgccctgggcctctgagtcactgggtgagtggtgggggtaagaggcggggtatggacgtgaagtggggagataagagttcagctagcttgttgattctctgccttttgtgttgattctgtgggggcaggtgctacctatctgcccgctccagggctgctgtctctgccatcctgggattgtgttcccagtgtcaccttcccccagactcggcactcttctctctcacccatatttctgcatagctgccccccacacgggtggttctcagcctgatattaattcactgtgtcccacagggaagatgcctgatgccgtgaacttctggctgggtgatgcatctgcagtgacatcctgtaggtgtggagactcctcaagtggggaggggcagagggaagaaaggtgggccccagaacgagtacctcagatttcttttggtattgtgggactaggagaccacagagtttctcctgatggcaggaacactggggacagggaatactgtaccttttaagtctctagaagcctcttcagaagtcaggtagatctgagagcaggggtcaggagataacgcccgcccattggtgatccagatgaaggggagtccacatccagtttttggtggccaagttctgtgattcttgcaagaaccttggaatctccacaagtctgctaatcctgtgtgggcttttcctttctctgtaggctctgctagggtgtggggtgtggggctgaggacagtgtggcctgcttccctccacttaactccaggccttccatgtgtcaacagtgcacaaggaccactatgagaatctgtactgtgtagtctctggcgagaaacacttcttgttacatc

SEQ ID NO: 124 - DNA, 1096 bp, Unknown Organism
  Description of Unknown Organism CPLA2-beta, EMBL No. q9ukv7
gtcccacagggaagatgcctgatgccgtgaacttctggctgggtgatgcatctgcagtgacatcctgtaggtgtggagactcctcaagtggggaggggcagagggaagaaaggtgggccccagaacgagtacctcagatttcttttggtattgtgggactaggagaccacagagtttctcctgatggcaggaacactggggacagggaatactgtaccttttaagtctctagaagcctcttcagaagtcaggtagatctgagagcaggggtcaggagataacgcccgcccattggtgatccagatgaaggggagtccacatccagtttttggtggccaagttctgtgattcttgcaagaaccttggaatctccacaagtctgctaatcctgtgtgggcttttcctttctctgtaggctctgctagggtgtggggtgtggggctgaggacagtgtggcctgcttccctccacttaactccaggccttccatgtgtcaacagtgcacaaggaccactatgagaatctgtactgtgtagtctctggcgagaaacacttcttgttacatccacccagcgaccggcccttcatcccttacagtaggtgacatgaactaaaagaagctagtagtcactggtgagggaagggcagcaagaggtccctgccatgggccccagccctctggtctctgtatgcagggaacgctgtgctggtcatggtgagagtgcctgacaggctgttggccccttgtctccctgaccttgccttcagatctctacacaccagcaacctaccagctgactgaagagggcacttttagggtggtggacgaggaagccatggagaaggtgtctgtcctgttcttgggctctggggagtgggtagacctctggggaataggcacaaagggcctggggaggtggcatcccactctgaaaatccctaggctgtgattcttcaagccgagaggagaggggacagagctggagatcttggggacctcagggcctgctctcaggcagccgctgtgtctctaggtaccctggatcccactggaccccttggctccagacctgacccagtaccccagttacagcca

SEQ ID NO: 125 - DNA, 1077 bp, Unknown Organism
  Description of Unknown Organism CPLA2-beta, EMBL No. q9ukv7
ggtcaggagataacgcccgcccattggtgatccagatgaaggggagtccacatccagtttttggtggccaagttctgtgattcttgcaagaaccttggaatctccacaagtctgctaatcctgtgtgggcttttcctttctctgtaggctctgctagggtgtggggtgtggggctgaggacagtgtggcctgcttccctccacttaactccaggccttccatgtgtcaacagtgcacaaggaccactatgagaatctgtactgtgtagtctctggcgagaaacacttcttgttacatccacccagcgaccggcccttcatcccttacagtaggtgacatgaactaaaagaagctagtagtcactggtgagggaagggcagcaagaggtccctgccatgggccccagccctctggtctctgtatgcagggaacgctgtgctggtcatggtgagagtgcctgacaggctgttggccccttgtctccctgaccttgccttcagatctctacacaccagcaacctaccagctgactgaagagggcacttttagggtggtggacgaggaagccatggagaaggtgtctgtcctgttcttgggctctggggagtgggtagacctctggggaataggcacaaagggcctggggaggtggcatcccactctgaaaatccctaggctgtgattcttcaagccgagaggagaggggacagagctggagatcttggggacctcagggcctgctctcaggcagccgctgtgtctctaggtaccctggatcccactggaccccttggctccagacctgacccagtaccccagttacagccaggcacaggcccttcactgcacagtgcgggccggcgagatgctgtacctgccagctctgtggttccaccatgtccagcagtcccacggctgtattgctggtaagtagcacctggagcatccagggggtcaggccttgtcatggcccaacaagggatcgggttggggtggctctggcttaaatctcacatccgggtccccctcctctccacagtgaatttctggtatgacatggagtatgacctcaagtac

SEQ ID NO: 126 - DNA, 1073 bp, Unknown Organism
  Description of Unknown Organism CPLA2-beta, EMBL No. q9ukv7
tggcatgggtttcagctcactcacagtgcctaagccccaatgtattcattcctgtcccttgaaggctaatctgacatctcagatcagccctaaagcctcttagtaggagtggggtgggatggtgaggcagggggataaaaagacaaaggataccatacccagaaatggtgggtgacactaactagcttgcctgtgttctgagccaaacagaacctaggagagtggcctcagtttcctcccagggctggccattgtcatagctcccttcataggatatgtgacaaataagtaaatgtatggagctggcacggtgttatataattatgggaaggtgtcatgtagatgtatcttgtgtcactctggcattctgatggctacaggtggacctggtccttttggtagagaattaagccttaagagtttgtccctgtcccctctcctgtggccctgtcatatttgaccccgtctacctatagcaaacctgcccagtctacatgcaggcaaaggtgccagagacatgcctgctcactgtacgcgtcctccgggccagtggcttgccttccaaggacctaggtaagcacactgctttcctgtcccttagctaagcaggcagtaggtgggctgcttcagagtagttaagtaccagtgggagggtctccagggaaggccgggctggtcttgcagatggctgccggagggacaatcccaaaagtggcagagggctcagtcctatggacatgaagtgtctgggagaaggaagtggactctggcagcctgggaaatttcttgatcatacccgacacatgttcctccttcctgcagtaacctcctctgactgctatgtgactctgaacctgcccacggcttccagccacacgctccagacacgcacagtcaagaacagccgaaaccctgtctggaatcagaacttccacttccggatccataggcagctcaaggtagaccaggcatcaagtctggccctgtccttgtccctgtgctccatcgtcacacgtgtctcctagtctgccattcccttccccgttcacctctcacccatctcattcctgtag

SEQ ID NO: 127 - DNA, 1137 bp, Unknown Organism
  Description of Unknown Organism CPLA2-beta, EMBL No. q9ukv7
tatgggaaggtgtcatgtagatgtatcttgtgtcactctggcattctgatggctacaggtggacctggtccttttggtagagaattaagccttaagagtttgtccctgtcccctctcctgtggccctgtcatatttgaccccgtctacctatagcaaacctgcccagtctacatgcaggcaaaggtgccagagacatgcctgctcactgtacgcgtcctccgggccagtggcttgccttccaaggacctaggtaagcacactgctttcctgtcccttagctaagcaggcagtaggtgggctgcttcagagtagttaagtaccagtgggagggtctccagggaaggccgggctggtcttgcagatggctgccggagggacaatcccaaaagtggcagagggctcagtcctatggacatgaagtgtctgggagaaggaagtggactctggcagcctgggaaatttcttgatcatacccgacacatgttcctccttcctgcagtaacctcctctgactgctatgtgactctgaacctgcccacggcttccagccacacgctccagacacgcacagtcaagaacagccgaaaccctgtctggaatcagaacttccacttccggatccataggcagctcaaggtagaccaggcatcaagtctggccctgtccttgtccctgtgctccatcgtcacacgtgtctcctagtctgccattcccttccccgttcacctctcacccatctcattcctgtagctgcttctctttgtgcctcttccagaatgttatggaactgaaagtctttgaccatgacctggtgaccagagatgacccagtattgtcagtgctgtttgacgtggggaccctgcaaattgggactcagcgccagagcttctccttgggtactcaggtaaagctcgggaaagtaggggtccccagtgctggggctggagcttcggagcctcccctgctccagggaggcccaggaatctcaagtgacttcaccaaggagcagtcctgtctctgactgctttctcacgtggtcagaagtccagggagtgtagacagagtgacctggaaagagacagttgcgcttccccactacgaaggcctcggcagcctccccgcccatctggtttgcc

SEQ ID NO: 128 - DNA, 1129 bp, Unknown Organism
  Description of Unknown Organism CPLA2-beta, EMBL No. q9ukv7
tagctaagcaggcagtaggtgggctgcttcagagtagttaagtaccagtgggagggtctccagggaaggccgggctggtcttgcagatggctgccggagggacaatcccaaaagtggcagagggctcagtcctatggacatgaagtgtctgggagaaggaagtggactctggcagcctgggaaatttcttgatcatacccgacacatgttcctccttcctgcagtaacctcctctgactgctatgtgactctgaacctgcccacggcttccagccacacgctccagacacgcacagtcaagaacagccgaaaccctgtctggaatcagaacttccacttccggatccataggcagctcaaggtagaccaggcatcaagtctggccctgtccttgtccctgtgctccatcgtcacacgtgtctcctagtctgccattcccttccccgttcacctctcacccatctcattcctgtagctgcttctctttgtgcctcttccagaatgttatggaactgaaagtctttgaccatgacctggtgaccagagatgacccagtattgtcagtgctgtttgacgtggggaccctgcaaattgggactcagcgccagagcttctccttgggtactcaggtaaagctcgggaaagtaggggtccccagtgctggggctggagcttcggagcctcccctgctccagggaggcccaggaatctcaagtgacttcaccaaggagcagtcctgtctctgactgctttctcacgtggtcagaagtccagggagtgtagacagagtgacctggaaagagacagttgcgcttccccactacgaaggcctcggcagcctccccgcccatctggtttgccagtccctctgatttgcttctcttcccaggagaaagggtgcttagaagttgagtttcggctacagactctgtaagttggtcttggagcaggggtgggggtgggggtggggatgggggtagggttggtgagtggcagtggaaagcccccttctttggagggacagtgccagctcaccaaggctagcacctctccctggtagaggaggagtgcctggcctcttgcagagagtcactggggcctctttccaggacagactgtgaggaacagc

SEQ ID NO: 129 - DNA, 1041 bp, Unknown Organism
  Description of Unknown Organism CPLA2-beta, EMBL No. q9ukv7
ccttgtccctgtgctccatcgtcacacgtgtctcctagtctgccattcccttccccgttcacctctcacccatctcattcctgtagctgcttctctttgtgcctcttccagaatgttatggaactgaaagtctttgaccatgacctggtgaccagagatgacccagtattgtcagtgctgtttgacgtggggaccctgcaaattgggactcagcgccagagcttctccttgggtactcaggtaaagctcgggaaagtaggggtccccagtgctggggctggagcttcggagcctcccctgctccagggaggcccaggaatctcaagtgacttcaccaaggagcagtcctgtctctgactgctttctcacgtggtcagaagtccagggagtgtagacagagtgacctggaaagagacagttgcgcttccccactacgaaggcctcggcagcctccccgcccatctggtttgccagtccctctgatttgcttctcttcccaggagaaagggtgcttagaagttgagtttcggctacagactctgtaagttggtcttggagcaggggtgggggtgggggtggggatgggggtagggttggtgagtggcagtggaaagcccccttctttggagggacagtgccagctcaccaaggctagcacctctccctggtagaggaggagtgcctggcctcttgcagagagtcactggggcctctttccaggacagactgtgaggaacagctcatcagcaatggcatcgtggtggtgagtgaggaacccagactccccttagtagctcatccctcgtgctgtctgctgctctccaaggcctgatgcaggtccacacagtctgtgtcatgtaggccgagttggggaccttggcatgcatgttcccaagagagtacatctcaagtcaatgcttagtgactcagggtgatgaaagaatattctgggaacagggaccagaagcccacaatgtacagcttgtgcctaagggggtctcttgtctgtaggcccgggaactctcctgcttgcacgttgaa

SEQ ID NO: 130 - DNA, 1043 bp, Unknown Organism
  Description of Unknown Organism CPLA2-beta, EMBL No. q9ukv7
gcttctccttgggtactcaggtaaagctcgggaaagtaggggtccccagtgctggggctggagcttcggagcctcccctgctccagggaggcccaggaatctcaagtgacttcaccaaggagcagtcctgtctctgactgctttctcacgtggtcagaagtccagggagtgtagacagagtgacctggaaagagacagttgcgcttccccactacgaaggcctcggcagcctccccgcccatctggtttgccagtccctctgatttgcttctcttcccaggagaaagggtgcttagaagttgagtttcggctacagactctgtaagttggtcttggagcaggggtgggggtgggggtggggatgggggtagggttggtgagtggcagtggaaagcccccttctttggagggacagtgccagctcaccaaggctagcacctctccctggtagaggaggagtgcctggcctcttgcagagagtcactggggcctctttccaggacagactgtgaggaacagctcatcagcaatggcatcgtggtggtgagtgaggaacccagactccccttagtagctcatccctcgtgctgtctgctgctctccaaggcctgatgcaggtccacacagtctgtgtcatgtaggccgagttggggaccttggcatgcatgttcccaagagagtacatctcaagtcaatgcttagtgactcagggtgatgaaagaatattctgggaacagggaccagaagcccacaatgtacagcttgtgcctaagggggtctcttgtctgtaggcccgggaactctcctgcttgcacgttgaactgaagaggacaggagatccaaagagtgagttctgtcctctgtaggcagcccttctgcccttcttctcctcttctgagggggtaaacttgttatactggctgctttaggtcccaaggttgggaggtaagcccatgccgggcctcccctgggctccatctctccggagatctggaccatgtcagagacgatttccctgcagacttgataaggcattccgtgga

SEQ ID NO: 131 - DNA, 1055 bp, Unknown Organism
  Description of Unknown Organism CPLA2-beta, EMBL No. q9ukv7
cttagaagttgagtttcggctacagactctgtaagttggtcttggagcaggggtgggggtgggggtggggatgggggtagggttggtgagtggcagtggaaagcccccttctttggagggacagtgccagctcaccaaggctagcacctctccctggtagaggaggagtgcctggcctcttgcagagagtcactggggcctctttccaggacagactgtgaggaacagctcatcagcaatggcatcgtggtggtgagtgaggaacccagactccccttagtagctcatccctcgtgctgtctgctgctctccaaggcctgatgcaggtccacacagtctgtgtcatgtaggccgagttggggaccttggcatgcatgttcccaagagagtacatctcaagtcaatgcttagtgactcagggtgatgaaagaatattctgggaacagggaccagaagcccacaatgtacagcttgtgcctaagggggtctcttgtctgtaggcccgggaactctcctgcttgcacgttgaactgaagaggacaggagatccaaagagtgagttctgtcctctgtaggcagcccttctgcccttcttctcctcttctgagggggtaaacttgttatactggctgctttaggtcccaaggttgggaggtaagcccatgccgggcctcccctgggctccatctctccggagatctggaccatgtcagagacgatttccctgcagacttgataaggcattccgtggacagatcccgtcctgagttttggttctggttcctgtttttagggtcagagcgcaaagttcaacttgtggttgctggggcctgtgagggcccacaggatgcctctgcgggcactggatctttccacttccattatccagcctgctgggagcaagagctgaatgttcatctgcaggtgctttgacttgctcccaggaccctagggccacagcctctcttgtctcagcctgagctgttctccccccgccccgccccctcatactgctcctgtctgtcaggtcacatgggcctttgttcctttccctg

SEQ ID NO: 132 - DNA, 1131 bp, Unknown Organism
  Description of Unknown Organism CPLA2-beta, EMBL No. q9ukv7
gtgctgtctgctgctctccaaggcctgatgcaggtccacacagtctgtgtcatgtaggccgagttggggaccttggcatgcatgttcccaagagagtacatctcaagtcaatgcttagtgactcagggtgatgaaagaatattctgggaacagggaccagaagcccacaatgtacagcttgtgcctaagggggtctcttgtctgtaggcccgggaactctcctgcttgcacgttgaactgaagaggacaggagatccaaagagtgagttctgtcctctgtaggcagcccttctgcccttcttctcctcttctgagggggtaaacttgttatactggctgctttaggtcccaaggttgggaggtaagcccatgccgggcctcccctgggctccatctctccggagatctggaccatgtcagagacgatttccctgcagacttgataaggcattccgtggacagatcccgtcctgagttttggttctggttcctgtttttagggtcagagcgcaaagttcaacttgtggttgctggggcctgtgagggcccacaggatgcctctgcgggcactggatctttccacttccattatccagcctgctgggagcaagagctgaatgttcatctgcaggtgctttgacttgctcccaggaccctagggccacagcctctcttgtctcagcctgagctgttctccccccgccccgccccctcatactgctcctgtctgtcaggtcacatgggcctttgttcctttccctgccggtcttctgtccttgccacagggagttagttggtggggtcttagcaaagaaaggagtaatgactcgcagggtcctttctctccctgtccctaaggatgacccccatgagcagctgaaggtgccactacgaactctaccctcctcgcagctggtgagacttgtcttccccacgtcccaggtacatctggtcatcgagagaagctgggttcatgcacagggctgctgctggccctggtctgcagtcagcctccttcaggtcatgcccgaggcgatcagtgggtgcaagggtggaccctgtgaaagtgcttggggcccaagggttcatggggatgttctggggtgcaccgaggccacagtggggtggagg

SEQ ID NO: 133 - DNA, 1084 bp, Unknown Organism
  Description of Unknown Organism CPLA2-beta, EMBL No. q9ukv7
gaggtaagcccatgccgggcctcccctgggctccatctctccggagatctggaccatgtcagagacgatttccctgcagacttgataaggcattccgtggacagatcccgtcctgagttttggttctggttcctgtttttagggtcagagcgcaaagttcaacttgtggttgctggggcctgtgagggcccacaggatgcctctgcgggcactggatctttccacttccattatccagcctgctgggagcaagagctgaatgttcatctgcaggtgctttgacttgctcccaggaccctagggccacagcctctcttgtctcagcctgagctgttctccccccgccccgccccctcatactgctcctgtctgtcaggtcacatgggcctttgttcctttccctgccggtcttctgtccttgccacagggagttagttggtggggtcttagcaaagaaaggagtaatgactcgcagggtcctttctctccctgtccctaaggatgacccccatgagcagctgaaggtgccactacgaactctaccctcctcgcagctggtgagacttgtcttccccacgtcccaggtacatctggtcatcgagagaagctgggttcatgcacagggctgctgctggccctggtctgcagtcagcctccttcaggtcatgcccgaggcgatcagtgggtgcaagggtggaccctgtgaaagtgcttggggcccaagggttcatggggatgttctggggtgcaccgaggccacagtggggtggaggtacccagcctttctcattaaggtactcatggtttccaggagccactgatgaggctggaactcaagaaggaagaagggtaagagcctgtctggggaacgggagggcaggctagcacccaggcagggtggtctgtcaggccaggaggatgagggggagacggagatgcagcacctcctgagcgcatagggctttgcatcaggggcttcagcatggacaggctgttcttagtgtccgcacttggattggctacagtgtcatcctgcccagtacatcatcctgggaaactgagggtcagtgtcttatctagataa

SEQ ID NO: 134 - DNA, 1038 bp, Unknown Organism
  Description of Unknown Organism CPLA2-beta, EMBL No. q9ukv7
tctcttgtctcagcctgagctgttctccccccgccccgccccctcatactgctcctgtctgtcaggtcacatgggcctttgttcctttccctgccggtcttctgtccttgccacagggagttagttggtggggtcttagcaaagaaaggagtaatgactcgcagggtcctttctctccctgtccctaaggatgacccccatgagcagctgaaggtgccactacgaactctaccctcctcgcagctggtgagacttgtcttccccacgtcccaggtacatctggtcatcgagagaagctgggttcatgcacagggctgctgctggccctggtctgcagtcagcctccttcaggtcatgcccgaggcgatcagtgggtgcaagggtggaccctgtgaaagtgcttggggcccaagggttcatggggatgttctggggtgcaccgaggccacagtggggtggaggtacccagcctttctcattaaggtactcatggtttccaggagccactgatgaggctggaactcaagaaggaagaagggtaagagcctgtctggggaacgggagggcaggctagcacccaggcagggtggtctgtcaggccaggaggatgagggggagacggagatgcagcacctcctgagcgcatagggctttgcatcaggggcttcagcatggacaggctgttcttagtgtccgcacttggattggctacagtgtcatcctgcccagtacatcatcctgggaaactgagggtcagtgtcttatctagataagcagactggaggggaggaccctgcccgaggctgaggcagagtgagcacaccaagaagggggagggatgcctcttggcgtggatgctgttgaggagttgccatcttgggatagcggttctttgtcacaaatccgtggaggcagtggccatctccgccctgtcgtcatcttttggagatgtgggtaaaggagagatttgacttggagatagagctcagccccggaccctgcacccagtcccaacatctgttttcccacaatcacgtg

SEQ ID NO: 135 - DNA, 1136 bp, Unknown Organism
  Description of Unknown Organism CPLA2-beta, EMBL No. q9ukv7
ctgttcttagtgtccgcacttggattggctacagtgtcatcctgcccagtacatcatcctgggaaactgagggtcagtgtcttatctagataagcagactggaggggaggaccctgcccgaggctgaggcagagtgagcacaccaagaagggggagggatgcctcttggcgtggatgctgttgaggagttgccatcttgggatagcggttctttgtcacaaatccgtggaggcagtggccatctccgccctgtcgtcatcttttggagatgtgggtaaaggagagatttgacttggagatagagctcagccccggaccctgcacccagtcccaacatctgttttcccacaatcacgtggtgcgcggttctgggttcccactccttccagagcaagtaatggtgaggactgagaagggagtagcgagagcaaggaagggcctggctggtgaagccaaccagtttcctgtaacttgcttctgggtctcttccttctccccagaccaaaggagctggctgtgcggctgggctgtgggccctgccctgaggaacaggccttcctaagcaagaggaagcaggtggtggctgccgcgctgaaaaaggccttacagctggaccaagacctgcacgaggatgaggtctgagggttgagcatggctgggtggagtgtttgccaggcctgcttggtcccagggttcggtgttgggacggttgtgcaagctctggtgcccactcaggcttgtcactggcaagccctgctgctccgttcctcttcctcaatgttgtcagagtgatacgtgaggtgcctgctgctcacagatgggagtttccacataggctgccctgccttccctgtttcttggtcccccacctgactgatgagtaaggtagactcactagtacgagggtctcttggctcagaatagcctcagctgtctcctagaaaataaattccagcactggcgagcaggacctttgggagacttttgaaggagccttggggcttccagaaagtgatcccagactccttatgggctgccaataggcaataaagcctggcccgtcaggctgttcttcctggagccctgatgtttgaaacagcaagacctttggaaggctgcttctgactagattttta

SEQ ID NO: 136 - DNA, 1146 bp, Unknown Organism
  Description of Unknown Organism CPLA2-beta, EMBL No. q9ukv7
gctccgttcctcttcctcaatgttgtcagagtgatacgtgaggtgcctgctgctcacagatgggagtttccacataggctgccctgccttccctgtttcttggtcccccacctgactgatgagtaaggtagactcactagtacgagggtctcttggctcagaatagcctcagctgtctcctagaaaataaattccagcactggcgagcaggacctttgggagacttttgaaggagccttggggcttccagaaagtgatcccagactccttatgggctgccaataggcaataaagcctggcccgtcaggctgttcttcctggagccctgatgtttgaaacagcaagacctttggaaggctgcttctgactagatttttaacaggggtaactcagagttggtccaagattcagtcacatgctgaccaaccatcatgccgtcttcccgtgtagctttctccatccctcaggccctggctttgaacctggctttcctcttccagatccccgtgattgccgtgatggccactggtggtgggatccgggccatgacgtctttgtatgggcagctggccggcctgcaggagctcggccttctcgactgcatctcctatatcacgggggcttcaggatccacctggtgggttggttgtgggcagagtgctggaggttgccgttggctggtggggtcgggaaagggaatgtagtgggaaggaattccgcggatccttctatagtgtcacctaaatgtcgacggccaggcggccgccaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaataca

SEQ ID NO: 137 - DNA, 1227 bp, Homo sapiens
gattgtaaacaacctaaatcagcaatacttactttttggttgttgttttgttttgtgttttgttcttttgagacagggtctcactgtgtagcctcggctgtcctggaattcactaggtacaccaggctggccttgaattcacagagatctacctgcctttgcctcccaagtgctgggatcaaaggtgtgcgccaccatgccccactcttgtttgttttttgacacagggttttatgtacttcaggctggcttttaaacttaccatatagccaagggtaacctggaactttctaccctcttgctttttaccccaagtgctgggggttatagacaggtgcagccgccctcgagttggctgtaaataagtttactagactgatggccagagagggctgatagagaaagaaatgccagtgagctcacatcaatgcatggaaaagataaagaccaccagggttcggttgtaaccgctgctgttttgatgctttaggggtgaatgagagggagtactggaaggagtcaaacaaagggcacagctccagggccatgatgctgtccagaccgaagcctggagagtcagaggtggacctgctgcgcttccagagccagttccttgaggctggtgcagccccggcggtgcagctggtgaaggggagtaggaggcatggtgatgctcctccagaccggctcccaccgcaggaccatcgggatgtggtgatgctggacagtgagtgcttgcgagcatgggcgtgaggagagagggctcagcctcagggaccctgcttccatcctgaggtggctgaatgctgctccaactgtaacctctgttgctgaaagtttgttttatttttattgctttcattcattcctctgtgtgtgtgtgtgtgtgtgtgtgtgtgtgtgtgtgtgtgtgtgtgtgagagagagagagagagagagagtgtgagtgtgagtgtttggaactcactattgagcacaagctaactttcagtttgtgacaatcttcttgtaaatactgagggaccagaggtgcaagccagcgctgcagcttcattatacagatgaagaaactgaggctcagagaggttaaatatcatacccaagataacacagctgaagctagggcccagagcagctgtcttttgttctttgtttctgcccccaatccatacttccttaggaacgtgtgaatatacttaatatctgcattccttttctctcagatct

SEQ ID NO: 138 - DNA, 1149 bp, Homo sapiens
gacagtgagtgcttgcgagcatgggcgtgaggagagagggctcagcctcagggaccctgcttccatcctgaggtggctgaatgctgctccaactgtaacctctgttgctgaaagtttgttttatttttattgctttcattcattcctctgtgtgtgtgtgtgtgtgtgtgtgtgtgtgtgtgtgtgtgtgtgtgtgagagagagagagagagagagagtgtgagtgtgagtgtttggaactcactattgagcacaagctaactttcagtttgtgacaatcttcttgtaaatactgagggaccagaggtgcaagccagcgctgcagcttcattatacagatgaagaaactgaggctcagagaggttaaatatcatacccaagataacacagctgaagctagggcccagagcagctgtcttttgttctttgtttctgcccccaatccatacttccttaggaacgtgtgaatatacttaatatctgcattccttttctctcagatctcccagatttgcccccagctctgcttcccgctcctgcaaaaagagcaagaccgagccctggtcgccccctgcctcacgatgaagaccctgaagagaggctgaacaggcatgatcagcacatcactgctgtcttgtctaagattgttgtacgtgtcagcccggctgggtggggcaaggcatcccatttgggcgagaaaagggtttataagtggagggtgttgtagaaaggtacacttttaacactccacggtcaggttggaacatggttagaaccacctagactagatccaacatgggttcctgaacctagactagatccaacatgggttcctgaacccctggaatgaggatcatgggggccactggtatcgggttcagcctggagaagcctgtagacagtgcctatccatggcctctgcatctgctcctgcctccaggttcctgccctgtttggggtcctgatgtgcattttccttactttgtgttgccacaggaacgagatacaagttcagtcactgtgactctgcctgtgcccagtggtgttgctttcccgcctgtgttccatcgctctcaggagagacaggtaagccagagttcccttaggtggactttcagttaggctttctattgctgcaccaaaacacca

SEQ ID NO: 139 - DNA, 1090 bp, Homo sapiens
tcagatctcccagatttgcccccagctctgcttcccgctcctgcaaaaagagcaagaccgagccctggtcgccccctgcctcacgatgaagaccctgaagagaggctgaacaggcatgatcagcacatcactgctgtcttgtctaagattgttgtacgtgtcagcccggctgggtggggcaaggcatcccatttgggcgagaaaagggtttataagtggagggtgttgtagaaaggtacacttttaacactccacggtcaggttggaacatggttagaaccacctagactagatccaacatgggttcctgaacctagactagatccaacatgggttcctgaacccctggaatgaggatcatgggggccactggtatcgggttcagcctggagaagcctgtagacagtgcctatccatggcctctgcatctgctcctgcctccaggttcctgccctgtttggggtcctgatgtgcattttccttactttgtgttgccacaggaacgagatacaagttcagtcactgtgactctgcctgtgcccagtggtgttgctttcccgcctgtgttccatcgctctcaggagagacaggtaagccagagttcccttaggtggactttcagttaggctttctattgctgcaccaaaacaccatgaccagaaacaacttggggatgaaaggcattatttcaattacattttcatcaggaccccaaacagggcaggaacctggaggcaggagcagatgcagaggccatggataggcactgtctactggcttgctcagcctgcttgtttatagaacccaggaccaccagctcaaggaagcacccctcacaatgggctgggccctccccatcaatcactaggaaatgccttagagctggatctcctttctcatgactctagcttgtgtcacgttgccgtaaaactatccagcacaggcgatgcttagggaatgagtggttctcagagcgcatcttccctagggagctctggacaatgataagcaagtcccttttattttcccttaaagaaaaccatatgtaagtatctatgtctgggccaggcaggcagatctctgagtttga

SEQ ID NO: 140 - DNA, 1121 bp, Homo sapiens
tttttgatgttggtgtcttttttttttaatttatttattattatatgtaagtacactgtagctatcttcagacacaccagaagagggtgtcagatctcataacagatggtagtgagccaccatgtggttgctgggatttgaactcaagaccttcagaagagtagtcagtgctcttaaccgcttgagccatctcaccagcaaacctgtttgttttttcttttctcttttcttttcttctcttttcttttcttttttttagacaaggtctcatttctcatcaccatcttcaagcttgctgtgtagcagagggtgtcttgaactcctcttatttctgcctctacctccagaatgctgattttagctgggtaccaccatatcccagaagtcctactttctctgagaaactttcttttctctttgttccagagccttggtttaaggagctaagaaagaatgggtaatgaatattgacaacttggttttttctctttgccctgtaggtgaaaccagcagcatctggtaaaaggagcatctttgcccaagagattgcagcaagaagggtgtcaggaaacagggtgacatcagctgagcaagttgtccccagcttagacacaccaaagggtgagtgctgccaggctgagggtgacagcgctcagtcatgttggctcgtagaaatgtattctctctttggtggtggtagagtggggcaaagtcaagattaatgtgtgccagtctggcctcgaactcactgtgtaatgaagggtgattttaaaattgtgattctcctgcttccacctctggtctgctggaaatacagacatgtgccaccatgccaggttatatggttatgggatcaaaccccgggctttgtgtatcctagcaaagatgcatgctagcaacagagctacatccctagcctctcaaatgtccatccatttttcattctcatattctctctctctcaaaactttttgctgggcagtggtggcgtatgcctttaatcccagcacttgggaggcagaggcaggcggatttctgagttcgaggccagcctggtctacaaagtgagctccaggacagccagagctatacagagaaaccctgtctcgaaaaaacaaaaa

SEQ ID NO: 141 - DNA, 1222 bp, Homo sapiens
attagcaaccaaggcactggggctggagagatggccagtggtgaagagtgaggattgctcttacagaaaaccctaacgtgattgctagctcctgtgtcaagtacttacaactgcttgtaatcccaactctaggggagccgatgcctcatttggaacacatgtgcacttggcatgtccacacacaacccatatatgtgtgcatgacacacccacacagacacagacccacatacacacacataattaaaataaaacttttaaaattaacctctgagccatctctccagctgtcccatgtaagtcttaagagaagaatgaatgagaagtggccatcttgcctcccccaaatgcctgaaatgctgtgtggtttcctttctgtgtccagggaagccccggggccatcgctggtccctgggactagttgccttatttaagaggccagagggtgtaggtagagctaggagagtagagcaataaaactgactgtctacttctttcaggtgctgtgccctgtgagacaccctccgtcagggacagaagcaaccagcttcctgggaggagtcatggcttccacagaccaaatctagtcacaggaaagggactcaggagcaaggtggctgagcaggaggtccagacaatccatgaagagaatgtagcaagactacaagccatggatcctgaggagatcctgaaggagcagcaacagttactggctcaactcggtatggatgttaagtcttgcagttagcttgctggggccacacgaagcaggggctgccacttgttggggaagacgtgtgtgtgtttgtgtgtgtgtggctattcttctactcctgggagagatatggtattaagttcattttacgttcattttactaatgaatgaatgaatgaatgaatgaatgaacgaacaaacgaacaaacaaacagagcaggctctgctacttggagtggtacatgcctttaatcccagcatgtggaactacttccaatcagaccagagttcgtgctcagctacatggctagtttgaggccagtctggctctgagacccttcctcaaaaacaaagcttgaagctgatggcatagctcagtggttaactaagcaagcctttgaccatgtaaaggtgggagagaactgactgcactaagttgtactcagacctgtacatgtacacatactacctccctgtgccccaatagtaactgaaaacaaaaaaatc

SEQ ID NO: 142 - DNA, 1185 bp, Homo sapiens
gctttatcacagctgtgctctgccctctctcctttaccctctcctgtaactacagtgacacatacaggagtgtgctgtgccacacctttcttctcagtcagtcttgttccacattatatgcggagcacttatgtgctgtatactcaatttttagtaaatgtatttacagactgagaggaaatgaagaagtttggctgcgtgctgtccttccggctctagtcaacattcacttgtttctcagacatctgacctggtagcacgcagaggttccagcataggaacctctgccagaggactgtggagggcaagggccatgggggatagtcaggctcaggggctacatgggtgtgggctggcagggtcattacggagccacaggtaggcctgagccaggacagggaagtagtttgtctggaccttagcacccagaggtgggaatcttccagcaggtgtcccatgaggagtcaccctagcctcttactctctgcctctaccctcagaccccagcttggttgccttcttgagatctcatagccaggtccaggaacaaacaggaacaaaggccaccaagaagcagagtccagagagaccctcagttcttgtcactaaagaagagcctgtcacatcaactcgtacaagggagcctaggactggggacaagctggaagaaaagccggaggccacggtggaggacaaaatggaagacaagctgcagccaagaaccccaggtatggagggcaggcaccatcatttcttcagagaggaaaaaatagccccgtgtggtggcgcatgcttttaattccagcatccttgagaggcaaagtctggcggatctctgtgagtttgaggccagcctaatgtctgcatatcaagttccaggacagccaaggctcagaaggggagtgtgcatgctaccaagccagacagcctgagttagatccctggaatctacatgatgaaagagaaaaacaacttgcacacgtacacacacacacacacacacactcacacacatactcctcatacacaaaataaataaatgttaaaaaaagaaaaaagggtaggaaaagtgtcctttgacccagcctgctgatggggaagatgctctctcgcccttggacatgggctgttcatcttcattagtgtctttaaattggctcaatggtcctcatttgctagcgcaca

SEQ ID NO: 143 - DNA, 1116 bp, Homo sapiens
agggattaacaaaagaggaaatgtaattatgacagcccggaacctcctgctcctgagggactgagggactgctgggccagccagggaggttgagtgggggtgaggtgggggtgaggacgcaggaggagatgatagaggtgttctataccctgccccactggagcagcatagctgcctccccttcttggtctccattctcttgctccttcagagggttgtaccagagagtggactcctatactttccttgatgtatggtgtcctatcttctttctctgtcccttcttccttttcttctcctcttcttcaaacacgtctttgaggtcagcatggggctagagccaggaccagctatgtttgctatagttgctatagttgaaacattcatccagttgctaggggtgccagcccccaagccgtaccatttagccatagctgaggcagctgctttctgccagtgtgctgagccccttgaggaacgcagtgcttctgcctccctagcactgaaactgcccatgacccccagcaaagactggctccacatggacaccgtagagctggacaagctccactggacccaggacctgcccccactccggaggcagcagacacaggaggtgaggacgaacaggtggggtggggtggggttactgctcttctgcccagacctggggcctgagaaatagctcttatttgttctcagcttctctcctgttaccctgcctggccctataggccccacaagcctcgtttccagtgagaggaagagcccagagctaatcctaaaaggcttgagattaaaggtgactgagtgtgaggaactgctgcagcacccaacacagtccatttcttttgtcttagagaatgcaggctcgattcagccttcagggcgagctcctggcgcctgatgtggatcttcccacacatctgggcctgcaccaccacggagaagaggccgaggtaagggatggggtcctaggaggactgggcaactcttggcaaacgtttagcaactgcaaaaactgtgcaaaacttggagctctggtcacttgcctttgacctcctgtgtgtcccgccctagctcagggccttgctgtcactcagtttcttagagtca

SEQ ID NO: 144 - DNA, 1099 bp, Homo sapiens
tatagttgctatagttgaaacattcatccagttgctaggggtgccagcccccaagccgtaccatttagccatagctgaggcagctgctttctgccagtgtgctgagccccttgaggaacgcagtgcttctgcctccctagcactgaaactgcccatgacccccagcaaagactggctccacatggacaccgtagagctggacaagctccactggacccaggacctgcccccactccggaggcagcagacacaggaggtgaggacgaacaggtggggtggggtggggttactgctcttctgcccagacctggggcctgagaaatagctcttatttgttctcagcttctctcctgttaccctgcctggccctataggccccacaagcctcgtttccagtgagaggaagagcccagagctaatcctaaaaggcttgagattaaaggtgactgagtgtgaggaactgctgcagcacccaacacagtccatttcttttgtcttagagaatgcaggctcgattcagccttcagggcgagctcctggcgcctgatgtggatcttcccacacatctgggcctgcaccaccacggagaagaggccgaggtaagggatggggtcctaggaggactgggcaactcttggcaaacgtttagcaactgcaaaaactgtgcaaaacttggagctctggtcacttgcctttgacctcctgtgtgtcccgccctagctcagggccttgctgtcactcagtttcttagagtcaagatgttgctccttctattgacatcctaccttcctgtaactcaagagccaggccttgcatgctttccctcaggctgttccctccctcacgagctgccatcctggtttcctctggcctaactcaagctgtccttcaccttggaaagttccccagccctcctttgctcactgactgaggtcctaggccttgcgcttggccgggcagtccgtagacccccaagctctgcctctccctcctctgaccactctactgatgcttctacatggaatccccattttcacagcctagcatggttcactccagagaatatctgtatccttagtccatgctgggccactgacgc

SEQ ID NO: 145 - DNA, 1102 bp, Homo sapiens
tccctccctcacgagctgccatcctggtttcctctggcctaactcaagctgtccttcaccttggaaagttccccagccctcctttgctcactgactgaggtcctaggccttgcgcttggccgggcagtccgtagacccccaagctctgcctctccctcctctgaccactctactgatgcttctacatggaatccccattttcacagcctagcatggttcactccagagaatatctgtatccttagtccatgctgggccactgacgccatacccatccactgagaccagattcactttccatggctgtcctctgattagaaattaaacagctattacatccctgtttgctgtgtggtgccattgctctgtgcatgacctataatttttattagcttgaggttccaaggagagccatgactagctgtcacttgtttatcttgcctttcccttgacaccttccctgtgtcttgattccttctgactctctgtccctgcatcagcgagcagggtattctctacaggagctgttccacctgacccgtagccaggtgtcccagcagagagcactagctttgcaagtgttgtcccggatcgtcggcagggtaagtgggctgctggcctggtcctaccaagccacaccccatccttcatcccttgatcttagccttttcctccttactgtgggtcagactaggaatggagcaaatgtacagtaatgggtcagcaaaggttctatccaagttctgcatcttgtacccaaggcccaggctggtgagtttggggaccgcctagtgggcagtgtcttgcgcctcctcttggatgctggtttcctcttcctgctgcgcttctctctggatgacagggtggacagtgtcatcgcagcagctgtccgggctcttcgtactctgctagtggctcctggagatgaggtatgtcagggtgacagggctctgctctgcgttctgggcataccctttgccaaggctttgaggcagactggagaatagggtggcaggtgtaagccccttctgaagaccacaagccttgcctccctgccgctgctgtgtgttggcgagggataattttgctctgtcctggacca

SEQ ID NO: 146 - DNA, 1168 bp, Homo sapiens
gacgccatacccatccactgagaccagattcactttccatggctgtcctctgattagaaattaaacagctattacatccctgtttgctgtgtggtgccattgctctgtgcatgacctataatttttattagcttgaggttccaaggagagccatgactagctgtcacttgtttatcttgcctttcccttgacaccttccctgtgtcttgattccttctgactctctgtccctgcatcagcgagcagggtattctctacaggagctgttccacctgacccgtagccaggtgtcccagcagagagcactagctttgcaagtgttgtcccggatcgtcggcagggtaagtgggctgctggcctggtcctaccaagccacaccccatccttcatcccttgatcttagccttttcctccttactgtgggtcagactaggaatggagcaaatgtacagtaatgggtcagcaaaggttctatccaagttctgcatcttgtacccaaggcccaggctggtgagtttggggaccgcctagtgggcagtgtcttgcgcctcctcttggatgctggtttcctcttcctgctgcgcttctctctggatgacagggtggacagtgtcatcgcagcagctgtccgggctcttcgtactctgctagtggctcctggagatgaggtatgtcagggtgacagggctctgctctgcgttctgggcataccctttgccaaggctttgaggcagactggagaatagggtggcaggtgtaagccccttctgaagaccacaagccttgcctccctgccgctgctgtgtgttggcgagggataattttgctctgtcctggaccatgtcttctttctgtctcttcccacctctaccccatcctctggcaggagctcctggaccgcaccttctcttggtatcatggagcttcggtgttccctctgatgcccagccaggacgataaagaggatgaagatgaagatgaggaactcacaacagaaaaggtgaagagaaagacacctgaggaaggaagccgccctcccccggacctggccagacatgatgtcatcaaggtagagggcactctgcatttccgtccttgacctacagggcaggctcgatgagcaggagaggtcaccaagacctgggtgtcccccttatgtggcaatggg

SEQ ID NO: 147 - DNA, 1183 bp, Homo sapiens
tcatcccttgatcttagccttttcctccttactgtgggtcagactaggaatggagcaaatgtacagtaatgggtcagcaaaggttctatccaagttctgcatcttgtacccaaggcccaggctggtgagtttggggaccgcctagtgggcagtgtcttgcgcctcctcttggatgctggtttcctcttcctgctgcgcttctctctggatgacagggtggacagtgtcatcgcagcagctgtccgggctcttcgtactctgctagtggctcctggagatgaggtatgtcagggtgacagggctctgctctgcgttctgggcataccctttgccaaggctttgaggcagactggagaatagggtggcaggtgtaagccccttctgaagaccacaagccttgcctccctgccgctgctgtgtgttggcgagggataattttgctctgtcctggaccatgtcttctttctgtctcttcccacctctaccccatcctctggcaggagctcctggaccgcaccttctcttggtatcatggagcttcggtgttccctctgatgcccagccaggacgataaagaggatgaagatgaagatgaggaactcacaacagaaaaggtgaagagaaagacacctgaggaaggaagccgccctcccccggacctggccagacatgatgtcatcaaggtagagggcactctgcatttccgtccttgacctacagggcaggctcgatgagcaggagaggtcaccaagacctgggtgtcccccttatgtggcaatgggtttctttctgcatcctaggggctcctggctaccaacctgcttcctcggctccgctatgtgctggaggtgacctgcccaggaccctctgtggtccttgacatcctggctgtgcttatccgcctggcccggcattccctggagtcggccatgagggtgagaagggaaggagggcactgagaatagacaaaaggtcccatgcactcttcttgttttattttttgggtctttttattggtgtttttgttactgctcttgttttttttttttgtttttttttgttttttttctgagacagggtttctctgtgtagccctggctgccctggaactcactttgtagaccaggctggcctcgaactcagaaatttgcctgcctctgcctcccaagtactgggattaaag

SEQ ID NO: 148 - DNA, 1135 bp, Homo sapiens
gctctgctctgcgttctgggcataccctttgccaaggctttgaggcagactggagaatagggtggcaggtgtaagccccttctgaagaccacaagccttgcctccctgccgctgctgtgtgttggcgagggataattttgctctgtcctggaccatgtcttctttctgtctcttcccacctctaccccatcctctggcaggagctcctggaccgcaccttctcttggtatcatggagcttcggtgttccctctgatgcccagccaggacgataaagaggatgaagatgaagatgaggaactcacaacagaaaaggtgaagagaaagacacctgaggaaggaagccgccctcccccggacctggccagacatgatgtcatcaaggtagagggcactctgcatttccgtccttgacctacagggcaggctcgatgagcaggagaggtcaccaagacctgggtgtcccccttatgtggcaatgggtttctttctgcatcctaggggctcctggctaccaacctgcttcctcggctccgctatgtgctggaggtgacctgcccaggaccctctgtggtccttgacatcctggctgtgcttatccgcctggcccggcattccctggagtcggccatgagggtgagaagggaaggagggcactgagaatagacaaaaggtcccatgcactcttcttgttttattttttgggtctttttattggtgtttttgttactgctcttgttttttttttttgtttttttttgttttttttctgagacagggtttctctgtgtagccctggctgccctggaactcactttgtagaccaggctggcctcgaactcagaaatttgcctgcctctgcctcccaagtactgggattaaaggcgtgcgccaccacgcccggctactgctcttgttttgagatagaatttcattatgctcaagcaggtctagacctcactgcatcccttatgctggcctgaaacttgcagaaatccttcctcagcttcctgagtactgagattataggcataagcctgtggtctatgacctgagagatgatggacctcatcttgatcttctttgcctccctttcccacctatctcccacaggtcctggagtgtcctcggctgat

SEQ ID NO: 149 - DNA, 1171 bp, Homo sapiens
attccctggagtcggccatgagggtgagaagggaaggagggcactgagaatagacaaaaggtcccatgcactcttcttgttttattttttgggtctttttattggtgtttttgttactgctcttgttttttttttttgtttttttttgttttttttctgagacagggtttctctgtgtagccctggctgccctggaactcactttgtagaccaggctggcctcgaactcagaaatttgcctgcctctgcctcccaagtactgggattaaaggcgtgcgccaccacgcccggctactgctcttgttttgagatagaatttcattatgctcaagcaggtctagacctcactgcatcccttatgctggcctgaaacttgcagaaatccttcctcagcttcctgagtactgagattataggcataagcctgtggtctatgacctgagagatgatggacctcatcttgatcttctttgcctccctttcccacctatctcccacaggtcctggagtgtcctcggctgatggagaccatagttcaagagttcctgcctaccagttggtcccctataggggtggggcctactcccagtctatataaagtgccttgtgcttctgccatgaaactgcttagagtcctggcttcagctgggaggaatattgctgctcgactggtaagctggacacatggcaagagatggagggcataagacagggctgatttgagctggatttactctgagtggtggatagctacctcagtccatcttcctcttcctctctggtctgtttgcttagaaattcaacagaaaccagcaggtgggaccctggtcacctgggtctgttggtattctggtctcatgatatggggagggttccctgaccccagcctgtccagagttgctgcatgcagacaggccttcatcacccatagctaagcagaaaaagtacctagcaaatactaactaaaaagaagggaagaccactggctagggagccactcagcaggtaaaggcacttgctgccaagctgatcacacaagtttagcccctggaactcacatggaaggagaaggaaaagactcctcaaggtttgtctgatttccatatgcattgtctgacaggtgagcgctgtggccccagataaatgctaaggaagaatgctgaaggagggg

SEQ ID NO: 150 - DNA, 1158 bp, Homo sapiens
atcttcctcttcctctctggtctgtttgcttagaaattcaacagaaaccagcaggtgggaccctggtcacctgggtctgttggtattctggtctcatgatatggggagggttccctgaccccagcctgtccagagttgctgcatgcagacaggccttcatcacccatagctaagcagaaaaagtacctagcaaatactaactaaaaagaagggaagaccactggctagggagccactcagcaggtaaaggcacttgctgccaagctgatcacacaagtttagcccctggaactcacatggaaggagaaggaaaagactcctcaaggtttgtctgatttccatatgcattgtctgacaggtgagcgctgtggccccagataaatgctaaggaagaatgctgaaggaggggaccagagacttagaatagggcagcttgagcctgtttccaggagagccagtactagctggatgaccctgccgctgtaccttctcatctctagctaagcggctttgatgtcaggagccgcctgtgccgattcatagctgaagcgccacatgacctggccttgccccccgaggaagcagagattctgaccactgaggccttccgtctgtgggccgtggctgcctcctatggccagggtggtgacctttacaggtgaggagaagccggagataggcttctcgcagggccctgctgtcctgcactgttgagttctgcaatacatccttcagttttgttgaaggccgtgaggaagggaggccaaatacgatgggggaatctctgctgggcatgaattgctaggaaaaagcggaccaactgcattctttgacaaaagcagaagtcagatatcagttcccagttgtggggtaacctaggaggtttgtagccttcccagttttcagggcctcgccttgcacaagtgcctctgtcctgggctgtccccctccggctcctcgagtttctctgggttgaagctcctggcagaattgtgctctcctcttgcctccctgtgtagtgaatgccagttgtcttccccagggagctgtacccagtgctgctgcgggccttgcagacactgcctacagagctcagcgctcatcccctacagcccctggccatgcagcgggtggccgctctgttcacactgcttaccc

SEQ ID NO: 151 - DNA, 1159 bp, Homo sapiens
gctgaagcgccacatgacctggccttgccccccgaggaagcagagattctgaccactgaggccttccgtctgtgggccgtggctgcctcctatggccagggtggtgacctttacaggtgaggagaagccggagataggcttctcgcagggccctgctgtcctgcactgttgagttctgcaatacatccttcagttttgttgaaggccgtgaggaagggaggccaaatacgatgggggaatctctgctgggcatgaattgctaggaaaaagcggaccaactgcattctttgacaaaagcagaagtcagatatcagttcccagttgtggggtaacctaggaggtttgtagccttcccagttttcagggcctcgccttgcacaagtgcctctgtcctgggctgtccccctccggctcctcgagtttctctgggttgaagctcctggcagaattgtgctctcctcttgcctccctgtgtagtgaatgccagttgtcttccccagggagctgtacccagtgctgctgcgggccttgcagacactgcctacagagctcagcgctcatcccctacagcccctggccatgcagcgggtggccgctctgttcacactgcttacccagctcaccctggcagctagcagcatacctcctgagcccgccaggtaggccagctccaagcctgtcagcaggcatgcccatgtggaatgctcctccacagggagtaaggcggcccatgcttacctttgcccctttggcactcctgcagtggccctgctgagtcctgtgtgccggccatcccttcctcagtcacctggacacaggtgtctggactcaagccactggtggagccatgtctaaagcagaccctaaagttcctgcccaggccggatgtgtggaatgccctgggcccagtgcccagtgcctgccttctgttcttgggtgcttactatcaggcctggagccggcaggtgagtgcagcctgcctccctgcctgcctccagcctacatacctcttccattccttctcagttggtcccttttctacctcttcccccgcgtgtggttctgagtgtaaacatggctgtgctgagtggggccctcataggccagacagttacttttctactgagaaccaccctcacctttcgcttctacctgttcc

SEQ ID NO: 152 - DNA, 1202 bp, Homo sapiens
gaaaaagcggaccaactgcattctttgacaaaagcagaagtcagatatcagttcccagttgtggggtaacctaggaggtttgtagccttcccagttttcagggcctcgccttgcacaagtgcctctgtcctgggctgtccccctccggctcctcgagtttctctgggttgaagctcctggcagaattgtgctctcctcttgcctccctgtgtagtgaatgccagttgtcttccccagggagctgtacccagtgctgctgcgggccttgcagacactgcctacagagctcagcgctcatcccctacagcccctggccatgcagcgggtggccgctctgttcacactgcttacccagctcaccctggcagctagcagcatacctcctgagcccgccaggtaggccagctccaagcctgtcagcaggcatgcccatgtggaatgctcctccacagggagtaaggcggcccatgcttacctttgcccctttggcactcctgcagtggccctgctgagtcctgtgtgccggccatcccttcctcagtcacctggacacaggtgtctggactcaagccactggtggagccatgtctaaagcagaccctaaagttcctgcccaggccggatgtgtggaatgccctgggcccagtgcccagtgcctgccttctgttcttgggtgcttactatcaggcctggagccggcaggtgagtgcagcctgcctccctgcctgcctccagcctacatacctcttccattccttctcagttggtcccttttctacctcttcccccgcgtgtggttctgagtgtaaacatggctgtgctgagtggggccctcataggccagacagttacttttctactgagaaccaccctcacctttcgcttctacctgttcctccctgctttgtttcccaggggagcctctgagagccatgttgatgggaccagggaccccgagcagcagctcagctttgtcagccctctcatgcacgctagatgttatgtactagagcaagttcagggctgtctccccttcagaaagatcaggctgtggaagcctccaaacagattttttgagaccctcagtatgatggggctggtctggatatatagggctagggtcttctagtccatcagctcagtgaaccccttggttctccagtcacacttgtgcccagaagattggcttcaggacatggagc

SEQ ID NO: 153 - DNA, 1110 bp, Homo sapiens
tctgttcttgggtgcttactatcaggcctggagccggcaggtgagtgcagcctgcctccctgcctgcctccagcctacatacctcttccattccttctcagttggtcccttttctacctcttcccccgcgtgtggttctgagtgtaaacatggctgtgctgagtggggccctcataggccagacagttacttttctactgagaaccaccctcacctttcgcttctacctgttcctccctgctttgtttcccaggggagcctctgagagccatgttgatgggaccagggaccccgagcagcagctcagctttgtcagccctctcatgcacgctagatgttatgtactagagcaagttcagggctgtctccccttcagaaagatcaggctgtggaagcctccaaacagattttttgagaccctcagtatgatggggctggtctggatatatagggctagggtcttctagtccatcagctcagtgaaccccttggttctccagtcacacttgtgcccagaagattggcttcaggacatggagcgcttgttggatgagtccttgctgccactgctgagccagcctcctctgggcagtctgtgggattccttaaggtacaatctgggacggccgctgggggagtgcgggatggaggcagttctcctgaagggcccctgggtctgtctccccagtccatcccaggacatggccctttaggggcctgaggaaacccaaatttccagcaggagcaaccctcagtactgtgaggaacatgaactctgatctgcacagccatgcacggcttgcactcacagcccagagtcccctcagcatgtctgttctccctgcagggactgctccccgctctgcaatccgctgtcctgtgcttccacccctgaagccctccccagccttgtgtcactgggctgtgcaggaggctgtccccctctcagtgtggctggctcagcctcacccttcccgttcctcactgccctcctctccctcatcaacactctggtccagagccacaaggggctctgtggacaggtgagcctcgggtgcccatttggaggccaggcatggggacaagatagcaggggtaggaaagagggag

SEQ ID NO: 154 - DNA, 1196 bp, Homo sapiens
gagcaagttcagggctgtctccccttcagaaagatcaggctgtggaagcctccaaacagattttttgagaccctcagtatgatggggctggtctggatatatagggctagggtcttctagtccatcagctcagtgaaccccttggttctccagtcacacttgtgcccagaagattggcttcaggacatggagcgcttgttggatgagtccttgctgccactgctgagccagcctcctctgggcagtctgtgggattccttaaggtacaatctgggacggccgctgggggagtgcgggatggaggcagttctcctgaagggcccctgggtctgtctccccagtccatcccaggacatggccctttaggggcctgaggaaacccaaatttccagcaggagcaaccctcagtactgtgaggaacatgaactctgatctgcacagccatgcacggcttgcactcacagcccagagtcccctcagcatgtctgttctccctgcagggactgctccccgctctgcaatccgctgtcctgtgcttccacccctgaagccctccccagccttgtgtcactgggctgtgcaggaggctgtccccctctcagtgtggctggctcagcctcacccttcccgttcctcactgccctcctctccctcatcaacactctggtccagagccacaaggggctctgtggacaggtgagcctcgggtgcccatttggaggccaggcatggggacaagatagcaggggtaggaaagagggagagaagtggctcgtagggggcagcagtctggggagtcccagaacccatcagtgtttgtttctagatctgaacaaacttgtttcctcagctgtctgctgtgttgactgccccaggactccagaagtactttctccagtgtgtggctcctgcgcctgccccacagctcacacccttctctgcctgggccctgcaccatgagtaccacctacagtatctggtgctttccctcgcccacaaagcggtgggtccctgtctgcagaatcagcctcaccctgttgtctctcctgcctctcagctgtagtccatattccctggttcatatcctccatctttctatcacttcctcactctgtcacttcctcactccctctgttctgtgtcctccatgctgcctccctacactgactagagccctgtctcctccctaggcaaca

SEQ ID NO: 155 - DNA, 1153 bp, Homo sapiens
ggacatggccctttaggggcctgaggaaacccaaatttccagcaggagcaaccctcagtactgtgaggaacatgaactctgatctgcacagccatgcacggcttgcactcacagcccagagtcccctcagcatgtctgttctccctgcagggactgctccccgctctgcaatccgctgtcctgtgcttccacccctgaagccctccccagccttgtgtcactgggctgtgcaggaggctgtccccctctcagtgtggctggctcagcctcacccttcccgttcctcactgccctcctctccctcatcaacactctggtccagagccacaaggggctctgtggacaggtgagcctcgggtgcccatttggaggccaggcatggggacaagatagcaggggtaggaaagagggagagaagtggctcgtagggggcagcagtctggggagtcccagaacccatcagtgtttgtttctagatctgaacaaacttgtttcctcagctgtctgctgtgttgactgccccaggactccagaagtactttctccagtgtgtggctcctgcgcctgccccacagctcacacccttctctgcctgggccctgcaccatgagtaccacctacagtatctggtgctttccctcgcccacaaagcggtgggtccctgtctgcagaatcagcctcaccctgttgtctctcctgcctctcagctgtagtccatattccctggttcatatcctccatctttctatcacttcctcactctgtcacttcctcactccctctgttctgtgtcctccatgctgcctccctacactgactagagccctgtctcctccctaggcaacactgcagccagaaccagctgccagcactgccctccaccatgctgtagccttggtcctactgagccggctattgcctggaagcgaataccttgcccaggagctgctgctgagctgtgtgttccggctggagttcctgccgtaagtccaggggtttcaggattcaggggtttggtacttttggatgcccctgaactttgttaacaaaacagcatcttggagctggaaacctgggttttagtctctatccttatacttaatttgttattccctcagtaaagtgaattgaggctattcatcttcaccccacc

SEQ ID NO: 156 - DNA, 1143 bp, Homo sapiens
ggacaggtgagcctcgggtgcccatttggaggccaggcatggggacaagatagcaggggtaggaaagagggagagaagtggctcgtagggggcagcagtctggggagtcccagaacccatcagtgtttgtttctagatctgaacaaacttgtttcctcagctgtctgctgtgttgactgccccaggactccagaagtactttctccagtgtgtggctcctgcgcctgccccacagctcacacccttctctgcctgggccctgcaccatgagtaccacctacagtatctggtgctttccctcgcccacaaagcggtgggtccctgtctgcagaatcagcctcaccctgttgtctctcctgcctctcagctgtagtccatattccctggttcatatcctccatctttctatcacttcctcactctgtcacttcctcactccctctgttctgtgtcctccatgctgcctccctacactgactagagccctgtctcctccctaggcaacactgcagccagaaccagctgccagcactgccctccaccatgctgtagccttggtcctactgagccggctattgcctggaagcgaataccttgcccaggagctgctgctgagctgtgtgttccggctggagttcctgccgtaagtccaggggtttcaggattcaggggtttggtacttttggatgcccctgaactttgttaacaaaacagcatcttggagctggaaacctgggttttagtctctatccttatacttaatttgttattccctcagtaaagtgaattgaggctattcatcttcaccccacctgagaatgataagactccacagtatgtcaatggctaaaaaaatgtcacataagctaagctagggatgtagttgagcagtaaagcacctacttggcatgtgtgaggctctgattcaaagtagggatctggttcactgatgcacacctctcatcctagtactcgggaggtcagcctgggctacgtgagagatagatagatagatagatagatagatagatagatagatagatagatagatagatagatagatagaggtcagcctgggctacatgatagatagatagatagatagatagatagatagatagatagatagatagatagatagat

SEQ ID NO: 157 - DNA, 1921 bp, Homo sapiens
tagatagatagatagatagatagatagatagatagatagatagatagaggtcagcctgggctacatgagagatagatagatggatggatagatagatagatagatagatagatagatagatagatagatagataggtaggtaggtagatagatgtatatagatataggtggataggtagagatagatgatagataagtgatggatgcatagatagatagatgatagattataagtaaataaataaataaataaacaaacaaacaaataaatagtaaagagctgggtgacaccttaaccccagcacagctagaggtcaagcatggaagtttaagtaaactttccctctgttccatctcctaatgctatgcaacattatggtgtgagaggctgtcaggggagcagagaggcacaggactaaacttagaaactctagatctgggacagctgtctggatgctgtgattctagcccttagttttggcttttgtatcatgtcacagggaaagtgcttcagggggcccagaggcagctgacttctccgatggactgtccttagggagcagtggggaccctcagtgtagacgaggggccctcctcgttcaagcttgccaggatctccccagcatccgcagctgctacctggcccattgttcaccagcccgagccagtctgctgagctcccaagccttgtaccgtggagagctactgcgagtctcaagtctgctactgcctgtacctaaggagccactgctggccactgactggcccttccagccgttgatacatctctaccaccgagcttcagatactccctctgggccacctgctgctgacactgtaggcgttgccatgcgggtcctacaatgggtgctagttctggagagctggcgccccgaggtcctctgggctgtgccccctgctgcccgcctagcacgtctcatgtgtgtgtacctggtggatagcgagctgtttcgagagactcccatacagcgactggtggcagctcttttagctcggctctgtcggccccaagtcttgccaaacctcaagctggactgccccctccctggcctgacctcttttcctgacctttatgccagcttcttagaccattttgaggctgtctcttttggggaccacttgtttggggccctggtcctccttcctctgcagcgtaggttcagtgttaccttgcgcctcgccctctttggggagcacgtgggagtgttgcgagcacttggcctgcctctgactcaggtatgtcctacactatcaaggccttcctaggacccttcactagacactaggggcagtcttgagcagtgtggcacttcggtgggcaccattacctcattacctctttgaaagcatcaagctgtaggaatagaagggtttttatcatggcatctggtagagagcaggtatacatattattactattgggttttttcaagtcagggtttgtctttgtagctatggctgttctggaacttgttctggagaccaggctggcctgaaactcacagagctgcctctgtctcctgagtgttggggttaaaggtgtggatcaccactgtctggcagggtttgtgttattaacaggtttggtttctgcttgtgaactcctacagtttagaagaagatatgctaagatcaaaaaacaaaaacatacttgttaattactcagatctttggtgtgttaagtcagaattcagagaatttttaaaagactggagtggggctggtgagatggctcagtgggtaagaacacctgactgctcttctgaaggtccagagttcaaatcccagcaagagatctgactccctcttctggagtgtctgaagacagctacagtatacttacatataataaataaataaataaatctttttttttttttttttttttggttttttgaga

SEQ ID NO: 158 - DNA, 1720 bp, Homo sapiens
  modified_base (697)..(895): a, t, c, g, other or unknown
tcagtgggtaagaacacctgactgctcttctgaaggtccagagttcaaatcccagcaagagatctgactccctcttctggagtgtctgaagacagctacagtatacttacatataataaataaataaataaatctttttttttttttttttttttggttttttgagacagggtttctctgtatagccctggctgtcctggagctcactttgtagaccaggctggcctcgaactcagaaatccgcctgcctctgcctcccgagtgctgggattaaaggcgtgcgccaccacgcccggcttaaataaatctttaaaaaaaaaaaaaaagattggagtggactttaccttaaggaaaagatgaggtgaaggagagaggttgtcttagtgctgaagtttgggaagaatttgactcttaagtgcttactagaaaaaggctatgaagcaaagccctaaagggtgatcagactaaagagtcagctcacaggttgtaaagagaaccaggcctggcaacacaggctctcgggagctgagtcaggaggattagaaggttttgtttggtttttagatttatttatacatatgaatacactgtagttgtctttagataccagaaaatggtatcagatcccattacagatggttgtgagccaccatgtggttgctgggaattgaactcaggacctctggaagagcagtcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccagagaaaccctgtcttgaacccccacccaaaaatataaataaaaacaatacttactgagctgggagtgggagcaggtgcctatcattcctgcacatacacagaaggccagggcaggggttacaaggctgagtccagcctgggtcacataggaaggcactgtctcaaaacctttattttcccttgaaacagggtctctttatgaagctctggctctgaacttctacataaaccaggctgtcttcagactcacagagactcaactgcctctgcgtcccaactgctgggattaaaggtgtgcgccactataccgggctctaaaatgtttttaaataaaaaaattactatgagtaactattaaatttttaatttttttatgttttaataaatgtacatgtgtgtatgtgtatgtgtgtgtgttttatgtacacgtgaatgcaggtggacatgctcatgcaggcatgaacttgaggacactaggcatcctgttctgtttctctgccttactcctttgagacagtggagctaggagtggctagcaagctctctatcaactgagctacaatccttagcacgctgaagactgtctggactcaccctagactggactagaactctgtatatagcctaggctgtccttaaacttcttcttcttcctttttttttttttttttttttttttttttttggtttttttcaagacagggtttctctgtgtagtcctggctgtcctggaactcactctgtagaccaggctggcctcgaactcaaaaatctgcctgcctctgcctcccaagtgctgggattaaaggtgtgcgccacca

SEQ ID NO: 159 - DNA, 1589 bp, Homo sapiens
tttctctgtgtagtcctggctgtcctggaactcactctgtagaccaggctggcctcgaactcaaaaatctgcctgcctctgcctcccaagtgctgggattaaaggtgtgcgccaccatgccccgcgtatttgtatatttttataaaattggaattactgcttaatgaatttttcatttaaccttacatcatgagcagttgccatcccttaaatactcttaagaaatcaatacttaaaccgggagtggtggcgcacgccaataatcccagcacttgggaggtagaggcaggtggatttctgagttcgaggccagcctggtctgcagagtgagtttcaggacagccagggctacacagagaaaccctgtcttgaaaaaaacaaaacaacaacaaaaaagaaatctactttgtgtgtgtgtgtttacgtgtgtgtgtgtgtgtgtgtgtgtgtgtgtgtgtgtgtgtgtgtgtgtgtatgcgcgcactagaaatggaacacagggcttcatgcatcctaggaaggtgcgctcaggctgtaagctatgccctaataccaaccagtctttgccctttagctttcaggcactaaagaagggaggtctttctaggacctgatttctgccctgtgatttgatggcatgtgaccaacagtcatccactgctgctactgatggacattcccacttggccctcagcatatcatttccttctgtcacagctgcctgtgcctctggagtgctacacagagcctgctgaagacagcctgcctctccttcagctctacttccgggccttggttactggctcactccgtgcacgttggtgccccatcctctacactgtagccgtggctcatgtcaacagtttcatcttctgccaagacccaaagagctcagtaagtcacctctctgtgttcagagggaaggagggagctggtggaccagttggggatgggctgagggttaagtgagtccatgggtgggtaccagtcctcggctttgtgtcatatctctcttccgtccactcccaggatgaggtaaagactgcccgaaggagtatgctgcagagaacgtggctgctgacagatgaggtagtattgtgggtgtcgggacagaaaagatacaggacgttaaggtaccaaacctcaacaaactgaccacccacttcatgggttttgtcttctctgcaggggctgcggcagcatctcctccactataagcttcccaactccagcctcccagagggctttgagctgtattcccagttacctcgcctgaggcagcagtgccttcagacactgcccacagaggggctccagaatggaggagtcaagacatagggcacttgcccccagctaaagatgactactctactctcctgggactgagcagtgagcctcccgagagaaggcaacacaagacttgccttcctgtgagcacattgtttgagccttttttagaacagaagagactcgtattacatcatgatactggagctcaggatctgggggctgagggctgtgccaggcgtggaaggagggtggcttcagtgtttaccctgcggtgcccaggacagtctgagcacagctct

SEQ ID NO: 160 - DNA, 1158 bp, Homo sapiens
cctcagcatatcatttccttctgtcacagctgcctgtgcctctggagtgctacacagagcctgctgaagacagcctgcctctccttcagctctacttccgggccttggttactggctcactccgtgcacgttggtgccccatcctctacactgtagccgtggctcatgtcaacagtttcatcttctgccaagacccaaagagctcagtaagtcacctctctgtgttcagagggaaggagggagctggtggaccagttggggatgggctgagggttaagtgagtccatgggtgggtaccagtcctcggctttgtgtcatatctctcttccgtccactcccaggatgaggtaaagactgcccgaaggagtatgctgcagagaacgtggctgctgacagatgaggtagtattgtgggtgtcgggacagaaaagatacaggacgttaaggtaccaaacctcaacaaactgaccacccacttcatgggttttgtcttctctgcaggggctgcggcagcatctcctccactataagcttcccaactccagcctcccagagggctttgagctgtattcccagttacctcgcctgaggcagcagtgccttcagacactgcccacagaggggctccagaatggaggagtcaagacatagggcacttgcccccagctaaagatgactactctactctcctgggactgagcagtgagcctcccgagagaaggcaacacaagacttgccttcctgtgagcacattgtttgagccttttttagaacagaagagactcgtattacatcatgatactggagctcaggatctgggggctgagggctgtgccaggcgtggaaggagggtggcttcagtgtttaccctgcggtgcccaggacagtctgagcacagctctcctcactcacctccagtcttcaccagaccagggtgtgcaattactgtgctacactctgaaataaattcttttctagtctgactgatctggactgttttgtcacattcagagttggtagcaaggatggattttcccgaggagagagatgggagaaaatgtcacaatgggaggtaaacacattaccatctgggggtggcagaggtgtgtgtcctgggagtcagtgacagtgagcctggagtgggtgtggtcctggagga

SEQ ID NO: 161 - DNA, 1180 bp, Unknown Organism
  Description of Unknown Organism Ltk
ttcaacaccttatccagtcatggcagtaagaaattgtccacaggaacagtgtgcaacaaactcagaaagtcatgattctgaggtactgggatgtcctgcctcatggtcagaaagggacacagcaatggcaagcaataatgccttacgggggacagtttaatcgccatcacccaggaggcagaggcaggtggatttttggaggctctacatacttcccatgtgtgatcatgcacttaaactaacactgacaccaataaagccattcactattgatgctatcctgagacccacaaaagctactgccatgtactagggtgctgtccagcctgctatcacaaaggtataatgtagagtcaactttttctcctaccaggaactcactagtttgttggtgtgccataggtggggactagagcccttcagctcagcaaggctcctcccagaagcctggggacctcgctgtcctcatgctccgccttttccttctatttcccgggtctgacgtgagtgtatacaggccagttggaagtcggcagggtagtggtgatagctctgagactgctgacacgcctataaactgcagctagggaatcgagttctcaaccatcatcctttgctggaaccaagggatcgggcgatgggctgctcccaccggctgctgctgtggctgggagctgctggtaagtgctgtttggtggtcagggtaccagccaaccagagacaactagatttggggctgcattagcaacagaccaggtctcaatggttccttctaaggtggccttgtgcttttagagtacagcaagaaaggtgcggaacacgtccaaatctaagaatcagatggggccttggcactacttgggaggaagctagagagaggagaggtggtggggtgctgtacaacagagagcggtgcagcccccaagtcactccatgagagctctggaatagaggtcatgatatatgtagataccttgcaaggcttgggaaacccggttgggtggtaggtgtctagcttcagcctctgtatgcaccagctgggcggcattcggggtgtggctggaagcagggtactgctgagtggctatccatctcttatgtctcccctgtcacattctgcatcatctaacccttcttcctgtcacctttcaggaactattctttgctccaactcggagtt

SEQ ID NO: 162 - DNA, 1144 bp, Unknown Organism
  Description of Unknown Organism Ltk
cggctgctgctgtggctgggagctgctggtaagtgctgtttggtggtcagggtaccagccaaccagagacaactagatttggggctgcattagcaacagaccaggtctcaatggttccttctaaggtggccttgtgcttttagagtacagcaagaaaggtgcggaacacgtccaaatctaagaatcagatggggccttggcactacttgggaggaagctagagagaggagaggtggtggggtgctgtacaacagagagcggtgcagcccccaagtcactccatgagagctctggaatagaggtcatgatatatgtagataccttgcaaggcttgggaaacccggttgggtggtaggtgtctagcttcagcctctgtatgcaccagctgggcggcattcggggtgtggctggaagcagggtactgctgagtggctatccatctcttatgtctcccctgtcacattctgcatcatctaacccttcttcctgtcacctttcaggaactattctttgctccaactcggagttccaggcaccttttctaacaccttcgcttttgccggtgctggtactcaattcccaggagcagaaagtcacccccacacccagtaaattggagccagcttccctcccaaatcctctaggtgagtttcaggctcctcccttttcttggttcaccatctccagatccctaggcctggagaggaactttttccttctccctttgtacctaggcacacgggggccttgggtgtttaacacctgtggcgccagcggcaggagcggacccacacaaacacagtgcgatggggcatacacaggaagcagcgtgatggtgacagtgggggctgccgggccgctcaaaggcgtgcagctgtggcgggcgccagacacaggccagtatctgtaagtgctcagcaggaagagggtggcaatcacctcttaggctgaggttttacagaccagggaagttatcttaaaacattccgcagagaagtctagtgtcagtcacttcgattgtctgtcctcctaagttaaccatcgtcaggacggccgcccaatcaacggtggctgaagatggggctggatgattccagccatctccgttaacgctcttgaaatactcgtggaacccaactgaaaa

SEQ ID NO: 163 - DNA, 1172 bp, Unknown Organism
  Description of Unknown Organism Ltk
ggtggggtgctgtacaacagagagcggtgcagcccccaagtcactccatgagagctctggaatagaggtcatgatatatgtagataccttgcaaggcttgggaaacccggttgggtggtaggtgtctagcttcagcctctgtatgcaccagctgggcggcattcggggtgtggctggaagcagggtactgctgagtggctatccatctcttatgtctcccctgtcacattctgcatcatctaacccttcttcctgtcacctttcaggaactattctttgctccaactcggagttccaggcaccttttctaacaccttcgcttttgccggtgctggtactcaattcccaggagcagaaagtcacccccacacccagtaaattggagccagcttccctcccaaatcctctaggtgagtttcaggctcctcccttttcttggttcaccatctccagatccctaggcctggagaggaactttttccttctccctttgtacctaggcacacgggggccttgggtgtttaacacctgtggcgccagcggcaggagcggacccacacaaacacagtgcgatggggcatacacaggaagcagcgtgatggtgacagtgggggctgccgggccgctcaaaggcgtgcagctgtggcgggcgccagacacaggccagtatctgtaagtgctcagcaggaagagggtggcaatcacctcttaggctgaggttttacagaccagggaagttatcttaaaacattccgcagagaagtctagtgtcagtcacttcgattgtctgtcctcctaagttaaccatcgtcaggacggccgcccaatcaacggtggctgaagatggggctggatgattccagccatctccgttaacgctcttgaaatactcgtggaacccaactgaaaactcggttcttccccatcccttcacagtccatctaagtcccttaggaagaagctttccggccgcgggttcccgaccgtgagccgccgcctggcccaggaccgaggcgcctaggactgagacgcggaccggtgctctctcctcctgcaggatctccgcctacggagcggcgggcggcaagggcgcccaaaaccacctgtcacgggcgcacggcatcttcctctcagcagtcttcttcctccgtcgcggggagccggtgtacatc

SEQ ID NO: 164 - DNA, 1151 bp, Unknown Organism
  Description of Unknown Organism Ltk
cacaaacacagtgcgatggggcatacacaggaagcagcgtgatggtgacagtgggggctgccgggccgctcaaaggcgtgcagctgtggcgggcgccagacacaggccagtatctgtaagtgctcagcaggaagagggtggcaatcacctcttaggctgaggttttacagaccagggaagttatcttaaaacattccgcagagaagtctagtgtcagtcacttcgattgtctgtcctcctaagttaaccatcgtcaggacggccgcccaatcaacggtggctgaagatggggctggatgattccagccatctccgttaacgctcttgaaatactcgtggaacccaactgaaaactcggttcttccccatcccttcacagtccatctaagtcccttaggaagaagctttccggccgcgggttcccgaccgtgagccgccgcctggcccaggaccgaggcgcctaggactgagacgcggaccggtgctctctcctcctgcaggatctccgcctacggagcggcgggcggcaagggcgcccaaaaccacctgtcacgggcgcacggcatcttcctctcagcagtcttcttcctccgtcgcggggagccggtgtacatccttgtggggcagcagggccaggacgcctgtcccggagtaagcggggcgagtgggacaggggcgcccggtccgagagctggaggaggagagagtccctggtcacgcagcatttcaccttggtgccacccaggggagccctgagagccaactcgtctgtctgggagagtctggggagcatgcaaccacctatgggaccgaaaggatcccaggctggagacgctgggccggcgggggcgggggtggcggaggcgccacctccatcttccgggtaggtgcaccgggtgctgtcgcctcgacccccagctgggtagcccaggctccgctctccctctgggttccccgggctcaggacccgggtgcagcccctgaccggccactggccccatagctgcgcgcgggggagccagagccgctgctggtggcggcgggaggcggcgggaggtcctaccggaggcgacctgaccgcggccggactcaggccgtccccgagaggctggagacccgcgcggcggcgccgggcagcggcgggagaggag

SEQ ID NO: 165 - DNA, 1138 bp, Unknown Organism
  Description of Unknown Organism Ltk
aaccatcgtcaggacggccgcccaatcaacggtggctgaagatggggctggatgattccagccatctccgttaacgctcttgaaatactcgtggaacccaactgaaaactcggttcttccccatcccttcacagtccatctaagtcccttaggaagaagctttccggccgcgggttcccgaccgtgagccgccgcctggcccaggaccgaggcgcctaggactgagacgcggaccggtgctctctcctcctgcaggatctccgcctacggagcggcgggcggcaagggcgcccaaaaccacctgtcacgggcgcacggcatcttcctctcagcagtcttcttcctccgtcgcggggagccggtgtacatccttgtggggcagcagggccaggacgcctgtcccggagtaagcggggcgagtgggacaggggcgcccggtccgagagctggaggaggagagagtccctggtcacgcagcatttcaccttggtgccacccaggggagccctgagagccaactcgtctgtctgggagagtctggggagcatgcaaccacctatgggaccgaaaggatcccaggctggagacgctgggccggcgggggcgggggtggcggaggcgccacctccatcttccgggtaggtgcaccgggtgctgtcgcctcgacccccagctgggtagcccaggctccgctctccctctgggttccccgggctcaggacccgggtgcagcccctgaccggccactggccccatagctgcgcgcgggggagccagagccgctgctggtggcggcgggaggcggcgggaggtcctaccggaggcgacctgaccgcggccggactcaggccgtccccgagaggctggagacccgcgcggcggcgccgggcagcggcgggagaggaggcgcggcaggtgagcgctccgcccggcggggagaggcggggagaggcgagcggctccgcaacgccactgctcgtcgcttgctgtcgcttcgcaggtggagggagcggctggacgtcgcgagcccactctccgcaggccggacgctcgccgcgggaaggggccgagggcggcgagggctgcgcggaggcctgggctgcgctccgctgggctgcggcgggaggcttcgggggcg

SEQ ID NO: 166 - DNA, 1157 bp, Unknown Organism
  Description of Unknown Organism Ltk
ctccgcctacggagcggcgggcggcaagggcgcccaaaaccacctgtcacgggcgcacggcatcttcctctcagcagtcttcttcctccgtcgcggggagccggtgtacatccttgtggggcagcagggccaggacgcctgtcccggagtaagcggggcgagtgggacaggggcgcccggtccgagagctggaggaggagagagtccctggtcacgcagcatttcaccttggtgccacccaggggagccctgagagccaactcgtctgtctgggagagtctggggagcatgcaaccacctatgggaccgaaaggatcccaggctggagacgctgggccggcgggggcgggggtggcggaggcgccacctccatcttccgggtaggtgcaccgggtgctgtcgcctcgacccccagctgggtagcccaggctccgctctccctctgggttccccgggctcaggacccgggtgcagcccctgaccggccactggccccatagctgcgcgcgggggagccagagccgctgctggtggcggcgggaggcggcgggaggtcctaccggaggcgacctgaccgcggccggactcaggccgtccccgagaggctggagacccgcgcggcggcgccgggcagcggcgggagaggaggcgcggcaggtgagcgctccgcccggcggggagaggcggggagaggcgagcggctccgcaacgccactgctcgtcgcttgctgtcgcttcgcaggtggagggagcggctggacgtcgcgagcccactctccgcaggccggacgctcgccgcgggaaggggccgagggcggcgagggctgcgcggaggcctgggctgcgctccgctgggctgcggcgggaggcttcgggggcggcggcggggcctgcgcggctggcggcggtggcggcggctaccggggtaggtgcacccgttgggagggttagtcggacagcccgggtgatgtgacggtagagcctaagaacctggcctgagctttccagaggatagatggggaatgccccactctccactgtgccaggccccagcaggtgctcttacaaaggaggtctgaccaggtcaggcctgggcggggtggcacactcgcctgcagcgcgtgctggaggcagaagtattccaagctaaataacta

SEQ ID NO: 167 - DNA, 1183 bp, Unknown Organism
  Description of Unknown Organism Ltk
gggagccctgagagccaactcgtctgtctgggagagtctggggagcatgcaaccacctatgggaccgaaaggatcccaggctggagacgctgggccggcgggggcgggggtggcggaggcgccacctccatcttccgggtaggtgcaccgggtgctgtcgcctcgacccccagctgggtagcccaggctccgctctccctctgggttccccgggctcaggacccgggtgcagcccctgaccggccactggccccatagctgcgcgcgggggagccagagccgctgctggtggcggcgggaggcggcgggaggtcctaccggaggcgacctgaccgcggccggactcaggccgtccccgagaggctggagacccgcgcggcggcgccgggcagcggcgggagaggaggcgcggcaggtgagcgctccgcccggcggggagaggcggggagaggcgagcggctccgcaacgccactgctcgtcgcttgctgtcgcttcgcaggtggagggagcggctggacgtcgcgagcccactctccgcaggccggacgctcgccgcgggaaggggccgagggcggcgagggctgcgcggaggcctgggctgcgctccgctgggctgcggcgggaggcttcgggggcggcggcggggcctgcgcggctggcggcggtggcggcggctaccggggtaggtgcacccgttgggagggttagtcggacagcccgggtgatgtgacggtagagcctaagaacctggcctgagctttccagaggatagatggggaatgccccactctccactgtgccaggccccagcaggtgctcttacaaaggaggtctgaccaggtcaggcctgggcggggtggcacactcgcctgcagcgcgtgctggaggcagaagtattccaagctaaataactacggagtgaagccaggctgggctacagacttgtctggttttgttttgttttaagactgggtttcttttttgagacaggctgacctggaactccctctgtaagccaggctggcctcgaactcacagatacccacctgcctttgcctcccaggagttgaaatgaaaggcttgcaccaccaccaagcaggctggtcttgttttgtcttttacaaagggccaaattacccatgcttggtcgcacattcctttaattctactatcaggaaacag

SEQ ID NO: 168 - DNA, 1099 bp, Unknown Organism
  Description of Unknown Organism Ltk
caaggcagctgcatgctcctcctcaaccagtctcctttttgtagttaaacctccatcagccgggcggtggtggtacacgccttaaatcccagcagaggatttaagagggaggcagaggcaggtggatttctgagtttgaggccagcctggtctacaaagtgagctccaggacagccagggctacacagagaaaccctgtctcaaaaaaacaaacaaacaaacaaacagacaaaaaaacaaaaccaaaacaaacaaacaaaaaaaagatgctaagcaccttttggggactggagagatggctcaggggttaagagcaatgagtgctcttccgaaagtcctgagttcaaatcccagcaaccatatagtggctcacaaccatctgtaatgagatctgatgccctcttctgctgtgtctgaaaacagctacactgtacttacatgtaataataataaaaacaaaaaaacaaaaaacaaaaacctctatcactctccatttcaaggcggtgatacttctgagtctgacctcctctgggctgatggggaagatggcacatcctttgtccaccccagtggtgagctctacctacagcctctggcaggtatttgtctggaacccctgattctcagcctggccagctcctcctcaatctggtaaaaccgttgcctctcaagcttggtgacctgacaccatagtagaagaagaaaagaaagtctgacttcacaagcatgtgtgtgcgtggcttggtctacctctgaatgaaatcagtcaacagaaattaagtccccggggctctgacatcaccctttgtgttgctgtggttgggaccactcctgactaggagtagccatatgactggaaagacctgtgtgtcccatccccatccccacccccacttcccaccccaccatgtctactcttttactctgcagtcacagagggccatggggaggtggagatccgaaagcatcccaactgcagtcactgccctttcaaagactgccagtggcaggcagagctctggacggccgaatgcacgtgcccagagggcacggagctagctgtggataatgtcacttgcatgggtatgtcttcatagag

SEQ ID NO: 169 - DNA, 1153 bp, Unknown Organism
  Description of Unknown Organism Ltk
gtacttacatgtaataataataaaaacaaaaaaacaaaaaacaaaaacctctatcactctccatttcaaggcggtgatacttctgagtctgacctcctctgggctgatggggaagatggcacatcctttgtccaccccagtggtgagctctacctacagcctctggcaggtatttgtctggaacccctgattctcagcctggccagctcctcctcaatctggtaaaaccgttgcctctcaagcttggtgacctgacaccatagtagaagaagaaaagaaagtctgacttcacaagcatgtgtgtgcgtggcttggtctacctctgaatgaaatcagtcaacagaaattaagtccccggggctctgacatcaccctttgtgttgctgtggttgggaccactcctgactaggagtagccatatgactggaaagacctgtgtgtcccatccccatccccacccccacttcccaccccaccatgtctactcttttactctgcagtcacagagggccatggggaggtggagatccgaaagcatcccaactgcagtcactgccctttcaaagactgccagtggcaggcagagctctggacggccgaatgcacgtgcccagagggcacggagctagctgtggataatgtcacttgcatgggtatgtcttcatagagcattggaaatggatcagggcacagtctcctgtcaagaacatgtaggccagtgtgggaggcaagatagatatttagatatagatatgtatctaaaaatggcagttgctatggaaggtatcaaatagttaaggggctctcgagagggagagttgcttttgtctggggacaactgtgggtggcactgtgggaaagtaaacactgagatggggcttgaagcatggataagatttaagggtacccagtcatggtagcatacacctctcatcccagcagtgagtaggctacagcagaattgagagtctgagcccagcctgggttattgggagaccctatctcaaaaacaacaagacaaaacaaaacacaagacaaacgagaaaaatacttgagggtagcaggacagaggaccctcctgctggaggtgacattgttctttctccctagacctgccaactaccgcaagccctctgatcctgatgggagctgt

SEQ ID NO: 170 - DNA, 1096 bp, Unknown Organism
  Description of Unknown Organism Ltk
ccagagggcacggagctagctgtggataatgtcacttgcatgggtatgtcttcatagagcattggaaatggatcagggcacagtctcctgtcaagaacatgtaggccagtgtgggaggcaagatagatatttagatatagatatgtatctaaaaatggcagttgctatggaaggtatcaaatagttaaggggctctcgagagggagagttgcttttgtctggggacaactgtgggtggcactgtgggaaagtaaacactgagatggggcttgaagcatggataagatttaagggtacccagtcatggtagcatacacctctcatcccagcagtgagtaggctacagcagaattgagagtctgagcccagcctgggttattgggagaccctatctcaaaaacaacaagacaaaacaaaacacaagacaaacgagaaaaatacttgagggtagcaggacagaggaccctcctgctggaggtgacattgttctttctccctagacctgccaactaccgcaagccctctgatcctgatgggagctgtagtggcagccttggcactgagtctcctaatgatgtgtgcagtcctgattctaggtatgggcatctagcatggctgatgcaggccgacccttctctcctaggctgtgatgggcttctctgtggttctaggctaaatgtggctgacatcatggcccttaccccacagagcaatggtggagtcctctcagcttttaggagtagtgccagggagtcctgggcctggggaagcagtgtgggcctgataaatacctctcttctcacccacagtgaaccagaagtgtcagggcctgtgggggaccaggctgccaggccctgagcttgagctaagcaagcttcgatcctctgccatcaggacagcacccaacccttactattgtcaggtgggactcagtcctgcccagccctggcctttgcccccagggctcactgaggtttcaccagccaatgtcactctactcaggtgagtcctcaacctccttctgcaatgatgtgcactttgaagccctcctccccattcttcctcctgtcatgatgcattatgaatcccagagggc

SEQ ID NO: 171 - DNA, 1193 bp, Unknown Organism
  Description of Unknown Organism Ltk
gcatacacctctcatcccagcagtgagtaggctacagcagaattgagagtctgagcccagcctgggttattgggagaccctatctcaaaaacaacaagacaaaacaaaacacaagacaaacgagaaaaatacttgagggtagcaggacagaggaccctcctgctggaggtgacattgttctttctccctagacctgccaactaccgcaagccctctgatcctgatgggagctgtagtggcagccttggcactgagtctcctaatgatgtgtgcagtcctgattctaggtatgggcatctagcatggctgatgcaggccgacccttctctcctaggctgtgatgggcttctctgtggttctaggctaaatgtggctgacatcatggcccttaccccacagagcaatggtggagtcctctcagcttttaggagtagtgccagggagtcctgggcctggggaagcagtgtgggcctgataaatacctctcttctcacccacagtgaaccagaagtgtcagggcctgtgggggaccaggctgccaggccctgagcttgagctaagcaagcttcgatcctctgccatcaggacagcacccaacccttactattgtcaggtgggactcagtcctgcccagccctggcctttgcccccagggctcactgaggtttcaccagccaatgtcactctactcaggtgagtcctcaacctccttctgcaatgatgtgcactttgaagccctcctccccattcttcctcctgtcatgatgcattatgaatcccagagggcatctaactcaaatcccttgtgcagaagaagttccaaaaggcaaagtagcatgggtagggtcagattttgccccagttctggggtgcagagatctgggaacatgactgttctggagcccacagcttgcctttctcccagcagggtctgcattagcacaaggagaggccttatcctacattatgaccctgactctggccccagctctgtgtgtcagcattagcttgtgtctgagagcacccatggcccagagagagctcagagaaggtcccctggacctatggggtgggagccaaagagataaaggggcattggctcttctatagaccctgtttgcctatgccatcctcttccttccatcttgtcttcttatagagcccttggccatggtgcctttggggaagtgtac

SEQ ID NO: 172 - DNA, 1091 bp, Unknown Organism
  Description of Unknown Organism Ltk
ctgaggtttcaccagccaatgtcactctactcaggtgagtcctcaacctccttctgcaatgatgtgcactttgaagccctcctccccattcttcctcctgtcatgatgcattatgaatcccagagggcatctaactcaaatcccttgtgcagaagaagttccaaaaggcaaagtagcatgggtagggtcagattttgccccagttctggggtgcagagatctgggaacatgactgttctggagcccacagcttgcctttctcccagcagggtctgcattagcacaaggagaggccttatcctacattatgaccctgactctggccccagctctgtgtgtcagcattagcttgtgtctgagagcacccatggcccagagagagctcagagaaggtcccctggacctatggggtgggagccaaagagataaaggggcattggctcttctatagaccctgtttgcctatgccatcctcttccttccatcttgtcttcttatagagcccttggccatggtgcctttggggaagtgtacgagggactagtgactggtcttcctggggactccagtcctcttccagtggctattaaggtgagaaggggcagggcttgcggttgggataggcatatggaaggaagggctgatgctagagcctttgttgttctccctcctaaagcactcaaagctcaggagtgctctgcgttccctccccagactctgccagagctctgctcccatcaggatgagctggattttctcatggaggctctgatcatcaggtgcagcccagggcaggcaggagggtatggtgggaggcaggatctgcctacaggaatggcctctctgctctttatggctgaggggcagccttttagggcacagagattttttgcactttaattggatctagaatatgcttatctccccactggtgatggggtctatgcgccatttccactcagcaagttcagccatcagaacattgtacgctgtgtggggctcagctttcggtctgccccgcgcctcattctgctggagctgatgtctggtggggacatgaagagctttttgaggcacagcagaccacaccc

SEQ ID NO: 173 - DNA, 1065 bp, Unknown Organism
  Description of Unknown Organism Ltk
agagatctgggaacatgactgttctggagcccacagcttgcctttctcccagcagggtctgcattagcacaaggagaggccttatcctacattatgaccctgactctggccccagctctgtgtgtcagcattagcttgtgtctgagagcacccatggcccagagagagctcagagaaggtcccctggacctatggggtgggagccaaagagataaaggggcattggctcttctatagaccctgtttgcctatgccatcctcttccttccatcttgtcttcttatagagcccttggccatggtgcctttggggaagtgtacgagggactagtgactggtcttcctggggactccagtcctcttccagtggctattaaggtgagaaggggcagggcttgcggttgggataggcatatggaaggaagggctgatgctagagcctttgttgttctccctcctaaagcactcaaagctcaggagtgctctgcgttccctccccagactctgccagagctctgctcccatcaggatgagctggattttctcatggaggctctgatcatcaggtgcagcccagggcaggcaggagggtatggtgggaggcaggatctgcctacaggaatggcctctctgctctttatggctgaggggcagccttttagggcacagagattttttgcactttaattggatctagaatatgcttatctccccactggtgatggggtctatgcgccatttccactcagcaagttcagccatcagaacattgtacgctgtgtggggctcagctttcggtctgccccgcgcctcattctgctggagctgatgtctggtggggacatgaagagctttttgaggcacagcagaccacacccagtaaggcccttttctgggctccccagacccttctaaaatcctatttgctctacgatctggatgaaatgatgtaccttctgcccaactcagggacaactggcacctctgaccatgcaggacctattgcagctggcccaggatatagcccagggctgccactacctggaggaaaatcacttcattcaca

SEQ ID NO: 174 - DNA, 1130 bp, Unknown Organism
  Description of Unknown Organism Ltk
ctatgccatcctcttccttccatcttgtcttcttatagagcccttggccatggtgcctttggggaagtgtacgagggactagtgactggtcttcctggggactccagtcctcttccagtggctattaaggtgagaaggggcagggcttgcggttgggataggcatatggaaggaagggctgatgctagagcctttgttgttctccctcctaaagcactcaaagctcaggagtgctctgcgttccctccccagactctgccagagctctgctcccatcaggatgagctggattttctcatggaggctctgatcatcaggtgcagcccagggcaggcaggagggtatggtgggaggcaggatctgcctacaggaatggcctctctgctctttatggctgaggggcagccttttagggcacagagattttttgcactttaattggatctagaatatgcttatctccccactggtgatggggtctatgcgccatttccactcagcaagttcagccatcagaacattgtacgctgtgtggggctcagctttcggtctgccccgcgcctcattctgctggagctgatgtctggtggggacatgaagagctttttgaggcacagcagaccacacccagtaaggcccttttctgggctccccagacccttctaaaatcctatttgctctacgatctggatgaaatgatgtaccttctgcccaactcagggacaactggcacctctgaccatgcaggacctattgcagctggcccaggatatagcccagggctgccactacctggaggaaaatcacttcattcacaggttggaagcaatcaggagacccttggcccacttctcccgtcagaacatccctctctatatatacaactttaaagaaatgtaacagaaagagccagagtgcctatgctccgtctggcatgaacattttatgttttacagagacattgctgcccgtaactgtctgcttagctgcagtggagccagccgagtggccaagattggagattttggaatggcaagagatatctaccagtaagcaggaactggagagctgggaaagctgctgttccacggaggttggcttgaccacactgggaaaactctgcacttttt

SEQ ID NO: 175 - DNA, 1098 bp, Unknown Organism
  Description of Unknown Organism Ltk
aagctcaggagtgctctgcgttccctccccagactctgccagagctctgctcccatcaggatgagctggattttctcatggaggctctgatcatcaggtgcagcccagggcaggcaggagggtatggtgggaggcaggatctgcctacaggaatggcctctctgctctttatggctgaggggcagccttttagggcacagagattttttgcactttaattggatctagaatatgcttatctccccactggtgatggggtctatgcgccatttccactcagcaagttcagccatcagaacattgtacgctgtgtggggctcagctttcggtctgccccgcgcctcattctgctggagctgatgtctggtggggacatgaagagctttttgaggcacagcagaccacacccagtaaggcccttttctgggctccccagacccttctaaaatcctatttgctctacgatctggatgaaatgatgtaccttctgcccaactcagggacaactggcacctctgaccatgcaggacctattgcagctggcccaggatatagcccagggctgccactacctggaggaaaatcacttcattcacaggttggaagcaatcaggagacccttggcccacttctcccgtcagaacatccctctctatatatacaactttaaagaaatgtaacagaaagagccagagtgcctatgctccgtctggcatgaacattttatgttttacagagacattgctgcccgtaactgtctgcttagctgcagtggagccagccgagtggccaagattggagattttggaatggcaagagatatctaccagtaagcaggaactggagagctgggaaagctgctgttccacggaggttggcttgaccacactgggaaaactctgcactttttttctccttcatatagggccagttattatcgcaagggtggccggaccttgctcccagtcaagtggatgccgccagaagctctcctggagggccttttcacatccaagacagactcctggtgatagcatgtcccccgctgtgctcccctcactctgccctgaagctggtcctatctcttcaccacctcat

SEQ ID NO: 176 - DNA, 1093 bp, Unknown Organism
  Description of Unknown Organism Ltk
tatctccccactggtgatggggtctatgcgccatttccactcagcaagttcagccatcagaacattgtacgctgtgtggggctcagctttcggtctgccccgcgcctcattctgctggagctgatgtctggtggggacatgaagagctttttgaggcacagcagaccacacccagtaaggcccttttctgggctccccagacccttctaaaatcctatttgctctacgatctggatgaaatgatgtaccttctgcccaactcagggacaactggcacctctgaccatgcaggacctattgcagctggcccaggatatagcccagggctgccactacctggaggaaaatcacttcattcacaggttggaagcaatcaggagacccttggcccacttctcccgtcagaacatccctctctatatatacaactttaaagaaatgtaacagaaagagccagagtgcctatgctccgtctggcatgaacattttatgttttacagagacattgctgcccgtaactgtctgcttagctgcagtggagccagccgagtggccaagattggagattttggaatggcaagagatatctaccagtaagcaggaactggagagctgggaaagctgctgttccacggaggttggcttgaccacactgggaaaactctgcactttttttctccttcatatagggccagttattatcgcaagggtggccggaccttgctcccagtcaagtggatgccgccagaagctctcctggagggccttttcacatccaagacagactcctggtgatagcatgtcccccgctgtgctcccctcactctgccctgaagctggtcctatctcttcaccacctcatatgacagcaggggacagtacacatgatgggtcttgtttgttccttccacctctgacacaggtcttttggggtcctgctctgggagatcttctcactggggtatatgccctaccctggacataccaaccaggaggttctagacttcattgccacagggaacaggatggaccctcctaggaactgtcctgggccagtgtgagctaacagtgctgcccagggagggaggcatcc

SEQ ID NO: 177 - DNA, 1102 bp, Unknown Organism
  Description of Unknown Organism Ltk
gggctccccagacccttctaaaatcctatttgctctacgatctggatgaaatgatgtaccttctgcccaactcagggacaactggcacctctgaccatgcaggacctattgcagctggcccaggatatagcccagggctgccactacctggaggaaaatcacttcattcacaggttggaagcaatcaggagacccttggcccacttctcccgtcagaacatccctctctatatatacaactttaaagaaatgtaacagaaagagccagagtgcctatgctccgtctggcatgaacattttatgttttacagagacattgctgcccgtaactgtctgcttagctgcagtggagccagccgagtggccaagattggagattttggaatggcaagagatatctaccagtaagcaggaactggagagctgggaaagctgctgttccacggaggttggcttgaccacactgggaaaactctgcactttttttctccttcatatagggccagttattatcgcaagggtggccggaccttgctcccagtcaagtggatgccgccagaagctctcctggagggccttttcacatccaagacagactcctggtgatagcatgtcccccgctgtgctcccctcactctgccctgaagctggtcctatctcttcaccacctcatatgacagcaggggacagtacacatgatgggtcttgtttgttccttccacctctgacacaggtcttttggggtcctgctctgggagatcttctcactggggtatatgccctaccctggacataccaaccaggaggttctagacttcattgccacagggaacaggatggaccctcctaggaactgtcctgggccagtgtgagctaacagtgctgcccagggagggaggcatcccaaggtttcaggaggcataaactcctgcctctccctaggtaccgaatcatgacccagtgttggcagcatcagccggagctccgccctgactttggcagcatcttggaacggattcagtactgcactcaggtgtgccccccacccaacccccagctccctttcgaccccctctaactaccctggttctgcagggcagga

SEQ ID NO: 178 - DNA, 1135 bp, Unknown Organism
  Description of Unknown Organism Ltk
atacaactttaaagaaatgtaacagaaagagccagagtgcctatgctccgtctggcatgaacattttatgttttacagagacattgctgcccgtaactgtctgcttagctgcagtggagccagccgagtggccaagattggagattttggaatggcaagagatatctaccagtaagcaggaactggagagctgggaaagctgctgttccacggaggttggcttgaccacactgggaaaactctgcactttttttctccttcatatagggccagttattatcgcaagggtggccggaccttgctcccagtcaagtggatgccgccagaagctctcctggagggccttttcacatccaagacagactcctggtgatagcatgtcccccgctgtgctcccctcactctgccctgaagctggtcctatctcttcaccacctcatatgacagcaggggacagtacacatgatgggtcttgtttgttccttccacctctgacacaggtcttttggggtcctgctctgggagatcttctcactggggtatatgccctaccctggacataccaaccaggaggttctagacttcattgccacagggaacaggatggaccctcctaggaactgtcctgggccagtgtgagctaacagtgctgcccagggagggaggcatcccaaggtttcaggaggcataaactcctgcctctccctaggtaccgaatcatgacccagtgttggcagcatcagccggagctccgccctgactttggcagcatcttggaacggattcagtactgcactcaggtgtgccccccacccaacccccagctccctttcgaccccctctaactaccctggttctgcagggcaggaagagagccataaaatagccatgtggttccctcttccccaactctcccattacaggaccctgatgtgctgaactcacccctgcccatggaacctgggcccattctagaggaggaagaggcctccaggctgggaaacaggtcactggagggtcttagatccccaaagcccctagagctgagttctcagaacttgaagagctggggaggaggccttcttggctcttggctgccctctggcctcaagaccctcaaacccaggtgcctcca

SEQ ID NO: 179 - DNA, 1091 bp, Unknown Organism
  Description of Unknown Organism Ltk
acggaggttggcttgaccacactgggaaaactctgcactttttttctccttcatatagggccagttattatcgcaagggtggccggaccttgctcccagtcaagtggatgccgccagaagctctcctggagggccttttcacatccaagacagactcctggtgatagcatgtcccccgctgtgctcccctcactctgccctgaagctggtcctatctcttcaccacctcatatgacagcaggggacagtacacatgatgggtcttgtttgttccttccacctctgacacaggtcttttggggtcctgctctgggagatcttctcactggggtatatgccctaccctggacataccaaccaggaggttctagacttcattgccacagggaacaggatggaccctcctaggaactgtcctgggccagtgtgagctaacagtgctgcccagggagggaggcatcccaaggtttcaggaggcataaactcctgcctctccctaggtaccgaatcatgacccagtgttggcagcatcagccggagctccgccctgactttggcagcatcttggaacggattcagtactgcactcaggtgtgccccccacccaacccccagctccctttcgaccccctctaactaccctggttctgcagggcaggaagagagccataaaatagccatgtggttccctcttccccaactctcccattacaggaccctgatgtgctgaactcacccctgcccatggaacctgggcccattctagaggaggaagaggcctccaggctgggaaacaggtcactggagggtcttagatccccaaagcccctagagctgagttctcagaacttgaagagctggggaggaggccttcttggctcttggctgccctctggcctcaagaccctcaaacccaggtgcctccaacctcagaacatttggaaccccacctatggctcctggaccccaaggggcccccagggtgaagatacaggcattgaacagtgcaatggctcctcctcaagttccattccaggcatccagtaggctctgcccctgccctgtgctgcatgttcaggcatgtttcaggg

SEQ ID NO: 180 - DNA, 1606 bp, Unknown Organism
  Description of Unknown Organism Ltk
tctcttcaccacctcatatgacagcaggggacagtacacatgatgggtcttgtttgttccttccacctctgacacaggtcttttggggtcctgctctgggagatcttctcactggggtatatgccctaccctggacataccaaccaggaggttctagacttcattgccacagggaacaggatggaccctcctaggaactgtcctgggccagtgtgagctaacagtgctgcccagggagggaggcatcccaaggtttcaggaggcataaactcctgcctctccctaggtaccgaatcatgacccagtgttggcagcatcagccggagctccgccctgactttggcagcatcttggaacggattcagtactgcactcaggtgtgccccccacccaacccccagctccctttcgaccccctctaactaccctggttctgcagggcaggaagagagccataaaatagccatgtggttccctcttccccaactctcccattacaggaccctgatgtgctgaactcacccctgcccatggaacctgggcccattctagaggaggaagaggcctccaggctgggaaacaggtcactggagggtcttagatccccaaagcccctagagctgagttctcagaacttgaagagctggggaggaggccttcttggctcttggctgccctctggcctcaagaccctcaaacccaggtgcctccaacctcagaacatttggaaccccacctatggctcctggaccccaaggggcccccagggtgaagatacaggcattgaacagtgcaatggctcctcctcaagttccattccaggcatccagtaggctctgcccctgccctgtgctgcatgttcaggcatgtttcagggatgtgcgggcaacctggcctcccacactggaaactagcccagaccctcttggggaaggtcctaggctactttcagtctttggtctttgaaaccagaggccatccacccactgcaggagtcagttgggaaccctggattgtccactcctcataagtatcttttctctgctcattccagggctacaaatgttttagtaataataatggtaataaaaacaataaacccttctaattcttcagttctctgagtgaatgtggaagagagaactggaagtgaaggggaggaggctggagtcacagaagctgaatctttgagttttattaggaagggtgtccactgtagtgcaggagttagaccaggaagtctatatacctgtgaagtacaattatggatcgggaccctctggagaggtgcaaagccacttgagaagagctgggctgggggagaagcccattgtttctccccctccctataaattagtgtcattcagctctgggaaacaggcttgtgtggtaggcaactcctgatgtactacacccccttagtggatggcacccggtcctgcaggaccatgtgaactgcccgcccagcggggttccagtctcttgccggcatgcatggtggggatgcagccacaggtgcacatctgtctgaccagccggcccacgttgagggaaaagggctcagcctcatctctcagccaggctggccaagatgccaatgagattg

SEQ ID NO: 181 - DNA, 728 bp, Unknown Organism
  Description of Unknown Organism lp3k
ggggggcgcggccggggcggggcgcggccgggcgggcgttgccgaggctccgccccggggcagtcccaagcccggggcgctgagcgtctacagtccccgccgcgccgcgggctggtgggctcggctgcggctccggtgccgggagatgaccctgcccgggcgcccgacgggcatggcgcggccacggggcgcggggccctgcagccccgggctggagagggctccgcgccggagcgtcggggagctgcgcctgctcttcgaagcgcgctgcgccgcagtcgccgctgcagcagccgcaggggagccccgggcccgcggggccaagcggcgtgggggacaagtgcccaacgggctcccgcgagctgcccctgccccggtgatccctcagctcactgtgacaagcgaggaggatgtgaccccggccagcccagggccgccagaccaggaggggaactggctcccagctgcggggtcgcacctgcagcagccacgccgcctctccacctcgtccctctcctccaccggctcctcgtcgctgctcgaggactcggaggacgatctgctgagcgacagtgagagcaggagccgcggcaacgtgcagctggaaaccagcgaggacgtggggcaggtacgggccgcgggggcggggccagcgggggctgcgcgccggggactccgctctctgctctgctctgaccggcttgcgttccccgccgagagcccaagcg

SEQ ID NO: 182 - DNA, 297 bp, Unknown Organism
  Description of Unknown Organism lp3k
gaccttaactgcttctagtgggatgaaaccaagctgaaatcctgctctagagggacattactgagacgctgacttgcacacctggacctgttgctttcagaaaagccactggcagaagatccgtaccatggtcaatctgcctgtcatgagtcctttcagaaagcgctactcctgggtgcagttagcagggcacacaggtgagcatcattgcaggtgggtgaggttgctgccacaggaaggcttgggcagttctaactggtgcccttgtgcagggagtttcaaagctgccggcaccag

SEQ ID NO: 183 - DNA, 417 bp, Unknown Organism
  Description of Unknown Organism lp3k
tgggtgcagttagcagggcacacaggtgagcatcattgcaggtgggtgaggttgctgccacaggaaggcttgggcagttctaactggtgcccttgtgcagggagtttcaaagctgccggcaccagtggcctgatcctgaaacgcagctcggagcctgaacactactgcctggtgcggctgatggctgacgtgctgcgtgggtgtgtgccggccttccatggcatagtcgagcgggatggtgaaagctacttgcagttacaggacctgctcgatggcttcgatgggccttgtgtgcttgactgcaagatgggtgtcaggtttgtgtcccctctctttgtcaggcagaatcagtccggaaccaaggactgggataaaagctcatatgccctggcccctacttgcacttcacagaactta

SEQ ID NO: 184 - DNA, 405 bp, Unknown Organism
  Description of Unknown Organism lp3k
tgtcaggtttgtgtcccctctctttgtcaggcagaatcagtccggaaccaaggactgggataaaagctcatatgccctggcccctacttgcacttcacagaacttacctggaagaggagctgaccaaagcccgagaacggcccaagctgcggaaggacatgtataagaagatgctggcagtggaccctgaggcacctactgaggaggagcatgcgcagcgcgccgtcaccaaaccacgctacatgcaatggcgcgaaggcatcagctccagcactacactcggctttcgcattgagggcatcaaggtgggtcaggctcccctcactttgcatggcctttcttctatcccaccttcagtgctcctgacctcctgcctcaaccatcaccaaaagacaccctccggta

SEQ ID NO: 185 - DNA, 302 bp, Unknown Organism
  Description of Unknown Organism lp3k
cagaggttaaggccccggggaggtaccactgccctttgcacgatcctctctcccctcagatgggtcctgggctctgacagtccttgcccacctccttcagaaagccgatggatcttgcagtactgatttcaaaactacacgaagccgagagcaagtgacccgtgtctttgaggagttcatgcaaggagatgcagaagtgctggtgagaggaggttcccaagccctggggtctgggcaccttgtttacaagggcgcctcttgcctatgtcctcctcgcctgtgctacccccagtggtagcact

SEQ ID NO: 186 - DNA, 272 bp, Unknown Organism
  Description of Unknown Organism lp3k
cagtgtttgctaggcaagttgaaataaatgcatggtgcttctaagagtcagtagagccgcccttggtgtgaaattaatctgtccactctgactcttccagaggaggtatctgaaccgcctacagcagatccgggataccctggagatctctgatttctttagacggcacgaggtaagtaagaggtgggggggggggggctcagggtgtgagtttgggctttatgagactcagaatgggaaaggggggtgttttggggaaaagcaggtggcct

SEQ ID NO: 187 - DNA, 789 bp, Unknown Organism
  Description of Unknown Organism lp3k
gggggctcagggtgtgagtttgggctttatgagactcagaatgggaaaggggggtgttttggggaaaagcaggtggcctgactgccgtggggatcggcaggtgattggcagctcactcctcttcgtgcatgaccattgccatcgtgctggtgtgtggctcatcgattttggcaagaccacgcctctccccgatggccagatcctggatcatcggaggccctgggaggagggcaaccgtgaggacggctatttgctggggctggacaatctcattggcatcttggccagcctggctgagagatgaggctgagcccttttccctcaacgtgggccggctggtcagacagatgtgcacctgtggctgcatccccaccatgcatgccggcaagagactggaaccccgctgggcgggcagttcacatggtcctgcaggaccgggtgccatccactaagggggtgtagtacatcaggagttgcctaccacacaagcctgtttcccagagctgaatgacactaatttatagggagggggagaaacaatgggcttctcccccagcccagctcttctcaagtggctttgcacctctccagagggtcccgatccataattgtacttcacaggtatatagacttcctggtctaactcctgcactacagtggacacccttcctaataaaactcaaagattcagcttctgtgactccagcctcctccccttcacttccagttctctcttccacattcactcagagaactgaagaattagaagggtttattgtttttattacc

SEQ ID NO: 188 - DNA, 498 bp, Unknown Organism
  Description of Unknown Organism Tyro 3
ggagggggagggcggcacgagctccgcggagggcgggcgggcgggccgggaggaggcggcggctcgcagaagaacatgaatcagcggcggcggcggcggctgtggaaggagcgcggtggcccagccgcagccccggggactcctcgctgctgacggcggtggccgcggctctaggcggccgcgggtccgggacgccccggccgagcgccgccccccgcccctcccgcgggcctcccgcccctcctccgccaccctcctctcagcgctcgcgggccgggcccggcatggtgcggcgtcgccgccgatggcgctgaggcggagcatggggtggccggggctccggccgctgctgctggcgggactggcttctctgctgctccccgggtctgcggccgcaggtaggggtggcccgggaggcggcgggaagcggggggctgctgagccgcacgactgaggggcgcagcccggggacggcagcgggcgagacccgggcctggg

SEQ ID NO: 189 - DNA, 384 bp, Unknown Organism
  Description of Unknown Organism Tyro 3
agcatagacaactgttttccagaagagaatgaaggggggatgttcccagaaatgagggctatttcagtcgtactgacacatcccctttcccatcctgcaggcctgaagctcatgggcgccccagtgaagatgaccgtgtctcaggggcagccagtgaagctcaactgcagcgtggaggggatggaggaccctgacatccactggatgaaggatggcaccgtggtccagaatgcaagccaggtgtccatctccatcagcgagcacagctggattggcttactcaggtgcaggagtgcggggagggagcgggaggactggcctgtgcctgaggctttgctcatgtctagaggtctgtggtcttaaaatttggcaaactgtggaacc

SEQ ID NO: 190 - DNA, 301 bp, Unknown Organism
  Description of Unknown Organism Tyro 3
ctgcctcccaagtgctggaattaaaggcgtgcgccaccacgcctggctaggatctggctctcttgactctgaaaagtaagctgctatcctgttttcacagcctaaagtcagtggagcggtctgatgctggcctgtactggtgccaggtgaaggatggggaggaaaccaagatctctcagtcagtatggctcactgtcgaaggtgaggaggcagtactagctgtatgggccattgggcttggagctaagacctataactctgcagaagagaacttaatgtaagctggactgccctaccgcct

SEQ ID NO: 191 - DNA, 371 bp, Unknown Organism
  Description of Unknown Organism Tyro 3
gcagctgacccaatcataagggtcctgtggtgactgcctgttacagagaactatagctaagaagcggcgaggctaactccccctcactttttctccctaggtgtgccattcttcacagtggaaccaaaagatctggcggtgccacccaatgccccttttcagctgtcttgtgaggctgtgggtcctccagaacccgtaaccatttactggtggagaggactcactaaggttgggggacctgctccctctccctctgttttaaatgtgacaggtgagcagtctcaggagggggctttgagccaagagtgagctgggtcaatgcctgtggggagcactatagggttgggatctgcataaatctgagtttgtcg

SEQ ID NO: 192 - DNA, 287 bp, Unknown Organism
  Description of Unknown Organism Tyro 3
agatgctctggcaagagcctgaattgaagtccctccttcgtaaactcagactcagtgggagttgctgcttaggggactgactaaggttttctggccccaggagtgacccagcgcacagagttttcttgtgaagcccgcaacataaaaggcctggccacttcccgaccagccattgttcgccttcaaggtagggggacgcaggctaggttgggaggggctgggggtgttgggaaaggacagaggcagcctcagccaactgtctagcagcgggattggctggcttttca

SEQ ID NO: 193 - DNA, 316 bp, Unknown Organism
  Description of Unknown Organism Tyro 3
ctccaggccttaggaggaaatcaagccttgactcccatgcctccctccctccctcccctcctattttcactcggtgctgacactaagtccctatccacagcaccgcctgcagctcctttcaacaccacagtaacaacgatctccagctacaacgctagcgtggcctgggtgccaggtgctgacggcctagctctgctgcattcctgtactgtacaggtaggctgagctgagcacagcagggtctggctgactgactgtgctggctttggctgcactttgtcgcacttactaggcctgcttgggaatctgcagctgc

SEQ ID NO: 194 - DNA, 378 bp, Unknown Organism
  Description of Unknown Organism Tyro 3
ctgccttccatctcatctcagagacctgactccttatctgagctcactgggtttgtccttttctggtttttgtcttttgcttctgttttgttttctgaaggtggcacacgccccaggagaatgggaggcccttgctgttgtggttcctgtgccaccttttacctgcctgcttcggaacttggcccctgccaccaactacagccttagggtgcgctgtgccaatgccttgggcccttctccctacggcgactgggtgccctttcagacaaagggcctaggtaagaaactcagcaggaggggcagggctggagagggaaggcatcactcattcagtgagcgcttattgagcacctactgcatgctactgcgtaccatgtt

SEQ ID NO: 195 - DNA, 346 bp, Unknown Organism
  Description of Unknown Organism Tyro 3
ctcaccctctgctttctgagctcttcagctcctggtatgtgtgggataatgatggtagggaaggcagggactcctaagagctttctttctttgggtacagcgccagccagagctcctcagaatttccatgccattcgtaccgactcaggccttatcctggaatgggaagaagtgattcctgaggaccctggggaaggccccctaggaccttataagctgtcctgggtccaagaaaatggaacccaggtaagagagacagttccttctgctccccctactgtgctggagagtctaccctgtacttctgagacttagaagagctttagaatgcttctggagggtctgg

SEQ ID NO: 196 - DNA, 345 bp, Unknown Organism
  Description of Unknown Organism Tyro 3
aaggttccctaattccttagttgaactttcttctggcctcccagctcagctggaggcttccccaggctgtgtgtaagggtggggcttttcctggttacaggatgagctgatggtggaagggaccagggccaatctgaccgactgggatccccagaaggacctgattttgcgtgtgtgtgcctccaatgcaattggtgatgggccctggagtcagccactggtggtgtcttctcatgaccatgcaggtaaggcatgcagggcaggcgtagggtgcaaggctttcagggattacaccgactgataggaggaggctggtctcacacacacacacacacacacacacac

SEQ ID NO: 197 - DNA, 330 bp, Unknown Organism
  Description of Unknown Organism Tyro 3
aaatcttttggctagattaggtaggtggccaccctaggacagttctaaaaggacattttggtgtcagcctggctcatgacaacgcctttccctctcccagggaggcagggccctccccacagccgcacatcctgggtgcctgtggtcctgggcgtgctcaccgccctgatcacagctgctgccttggccctcatcctgcttcggaagagacgcaaggagacgcgtttcgggtaaggaagtgggacagcggccagtgaagaaggtggtaacagggggccttgtgatttacctgatgtctgggtttctctttaggcaagcctttgacagtgt

SEQ ID NO: 198 - DNA, 301 bp, Unknown Organism
  Description of Unknown Organism Tyro 3
caaggagacgcgtttcgggtaaggaagtgggacagcggccagtgaagaaggtggtaacagggggccttgtgatttacctgatgtctgggtttctctttaggcaagcctttgacagtgtcatggcccgaggggagccagctgtacacttccgggcagcccgatctttcaatcgagaaaggcctgaacgcattgaggccacatgtgagtgctggagcatcacagaagggaagagacagactatcttgtgttcctacctcttaccaactcctttatccagctaaagagcctgaacctttctaag

SEQ ID NO: 199 - DNA, 296 bp, Unknown Organism
  Description of Unknown Organism Tyro 3
ggagatccaggctctgctgagggcagaaagccgggtggagctgtaggctggagtgtggatttgactgcagccttgcttccctttcacctcctccctgcagtggatagcctgggcatcagcgatgaattgaaggaaaagctggaggatgtcctcattccagagcagcagttcaccctcggtcggatgttgggcaaaggtgcgggggctgtgggggactagcatgagtttgccctctctgcatggcttgttccagttgcttccagggtatgaatttgggtcagctcctataaacaaga

SEQ ID NO: 200 - DNA, 281 bp, Unknown Organism
  Description of Unknown Organism Tyro 3
gccttaattgatgattttatgaccatatgaagagtcacctgagagggaagggtgtggggagcctgaggtgtctcacgctgtccattgatttcttgcttaggagagtttggatcagtgcgggaagcccagctaaagcaggaagatggctccttcgtgaaagtggcagtgaagatgctgaaaggtgagttagacagtagggtttgttgccaggtagggaagagggcaaagggagggactgatctggagcaggcgccactctgcctgagatttcccaagaagct

SEQ ID NO: 201 - DNA, 293 bp, Unknown Organism
  Description of Unknown Organism Tyro 3
gcccctgtgaagcccagggcttctgtgagtcctaggagtcctgggagggtggcacgagtggtgagtcctgtttccattccttctccttcctttctcccagctgacatcattgcctcaagcgacatagaagagttcctccgggaagcagcttgcatgaaggagtttgaccatccacacgtggccaagcttgttggtgagcccgtttggaggaggcagatatagaatagggctaaaaatatgctccaccaacgtgacagggcagcctggaggagaaaagtttggctacccagtga

SEQ ID NO: 202 - DNA, 322 bp, Unknown Organism
  Description of Unknown Organism Tyro 3
gctagagatggaagccagcaggtagggaggcactgttgcagatacctagctgtccttcggccttcctgtgcccagttcttgacgctcccttgtctcctaggggtgagcctccggagcagggctaaaggtcgtctccccattcccatggtcatcctgcccttcatgaaacatggagacttgcacgcctttctgctcgcctcccgaatcggggagaacccttttgtgagtgcctcaggtcggggtgggagactggtgcctggaaaagggagagaacctggggagaagaggctttgttgggcaggactcggggagctgatggggt

SEQ ID NO: 203 - DNA, 310 bp, Unknown Organism
  Description of Unknown Organism Tyro 3
cttacgatagtctttaggctttctgaccagagaatttcattcattacgccccacacagagtggggaggttcaacctgattttgccctgtctgcctaccagaacctgcccctgcagaccctggtccggttcatggtggacattgcctgtggcatggagtacctgagctcccggaacttcatccaccgagacctagcagctcggaattgcatgtaggaatggattctggtggcctgctgggtgggagacagctatcgcctcctaccttggatagaacctcctatgcacggggatagcctcgcttttagtaag

SEQ ID NO: 204 - DNA, 360 bp, Unknown Organism
  Description of Unknown Organism Tyro 3
cctaccttggatagaacctcctatgcacggggatagcctcgcttttagtaaggctctgggcctggggaagactgagtctgtctttcaacacccctctcaggctggccgaggacatgacagtgtgtgtggctgattttggactctctcggaaaatctatagcggggactattatcgtcagggctgtgcctccaaattgcccgtcaagtggctggccctggagagcttggctgacaacttgtatactgtacacagtgatgtggtgagcagggtggcccagtggggcttggtaatgtgaatagggtgaattaatgtgcgggtggatgtgcaggacccaaaaaggaccaccaaagttctgttct

SEQ ID NO: 205 - DNA, 337 bp, Unknown Organism
  Description of Unknown Organism Tyro 3
ttgagctttgtctattgacgagacccttgaatcctgggatatggctctgcttggctcaggaaaccaataacattccccctcaaccctgattctataccagtgggccttcggggtgaccatgtgggagatcatgactcgtgggcagacgccatatgctggcattgaaaatgctgagatttacaactacctcatcggcgggaaccgcctgaagcagcctccggagtgcatggaggaagtgtgagtatcctgggtcgtgggttagggaaggagctttggctcgaggctgctaccatagcagctcagaacctccacctgacttctgtttgtccttcttttg

SEQ ID NO: 206 - DNA, 1148 bp, Unknown Organism
  Description of Unknown Organism Tyro 3
agtactgttaaaagaatgagttattttcccatctcagcgtcatatggtcgggaggtcctctgctttgtggctactatgtccccgtattcttttacctcaggtatgatctcatgtaccagtgctggagcgccgaccccaagcagcgcccaagcttcacgtgtctgcgaatggaactggagaacattctgggccacctgtctgtgctgtccaccagccaggaccccttgtacatcaacattgagagagctgagcagcctactgagagtggcagccctgagctgcactgtggagagcgatccagcagcgaggcaggggacggcagtggcgtgggggcagtaggtggcatccccagtgactctcggtacatcttcagccccggagggctatccgagtcaccagggcagctggagcagcagccagaaagccccctcaatgagaaccagaggctgttgttgctgcagcaagggctactgcctcacagtagctgttaaccctcagccagaggaaagttggggcccctggctctgctgaccactgtgctgcctgactaggcccagtctgatcacagcccaggcagcaaggtatggaggctcctgtggtagccctcccaagctgtgctggcgcctggacggaccaaattgcccaatcccagttcttcctgcagccgctctggccagcctggcatcagttcaggccttggcttagaggaggtgagccagagctggttgcctgaatgcaggcagctggcaggaggggagggtggctatgtttccatgggtaccatgggtgtggatggcagtaagggagggtagcaacagccctgtgggcccctaccctcctggctgagctgctcctactttagtgcatgcttggagccgcctgcagcctggaactcagcactgcccaccacacttgggccgaaatgccaggtttgcccctcttaagtcacaaagagatgtccatgtattgttcccttttaggtgatgattaggaagggattggcacacttgggtccctaagccctatggcaggaaatggtgggatattctcaggtctgaatcctcatcatcttcctgattccccaccctgcaaaggcctggaactggctgtggggctctgaggcatgctgaaggacaaaagattacaga

(19) United States
(12) Patent Application Publication
(10) Pub. No.: US 2002/0150971 A1
(43) Pub. Date: Oct. 17, 2002

(54) Nucleic acids and polypeptides for controlling food intake and/or body weight

(76) Inventors: Jeanette Elisabeth Johansen, Stockholm (SE); Martin Schalling, Spaenga (SE)

Correspondence Address: Stephen A. Bent, FOLEY & LARDNER, Washington Harbour, 3000 K Street, N.W., Suite 500, Washington, DC, 20007-5109, US

(21) Appl. No.: 09/778,844

(22) Filed: Feb. 8, 2001

(51) Int. Cl. C07K 14/705; C07H 21/04; C12P 21/02; C12N 5/06

(52) U.S. Cl. 435/69.1; 435/325; 435/320.1; 530/350; 536/23.2

(57) ABSTRACT

An isolated nucleic acid molecule comprising a nucleotide sequence derived from a vertebrate animal, said nucleotide sequence codes for a polypeptide that is capable of at least partially complementing the phenotype of an animal having the anx/anx genotype as described in Maltais et al., J. Hered., 1984, 75:468-472, or a similar genotype and the polypeptide encoded by the nucleotide sequence is useful for controlling food or feed intake or body weight in animals including humans. Antibodies to the polypeptide can be used as diagnostic agents or for therapeutic purposes. Receptors for the polypeptide or antibodies to the receptors and/or polypeptide can be used to block, inhibit or enhance the biological activity of the polypeptide.

### FIELD OF INVENTION

The invention relates to the field of controlling food or feed intake and body weight in vertebrate animals including humans. In particular there is provided an isolated nucleic acid molecule that comprises a sequence coding for a polypeptide that is capable of complementing the phenotype of an animal having an anx/anx genotype, or a similar genotype. The invention is based on the findings that an anx/anx genotype animal has a phenotype implying that significant alterations occur in the hypothalamic distribution of several substances that are directly or indirectly involved (both anorexically and orexically) in feed or food intake regulation including neuropeptide Y (NPY), the agouti gene-related protein (AGRP), pro-opiomelanocortin (POMC) and its products, the cocaine and amphetamine regulated transcript (CART) and leptin. The implication of these findings is that the gene product(s) of the mutated gene(s) causing the anx/anx phenotype appear(s) to play a significant and possibly, a central role in the regulation of body weight.

Having isolated a nucleic acid molecule comprising a sequence coding for the polypeptide that is capable of complementing the phenotypic expressions of the anx/anx genotype, it becomes possible to provide for recombinant production of such a polypeptide and to utilise the polypeptide as such or substances inhibiting or enhancing the biological activities of the polypeptide as body weight controlling agents. Based on the provision of the isolated nucleic acid and the polypeptide of the invention and/or substances inhibiting or enhancing its activities, diagnostic means for detecting genetic and physiological disorders become readily accessible.

### TECHNICAL BACKGROUND AND PRIOR ART

Inappropriate or abnormal food intake patterns resulting in clinically recognisable disorders such as anorexia and obesity constitute an increasing medical health problem, in particular in the Western world. However, in spite of major research efforts, little is known about the genetic and biochemical mechanisms that control eating behaviour. The regulation of food intake is a complex and poorly understood process. In this connection, an important aspect is the interactions between the central nervous system (CNS) and food intake and it is generally believed that appetite, energy balance and body weight gain are regulated or modulated by several neurochemical and neuroendocrine signals from different organs in the body and diverse regions in the brain. It is known that the hypothalamus has an important function in these processes, acting through a variety of systems that involve a close interaction between nutrients, amines, neuropeptides and hormones (Leibowitz, Trends in Neurosciences, 1992, 15:491-497).

One approach to gaining understanding of the regulation of food intake and energy output is by genetic analysis of animals carrying mutant genes. The first recessive obesity mutation, the obese mutation (ob) was described by Ingall et al, 1950, J. Hered. 41:317-318. Subsequently, several single-gene mutations in mice have been found to produce an obese phenotype as described by Friedman et al., 1990, Cell 69:217-220. The mouse obese gene and its human homologue have been cloned as described by Zhang et al., 1994, Nature 372:425. In WO 96/35787 is described the production of an ob polypeptide using recombinant DNA technology and pharmaceutical compositions comprising ob polypeptides for the control of obesity. Another approach to regulating obesity is suggested in WO 97/48806 based upon delivery of an obesity regulating gene, preferably a gene coding for leptin or a leptin receptor, to a mammal.

Anorexia (anx) is a spontaneous recessive lethal mutation located on mouse Chromosome 2 that causes starvation in mice including mice in the preweanling period (Maltais et al., 1984, J. Hered. 75:468-472). It was first discovered in 1976 at the Jackson Laboratories, U.S.A. in the F2 generation of DW/J × (M. m. poschiavinus × Swiss). These mutant animals of the anx/anx genotype appear normal at birth but they develop symptoms of growth failure and emaciation as well as head weaving, body tremors and uncoordinated gait. The animals die prematurely. No abnormalities in respect of histology and anatomy of the gastrointestinal tract or in respect of biochemical parameters in the blood have been reported. Mutant mice (anx/anx) are characterised by poor appetite with reduced stomach contents compared to normal littermates as from postnatal day 5 (Maltais et al., 1984). Interestingly, the pattern of food intake, measured as daily changes in stomach content, for anx/anx mice is very similar to that observed in normal littermates from birth to 20 days of age (Maltais et al., 1984). These data indicate that anx/anx mice fail to properly regulate the amount of food consumed rather than failing to eat for other reasons.

The neurological symptoms involved in the phenotype caused by the anx/anx genotype, i.e. head weaving, body tremors and uncoordinated gait can be partially suppressed by serotonin antagonists. Increasing the serotonin levels by injecting serotonin agonists produces severe neurological symptoms both in anx/anx mice and healthy litter mates (Maltais et al., 1984). It has been reported that mutant mice show hyperinnervation of serotonergic fibres in several structures in the brain including the hippocampus, cortex, olfactory bulb and cerebellum (Son et al., 1994, Mol. Brain Res. 25:129-134).

It is known that the hypothalamus plays an important role in regulation of food intake that is effected by a range of neurochemical messenger molecules that stimulate food intake such as neuropeptide Y (NPY), the agouti gene-related protein (AGRP), the melanin concentrating hormone (MCH) and orexin (ORX), and molecules having an inhibiting effect on food intake such as leptin, the cocaine and amphetamine regulated transcript (CART) and the α-melanocyte stimulating hormone (α-MSH).

In recent studies, the distribution of several of such neurochemical messenger molecules (including NPY and its Y1, Y2 and Y5 receptors, cholecystokinin, galanin, serotonin, somatotostatin, acetylcholinesterase, AGRP, the pro-opiomelanocortinergic (POMC) peptides and CART), in mice that are homozygous for the anorexia mutation has been studied by using immunohistochemistry and in situ hybridisation (Broberger et al., 1997, 1998, 1999 and Johansen et al., 2000). Among the significant findings in these studies are: NPY-like immunoreactivity is increased markedly in arcuate nucleus cell bodies and decreased in terminals of the arcuate nucleus and other hypothalamic regions of anx/anx mice as compared to normal litter mates. Similar observations were made in respect of AGRP. No differences in mRNA expression for NPY and AGRP, respectively was found in the arcuate nucleus of the anx/anx mice. However, a decreased number of POMC mRNA-expressing neurons in the anx/anx arcuate nucleus was observed and, in parallel, decreased mRNA levels for the Y1 and Y5 receptors for NPY which are expressed in POMC-neurons. Additionally, anx/anx mice were found to have significantly decreased levels of CART mRNA label and peptide-immunoreactive cell bodies and fibres in the arcuate nucleus and a lower number of detectable CART-expressing cells in the dorsomedial hypothalamic nucleus/lateral hypothalamic area.

The results of the above studies clearly indicate that the anx/anx genotype confers phenotypic traits that are directly or indirectly involved in food intake regulation. A primary objective of the present invention is to provide the gene product(s) of the gene or genes which is/are mutated in the anx/anx genotype or similar genotypes conferring phenotypic traits similar to the anx/anx genotype as the basis for a novel approach to regulating food intake and weight gain.

### SUMMARY OF THE INVENTION

Accordingly, the present invention pertains in a first aspect to an isolated nucleic acid molecule comprising a nucleotide sequence derived from a vertebrate animal, said nucleotide sequence codes for a polypeptide that is capable of at least partially complementing the phenotype of an animal having the anx/anx genotype as described in Maltais et al., J. Hered., 1984, 75:468-472, or a similar genotype.

In further aspects the invention provides an expression vector comprising the nucleic acid molecule as defined above and a host cell comprising such a vector.

In a still further aspect the invention relates to a method of producing a polypeptide that is capable of at least partially complementing the phenotype of an animal having the anx/anx genotype as described in Maltais et al. (1984), or a similar genotype, the method comprising the steps of: (i) providing a nucleic acid molecule as defined above, (ii) introducing the nucleic acid molecule into a host cell that is capable of expressing the nucleotide sequence coding for the phenotype complementing polypeptide, (iii) culturing the host cell under conditions permitting expression of the nucleotide sequence coding for the phenotype complementing polypeptide, and (iv) harvesting the polypeptide.

In other aspects there is provided a method of inducing, in a vertebrate animal, the production of a polypeptide that is capable of at least partially complementing the phenotype of an animal having the anx/anx genotype as described in Maltais et al. (1984) or a similar genotype, the method comprising administering to the animal the nucleic acid molecule as defined herein; and an isolated polypeptide that is capable of at least partially complementing the phenotype of an animal having the anx/anx genotype as described in Maltais et al. (1984), or a similar genotype, said polypeptide is obtainable by the above method for producing the polypeptide.

In yet further aspects the invention pertains to methods of regulating food or feed intake or weight gain in a vertebrate animal, the method comprising administering to the animal a food or feed intake regulating amount of the polypeptide as defined herein.

There is also provided a method of regulating food or feed intake in a vertebrate animal, the method comprising administering to the animal an effective amount of a molecule that blocks or inhibits or enhances the biological activity of the polypeptide according to the invention.

In still further aspects, the invention relates to a pharmaceutical composition comprising a polypeptide that is capable of at least partially complementing the phenotype of an animal having the anx/anx genotype as described in Maltais et al. (1984), or a similar genotype, or a pharmaceutically active part thereof, and a pharmaceutically acceptable carrier and to a pharmaceutical composition comprising a molecule that blocks or inhibits or enhances the biological activity of a polypeptide that is capable of at least partially complementing the phenotype of an animal having the anx/anx genotype as described in Maltais et al. (1984), or a similar genotype, said molecule is selected from the group consisting of an antibody capable of binding to the polypeptide, a receptor capable of binding to the polypeptide, an antagonist that prevents the polypeptide from binding to its receptor and any other molecule that is capable of binding to the polypeptide or a pharmaceutically active part thereof, and a pharmaceutically acceptable carrier.

In yet another aspect there is provided a method of isolating a molecule that is capable of interacting with a polypeptide that is capable of at least partially complementing the phenotype of an animal having the anx/anx genotype as described in Maltais et al. (1984), or a similar genotype, the method comprising the steps of: (i) providing the polypeptide of the invention, (ii) permitting the polypeptide to react with cells or fragments or extracts thereof to form a binding pair, and (iii) separating from the binding pair a molecule that binds to the polypeptide.

There is also provided an antibody comprising at least one binding site that is capable of binding to the polypeptide encoded by the above nucleotide sequence derived from a vertebrate animal, said nucleotide sequence codes for a polypeptide that is capable of at least partially complementing the phenotype of an animal having the anx/anx genotype as described in Maltais et al. (1984), or a similar genotype, and a method of detecting the presence of a polypeptide that is capable of at least partially complementing the phenotype of an animal having the anx/anx genotype as described in Maltais et al. (1984), or a similar genotype, or a homologue of said polypeptide, the method comprising contacting an antibody capable of binding to the polypeptide with a sample suspected of containing the polypeptide or the homologue thereof to allow formation of a polypeptide/antibody complex and detecting the complex.

In yet another aspect the invention relates to an isolated molecule that interacts with a polypeptide that is capable of at least partially complementing the phenotype of an animal having the anx/anx genotype as described in Maltais et al. (1984), or a similar genotype, said isolated molecule is provided by the above method of isolating such a molecule.

In another further aspect there is provided a method of producing an antibody to the above isolated molecule that interacts with a polypeptide that is capable of at least partially complementing the phenotype of an animal having the anx/anx genotype as described in Maltais et al. (1984), or a similar genotype, the method comprising administering an immunologically effective amount of the molecule to an animal and collecting from the animal serum containing antibodies against the receptor or spleen cells for the production of monoclonal antibodies against the molecule.

In a further aspect the invention pertains to a method for detection of a molecule that is capable of binding to a polypeptide capable of at least partially complementing the phenotype of an animal having the anx/anx genotype as described in Maltais et al. (1984), or a similar genotype, the method comprising the steps of: (i) providing the antibody produced by the above method of producing an antibody to the above isolated molecule that interacts with a polypeptide that is capable of at least partially complementing the phenotype of an animal having the anx/anx genotype as described in Maltais et al. (1984), or a similar genotype, (ii) reacting the antibody with cells or fragments or extracts thereof to form a binding pair, and (iii) detecting the presence of the binding pair.

In further aspects the invention provides a kit for detection of a polypeptide that is capable of at least partially complementing the phenotype of an animal having the anx/anx genotype as described in Maltais et al. (1984), or a similar genotype, said kit comprising an antibody comprising at least one binding site that is capable of binding to the polypeptide encoded by the above nucleotide sequence derived from a vertebrate animal, said nucleotide sequence codes for a polypeptide that is capable of at least partially complementing the phenotype of an animal having the anx/anx genotype as described in Maltais et al. (1984), or a similar genotype; a kit for detection of a receptor for a polypeptide that is capable of at least partially complementing the phenotype of an animal having the anx/anx genotype as described in Maltais et al. (1984), or a similar genotype, said kit comprising said polypeptide; and a kit for detection of antibodies to a polypeptide that is capable of at least partially complementing the phenotype of an animal having the anx/anx genotype as described in Maltais et al. (1984), or a similar genotype, said kit comprising the polypeptide.

The invention pertains in a still further aspect to a method of identifying in a sample a nucleotide sequence that codes for a putative polypeptide that is capable of at least partially complementing the phenotype of an animal having the anx/anx genotype as described in Maltais et al. (1984), or a similar genotype. In presently preferred embodiments, the method comprises the steps of: (i) providing the coding nucleotide sequence as defined above or its complementary strand, or a part thereof, (ii) contacting the sample with said sequence or part thereof under hybridising conditions to form a binding pair, and (iii) detecting the presence of the binding pair. In other useful embodiments, the coding sequence is identified by PCR or other similar nucleic acid amplification methods.

In a still further aspect the invention relates to a method of controlling in an animal the expression of a nucleotide sequence coding for a polypeptide that is capable of at least partially complementing the phenotype of an animal having the anx/anx genotype as described in Maltais et al. (1984), or a similar genotype, the method comprising the steps of: (i) providing a molecule that inhibits the translation of a transcript of the coding sequence, and (ii) administering to the animal a translation inhibiting effective amount of said molecule.

### DETAILED DISCLOSURE OF THE INVENTION

It is a primary objective of the present invention to provide a nucleic acid molecule that comprises a nucleotide sequence coding for a polypeptide that is capable of at least partially complementing the phenotype of an animal having the anx/anx genotype as dscribed in Maltais et al. (1984), or a similar genotype. In this context, the expression “nucleic acid molecule” refers to DNA, cDNA or RNA and “coding nucleotide sequence” refers to a DNA or a RNA that encodes a specific amino sequence, or their complementary strand. As used herein, the expression “capable of at least partially complementing the phenotype of an animal having the anx/anx genotype as described in Maltais et al. (1984), or a similar genotype” implies that the polypeptide, when it is introduced into or produced in an animal having the anx/anx genotype or a similar genotype, is capable of eliminating or reducing at least some of the phenotypic traits associated with that genotype. Such phenotypic traits include not only those described in Maltais et al. (1984), which is incorporated herein by reference, but they include any other traits, whether they have been discovered or will be discovered in the future, which can be associated with the anx/anx genotype, or with a similar genotype. Accordingly, the phenotypic traits include any detectable behavioural, biochemical, histological and physiological changes, relative to an animal not having the genotype, including an animal of the same species such as a litter mate, in an animal having the anx/anx genotype or the similar genotype such as those changes published by Broberger et al. (1997, 1998 and 1999) and Johansen et al. (2000), which publications are also incorporated herein by reference. As used herein, the term “animal” include any species of vertebrate animals including rodents such as a mouse or a rat, carnivores such as canine and feline animals, ruminants, pigs, horses, birds, fish, and humans.

Accordingly, the phenotypic traits as referred to above include the following (as compared to an animal not having the genotype):

1. Traits Described in Maltais et al. (1984)

(i) growth failure and emaciation;

(ii) blood parameters including total RBC, haematocrits, haemoglobin, mean cell volume within normal ranges;

(iii) higher serum cholesterol level;

(iv) lower blood glucose level;

(v) elevated alkaline phosphatase level;

(vi) abnormal behaviour including head weaving, body tremors, uncoordinated gait and hyperactivity, which can be diminished by injection of 5,7-dihydroxytryptamine;

2. Traits Described in Broberger et al. (1997, 1998 and 1999) and Johansen et al. (2000)

(viii) Increased agouti gene-related protein (AGRP) immunoreactivity in arcuate nucleus cell bodies and decreased immunoreactivity in hypothalamic terminals, no difference in AGRP mRNA expression in the arcuate nucleus (Broberger et al., 1998);

(ix) increase of neuropeptide Y-like immunoreactivity in hypothalamic arcuate cell bodies and decrease of said immunoreactivity in terminals in the arcuate nucleus, but essentially no difference in neuropeptide Y (NPY) mRNA in the arcuate nucleus (Broberger et al., 1997);

(x) decreased numbers of Pro-Opiomelanocortin (POMC) mRNA-expressing neurons in the arcuate nucleus (Broberger et al., 1999);

(xi) decreased mRNA levels for NPY Y1 and Y5 receptors in POMC neurons (Broberger et al., 1999);

(xii) decreased immunoreactivities in the hypothalamus for adrenocorticotropic hormone (marker for POMC cell bodies), α-melanocyte-stimulating hormone (marker for axonal projections) and NPY Y1 receptor (marker for dendritic arborisations) (Broberger et al., 1999);

(xiii) cocaine and amphetamine regulated transcript (CART) in the arcuate nucleus and serum leptin levels are reduced and a decreased number of CART expressing cells in the dorsomedial and lateral hypothalamic areas.

3. Further, unpublished phenotypic traits

(xiv) the Na⁺, K⁺-ATPase activity is upregulated in the striatum and striatal dopamine—and dopamine metabolite levels are lower;

(xv) the insulin content in pancreatic β-cells is increased at a statistically significant level and insulin is not secreted.

As used herein, the expression “similar genotype” refers to a genetic disorder in a vertebrate animal that confers one or more of the above phenotypic traits such as e.g. having at least half of the traits (i) to (vi), optionally combined with at least one of the above traits (vii) to (xv) such as at least two of these traits including at least three, four, five or six of such traits (vii) to (xv).

It will be appreciated that in the present context “a genetic disorder” in an anx/anx genotype or in a similar genotype can be a mutation in a coding sequence resulting in the expression of a gene product having, relative to the wildtype product, a reduced biological activity, a mutation in a coding sequence that results in a reduced expression or even no expression of a biologically active polypeptide or a mutation in a nucleic acid sequence that codes for a gene product having a regulatory effect on the expression of a coding sequence e.g. causing a down regulation of the expression of a biologically active polypeptide such as the Tyro 3 gene product. When the genetic disorder is one that results in lower or higher expression level, relative to a wildtype individual, of a gene product, it can e.g. be detected by comparing the level of mRNA species in total mRNA determined in vitro or in vivo in anx/anx genotype animals and wildtype litter mates and identifying mRNA polymorphisms. An example of such an approach is described in Example 3 below. The genetic disorder may also be one that results in a changed activity of the gene product.

As a matter of clarification, it should be noted that the expression “capable of at least partially complementing the phenotype of an animal having the anx/anx genotype as described in Maltais et al. (1984), or a similar genotype” as defined above is used herein to characterise the effects and functionalities of the polypeptide expressed by the nucleotide sequence and this use does not imply any limitations in respect of the use of the polypeptide as can be appreciated from the following description of the invention.

The nucleic acid molecule comprising the nucleotide sequence that codes for a polypeptide as defined herein can be derived from any vertebrate animals. One preferred approach to providing the nucleic acid molecule is to derive it from an animal having the anx/anx genotype or a similar genotype as defined above by isolating a nucleic acid molecule from such an animal comprising one or more mutations as defined above that confer(s) the phenotype as defined above and isolating and characterising the coding sequence(s) or the regulatory sequence(s) containing the mutation or mutations. The coding sequence for the putative polypeptide as defined above can then be derived synthetically e.g. by modifying the mutant gene to its wild type sequence, or the isolated mutant sequence or a part hereof can be used as a hybridisation probe to identify the corresponding wildtype sequence in any vertebrate species or such a wild type sequence can alternatively be identified by comparing the mutant sequence with published gene sequence data.

It will be appreciated that the expression “nucleic acid molecule that comprises a nucleotide sequence coding for a polypeptide” includes a nucleic acid molecule that consists only of the nucleotide sequence coding for a polypeptide as defined herein. However, the nucleic acid may also comprise further nucleotides including nucleotide sequences that have a regulatory effect on the expression of the polypeptide and may be of a size which is typically in the range of 1 kilobase (kb) to 1 megabase, such as in the range of 10 kb to 0.75 megabase including the range of 50 kb to 0.5 megabase.

The nucleic acid molecule of the invention can, as it is mentioned above, comprise a regulatory nucleotide sequence that regulates the expression of the nucleotide sequence coding for the phenotype complementing polypeptide. As used herein, the expression “regulatory nucleotide sequence” includes any expression control sequence, i.e. a sequence that is conventionally used to affect expression of a nucleotide sequence or a gene that encodes a polypeptide and includes one or more components that affect(s) expression, including transcription and translation signals. Such a sequence includes e.g. one or more of the following: a promoter sequence, an enhancer sequence, an upstream activation sequence, a downstream termination sequence, a polyadenylation sequence, an optimal 5′ leader sequence to optimise initiation of translation in mammalian host cells, a secretion leader sequence that provides for secretion of the phenotype complementing polypeptide upon expression of the nucleotide sequence coding for the polypeptide and a Shine-Delgarno sequence. Appropriate regulatory nucleotide sequences differ depending on the host system in which the polypeptide is to be expressed. E.g. in prokaryotes, such a regulatory sequence can include one or more of a promoter sequence, a ribosomal binding site and a transcription termination sequence. In eukaryotes such a sequence can include a promoter sequence and a transcription termination sequence. In accordance with the present invention, any appropriate regulatory nucleotide sequence can be a sequence that is naturally associated with the sequence coding for the polypeptide as defined herein, but it is also contemplated to use such sequences that are not naturally associated with the nucleotide sequence coding for the phenotype complementing polypeptide. Use of viral promoters to regulate the expression of the polypeptide of the invention is also contemplated.

As used herein the term “polypeptide” includes any polypeptide that is capable of at least partially complementing the phenotype of an animal having the anx/anx genotype as described in Maltais et al. (1984), or a similar genotype. Accordingly, the term includes the putative polypeptide(s) encoded by the wild type of the gene or genes carrying the anx mutation in a mouse such as is described in the above reference and any phenotype complementing functional homologue of such a polypeptide, including both a mature polypeptide and a polypeptide provided with a signal peptide sequence and any truncations, variants, alleles, analogues and derivatives thereof. In this context, the expression “phenotype complementing functional homologue” indicates a polypeptide that is capable of complementing at least one of the phenotypic traits conferred by the anx/anx genotype in mice, or a similar genotype.

The term “polypeptide” is not limited to a specific length of the product of the coding sequence. Thus, polypeptides that are identical or are at least 60%, preferably at least 70% such as at least 80% including at least 90% identical to the polypeptide encoded by the mutated gene in an anx/anx genotype animal or to its wild type, irrespective of the human or non-human source from which it is derived, are included within the definition of the polypeptide of the invention. Also included are therefore alleles and variants of the product of the gene carrying the anx mutation that contain amino acid substitutions, deletions or insertions. The amino acid substitutions can be conservative amino acid substitutions or substitutions to eliminate non-essential amino acid residues such as to alter a glycosylation site, a phosphorylation site, an acetylation site, or to alter the folding pattern by altering the position of cysteine residues that are not necessary for function. Conservative amino acid substitutions are those that preserve the general charge, hydrophobicity/hydrophilicity and/or steric bulk of the amino acid substituted, e.g. substitutions between the members of the following groups are conservative substitutions: Gly/Ala, Val/Ile/Leu, Asp/Glu, Lys/Arg, Asn/Gln and Phe/Trp/Tyr. Furthermore, the term “polypeptide” as used herein does not exclude post-expression modifications of the polypeptide such as e.g. glycosylations, acetylations and/or phosphorylations. It will be appreciated that a polypeptide as defined herein can be a component of a fusion protein where it is combined with one or more polypeptides or part(s) hereof.

In one useful embodiment, the nucleic acid molecule of the invention is derived from a rodent such as a mouse, including a mouse having the genotype anx/anx, e.g. as it is described in Maltais et al. (1984). The molecule may consist only of the gene which carries the anx-associated mutation(s) or it may comprise further nucleotides and be of a size as it is defined above. In a specific embodiment, the nucleic acid molecule is a molecule which is derived from a fragment of the mouse Chromosome 2 that is delimited by:

(i) the locus D2Mit133 that is published in:

http://carbon.wi.mit.edu:8000/cgi-bin/mouse/sequence_info?database=&sts=D2Mit133,

and by

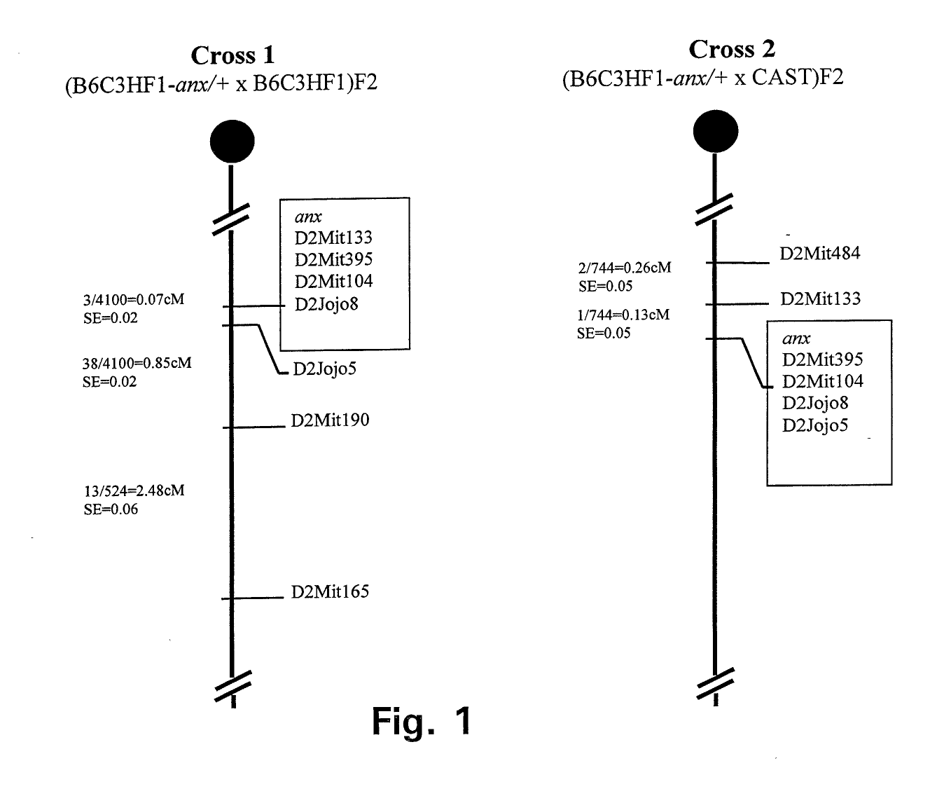

(ii) the locus D2Jojo5 (see FIG. 1). The size of this fragment is about 0.2 centiMorgan or 400 kilobase. This fragment, which is further described in the following examples, is further characterised in that it comprises a nucleotide sequence selected from the group consisting of the sequences D2Dcr14 (accession No. G36398), D2Mit104 published in:

http://carbon.wi.mit.edu:8000/cgi-bin/mouse/sequence info?database=&sts=D2Mit104,

D2Mit395 published in

http://carbon.wi.mit.edu:8000/cgi-bi n/mouse/sequence_info?database=&sts=D2M it395,

Ltk (accession Nos. Mus.musculus: X52621;×07984), Tyro 3A (accession No. Mus.musculus: U18342), Tyro 3B (accession No. Mus.musculus: U18343) and Tyro3 (accession Nos. Mus.musculus: X78103; AH006738; U23718; U 23719; U 23720; U23721).

The sequence of the above nucleic acid molecule and any other nucleic acid molecule as defined herein that codes for the phenotype complementing polypeptide according to the invention can e.g. be obtained by determining the nucleotide sequence of the molecule and subsequently searching for ORFs against known genes in databases or by using computer software that predicts genes in novel sequences. Examples of useful databases include Genscan (http://CCR-081.mit.edu/GENSCAN.htmi) and FGENE (http://dot.imgen.bcm.tmc.edu:9331/gene-finder/gf.html).

In a further aspect, the invention provides an expression vector comprising the nucleic acid molecule as described above. Such vectors can be provided by ligating the nucleic acid molecule of the invention to an appropriate plasmid vector containing an appropriate promoter for expression in the selected host expression system. Depending on the expression system, the promoter is preferably a prokaryotic cell promoter, a eukaryotic cell promoter or a viral promoter. Expression plasmids with various promoters are currently available commercially. One example of a suitable plasmid is plasmid pET23 that can be purchased from Novagen (Madison, Wis.). This plasmid utilises a T7 promoter sequence for expression in bacterial cells. Commercially available mammalian expression plasmids can also be used for the present purposes. Accordingly, the expression vector of the invention includes a vector further comprising a regulatory nucleotide sequence as defined hereinbefore that regulates the expression of the nucleotide sequence coding for the phenotype complementing polypeptide of the invention. Such a regulatory nucleotide sequence can be one that is naturally associated with the nucleotide sequence coding for the phenotype complementing polypeptide, but is preferably a nucleotide sequence that is not naturally associated with said sequence. As it is generally preferred that the phenotype complementing polypeptide, when produced industrially, is secreted into the growth medium for the host cell comprising the expression vector, the vector is preferably one that further comprises a nucleotide sequence encoding a secretion leader sequence that provides for secretion of the phenotype complementing polypeptide upon expression of the nucleotide sequence coding for the polypeptide.

In accordance with the above description of the nucleic acid of the invention, the expression vector is in one embodiment a vector wherein the nucleotide sequence expressing a polypeptide that is capable of at least partially complementing the phenotype of an animal having the anx/anx genotype as described in Maltais et al. (1984), or a similar genotype, is derived from a vertebrate animal including mammals such as rodents, e.g. a mouse including a mouse having the genotype anx/anx, or humans, fish or birds. In one specific embodiment, the expression vector comprises a nucleotide sequence expressing a polypeptide that is capable of at least partially complementing the phenotype of an animal having the anx/anx genotype as described in Maltais et al. (1984), or a similar genotype, which is derived from a fragment of the murine Chromosome 2 that is delimited by locus D2Mit133 and by locus D2Jojo5 and which optionally comprises a nucleotide sequence selected from D2Dcr14, D2Mit104, D2Mit395, Tyro 3A, Tyro 3B and Tyro 3, as defined above and any of the further genes within the fragment delimited by locus D2Mit133 and by locus D2Jojo5 as described in the following. If appropriate, the expression vector may further comprise a nucleotide sequence coding for a selective marker.

In another aspect, the invention relates to a host cell comprising the above vector, i.e. to an expression system for the phenotype complementing polypeptide. The host is preferably selected from a prokaryotic cell and a eukaryotic cell. In preferred embodiments, the host cell expression system is a bacterial expression system. Control elements for use in bacterial host cells include promoters, optionally containing operator sequences, and ribosome binding sites. Useful promoters include sequences derived from sugar metabolising enzymes such as galactose, lactose (lac) and maltose metabolising enzymes. Further examples include promoter sequences derived from biosynthetic enzymes such as the trp, the β-lactamase (bla) promoter systems, bacteriophage λPL and T7 promoters. In addition, synthetic promoters such as the tac promoter can be used.

Useful host cells can be selected from gram negative bacteria and gram positive bacteria. Examples of gram negative host cells include E. coli, Pseudomonas spp., Salmonella spp. and Serratia spp and examples of gram positive bacteria that may be used in the invention include Bacillus spp., Streptomyces spp and lactic acid bacterial species such as Streptococcus spp. and Lactococcus spp. Methods for introducing exogenous DNA into such host cells typically include the use of CaCl₂ or other agents such as divalent cations and DMSO. DNA can also be introduced into bacterial cells by electroporation, nuclear injection or protoplast fusion as described generally in Sambrook et al. (1989). Preferably, the host cell should secret minimal amounts of proteolytic enzymes. Alternatively, in vitro methods of cloning, e.g. PCR or other nucleic acid polymerase reactions are suitable. Prokaryotic cells used to produce the phenotype complementing polypeptide of the invention are cultured in suitable media as described generally in Sambrook et al., supra.

Suitable eukaryotic host cells can also be selected from fungal cells including yeast cells, mammalian cells and insect cells.

Expression vectors and transformation vectors, either extrachromosomal replicons or integrating vectors, have been developed for transformation into yeast species including Saccharomyces cerevisiae, Candida albicans, Candida maltosa, Hansenula polymorpha, Kluyveromyces lactis, Pichia pastoris and Schizosaccharomyces pombe, and into species of filamentous fungi such as Aspergillus spp., Trichoderma spp., Neurospora spp. or Penicillium spp.

Control sequences for yeast vectors include as examples promoter regions from genes such as alcohol dehydrogenase (ADH), endolase, glucokinase, glucose-6-phosphate isomerase and pyruvate kinase. Inducible yeast promoters having the additional advantage of transcription controlled by growth conditions are available. Yeast enhancers are also advantageously used with yeast promoters. E.g. can upstream activating sequences (UAS) of one yeast promoter be joined with the transcription activation regions of another yeast promoter to create a synthetic hybrid promoter. Furthermore, a yeast promoter may include naturally occurring promoters of non-yeast origin having the ability to bind yeast RNA polymerase and initiate transcription. Other control elements that may be included in yeast expression vectors are terminators and leader sequences which encode signal sequences for secretion such as e.g. the leader sequence derived form the yeast invertase gene and the α-factor gene.

Methods of introducing exogenous DNA into yeast hosts are well known in the art and typically include transformation of spheroplasts or intact yeast cells treated with alkali cations. The introduction can be carried out by transformation, nuclear injection, electroporation or protoplast fusion as described generally in Sambrook et al., supra.

For yeast secretion of the polypeptide of the invention, the native signal sequence can be substituted by another leader such as the yeast invertase, a-factor or acid phosphatase leaders. For intracellular production of the polypeptide in yeast, a sequence encoding a yeast protein can be linked to the coding sequence for the phenotype complementing polypeptide to produce a fusion protein that can be cleaved intracellularly upon expression.

Particularly useful insect cell expression systems are based upon baculovirus expression vectors (BEVs), recombinant insect viruses in which the coding sequence for a foreign gene is inserted behind a baculovirus promoter in place of a viral gene, e.g. polyhedrin such as it is described in U.S. Pat. No. 4,745,051. A typical useful insect cell expression vector includes a DNA vector useful as an intermediate for the infection or transformation of an insect cell system, the vector generally containing DNA coding for a baculovirus transcriptional promoter, followed downstream by an insect signal DNA sequence capable of directing secretion of a desired polypeptide, and a site for insertion of the foreign gene encoding the foreign polypeptide and the foreign gene placed under transcriptional control of a baculovirus promoter, the foreign gene herein being the nucleotide sequence coding for the phenotype complementing polypeptide of the invention. Useful promoters for an insect cell expression system can be derived from any baculovirus infecting cells such as e.g. a baculovirus immediate-early gene IEI or IEN promoter or a strong polyhedrin promoter of baculovirus. The insect expression vector for use herein may also include the polyhedrin polyadenylation signal and a selective marker. DNA encoding suitable signal sequences may also be included such as e.g. the signal sequence of the baculovirus polyhedrin gene or mammalian signal sequences.

The phenotype complementing polypeptide of the invention may also be expressed in mammalian cells such as e.g. adipocytes, using promoters and enhancers that are functional in such cells. Typical promoters for mammalian cell expression include as examples, the SV40 early promoter, the CMV promoter, the mouse mammary tumour virus LTR promoter, the adenovirus late promoter and the herpes simplex virus promoter. Mammalian expression may be either constitutive or regulated (inducible).

Mammalian cell lines available as hosts for expression are also known and include many immortalised cell lines available from the ATCC, including Chinese hamster ovary (CHO) cells, HeLa cells, baby hamster kidney (BHK) cells, monkey kidney (COS) cells, human hepatocellular carcinoma cells, human embryonic kidney cells, human lung cells and human liver cells.

It is another objective of the invention to provide a method of producing a polypeptide that is capable of at least partially complementing the phenotype of an animal having the anx/anx genotype as described in Maltais et al. (1984), or a similar genotype. The method comprises as a first step that a nucleic acid molecule as described herein is provided, followed by introducing the nucleic acid molecule into a host cell that is capable of expressing the phenotype complementing polypeptide and culturing the host cell under conditions permitting expression of the nucleotide sequence coding for the phenotype complementing polypeptide, and harvesting the polypeptide. The method of introducing the nucleic acid molecule into the host cell will, as those of skill in the art will readily recognise, depend on the selected host cell system and the expression vector used as it is described hereinbefore,

Likewise, the method of culturing the host cells including the composition of the cultivation medium and temperature conditions, and the harvesting processes, will depend on the host cell type and can be determined by those of skill in the art.

Whereas the polypeptide of the invention can be used as such for pharmaceutical purposes as the active component of pharmaceutical compositions, its therapeutic use by genetic therapy is also contemplated. Accordingly, the invention relates in one aspect to a method of inducing, in a vertebrate animal, the production of a polypeptide that is capable of at least partially complementing the phenotype of an animal having the anx/anx genotype as described in Maltais et al. (1984), or a similar genotype, the method comprising administering to the animal the nucleic acid molecule or at least the sequence coding for the complementing polypeptide as described herein.

Accordingly, the nucleic acid molecule that comprises the sequence coding for the phenotype complementing polypeptide, with or without a signal sequence, can be used for regulation of food or feed intake and/or for regulating body weight, such as for treatment of obesity, by administration thereof via gene therapy. Gene therapy strategies for delivery of the nucleic acid molecule construct can utilise viral or non-viral vectors in in vivo or ex vivo modality. Expression of the coding sequence can be induced using endogenous mammalian or heterologous promoters. Expression of the coding sequence in vivo can be either constitutive or inducible.

For delivery of the nucleic acid molecule comprising the coding sequence using viral vectors, any of a number of viral vectors may be used including those described in Jolly, Cancer Gene Therapy, 1994, 1:51-64. The coding sequence can be inserted into plasmids designed for expression in retroviral vectors, adenoviral vectors, adeno-associated viral vectors or sindbis vectors. Promoters that are suitable for use with these vectors include as examples the Moloney retroviral LTR, CMV promoter and the mouse albumin promoter. Replication competent free virus can be produced and injected directly into the animal or human or by transduction of an autologous cell ex vivo, followed by injection in vivo.

The coding sequence can also be inserted into a non-viral delivery system, such as a plasmid, for expression of the polypeptide in vivo or ex vivo. For in vivo administration the coding sequence can be delivered by direct injection or by intravenous infusion. Promoters suitable for use in this manner include endogenous and heterologous promoters such as e.g. CMV. The coding sequence can be injected in a formulation comprising buffers or other agents that can stabilise the coding sequence and facilitate transduction thereof into cells and/or provide targeting.

After it has been expressed and harvested according to method described hereinabove, the phenotype complementing polypeptide can be purified to provide an isolated polypeptide that is capable of at least partially complementing the phenotype of an animal having the anx/anx genotype as described in Maltais et al. (1984), or a similar genotype. The purification and, optionally, folding of the polypeptide can be carried out using conventional methods of isolating and purifying proteins including, but not limited to, precipitation, chromatography, filtration, ultrafiltration and reverse osmosis procedures. If required, the purification of the polypeptide may imply a degree of purity where the polypeptide preparation is essentially free of other polypeptides.

The isolated polypeptide can be used in a variety of ways as it will be described in the following. For example, the polypeptide, optionally in conjunction with suitable carriers, can be administered to animals and humans intravenously, subcutaneously or orally in a therapeutically or prophylactically effective amount in methods of regulating food or feed intake or body weight including weight gain inhibition, in a vertebrate animal, the method comprising administering a food or feed intake regulating amount of the polypeptide according to the invention. The specific amount to be administered depends on the condition to be treated.

The expression “therapeutically or prophylactically effective amount” is generically the amount of the polypeptide that will provide a desired therapeutic or prophylactic result either in respect of regulating food or feed intake or in respect of a desired control of body weight, i.e. weight gain inhibition or, optionally weight gain enhancement. The precise inhibitory amount varies depending on the health and physical conditions of the individual to be treated, the capacity of the individual's ability to adjust to the change in regulation of food intake and metabolism and body size, the formulation and other relevant factors.

As an alternative to administering the polypeptide of the invention as described above, it is also contemplated that food or feed intake in a vertebrate animal can be regulated by administering to the animal an effective amount of a molecule that blocks or inhibits or enhances the biological activity of the phenotype complementing polypeptide of the invention. Such a blocking, inhibiting or enhancing molecule can be selected from an antibody capable of binding to the polypeptide, a receptor capable of binding to polypeptide, an antagonist that prevents the polypeptide from binding to its receptor, an agonist substance that enhances the biological activity of the polypeptide, and any other molecule that blocks, inhibits or enhances the biological activity of the polypeptide. In the present context, the term “receptor” indicates a structure, generally a protein, located on or in a cell, e.g. in the membrane, that specifically recognises a sequence of amino acids of the phenotype complementing polypeptide so as to bind to it with a higher affinity than to a random polypeptide. Such an interaction between the polypeptide and the receptor is generally expected to trigger an intracellular response.

As it is mentioned above, the polypeptide of the invention can be used in a pharmaceutical composition comprising the polypeptide as defined herein, and a pharmaceutically acceptable carrier. The expression “pharmaceutically acceptable carrier” refers to a carrier for administration of a therapeutic or prophylactic agent, such as the polypeptide of the invention or antibodies, and refers to any pharmaceutical carrier that does not in itself induce the production of antibodies harmful to the individual receiving the composition, and which can be administered without undue toxicity effects. Suitable carriers may be large, slowly metabolised macromolecules such as proteins, polysaccharides, polylactic acids, polyglycolic acids, polymeric amino acids, amino acid copolymers, and inactive virus particles. Such carriers are well known to those of ordinary skill in the art. The carrier may be a pharmaceutically acceptable salt such as e.g. mineral acid salts such as hydrochlorides, hydrobromides, phosphates, sulphates and the like, and the salt of organic acids such as acetates, propionates, malonates, benzoates and the like. A discussion of pharmaceutically acceptable excipients can be found in Remington's Pharmaceutical Sciences (Mack Publ. Co., N.J. 1991). Pharmaceutically acceptable carriers in therapeutic compositions may contain liquids such as water, saline, glycerol and ethanol. In addition, auxiliary substances such as wetting or emulsifying agents, pH buffering substances and the like, may be present in such vehicles. Typically, the therapeutic compositions of the invention are provided as injectables, either as liquid solutions or suspensions, solid forms suitable for solution in, or suspension in, liquid vehicles prior to injection may also be used.

The pharmaceutically acceptable carrier as defined above can also be used in pharmaceutical compositions that comprise a molecule that blocks, inhibits or enhances the biological activity of the polypeptide according to the invention where the molecule is selected from the group consisting of an antibody capable of binding to the polypeptide, a receptor capable of binding to the polypeptide, an antagonist that prevents the polypeptide from binding to its receptor, an agonist molecule that enhances the biological activity of the polypeptide, and any other molecule that is capable of binding to the polypeptide or a pharmaceutically active part thereof and which blocks, inhibits or enhances its biological activity.

In a further aspect, the invention provides a method of isolating a molecule that is capable of interacting with the phenotype complementing polypeptide as defined herein. This method comprises as the first step that the polypeptide is provided as described above followed by permitting the polypeptide to react with cells or fragments or extracts thereof to form a binding pair, and separating from the binding pair the molecule member that binds to the polypeptide. As used herein, the term “binding pair” refers to a pair of molecules such as e.g. a protein/protein pair, a protein/RNA pair or a protein/DNA pair in which the components of the pair bind specifically to each other with a higher affinity than to a random molecule such that upon binding, e.g. in case of a ligand/receptor interaction. Specific binding indicates a binding interaction having a low dissociation constant, which distinguishes specific binding from non-specific, background binding. The molecules which can be isolated using the method include an antibody capable of binding to the polypeptide or a fragment hereof, a receptor capable of binding to the polypeptide or to a fragment of the polypeptide, an antagonist that prevents the polypeptide from binding to its receptor, e.g. an antibody to the receptor, an agonist molecule that enhances the biological activity of the polypeptide, and any other molecule that is capable of binding to the polypeptide.

The phenotype complementing polypeptide of the invention can be used to generate monoclonal or polyclonal antibodies. Accordingly, the invention provides in a further aspect a monoclonal or a polyclonal antibody comprising at least one binding site that is capable of binding to the polypeptide encoded by the nucleotide sequence as defined above. Such antibodies to the polypeptide can be prepared by conventional methods, e.g. by immunising a suitable animal such as a mouse, rat, rabbit or goat. Immunisation is generally performed by mixing or emulsifying the polypeptide in saline, preferably in combination with an adjuvant such as Freund's complete adjuvant, and injecting the mixture or emulsion. The immunisation may be boosted 2-6 weeks with one or more injections.

Like any other antibodies, the resulting antibodies, whether polyclonal or monoclonal, can be labelled using conventional techniques. Suitable labels include chromophores, fluorophores, radioactive labels, electron-dense reagents, enzymes and ligands having specific binding partners. In this context, “specific binding partner” refers to a protein capable of binding a ligand molecule with high specificity such as in the case of an antigen and an antibody specific herefor. Other specific binding partners include biotin, avidin or streptavidin, IgG and protein A.

The antibodies generated in this manner can be used in any conventional applications including for diagnostic and therapeutic purposes. E.g., as a diagnostic, they can be used in an immunoassay for identification or detection of a polypeptide of the invention or a homologue thereof in a sample suspected of containing the polypeptide. For this purpose, the antibodies can be labelled with a suitable marker, such as a radioactive label, and allowed to react with the sample. After an appropriate reaction time, the sample is examined for the presence of specific binding pairs. Presence of specific binding suggests that a polypeptide of the invention is present in the sample. The antibodies, when used for diagnostic purposes, can be a part of kits for detection of the phenotype complementing polypeptide of the invention.

Additionally, the antibodies to the polypeptide can be used for therapeutic purposes for blocking the in vivo biological activity of the polypeptide. When used as therapeutics, the antibodies are preferably compatible with the host subjected to such treatment. E.g. when used for humans, the antibodies can be human antibodies or humanised antibodies, as this term is generally known in the art. The humanised antibodies can be made by any conventional methods for that purpose. e.g. by complementary determining region grafting, veneering, phage library display or by use of xeno-mice.

As mentioned above, the invention pertains in one aspect to a method of detecting the presence of a polypeptide as defined herein, or a homologue of said polypeptide, the method comprising contacting an antibody capable of binding to the polypeptide with a sample suspected of containing the polypeptide or the homologue thereof to allow formation of a polypeptide/antibody complex, i.e. a binding pair as defined hereinbefore, and detecting the complex. The immunoassays which can be used for that purpose can be designed to meet specific requirements, e.g. determined by the nature of the sample to be assayed. Generally, protocols for the immunoassay can e.g. be based on competition, direct reactions for sandwich type assays. Protocols may also include the use of solid supports or they may be by immunoprecipitation. The assays which can be used in accordance with the invention may involve the use of labelled antibodies to the polypeptide or labelled target polypeptides. Useful labels include fluorescent, chemiluminescent, radioactive or dye molecules. Assays which amplify the signals from the probe can also be used. Examples hereof include assays which utilise biotin or avidin and enzyme labelled and mediated immunoassays such as ELISA assay. The immunoassays can, if desired, be designed so as to determine the amount of polypeptide antigen that is present in a sample.

An immunoassay utilising the antibodies to the phenotype complementing polypeptide can be used for any biological sample, such as a blood sample, a serum sample, a tissue sample including as examples a sample of brain tissue or a sample of pancreas tissue.

In yet another aspect the invention relates to an isolated molecule that interacts with a polypeptide according to the invention including such a polypeptide that is produced by the above method of producing the polypeptide. Such a molecule includes a monoclonal or a polyclonal antibody as described above, and a receptor molecule as also defined above.

A receptor molecule can e.g. be identified and isolated by reacting cells, cell membranes or extracts with a suitably labelled polypeptide of the invention. The mixture is then examined for presence of specific binding to the labelled polypeptide and the binding pairs formed can be separated by conventional techniques, such as by use of solvents or denaturing reagents for by passage through a column that selectively binds one member of the binding pair, and eluting the opposite member, i.e. the receptor molecule, with an appropriate solvent. The receptor molecule can be purified by conventional techniques and the amino acid sequence thereof can then be determined. Based on the amino acid sequence identified, an oligonucleotide probe can be made to probe a cDNA or genomic library and clones that hybridise to the probe can be amplified and sequenced. A cDNA clone that encodes a full length receptor can be used for recombinant production of large quantities of the receptor, useful for further studies into the mechanism of regulation of food intake and/or body weight and to obtain agonists and antagonists thereto.

In a useful aspect of the present invention there is provided a method of producing an antibody to the above molecule that is capable of interacting with the polypeptide of the invention, including an antibody or a receptor molecule. Such an antibody can be produced by any conventional method as it is described above including that an immunologically effective amount of the molecule is administered to an animal and that sera containing antibodies against the receptor or spleen cells for the production of monoclonal antibodies against the molecule are collected from the immunised animals.

Such antibodies, whether monoclonal or polyclonal can be used in immunoassays, such as it is described above, for detecting molecules that are capable of binding to the polypeptide of the invention. Generally, an immunoassay for such purposes involves that the above antibody is reacted with cells or fragments or extracts thereof to form a binding pair, and detecting the presence of the binding pair.

A particularly interesting use of antibodies to a receptor for the polypeptide of the invention is to block, reduce or inhibit the biological activity of the polypeptide in vivo. Accordingly, such antibodies can be used as therapeutic agents and be provided in formulations or pharmaceutical compositions that are suitable for that purpose.

The polypeptide of the invention, optionally in a form where it is provided with a detectable label including those mentioned above, can be provided as a part of a kit for detection of a receptor for the polypeptide or for detection of antibodies to the polypeptide.

It is a further objective of the invention the provide a method of identifying in a sample a target nucleotide sequence that codes for a putative phenotype complementing polypeptide as defined herein. For that purpose any DNA- or RNA-containing sample can be collected from an animal of interest including a human. The method involves that a nucleotide sequence as defined hereinbefore or a complementary strand hereof, or a part or fragment thereof, which is preferably provided with a detectable label, e.g. a radioactive label, is contacted with the sample to be tested under hybridising conditions to form a binding pair and subsequently detecting the presence of the binding pair. The members of the binding pair can then be separated e.g. by a denaturing treatment. The isolated target member can be used as the starting point for a recombinant production of a polypeptide according to the invention.

It is also, as mentioned above, an objective of the invention to provide a method of controlling in an animal the expression of a nucleotide sequence coding for a phenotype complementing polypeptide of the invention. This method comprises as a first step that a molecule that inhibits the translation of a transcript of the coding sequence is provided, followed by administering to the animal a translation inhibiting effective amount of said molecule. In accordance with the invention, presently preferred translation inhibiting molecules include antisense RNA and PNA.

The invention is further described in the following examples and the drawings wherein:

FIG. 1 illustrates genetic linkage maps of the anx interval of mouse Chromosome 2. The percentage recombination ±1 SE is indicated for each genetic interval. Centromere at the top (filled circle) and telomere at the bottom;

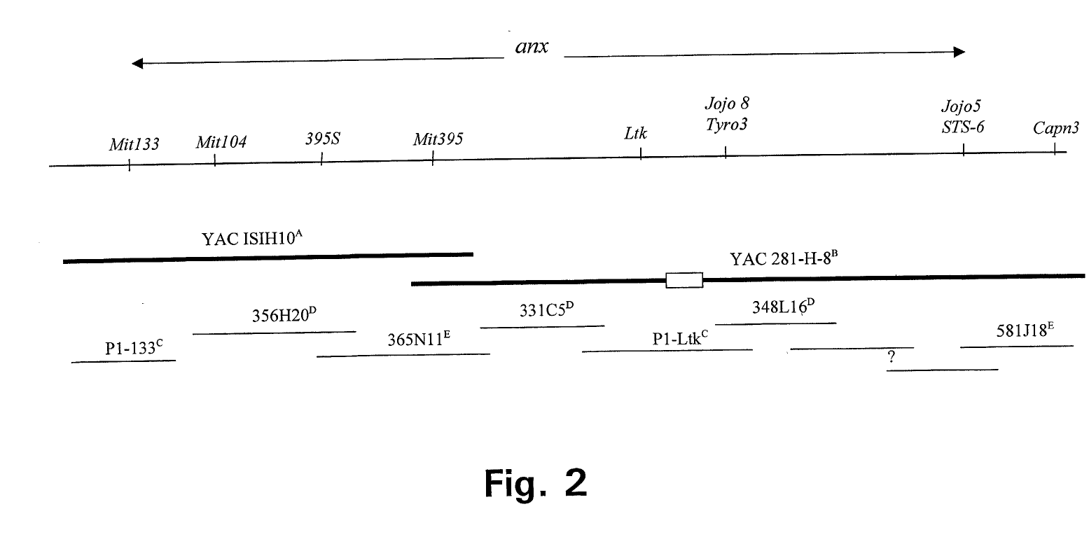

FIG. 2 is a physical map spanning the anx interval on mouse Chromosome 2 as described and defined in Example 1. The map is not drawn to scale. The relative positions of genes and markers, shown above the map, were deduced form the genetic linkage map and their presence and absence in clones. Chromosome centromere to the left and telomere to the right. Clones are identified by type of clone and number. Open box indicates internal deletion within the clone. ^{A}Chartier et al. (1990) Nature Genet. 1, 132; ^{B}http://carbon.wi.mit.edu:8000/cgi-in/mouse/yac_info?yac=281_H_{—}8&database=mouserelease;^{C}P1 library derived from E14 (129/Ola) ES cells; ^{D}BAC library RPCI-23, segment two (BACPAC Resources, Oakland, Calfi.); ^{E}PAC library RPCI-21 (BACPAC Resources, Oakland, Calif.). Genes identified: lp3k (clone ID P1-Ltk); EMBL No. baa92641 (clone ID P1 Ltk); DII4, EMBL No. aaf76428 (clone ID 356H20); Vps18, EMBL No. baa95999 (clone ID 356H20); Epa6, EMBL No. epab_mouse (clone ID 356H20); EMBL No. mmaa11531 (clone ID 356H20); EMBL No. q9ulg1 (clone ID 356H20); EMBL No. q9vci9 (clone ID 356H20); Chp, EMBL No. q9z1y0 (clone ID 356H20); S27-1, EMBL No. aad56582 (clone ID 356H20); Fabe, EMBL No. fabe_mouse (clone ID 348 L16); Mga, EMBL No. q9qxj5 (clone ID 348 L16); JNK, EMBL No. q9r010 (clone ID 348 L16); CPLA2-beta, EMBL No. q9ukv7 (clone ID 348L16); p22, EMBL No. rn39875 (clone ID 365N11);

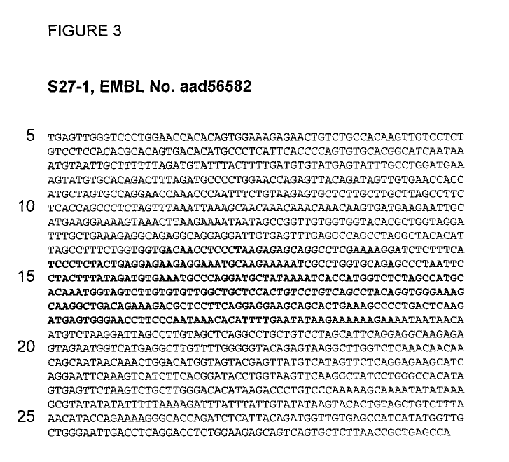

FIG. 3 is the sequence of gene S27-1, EMBL No aad56582, exon sequences are indicated with letters in bold (also in the following figures);

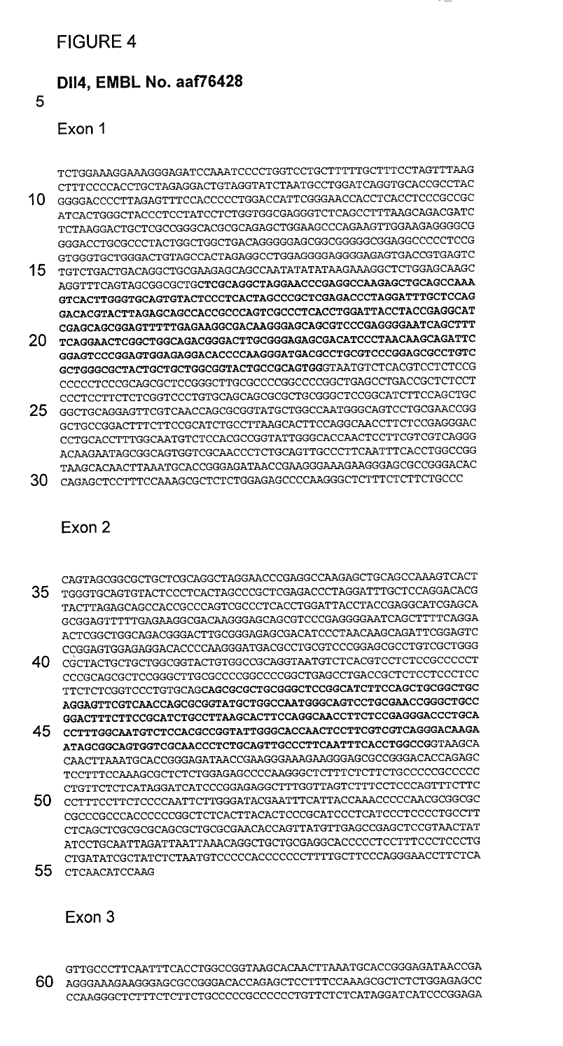

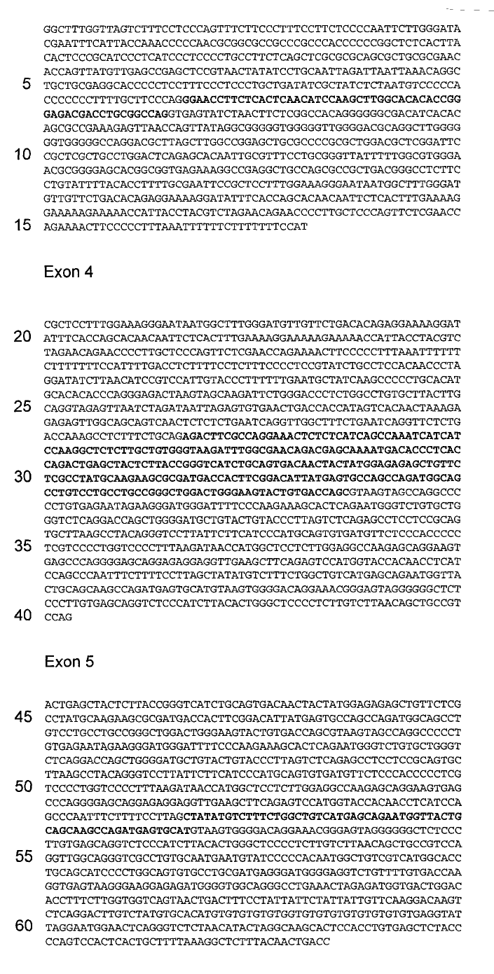

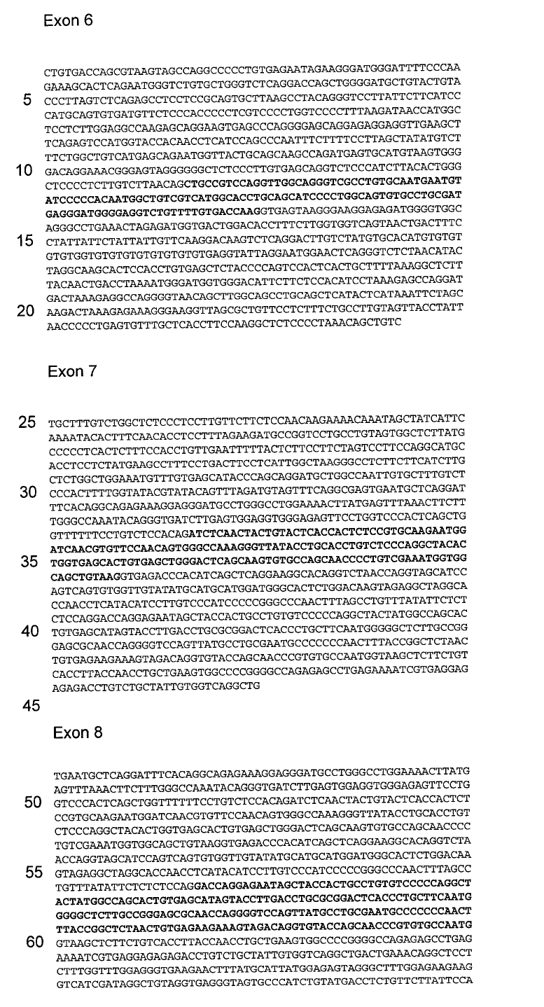

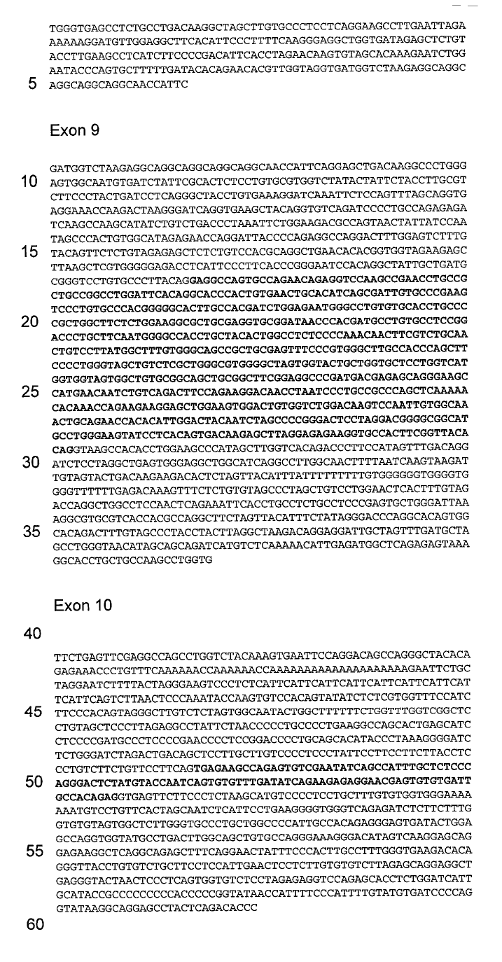

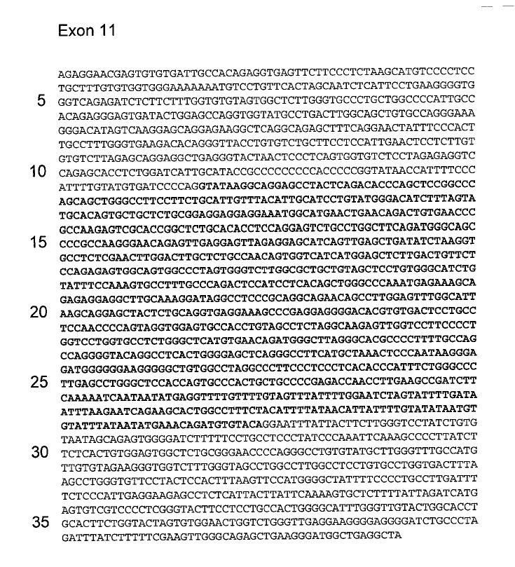

FIG. 4 is the sequence of gene DII4, EMBL No. aaf76428;

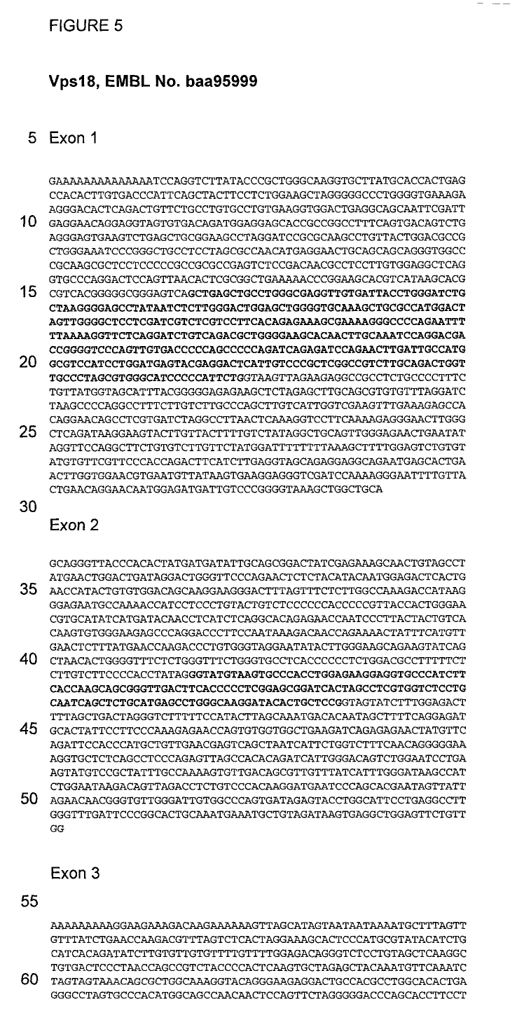

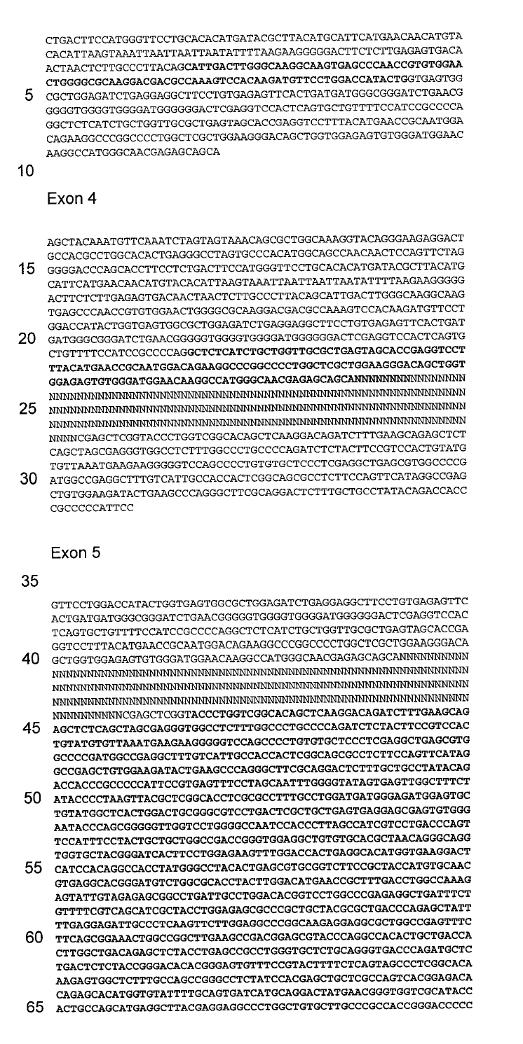

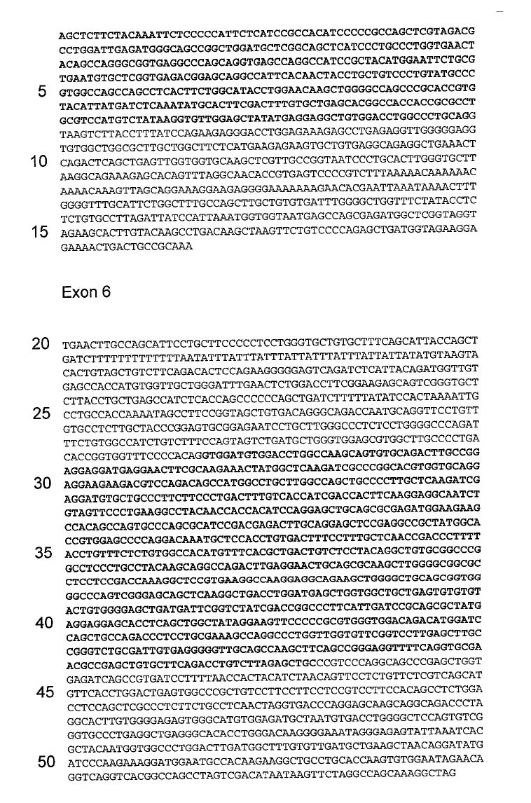

FIG. 5 is the sequence of gene Vps18, EMBL No. baa95999;

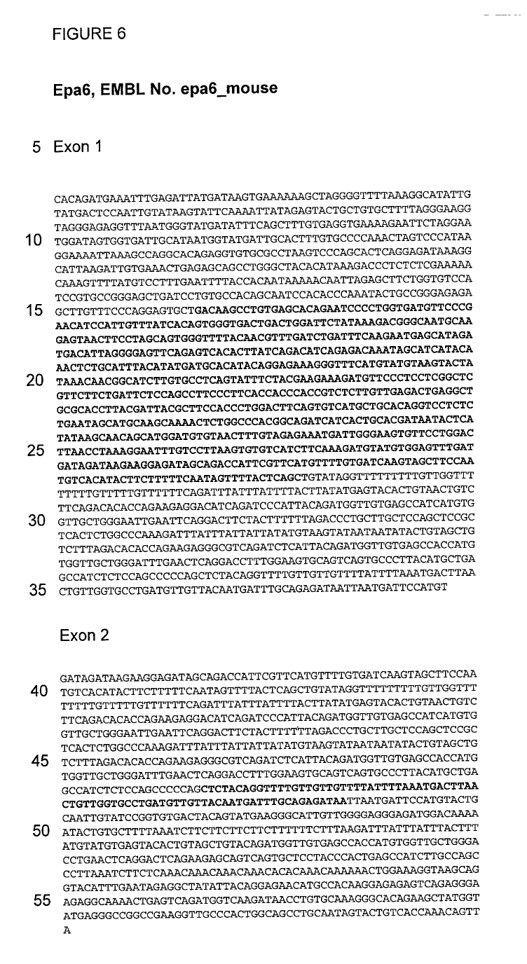

FIG. 6 is the sequence of gene Epa6, EMBL No. epab_mouse;

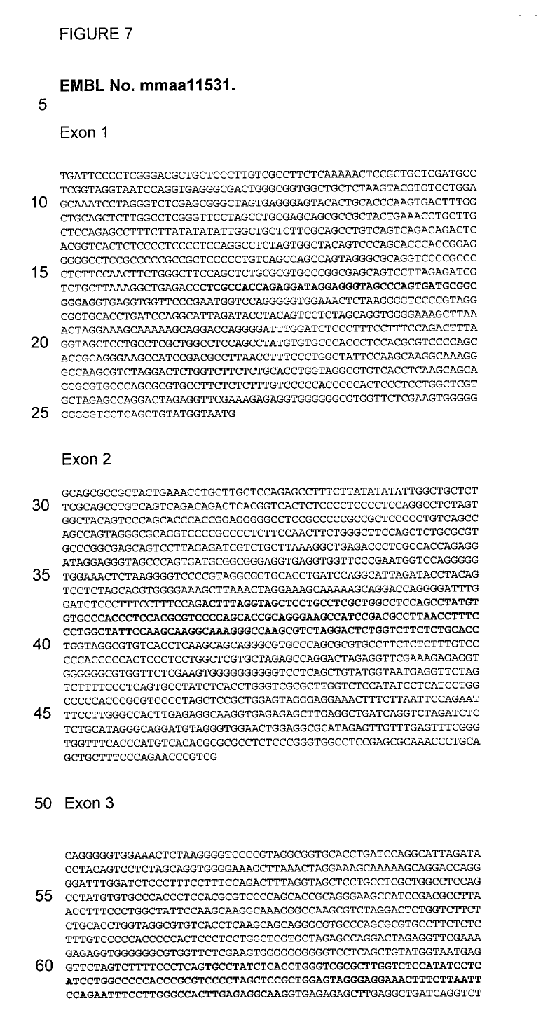

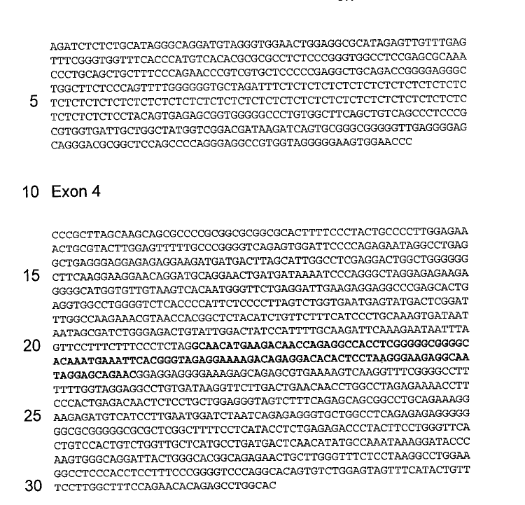

FIG. 7 is the sequence of gene EMBL No. mmaa1531;

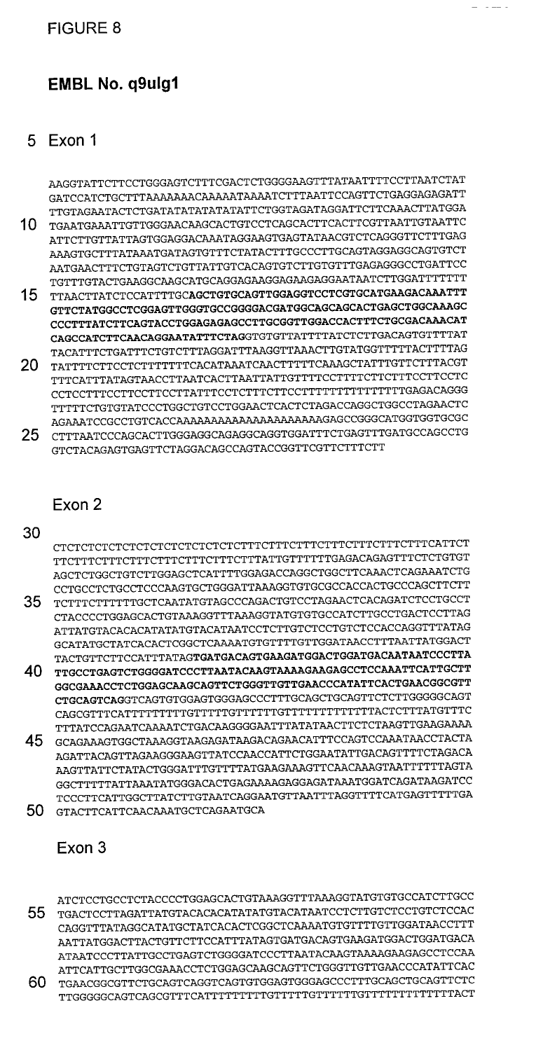

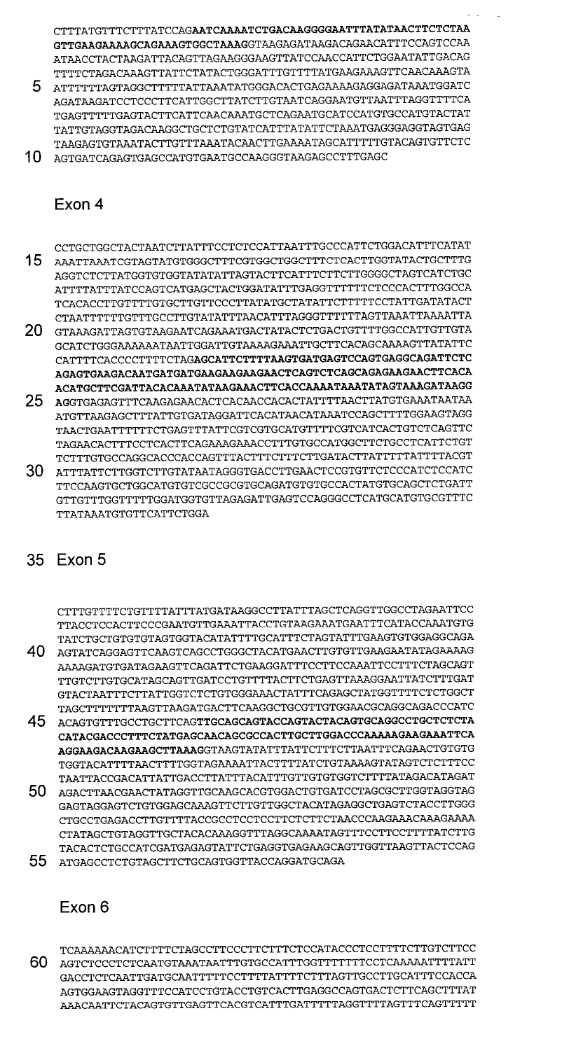

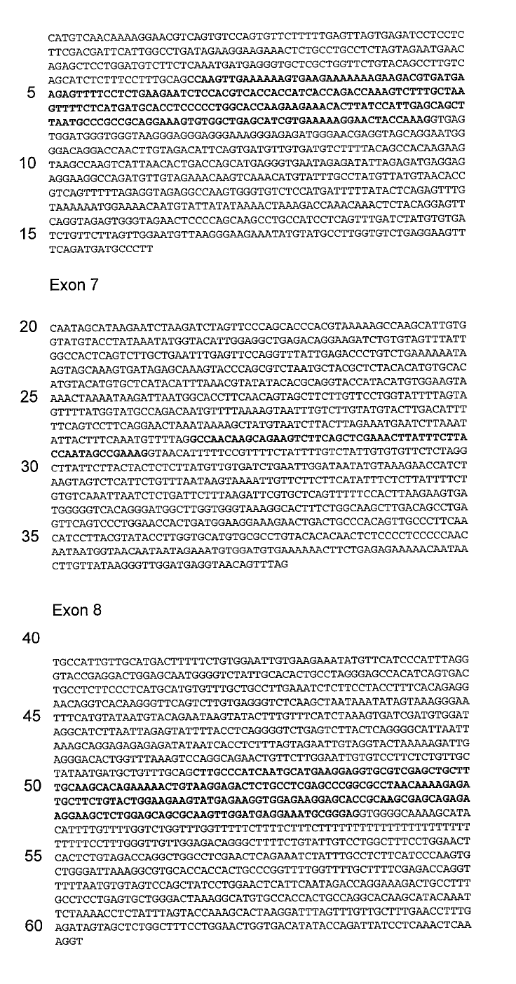

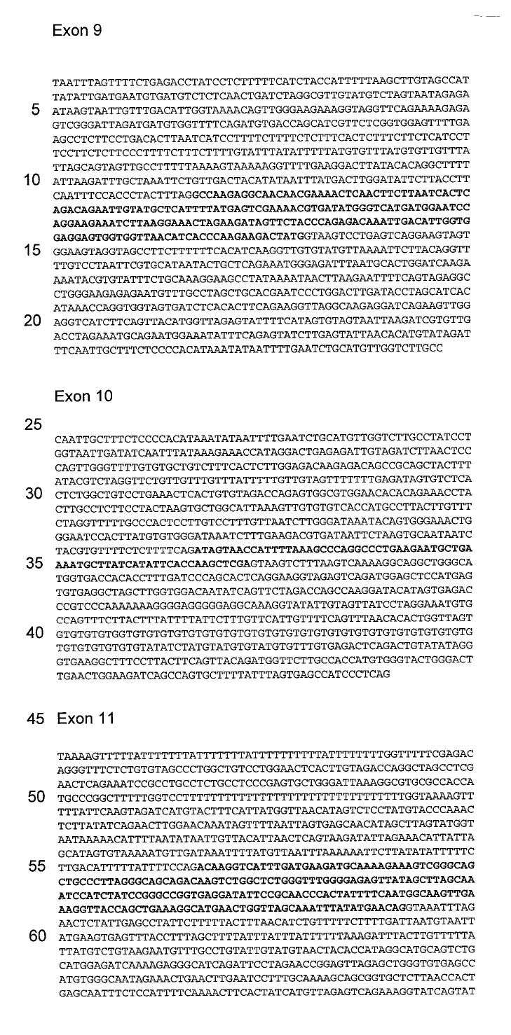

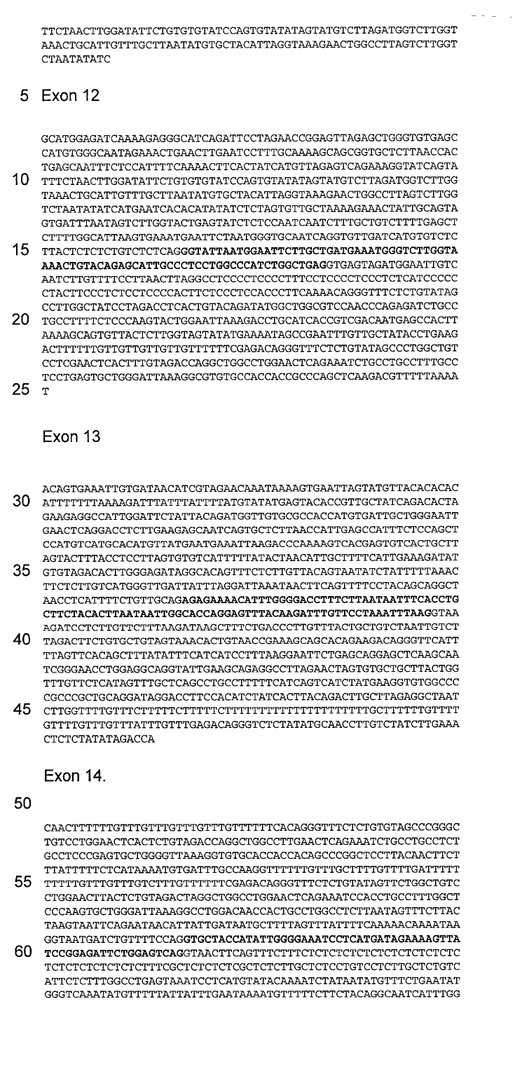

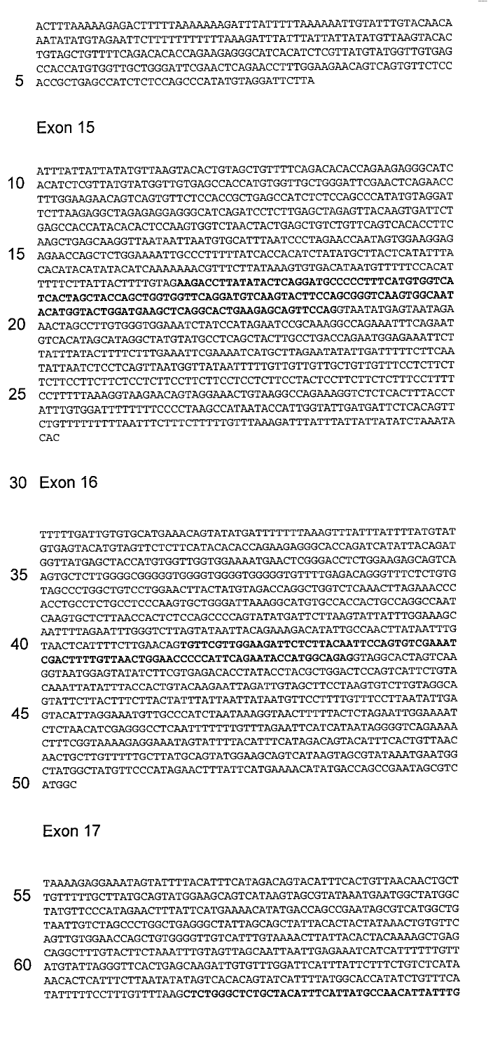

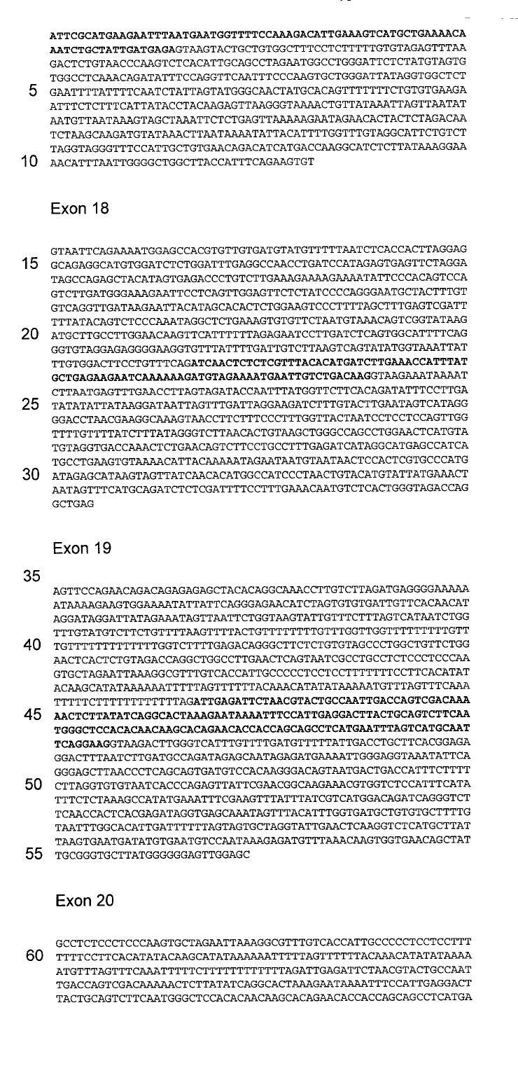

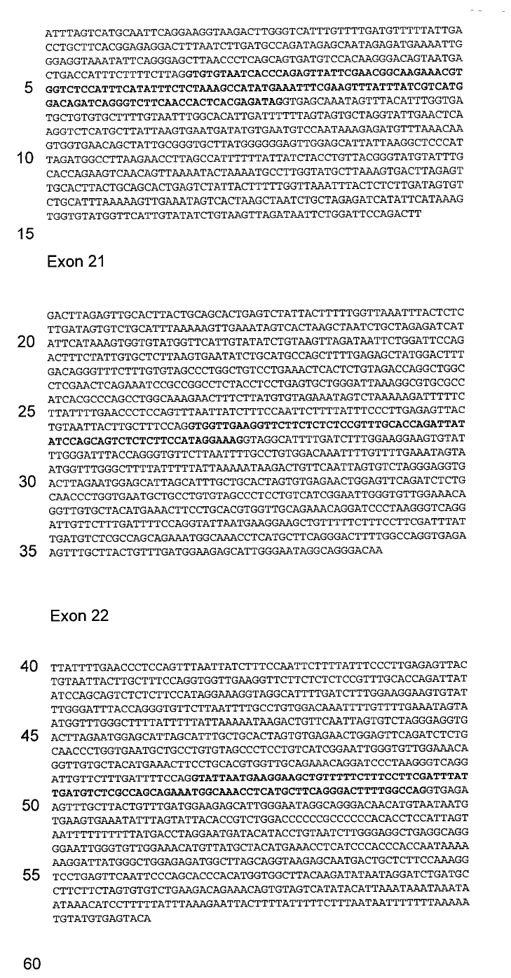

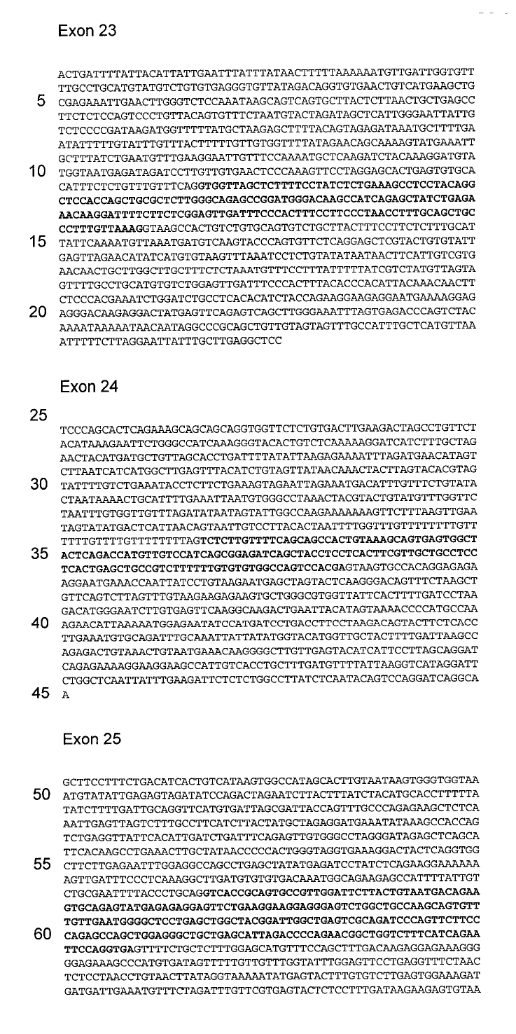

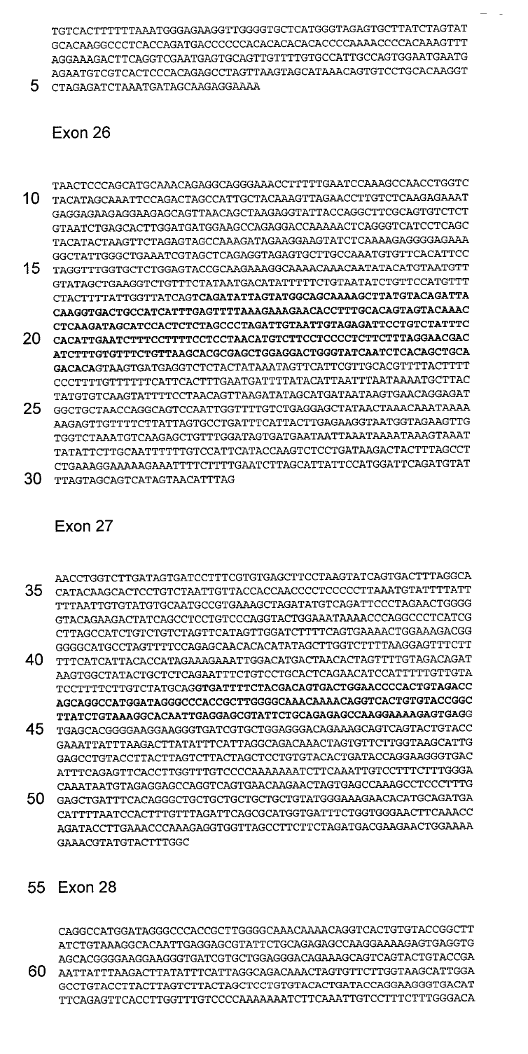

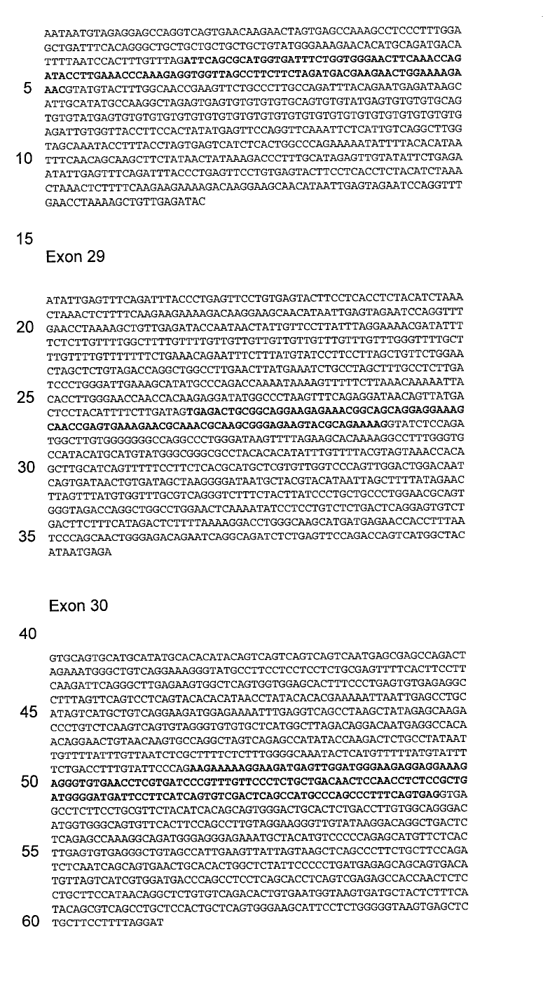

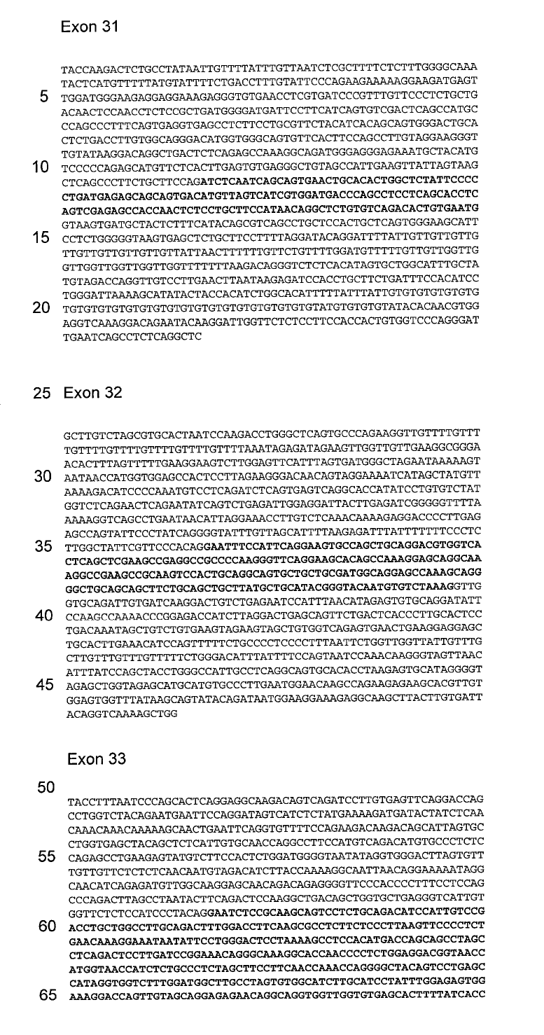

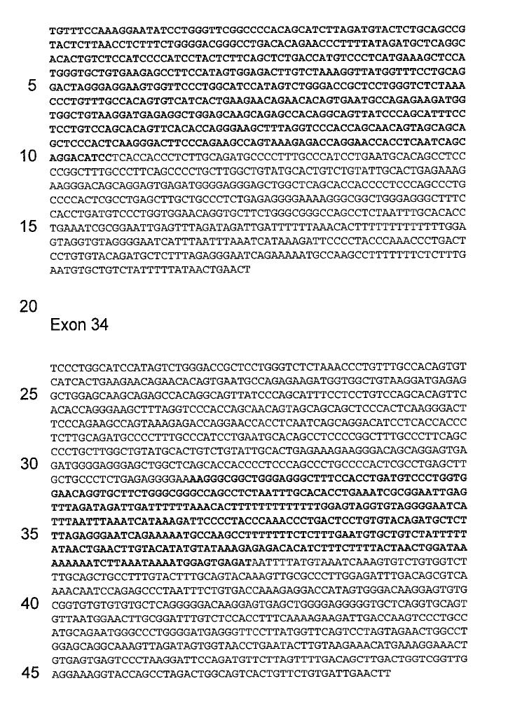

FIG. 8 is the sequence of gene EMBL No. q9ulg1;

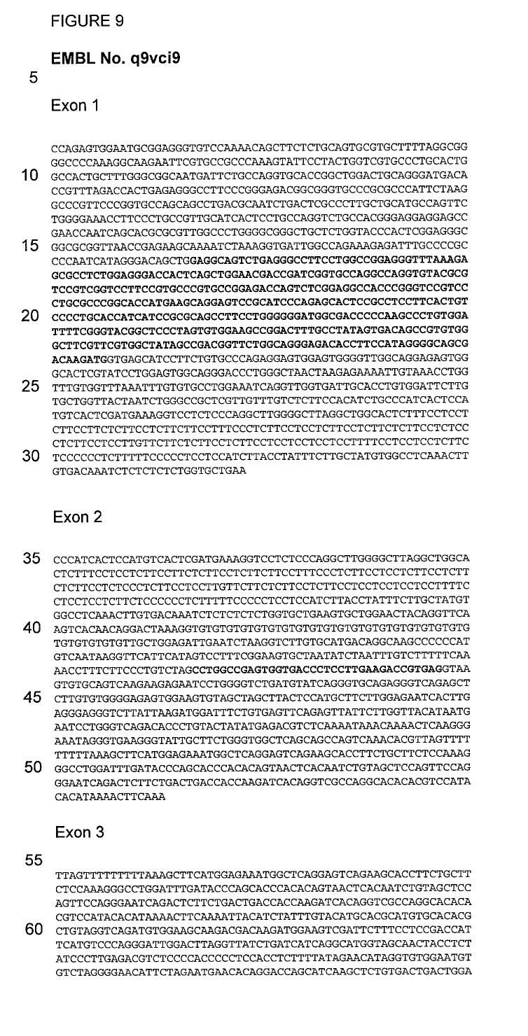

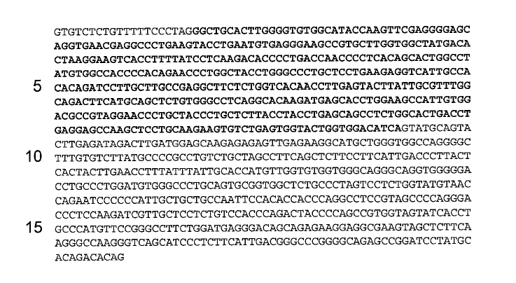

FIG. 9 is the sequence of gene EMBL No. q9vci9;

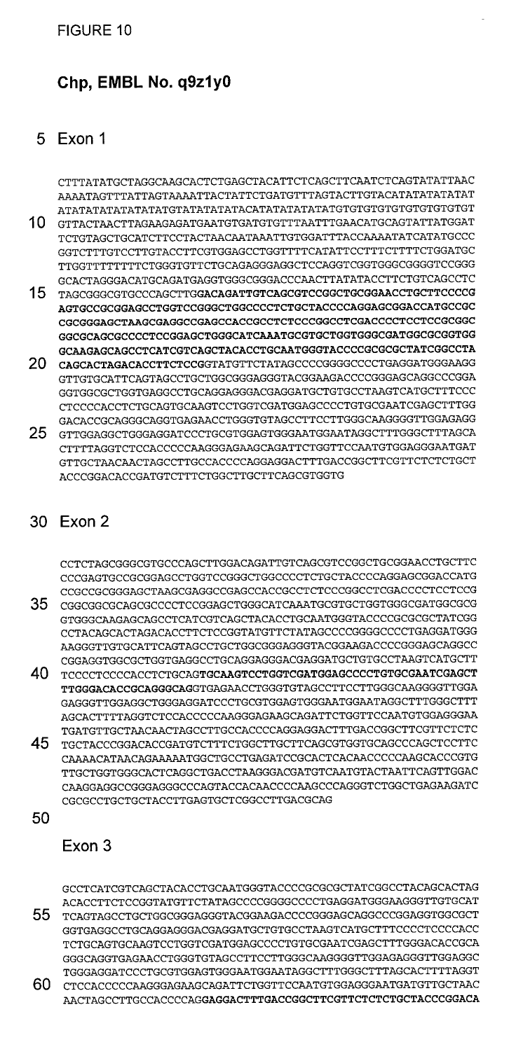

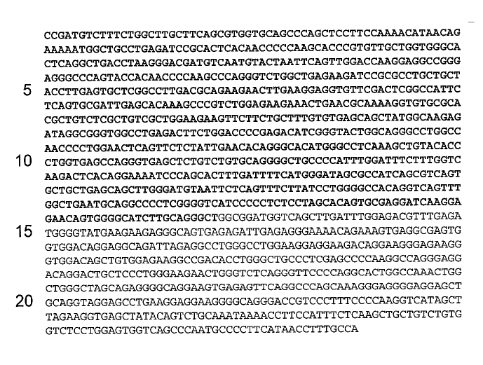

FIG. 10 is the sequence of gene Chp, EMBL No. q9z1y0;

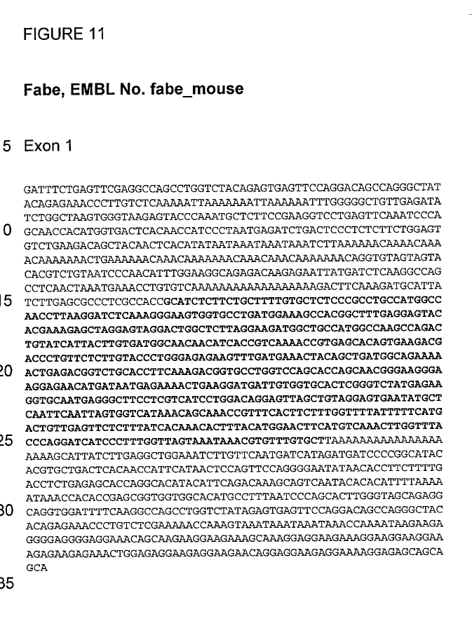

FIG. 11 is the sequence of gene Fabe, EMBL No. fabe_mouse;

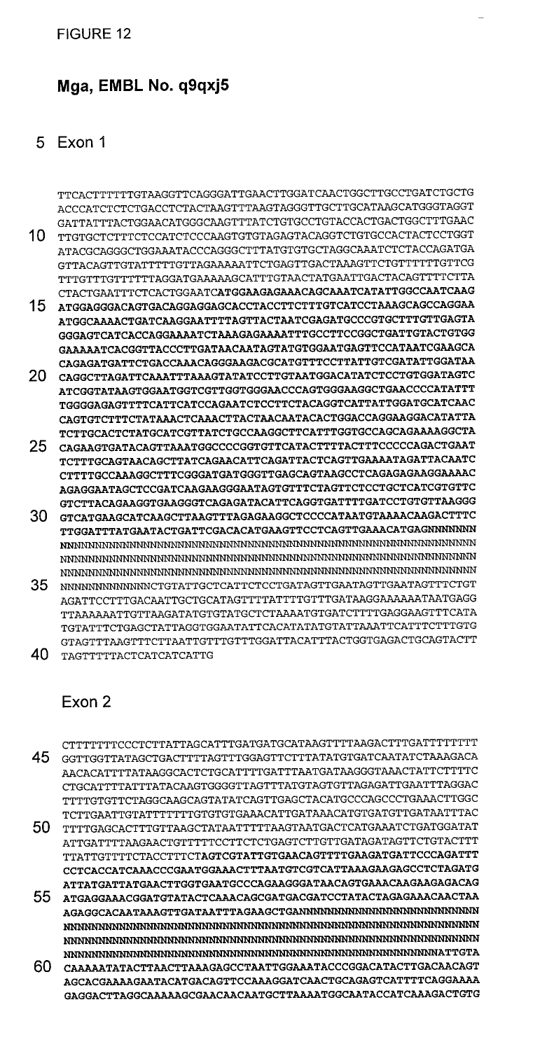

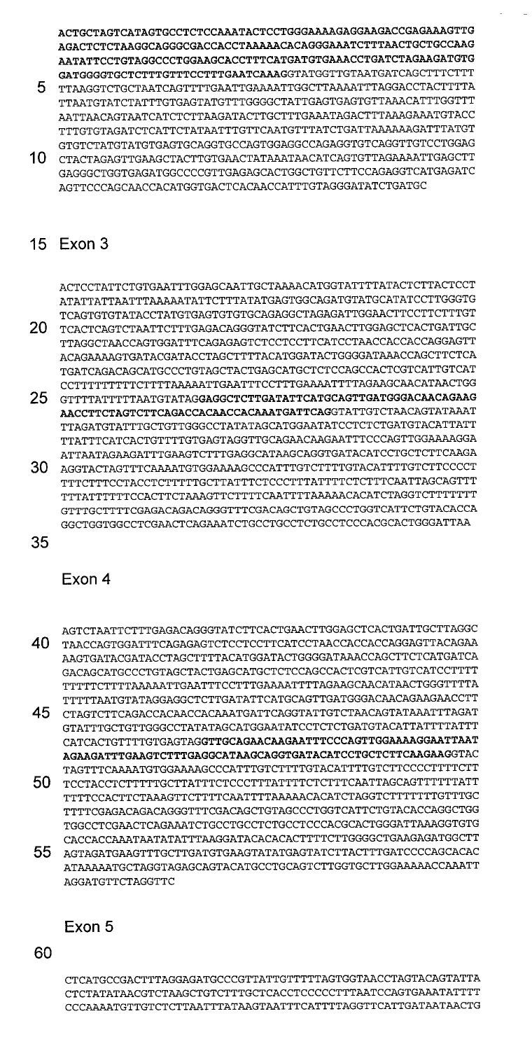

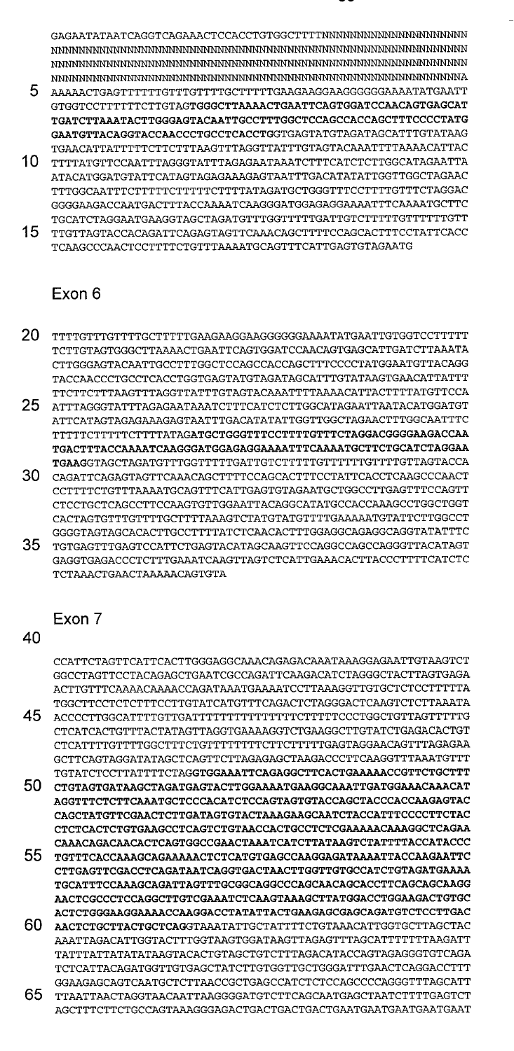

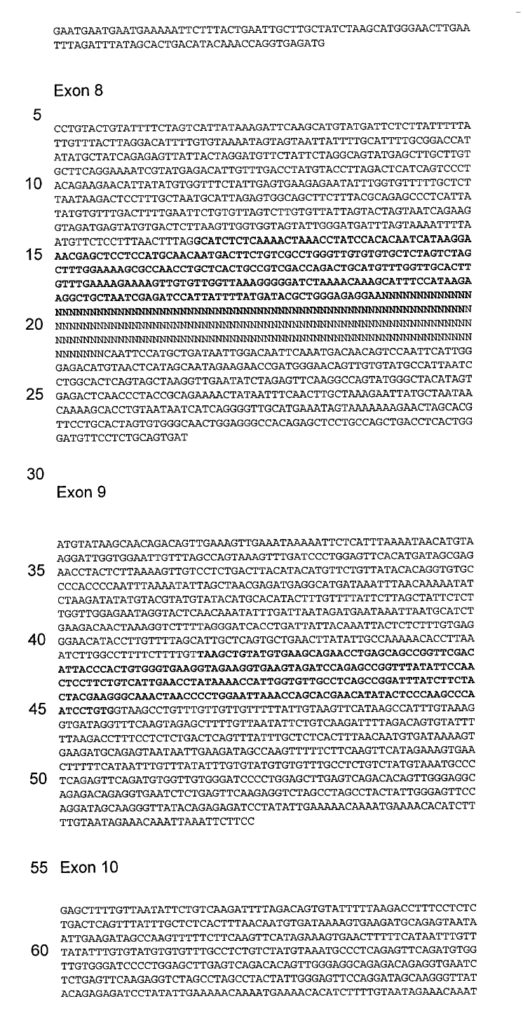

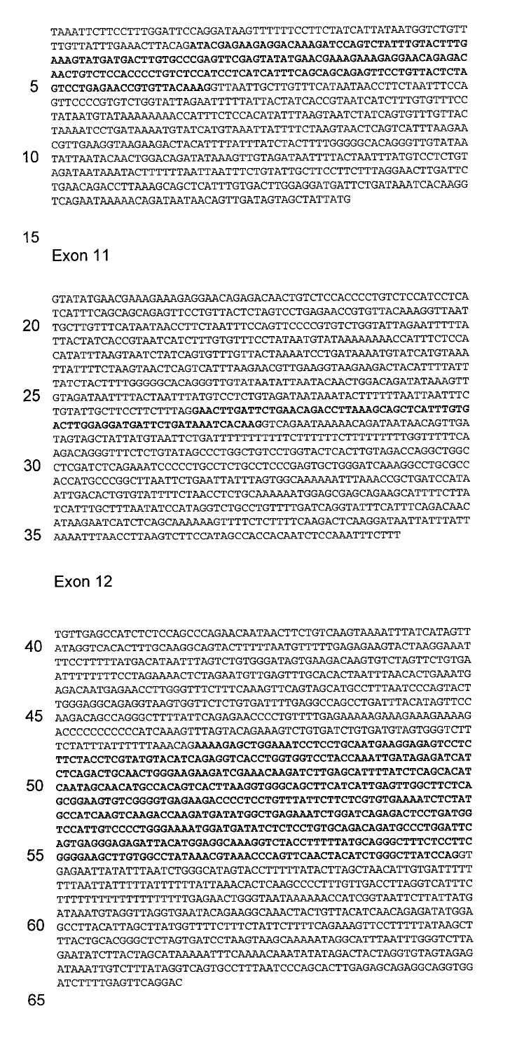

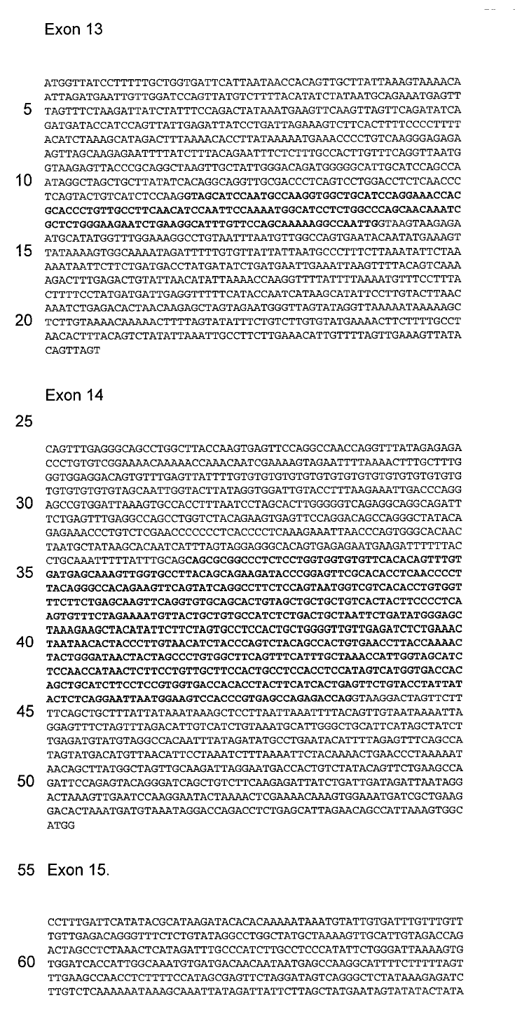

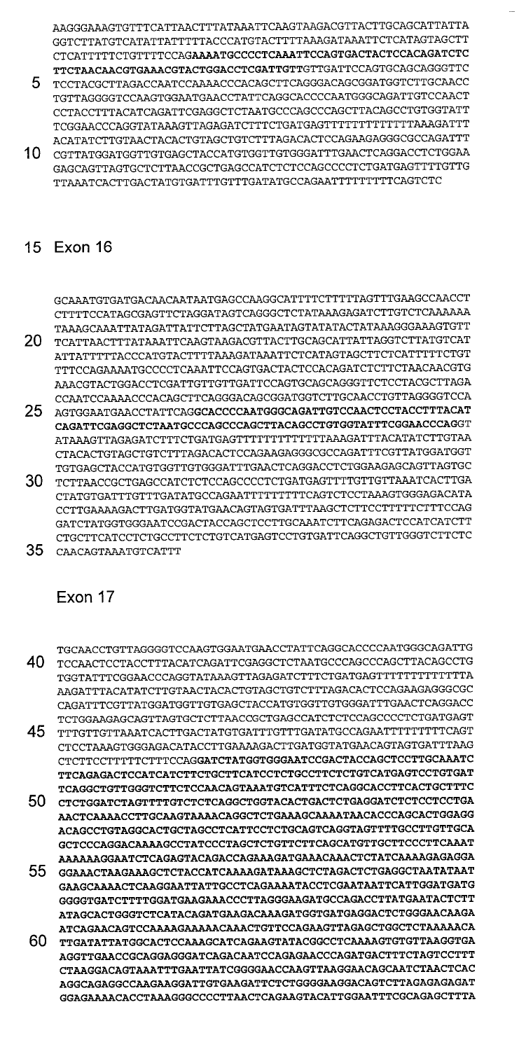

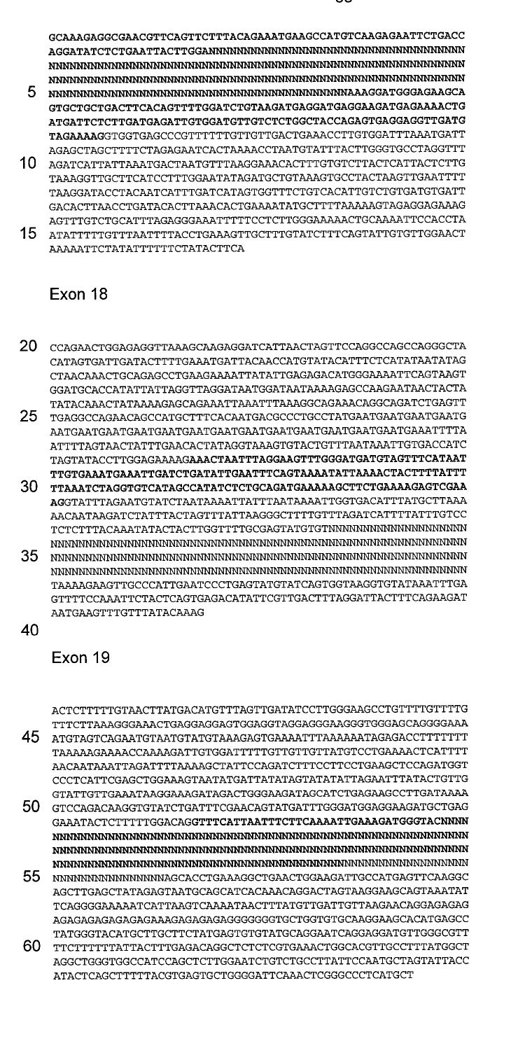

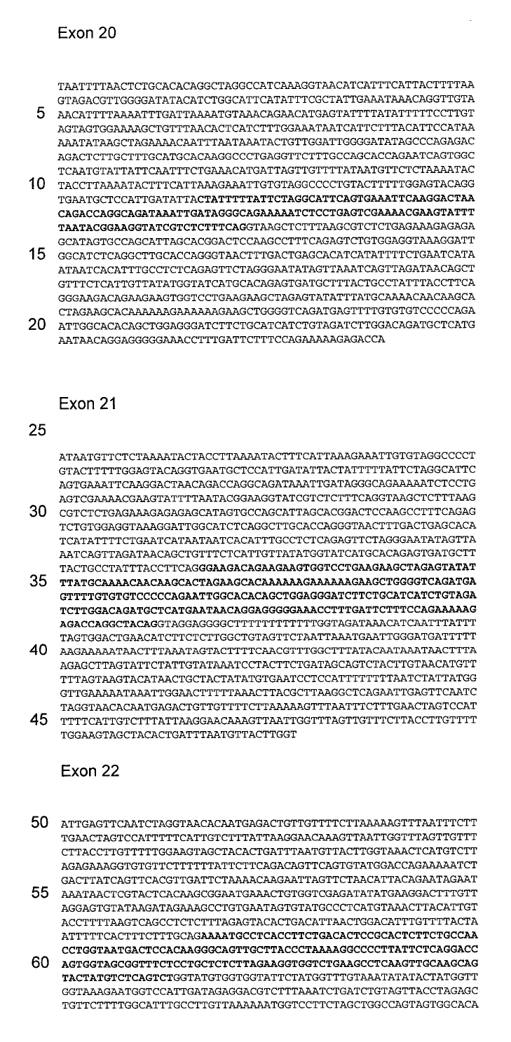

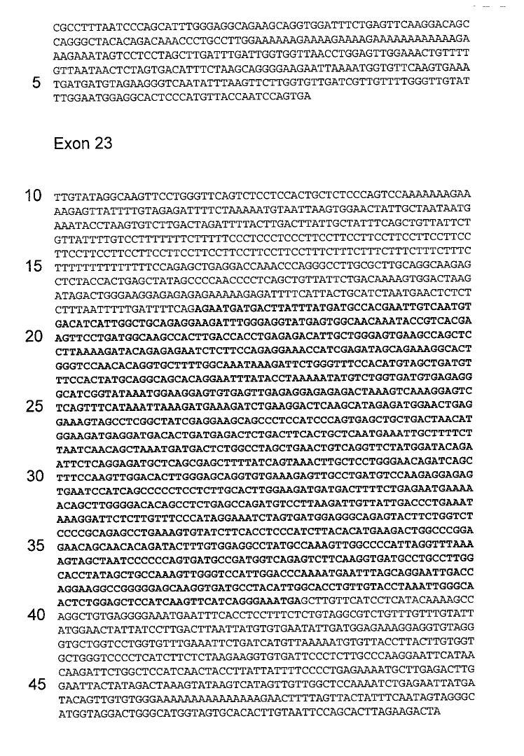

FIG. 12 is the sequence of gene Mga, EMBL No. q9qxj5;

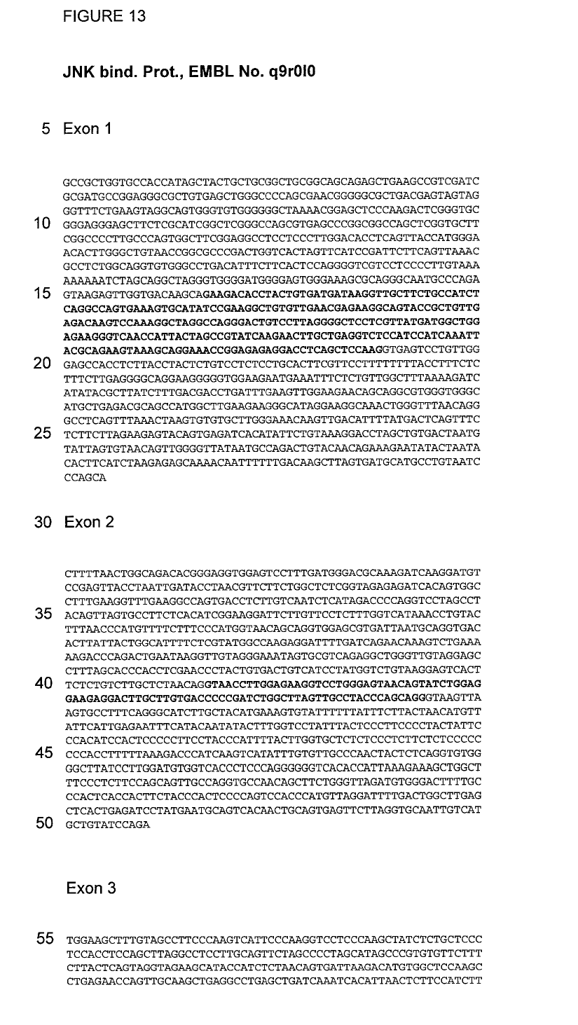

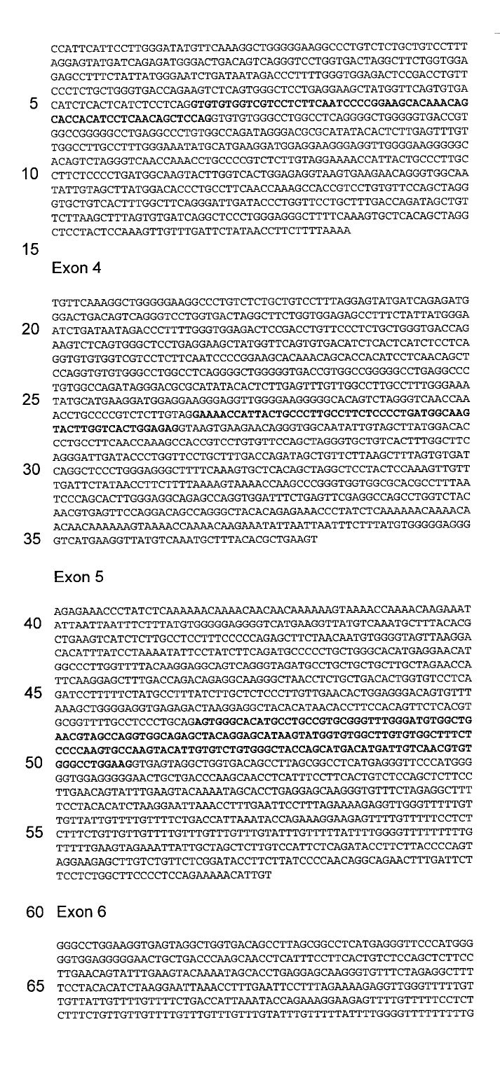

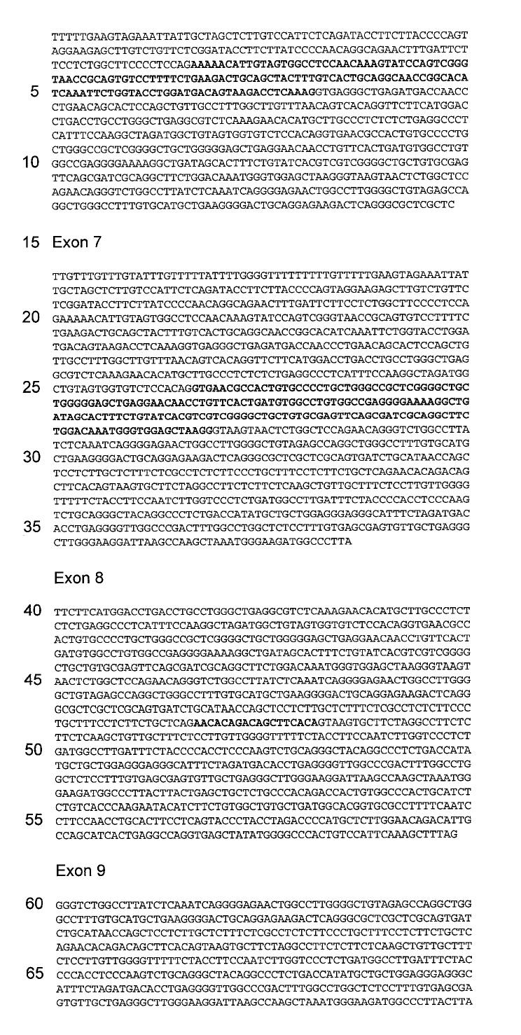

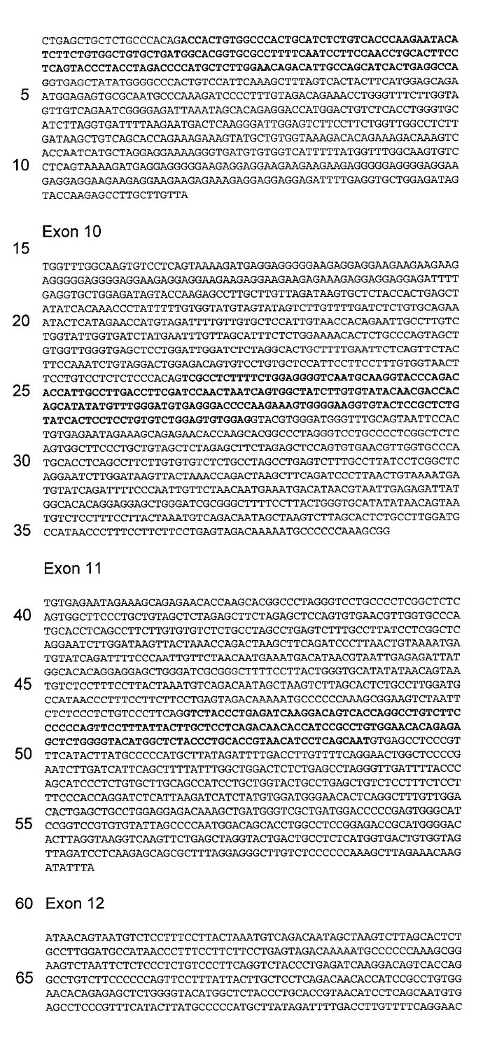

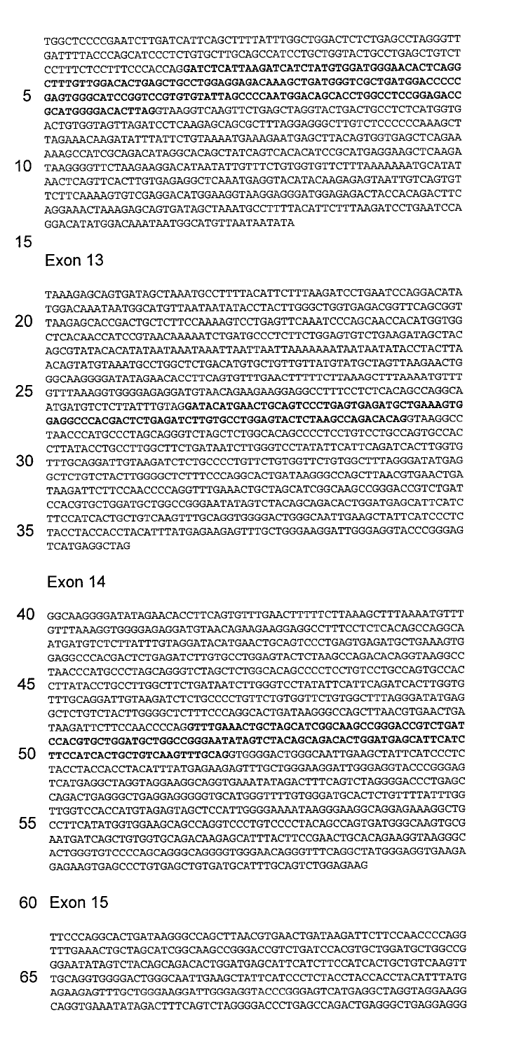

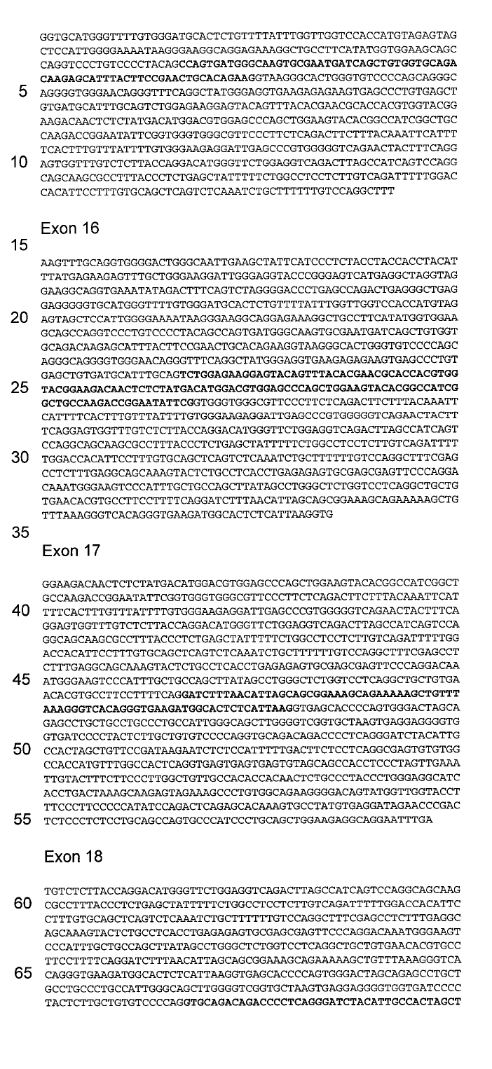

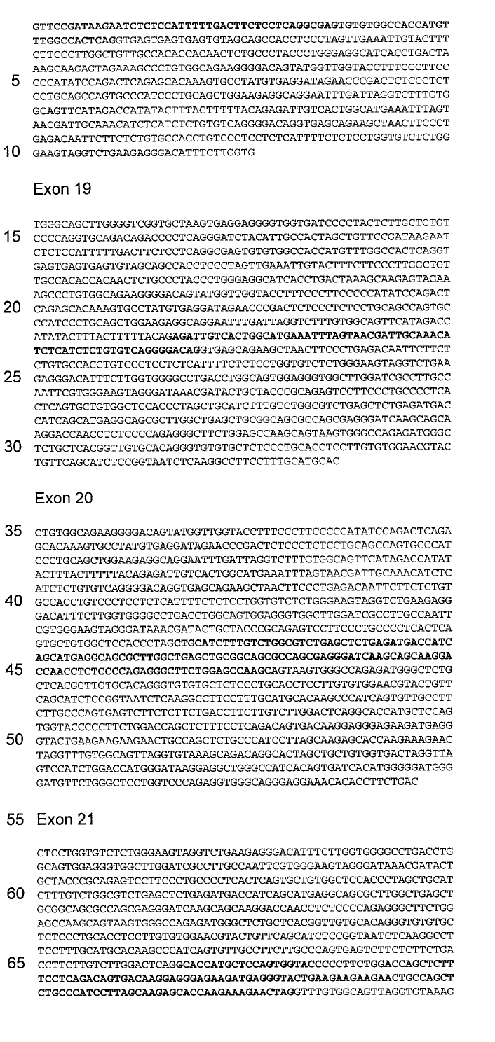

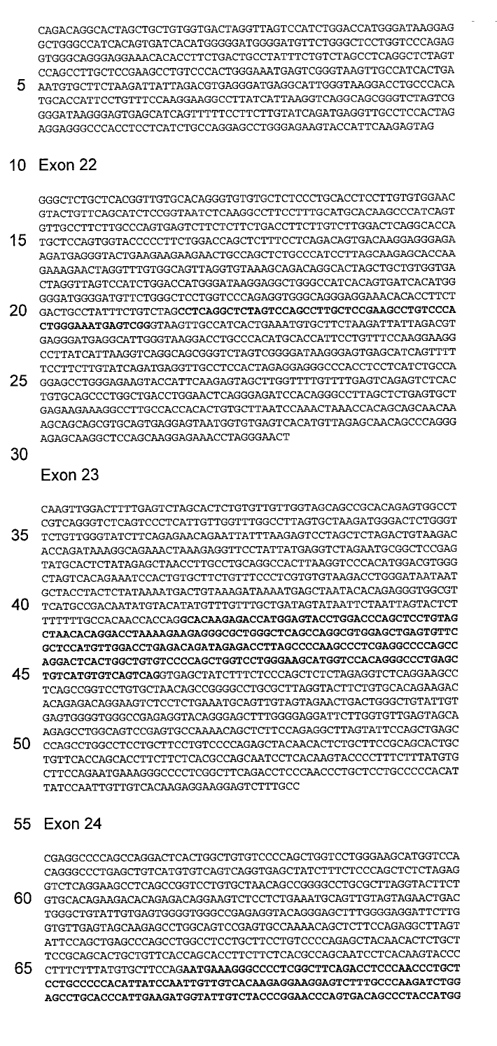

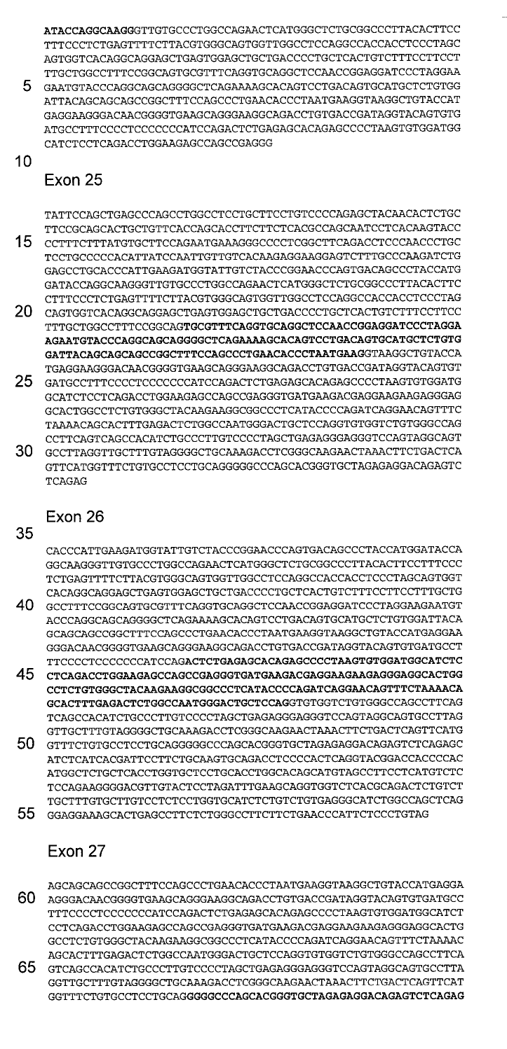

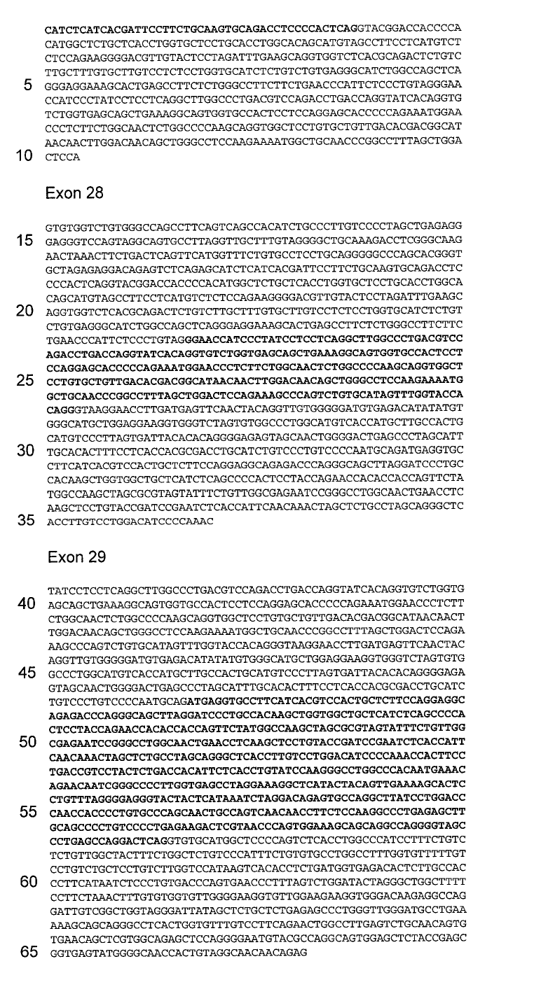

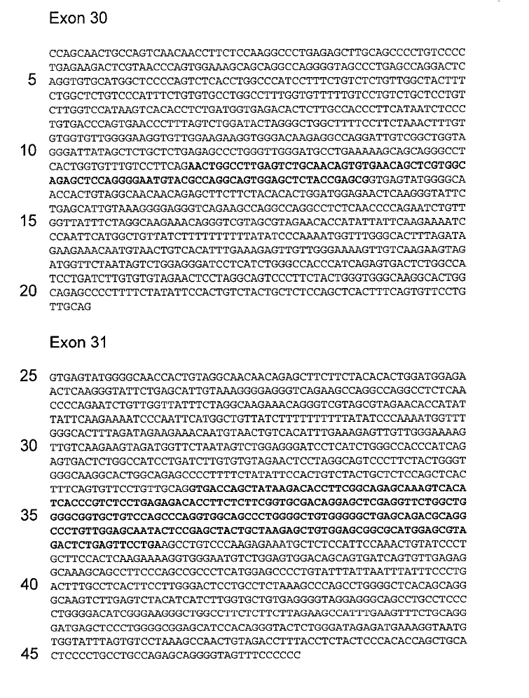

FIG. 13 is the sequence of gene JNK, EMBL No. q9r010;

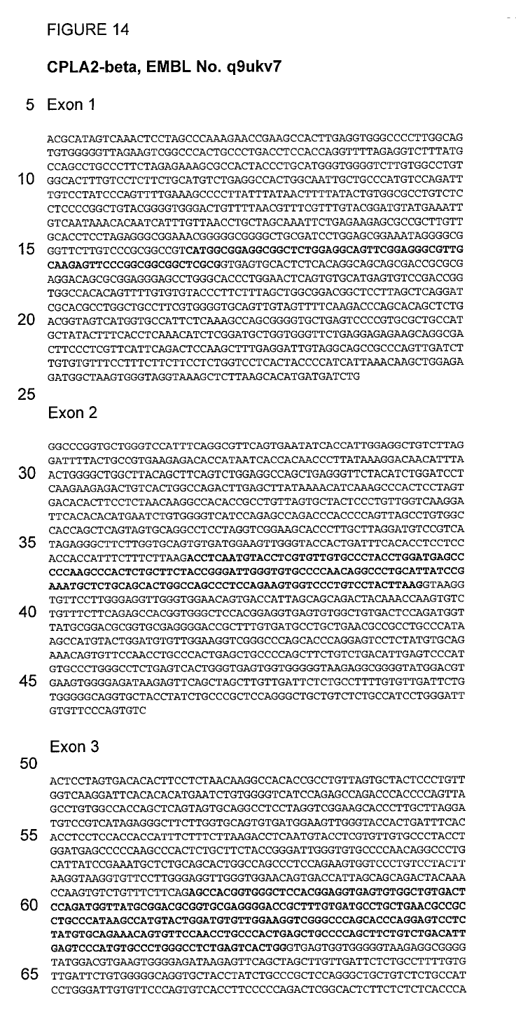

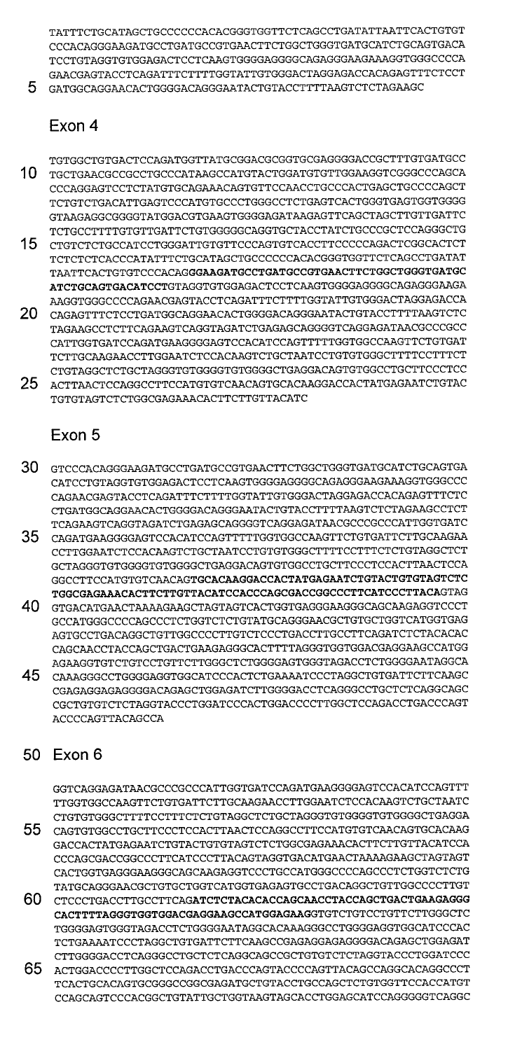

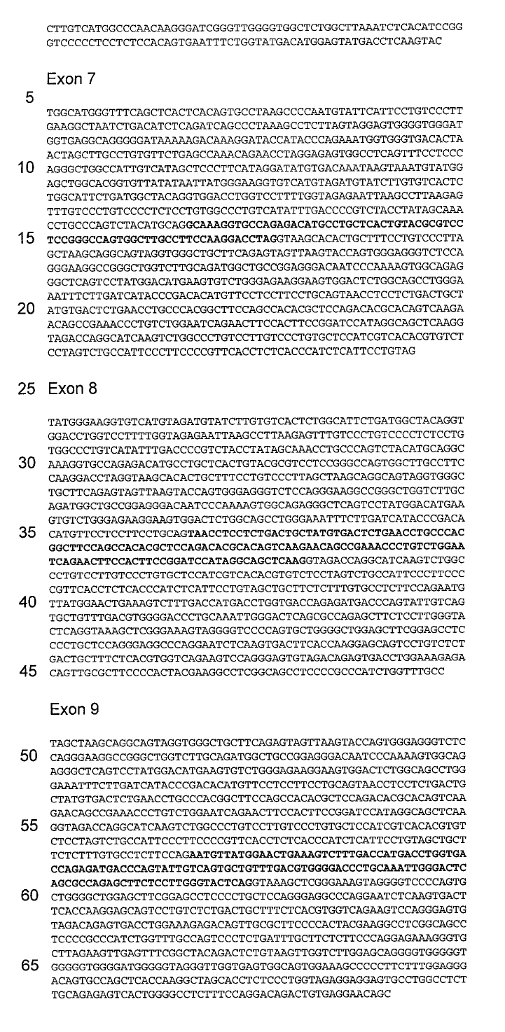

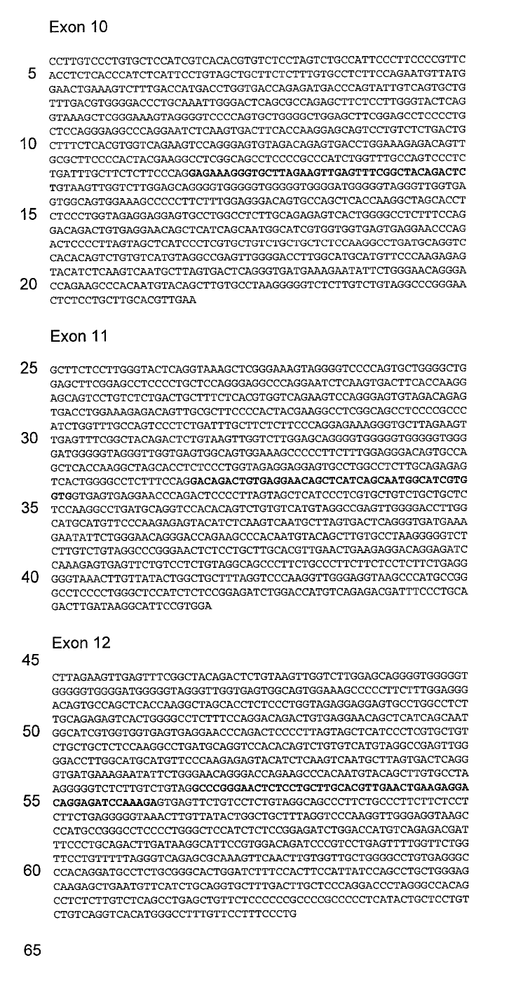

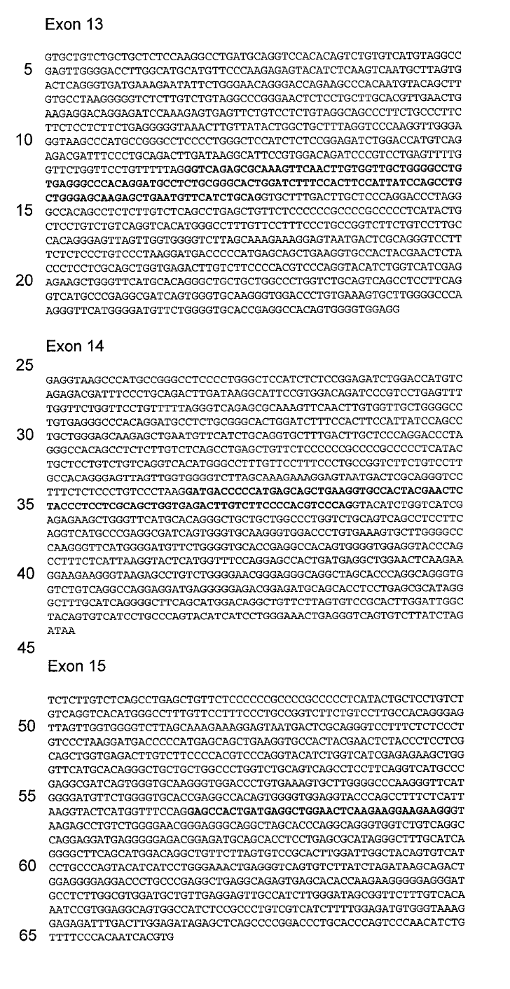

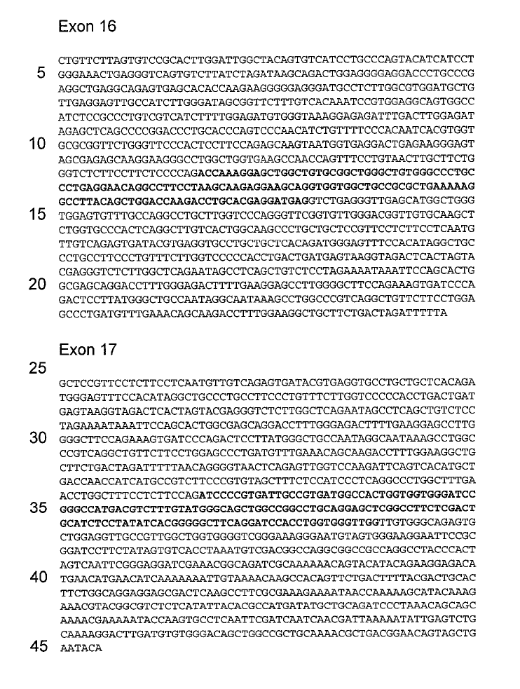

FIG. 14 is the sequence of gene CPLA2-beta, EMBL No. q9ukv7;

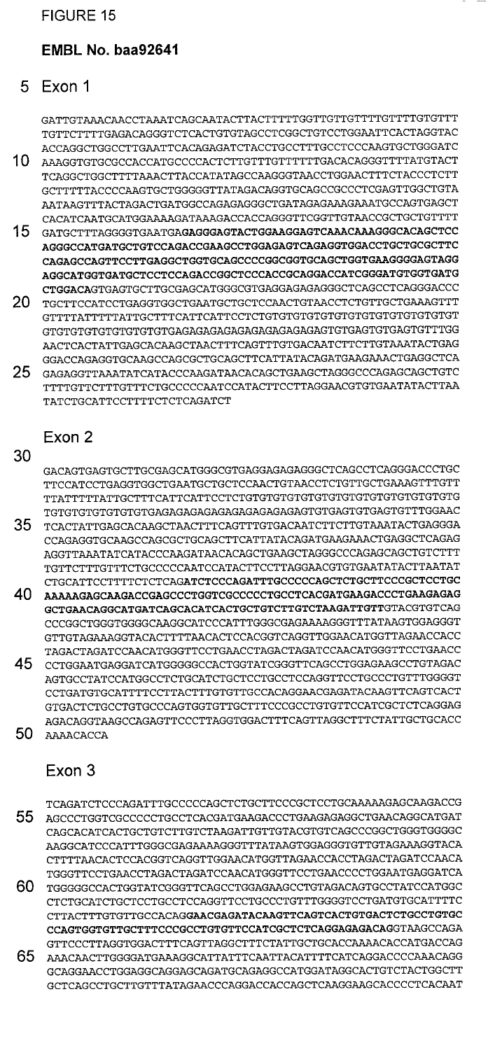

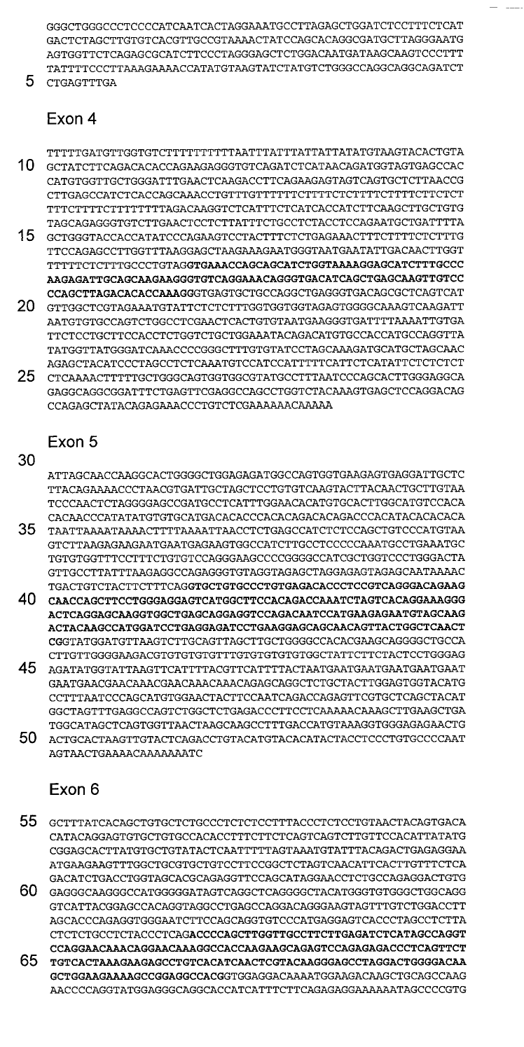

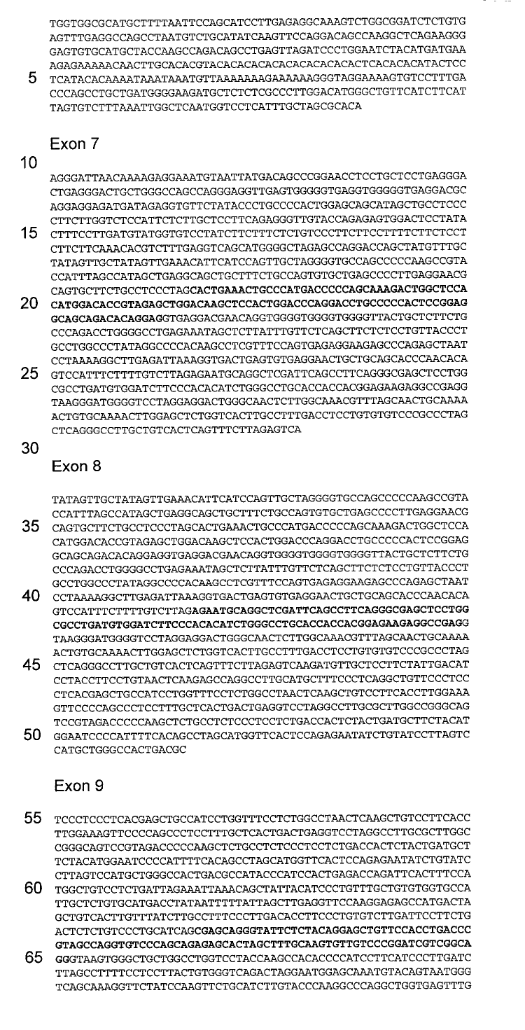

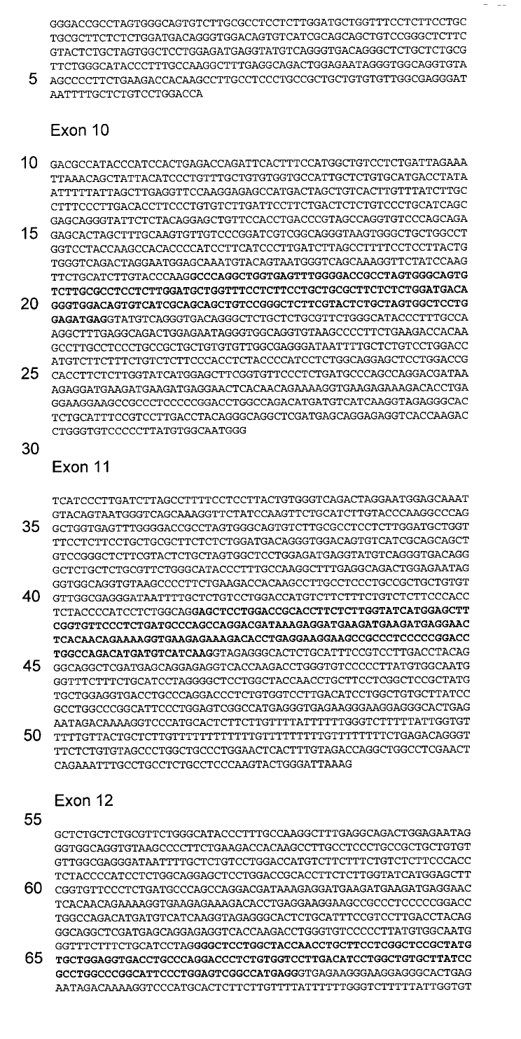

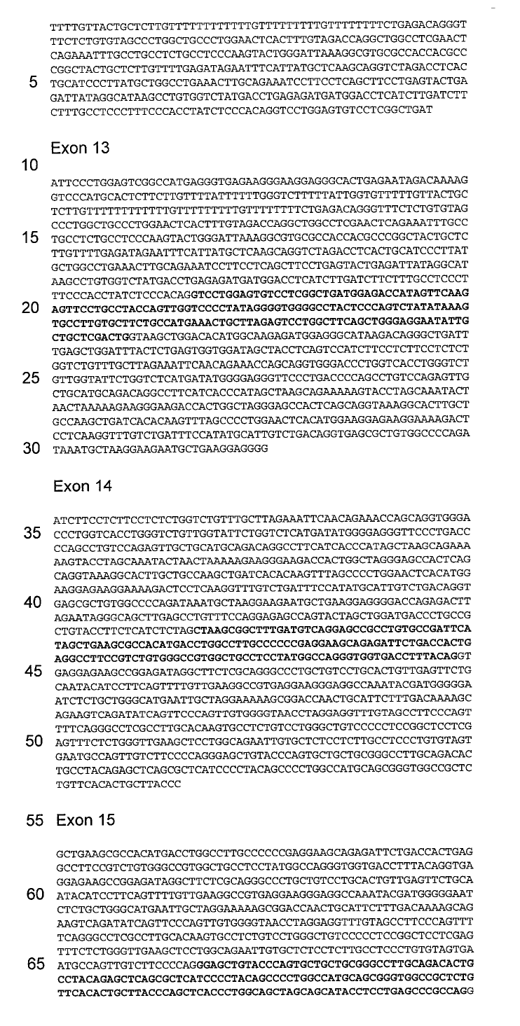

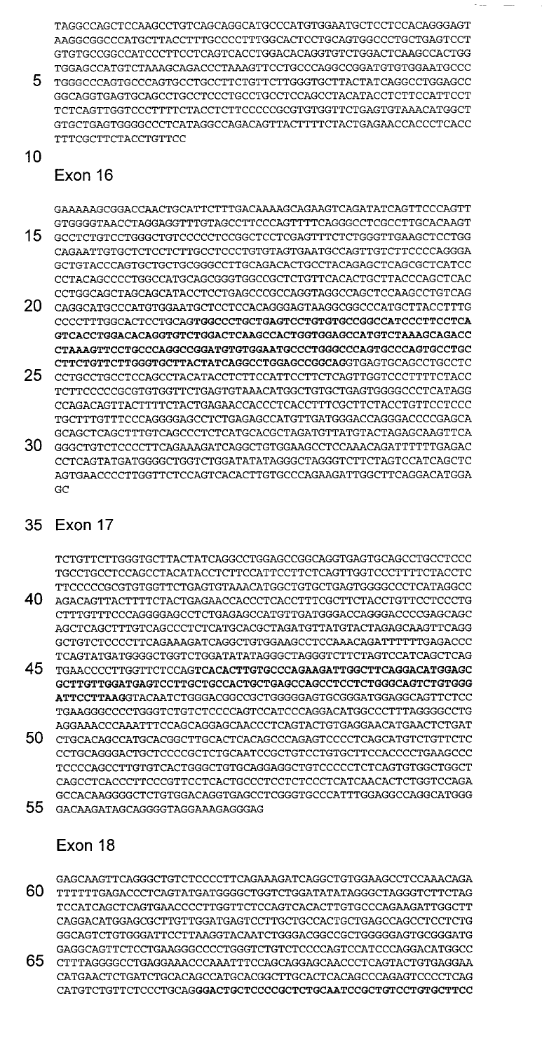

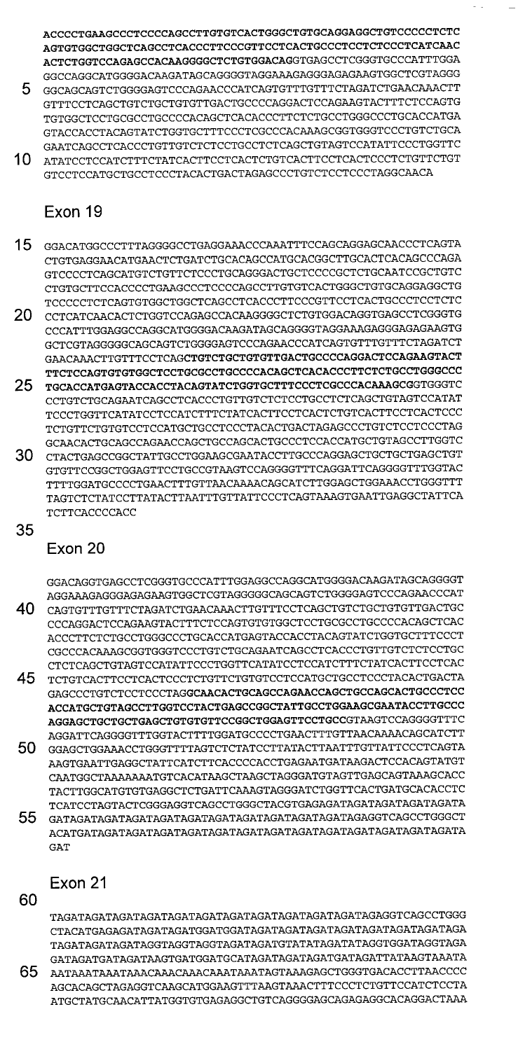

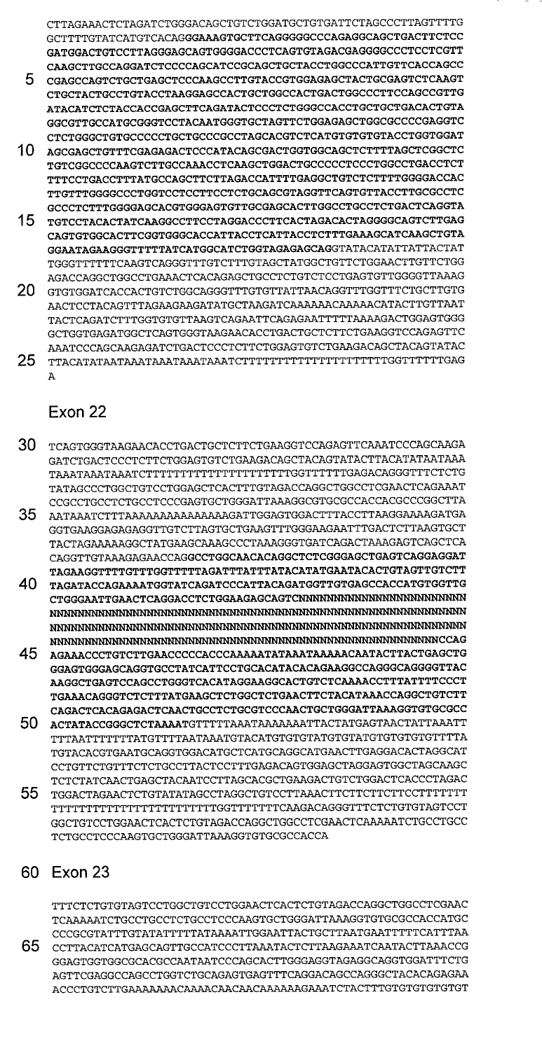

FIG. 15 is the sequence of gene EMBL No. baa92641;

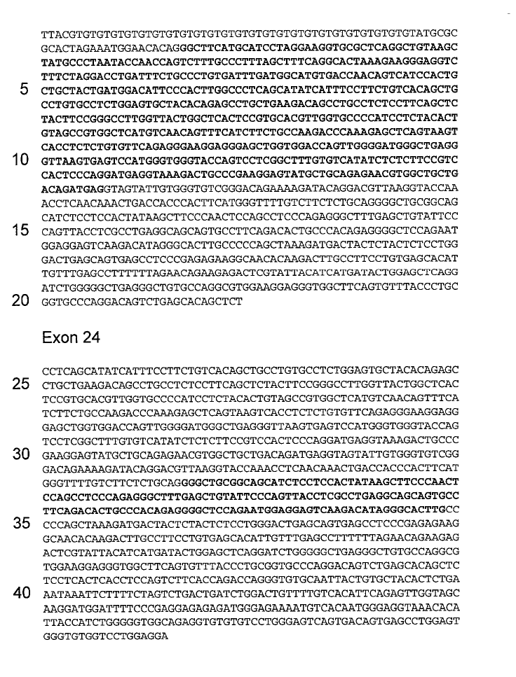

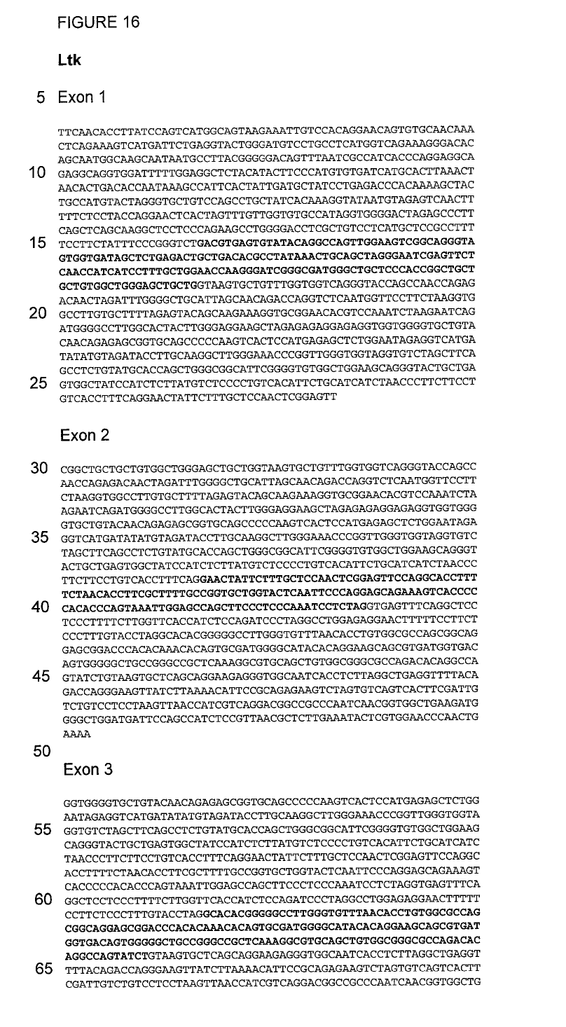

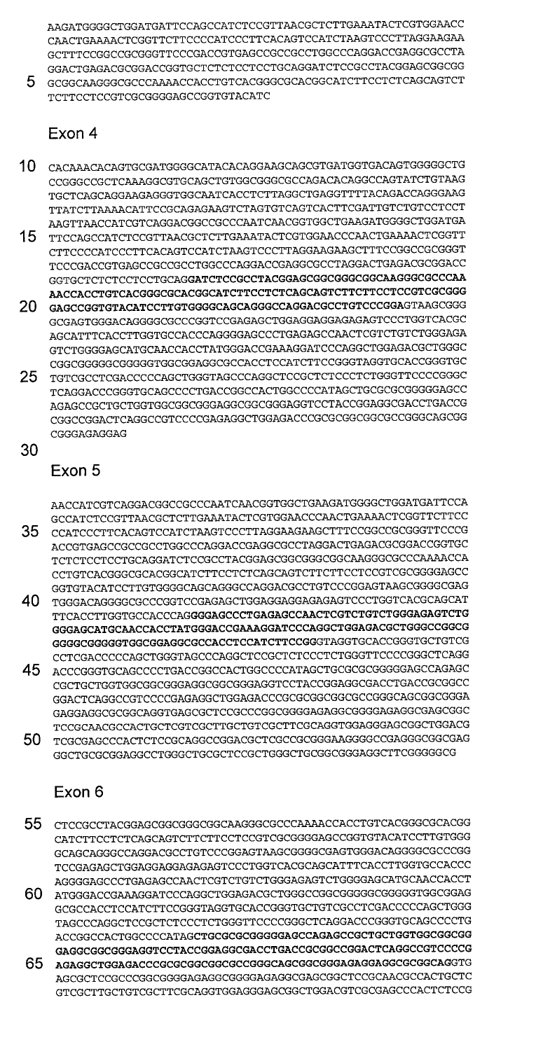

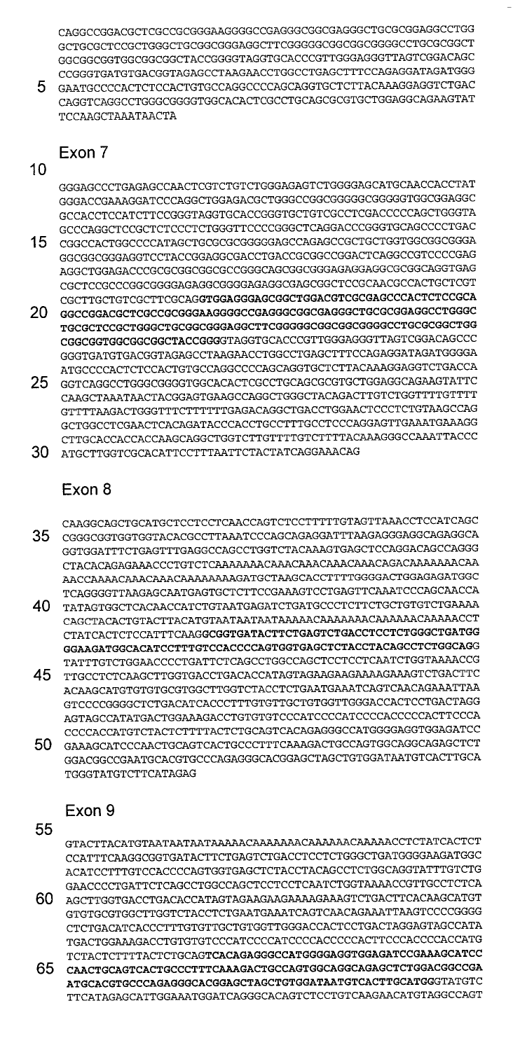

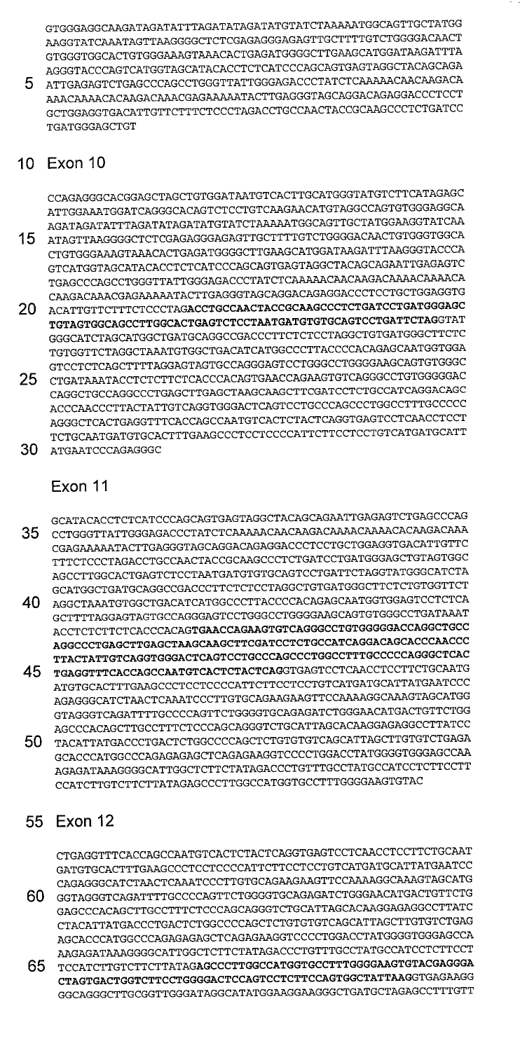

FIG. 16 is the sequence of gene Ltk;

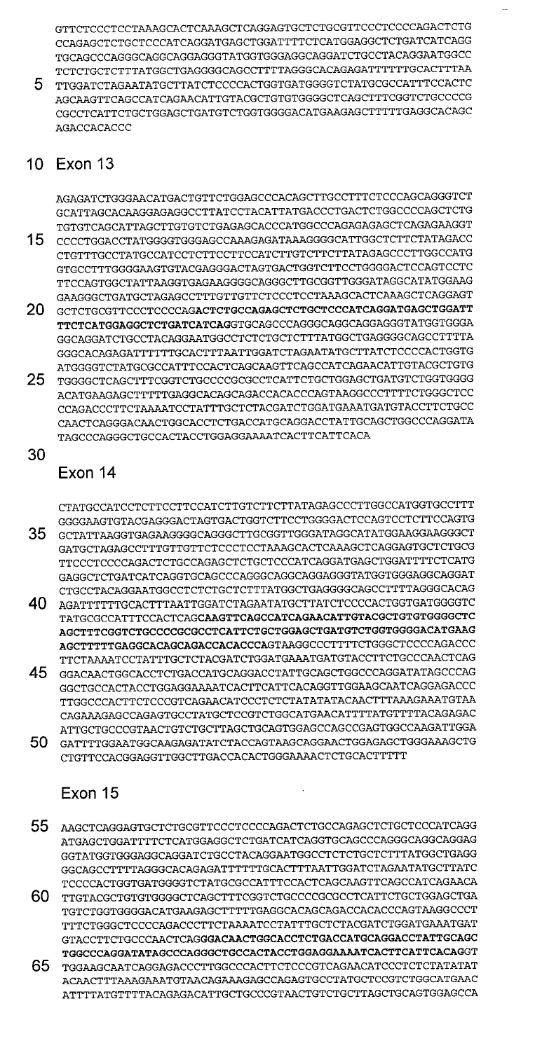

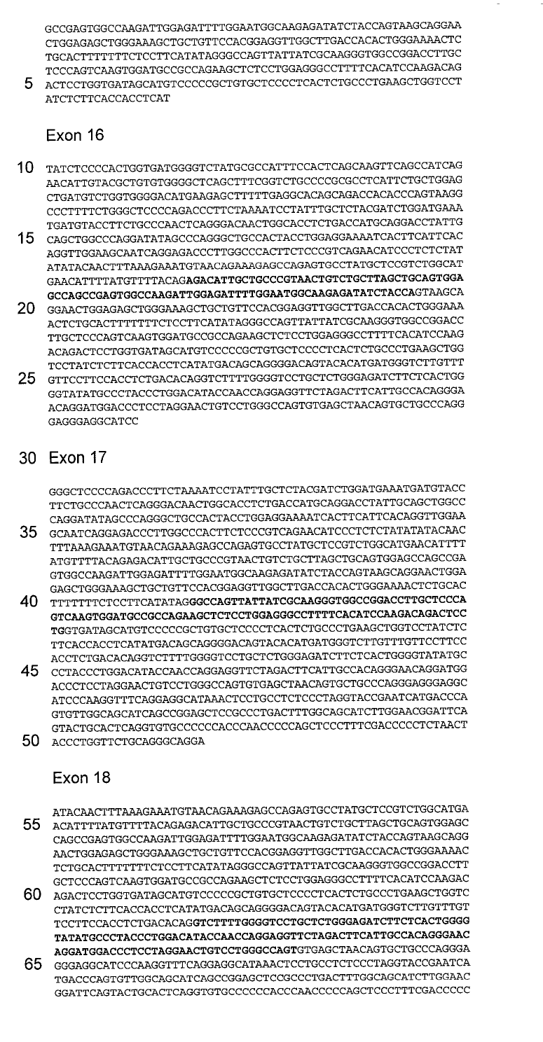

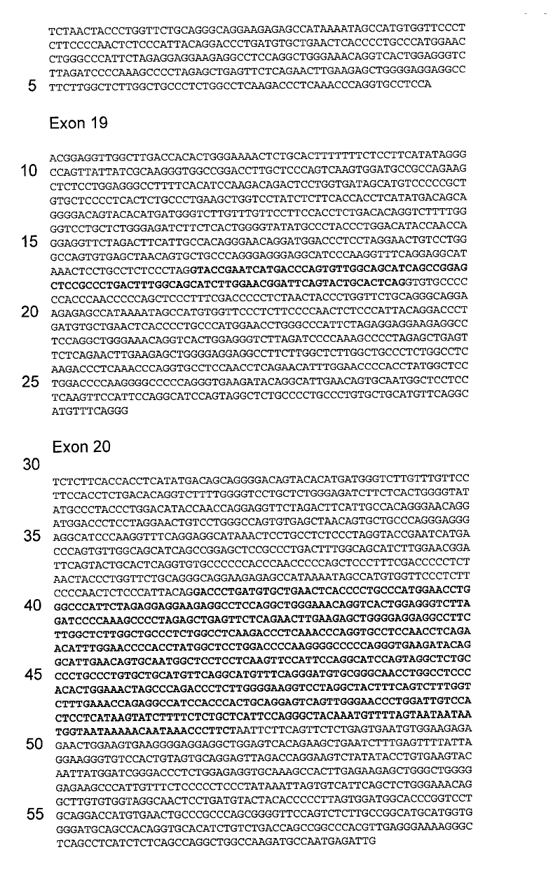

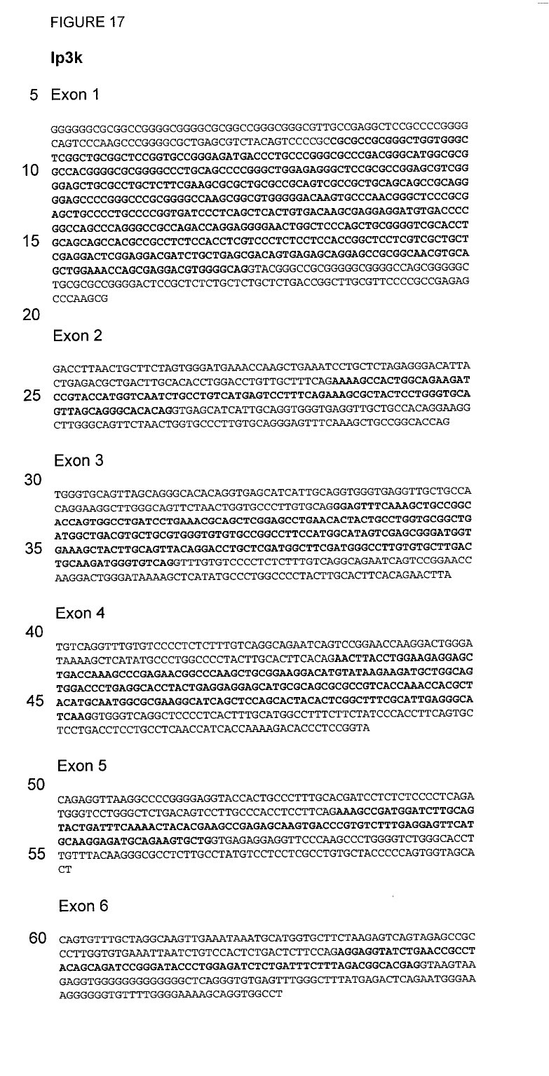

FIG. 17 is the sequence of gene Ip3k; and

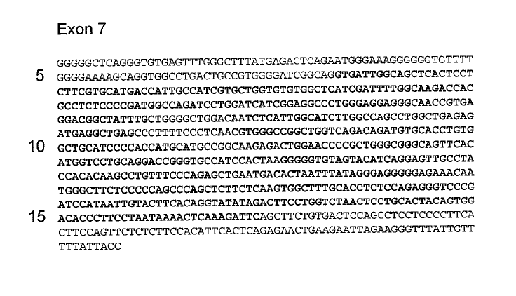

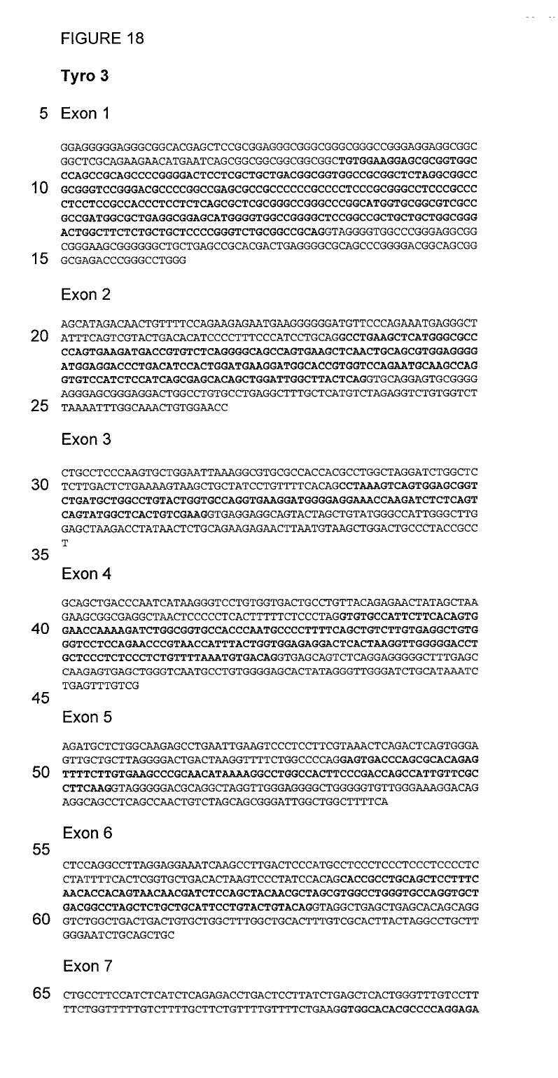

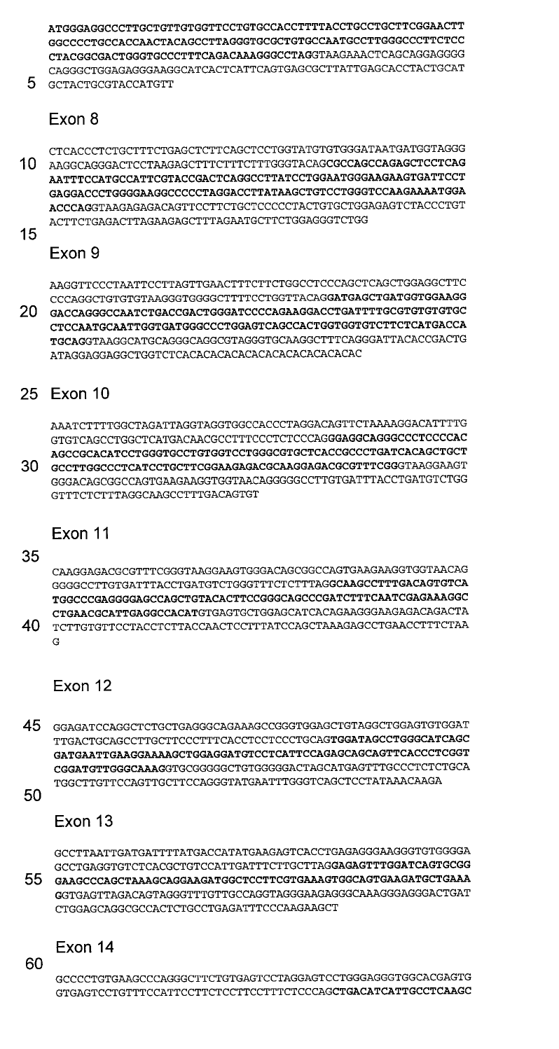

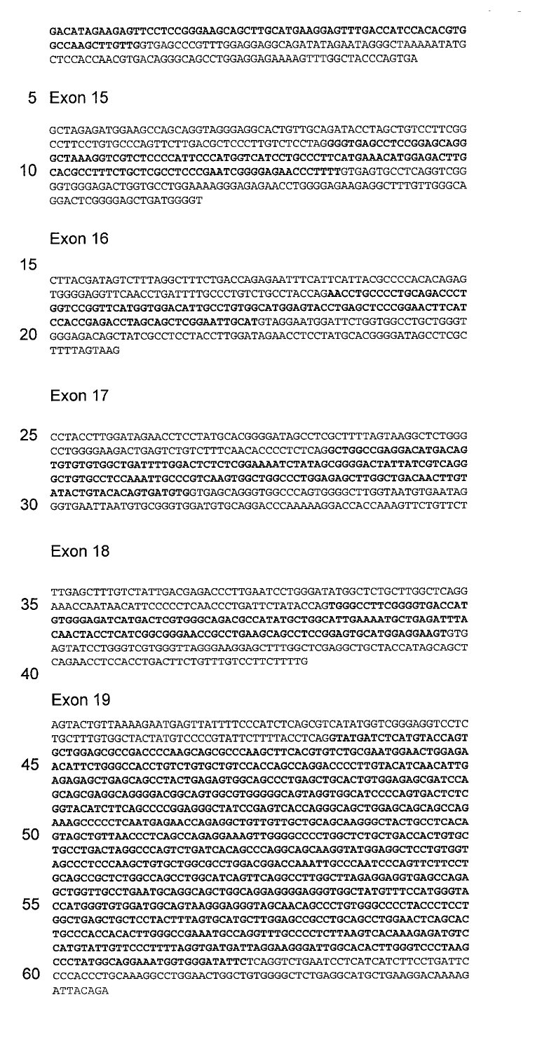

FIG. 18 is the sequence of gene Tyro 3.

### EXAMPLE 1

### Identifying the anx Interval in Mouse Chromosome 2

Material and Methods.

1. Markers and primers

All markers were simple sequence length polymorphisms (SSLPs) and were tested by PCR. The D2Mit markers were obtained from Research Genetics, Huntsville, Ala. The markers were chosen based on the chromosomal map position reported in the Chr2:Integrated MIT SSLP and Copeland/Jenkins RLFP Genetic Maps (http://carbon.wi.edu:8000/cgi-bin/mouse/sts_by_chrom). The D2Jojo primer pairs, with predicted annealing temperatures of 55° C. or higher, were designed around simple repeats located in the clones covering the anx interval.

The forward primer was labelled with γ-³²P-dATP as follows:

F primer (6,7 μM), 0.125 μl/sample; 10× PNK buffer,1.6 μl; T4 PNK (10U/μl), 1 μl/16 μl reaction; γ-³²P (6000 Ci), 0.03 μl/sample; H₂O to 16 μl final volume.

2. PCR protocol

The PCR was performed according to the following protocol.

Labelling reaction mixture, 16 μl; R primer (6,7 μM), 0.125 μl/sample, dNTPs (5 mM), 0.4 μl/sample; 10× PCR buffer (0.5M KCl, 15 mM MgCl₂, 100 mM Tris, 0.1% gelatin),1 μl/sample; Taq (5U/μl), 0.1 μl/sample; DNA, 2 μl; H₂O to 10 μl final volume.

95° C., 5 minutes; 95° C., 30 seconds; 50-60° C., 30 seconds (temperature optimised for each marker); 72° C., 30 seconds; 35 cycles; 72° C., 10 minutes; 4° C.

After the PCR had been carried out, the products were separated on a 6% denaturing polyacrylamide gel, National Diagnostics, Atlanta, GE, at 90V for approximately 1.5 hrs. The gel was then transferred to an X-ray film that was exposed for a period of time in the range of two hours to a couple of days.

3. DNA Preparation

DNA was prepared from 2-5 mm mouse tail snips as follows:

Add 1 ml of Tail Solubilisation Buffer (TSB) (1×SET [10×SET: 10% SDS, 50 mM EDTA, 100 mM Tris (pH 8.0)], 100 mM NaCl, 200 μg/ml proteinase K) to the tail snip. Incubate for about 4 hrs at 55° C. to digest tail.

Add 400 μl Tail Salts [4.21M NaCl, 0.63M KCl, 10 mM Tris (pH 8.0)] and vortex thoroughly. Incubate overnight at 4° C. to precipitate proteins.

Centrifuge the tubes for 10 minutes at maximum speed in a microfuge.

Remove 100 μl supernatant and precipitate the DNA with 200 μl 95% EtOH for 20 minutes at −70° C. or 4 hrs at −20° C.

Pellet the DNA in a microfuge at maximum speed for 20 minutes.

Remove supernatant and resuspend the DNA in 100 μl water or TE.

2 μl of DNA suspension was used in a PCR reaction.

4. Breeding and Mapping Scheme and Results

The anx mutation arose at The Jackson Laboratory (TJL), Bar Harbor, Me., in 1976 in the F2 generation of a cross between strain DW/J and an inbred strain derived from a cross of M. m. poschiavinus to a Swiss stock. The anx mutation has since then been kept on a nonagouti hybrid background referred to as B6C3-a/a F1. A general treatise of mouse genetics can be found in Mouse Genetics, Lee M. Silver, Oxford University Press, New York, 1995.

When starting a new linkage study in mice, two questions must be addressed. First, what strains should be used and second, what type of breeding scheme is suitable. To map a mutationally defined locus it is required to start with one strain that carries the mutation. This strain is then crossed with another strain that should be selected based on the genetic distance between the two strains—the larger the distance, the bigger chance is there to identify polymorphisms at DNA marker loci. The choice of breeding scheme is usually limited to one of two approaches: backcross or intercross. The nature of the phenotype may limit this choice even further. With both approaches, the first mating will always be an outcross between the two parental strains. Once F1 hybrid animals have been generated, it must be decided whether to backcross them to one of the parental strains or to intercross them with each other. There are disadvantages and advantages with both breeding scheme approaches. In this study it was decided to use the intercross approach to map the anx gene. The intercross has two major advantages over the backcross approach. The first is that it can be used to map recessive lethal mutations, such as the anx mutation, since both heterozygous F1 parents will be normal. The second advantage is that informative meiotic events will occur in both parents leading to twice as much information on a “per animal” basis as compared to the backcrossing.

anx breeding mice (B6C3-a/a-a +/+anx) were obtained from TJL. The anx gene had already been mapped to Chromosome 2 and two intercrosses were set up to narrow the anx interval further. Cross 1 comprises 2050 F2 progeny (4100 meioses) of an (B6C3Fe-anx A/+a×B6C3H Fl) intercross. Cross 2 was set up between B6C3Fe-anx A/+a and CAST/Ei and 372 F2 progeny (744 meioses) from this cross were analysed.

The mapping project was initiated by identifying polymorphic markers in the area to which the anx locus had been mapped, approximately 20cM proximal of the agouti locus on mouse Chromosome 2 (Maltais et al., 1984). Approximately 100 markers from Research Genetics, Huntsville, Ala., were tested and about 20% of these turned out to be polymorphic. The polymorphic markers were tested on the F2 progeny from both of the above crosses. As all of the anorexic mice had to have the genotype anx/anx at the mutation site, it was possible to work out how closely linked to the mutation the markers were, based on how many anorexic mice were recombined (showed an anx/+or +/+genotype) at those specific loci. A marker whose variant co-segregates with the anorexic phenotype is close to the gene controlling the phenotype. The order of the markers closest to the mutation is shown in FIG. 1. Three animals showed recombination between markers D2Jojo5 and anx (FIG. 1) thus defining D2Jojo5 as the distal flanking marker.

One F2 progeny from cross 2 identified D2Mit133 as the proximal flanking marker. The anx mutation was found to co-segregate with the markers D2Mit104, D2Mit395 and D2Jojo8. Recombination frequencies and standard errors were calculated as described by Green (1981). Genetic distances are shown in FIG. 1 as percentage recombination ±1 SE.

These results are backed up by a large number of recombinations on either side of the interval. All markers within the interval are non-recombinant and closely linked to the mutation.

### EXAMPLE 2

Establishing a Contig Covering the Identified anx Interval.

The distal end of the anx interval was covered by a Yeast Artificial Chromosome (YAC) spanning from Capn3 (distal to D2Jojo5) to D2Mit395, including Ltk and Tyro3 (Aamir Zuberi, The Jackson Laboratory, personal communication). When searching the Whitehead database (http://carbon.wi.mit.edu:8000/cgi-bin/mouse/index) for YACs containing the other loci known to map within or close to the anx interval, one YAC was identified that not only spans the proximal end of the anx interval, but also overlaps with the distal YAC (FIG. 2).

In order to further characterise the interval, three libraries (a P1 library derived from E14 (129/Ola) ES cells, PAC-RPCI-21 and BAC-RPCI-23) were screened for clones spanning the anx interval. The P1 library was screened by successive rounds of PCR with primers specific for D2Mit133, D2Mit104, D2Mit395 and Ltk. Four P1 clones were recovered (one for each locus). These clones were partially sequenced to obtain STS markers to be used as probes in the screening of the two RPCI-libraries. The clones were assembled in the order established from the genetic linkage map. The clones were linked together with STS markers generated from the ends of the clone inserts. In some cases the libraries were rescreened to find overlapping clones. FIG. 2 shows the relative order of markers, STS loci and the clones that span the interval between D2Mit133 and Capn3. Total DNA was isolated from clones using a CsCl gradient ultra-centrifugation or a modified Qiagen midi-prep protocol (Sambrook et al., 1989; Pinkel et al., 1998).

Materials and methods.

1. Partial sequencing of clone inserts

Partial sequences from the clones were generated using appropriate primers using the following reaction mixture: 2 μg DNA; 8 μl BIG Dye (Amersham); 20 pmol primer; H₂O to 20 μl final volume.

The sequencing reaction was performed during 100 cycles of ramping at temperatures in the range of 50° C. to 96° C. as follows:

$$\begin{matrix}{96}^{∘} C . , 30 seconds \\ {1}^{∘} C . \text{/} sec to {50}^{∘} C . \\ {50}^{∘} C . , 5 seconds \\ {1}^{∘} C . \text{/} sec to {60}^{∘} C .\end{matrix} \vert \} 100 x$$

60° C., 4 minutes

4° C

2. Purification of Samples

The samples were then purified according to the below protocol using Sephadex G50 microtiter filter plates (Millipore). Sephadex G50 (Pharmacia Biotech), 10 g/l 30 ml.

Fill the wells with Sephadex. Place a collection microtiter plate under the filter plate.

Centrifuge at 1500 rpm for 2 minutes. Empty the collection plate.

Fill the wells again and repeat centrifugation.

Add the sequencing reactions. Place a clean collection plate under the filter plate.

Centrifuge as above.

Vacuum dry the samples collected and store at −20° C. until needed.

Add loading dye and load the samples onto the ABI gel.

3. Providing labelled probes

The STS loci generated were PCR amplified and used as probes when screening the PAC-RPCI-21 and BAC-RPCI-23 libraries (BACPAC Resources, Oakland, Calif.). The probes were labelled using a standard Random Priming protocol (Current Protocols in Molecular Biology, 3.5.9-3.5.10). Finally, the probes were purified using QiaQuick Nucleotide removal kit (Qiagen, Venlo, the Netherlands).

4. Hybridization

The following hybridisation conditions were used:

The filters containing the libraries were pre-hybridised in hybridisation solution at 42° C. for 2 hrs. Then new hybridisation solution including labelled probes (denatured at 95° C., 5 minutes) was added and the filters were left to hybridise overnight. The filters were washed once for 15 minutes at room temperature (2× SSC, 0.1% SDS) followed by another wash for 15 minutes (0.1× SSC, 0.1% SDS). If the filters were still very hot the last wash was repeated.

Hybridisation solution: 2% SDS, 5× SSC, 10× Denhardt's solution, 50 mM P-buffer (pH 6.5), 50% formamide, salmon sperm DNA.

The filters were wrapped in saran wrap and applied onto an X-ray film that was exposed overnight and optionally for several days. The positive clones were obtained from Roswell Park, Buffalo, N.Y. Which clone had which marker was determined as follows:

Each clone was inoculated on a Nylon N⁺ membrane on top of a LB+50 μl/ml kanamycin plate. The clones were allowed to grow at 37° C. overnight. Then the membrane was treated as follows:

1. 0.5M NaOH, 1.5 M NaCl for 15 minutes, then dry.

2. 0.5M Tris pH 7.5, 1.5M NaCl for 10 minutes, then dry.

3. 0.5M Tris pH 7.5,1.5M NaCl, 50 μg/ml proteinase K for 30 minutes, then dry.

4. 2× SSC for 10 minutes, scrape off the colonies, then dry.

5. Stratalink.

The filters were then hybridised (conditions see above) with each of the probes separately to determine which clone corresponded with the different probes. The other markers within the interval were tested on the new PACs by PCR. The results are summarised in FIG. 2.

### EXAMPLE 3

### Showing that Tyro3 is Down Regulated in anx/anx Mice

3.1. Summary

A differential display analysis was performed on total RNA extracted from whole brain from newborn anx/anx (n=2) and +/+ (n=3) mice, and three weeks old anx/anx (n=3) and +/+ (n=3) mice. The analysis was done by Lark Technologies, Inc. using the Hieroglyph mRNA Profile Kit (Beckman Coulter, Inc., Fullerton, Calif.) and the genomyx LR System (Beckman Coulter, Inc., Fullerton, Calif.) to generate and display large amplified fragments. Differently expressed bands were excised from the gel, reamplified and, when necessary, subcloned prior to sequencing by Lark Technologies, Inc.

The differential display analysis identified 102 bands differing in expression levels between anx/anx and +/+ mice of one or both ages. When analyzing the bands it was discovered that one band that was missing in anx/anx mice of both ages was identical to the 3′UTR of Tyro3. To confirm this, a Northern blot and mRNA in situ hybridization was performed on brain tissue from both anx/anx and +/+ mice. These experiments confirmed a down regulation of Tyro3 mRNA in cerebellum of anx/anx mice.

3.2. Materials and Methods

1. Northern blotting

The Northern blot was performed using the following protocols:

(i) Providing a total RNA preparation

Total RNA was isolated from whole brain using Trizol (Life Technologies) according to the manufacturer's protocol.

(ii) Northern Blotting Gel

In the Northern blotting a 1,5% agarose gel was used. 200 ml of the gel was prepared by adding 3 g agarose to 150 ml DEPC-treated water followed by boiling to dissolve the agarose. To the solution 40 ml 5× MOPS (see below) was added and when cooled to 50° C., 10.8 ml formaldehyde was added. Prior to blotting the RNA, the gel was treated by rinsing in DEPC-treated water at least two times, in 50 mM NaOH for 20 minutes followed by rinsing in DEPC-treated water and in 20× SSC for 45 minutes.

(iii) Northern Blotting Procedure

The gel was prerun for 5 minutes at 100V in 1× MOPS. Total RNA samples were prepared by adding 10-12 μg of total RNA in 5 μl transfer buffer and vacuum dried if necessary, followed by adding 10 μl loading buffer. The resulting sample mixtures were denatured at 65° C. for 3-5 minutes.

The samples and an RNA ladder (3 μg) were loaded on the side leaving at least two empty lanes between ladder and samples and the gels were run at 40-50V. The BPB band was permitted to run approximately 10 cm. After running, the ladder was cut off, washed in 1× MOPS followed by staining with EtBr for 20-30 minutes and destaining in 0.2M NH40ac for 2×30 minutes.

Prior to blotting the RNA, the gel was treated by rinsing in DEPC-treated water at least twice in 50 mM NaOH for 20 minutes followed by rinsing in DEPC-treated water and in 20× SSC for 45 minutes.

Set up the blot (see Maniatis). Use 7,5 mM NaOH as transfer buffer and Hybond N+ membrane. Let it sit for at least 4,5 hours.

Rinse the membrane in 2× SSC, 0.1% SDS.

Stratalink.

(iv) Reagents Used

5× MOPS buffer:

The buffer contains 0.1 M MOPS (pH 7.0), 40 mM NaOAc, 5 mM EDTA (pH 8.0) and is prepared as follows: For 1 liter 20,6 g MOPS is added to 800 ml DEPC-treated NaOAc. pH is adjusted to 7.0 with 2M NaOH. 10 ml DEPC-treated EDTA is added. Bring to 1 liter with DEPC-treated water. Filter sterilise (Nalgene 0.2 μm) and store at room temperature protected from light.

Loading buffer:

The loading buffer used had the following composition: 7.2 ml deionized formamide, 3.2 ml 5× MOPS, 2.6 ml 37% formaldehyde, 1 ml 80% glycerol, 800 μl saturated bromphenol blue, 200 μl water.

2. Northern Hybridization

(i) Preparation of probe

The probe was labelled using a standard Random Priming protocol (Current Protocols in Molecular Biology, 3.5.9-3.5.10). Finally, the probe was purified using OiaQuick Nucleotide removal kit (Qiagen, Venlo, the Netherlands).

(ii) Hybridization Procedure

The hybridisation solution used contained per 100 ml: 8 ml 25% SDS, 20 ml 25× SSC, 10 ml 100× Denhardt solution, 5 ml 1 M phosphate buffer (pH 6,5), 50 ml formamide, 50% 1 ml ssDNA (denatured) and 6 ml water.

Pre-hybridisation of the sample was carried out for at least 1 hour at 42° C. (6 hours if the membrane has never been used before). Use a Tupperware container and just enough solution to cover the membrane. The hybridisation solution was replaced and the probe previously denatured at 95° C. for 5 minutes was added. Hybridisation was done overnight. Following hybridisation, the membranes were rinsed in 2× SSC, 0,1% SDS followed by washing in 2× SSC, 0,1% SDS for 15 minutes at room temperature and in 0,1× SSC, 0,1% SDS for 15 minutes at room temperature. If the membrane is still very hot a further was in 0,1× SSC, 0,1% SDS at 65° C. for 5-15 minutes may be carried out.

After a final wash in 2× SSC for 10-30 minutes, the membrane is wrapped in saran wrap and an autoradiography film exposed overnight with the membrane.

3. mRNA in Situ Hybridization

Brains to be tested were collected on postnatal day 20-22. Both male and female mice were analyzed. For in situ hybridization, the brains were rapidly dissected from decapitated mice and immersed in phosphate-buffered saline (PBS). Brains were mounted on chucks and rapidly frozen at −70° C.

A 45-mer antisense oligonucleotide, complementary to nucleotides 2411-2455 of Tyro 3 (Acc.no. U18343) was used. The oligonucleotide was subsequently labeled with ³⁵S-dATP (NEG 034H, NEN DuPont, Zaventem, Belgium) at the 3′ end using terminal deoxydinucleotidyl transferase to a specific activity of 2×10⁹ cpm/μg and purified using QIAquick Nucleotide Removal Kit (Qiagen, Venlo, the Netherlands).

The frozen brains were cut in 14 μm serial sections at −17° C. in a cryostat (Jung CM 3000, Leica Instruments GmbH, Nussloch, Germany). The sections were thaw-mounted onto ProbeOn slides (Fisher Biotech, Springfield, N.J.), and processed for in situ hybridization as described elsewhere (Schalling et al., 1988). In brief, the sections were incubated at 42° C. for 15-18 hr with 10⁶ cpm of labeled probe per 100 μl of a solution containing 50% formamide, 4× SSC (1× SSC: 0.15 M NaCl, 0.015 M sodium citrate), 1× Denhardt's solution (0.02% polyvinyl pyrrolidone, 0.02% bovine serum albumin and 0.02% Ficoll), 1% sarcosyl, 0.02 M sodium phosphate (pH 7.0), 10% dextran sulfate, 500 μg/ml sonicated salmon sperm DNA and 200 mM dithiothreitol. The sections were subsequently rinsed in 1× SSC at 55° C. for one hour with five changes of buffer, following which they were dried and exposed to Hyperfilm β-max X-ray film (Amersham International, Little Chalfront, UK) for seven days before development and fixation. Some of the sections were subsequently dipped in NTB 2 nuclear track emulsion (Kodak, Rochester, N.Y.), followed by two weeks of exposure, developed in Kodak D19, fixed in Kodak 3000A and analyzed using a microscope equipped for darkfield illumination (Axiophot, Carl Zeiss, Oberkochen, Germany). Pictures from these emulsion radiographs were taken using T-MAX 100 film (Kodak).

4. Mutation Analysis

Mutation analysis was performed by exon sequencing of genomic DNA. In short: each exon was PCR amplified and cycle-sequenced using an ABI 377 equipment. No mutations could be found in any of the exons. The promoter sequence was also searched for mutations but without success.

5. Conclusions

Tyro3 is down regulated in anx/anx mice compared to normal litter mates. While no mutations were found in this gene it is still possible that its down regulation contributes to the phenotype of anx/anx mice.

### REFERENCES

1. Broberger, C., J. Johansen, M. Schalling and T. Hökfelt (1997). Hypothalamic Neurohistochemistry of the Murine anorexia (anx/anx) Mutation: Altered Processing of Neuropeptide Y in the Arcuate Nucleus, J. Comp. Neurol. 387:124-135.

2. Broberger, C., J. Johansen, C. Johansson, M. Schalling and T. Hökfelt (1998). The neuropeptide Y/agouti gene-related protein (AGRP) brain circuitry in normal, anorectic, and monosodium glutamate-treated mice, Proc. Natl. Acad. Sci. USA 95:15043-15048.

3. Broberger, C., J. Johansen, H. Brismar, C. Johansson, M. Schalling and T. Hökfelt (1999). Changes in Neuropeptide Y Receptors and Pro-Opiomelanocortin in the Anorexia (anx/anx) Mouse Hypothalamus, J. Neurosci 19:7130-7139.

4. Green, M. C. (1981). Gene mapping, In “The mouse in Biomedical Research” (H. L. Foster, J. D. Small and J. D. Fox, Eds.), pp. 105-117, Academic Press, New York.

5. Johansen J. E., C. Broberger, C. Lavebratt, C. Johansson, M. J. Kuhar, T. Hökfelt, M. Schalling (2000). Hypothalamic CART and serum leptin levels are reduced in the anorectic (anx/anx) mouse. Mol. Brain Res. 84:97-105.

6. Pinkel, D., R. Segraves, D. Sudar, S. Clark, I. Poole, D. Kowbel, C. Collins, W. L. Kuo, C. Chen, Y. Zhai, S. H. Dairkee, B. M. Ljung, J. W. Gray, D. G. Albertson (1998). High resolution analysis of DNA copy number variation using comparative genomic hybridization to microarrays. Nature Genet. 20:207-211.

7. Sambrook, J., E. F. Fritsch, T. Maniatis. Molecular cloning; a laboratory manual. Cold Spring Harbor Laboratory Press, Cold Spring Harbor, N.Y.

8. Schalling, M., K. Seroogy, T. Hökfelt, S -Y. Chai, H. Hallman, H. Persson, D. Larhammar, A. Ericsson, L. Terenius, J. Graffi, J. Massoulié and M. Goldstein (1988). Neuropeptide tyrosine in the rat adrenal gland—Immunohistochemical and in situ hybridization studies, Neuroscience, 24:337-349.

1. An isolated nucleic acid molecule comprising a nucleotide sequence derived from a vertebrate animal, said nucleotide sequence codes for a polypeptide that is capable of at least partially complementing the phenotype of an animal having the anx/anx genotype as described in Maltais et al., J. Hered., 1984, 75:468-472, or a similar genotype.

2. A nucleic acid molecule according to claim 1 wherein the nucleotide sequence that codes for a polypeptide that is capable of complementing the phenotype of an animal having the anx/anx genotype as described in Maltais et al., J. Hered., 1984, 75:468-472, or a similar genotype, is constructed synthetically.

3. A nucleic acid molecule according to claim 1, further comprising a regulatory nucleotide sequence that regulates the expression of the nucleotide sequence coding for the phenotype complementing polypeptide, said regulatory nucleotide sequence is not naturally associated with the nucleotide sequence coding for the phenotype complementing polypeptide.

4. A nucleic acid molecule according to claim 1, further comprising a nucleotide sequence that encodes a secretion leader sequence that provides for secretion of the phenotype complementing polypeptide upon expression of the nucleotide sequence coding for the polypeptide.

5. A nucleic acid molecule according to claim 1 wherein the expression control sequence is one selected from the group consisting of a prokaryotic cell promoter, a eukaryotic cell promoter and a viral promoter.

6. A nucleic acid molecule according to claim 1 wherein the nucleotide sequence coding for a polypeptide that is capable of at least partially complementing the phenotype of an animal having the anx/anx genotype as described in Maltais et al., J. Hered., 1984, 75:468-472, or a similar genotype, is derived from a vertebrate animal including an animal selected from the group consisting of a rodent and a human.

7. A nucleic acid molecule according to claim 6 wherein the nucleotide sequence coding for a polypeptide that is capable of at least partially complementing the phenotype of an animal having the anx/anx genotype as described in Maltais et al., J. Hered., 1984, 75:468-472, or a similar genotype, is derived from a mouse.

8. A nucleic acid molecule according to claim 7 wherein the nucleotide sequence coding for a polypeptide that is capable of at least partially complementing the phenotype of an animal having the anx/anx genotype as described in Maltais et al., J. Hered., 1984, 75:468-472, or a similar genotype, is derived from a mouse having the genotype anx/anx.

9. A nucleic acid molecule according to claim 8 which is derived from a fragment of Chromosome 2 that is delimited by D2Mit133 and by D2Jojo5 as defined hereinbefore.

10. A nucleic acid molecule according to claim 1 comprising a nucleotide sequence selected from the group consisting of D2Dcr14, D2Mit104, D2Mit395, Tyro 3A, Tyro 3B and Tyro 3, as defined hereinbefore.

11. An expression vector comprising the nucleic acid molecule according to claim 1.

12. An expression vector according to claim 11 further comprising a regulatory nucleotide sequence that regulates the expression of the nucleotide sequence coding for the phenotype complementing polypeptide.

13. An expression vector according to claim 12 wherein the regulatory nucleotide sequence is not naturally associated with the nucleotide sequence coding for the phenotype complementing polypeptide.

14. An expression vector according to claim 11, further comprising a nucleotide sequence that encodes a secretion leader sequence that provides for secretion of the phenotype complementing polypeptide upon expression of the nucleotide sequence coding for the polypeptide.

15. An expression vector according to claim 12 wherein the expression control sequence is one selected from the group consisting of a prokaryotic cell promoter, a eukaryotic cell promoter and a viral promoter.

16. An expression vector according to claim 11 wherein the nucleotide sequence coding for a polypeptide that is capable of at least partially complementing the phenotype of an animal having the anx/anx genotype as described in Maltais et al., J. Hered., 1984, 75:468-472, or a similar genotype, is derived from a vertebrate animal including an animal selected from the group consisting of a rodent and a human.

17. An expression vector according to claim 16 wherein the nucleotide sequence coding for a polypeptide that is capable of at least partially complementing the phenotype of an animal having the anx/anx genotype as described in Maltais et al., J. Hered., 1984, 75:468-472, or a similar genotype, is derived from a mouse.

18. An expression vector according to claim 17 wherein the nucleotide sequence coding for a polypeptide that is capable of at least partially complementing the phenotype of an animal having the anx/anx genotype as described in Maltais et al., J. Hered., 1984, 75:468-472, or a similar genotype, is derived from a mouse having the genotype anx/anx.

19. An expression vector according to claim 18 wherein the nucleotide sequence coding for a polypeptide that is capable of at least partially complementing the phenotype of an animal having the anx/anx genotype as described in Maltais et al., J. Hered., 1984, 75:468-472, or a similar genotype, is derived from a fragment of the murine chromosome 2 that is delimited by D2Mit133 and by D2Jojo5 as defined hereinbefore.

20. An expression vector according to claim 11 comprising a nucleotide sequence selected from the group consisting of D2Dcr14, D2Mit104, D2Mit395, Tyro 3A, Tyro 3B and Tyro 3, as defined hereinbefore.

21. An expression vector according to claim 11, further comprising a nucleotide sequence coding for a selective marker.

22. A host cell comprising the vector of claim 11.

23. A host cell according to claim 22 wherein the vector further comprises a selective marker.

24. A host cell according to claim 23 wherein the expression vector comprises a regulatory nucleotide sequence that regulates the expression of the nucleotide sequence coding for the phenotype complementing polypeptide.

25. A host cell according to claim 24 wherein the regulatory nucleotide sequence is not naturally associated with the nucleotide sequence coding for the phenotype complementing polypeptide.

26. A host cell according to claim 22 wherein the expression vector comprises a nucleotide sequence that encodes a secretion leader sequence that provides for secretion of the phenotype complementing polypeptide upon expression of the nucleotide sequence coding for the polypeptide in the host cell.

27. A host cell according to claim 22 which is selected from the group consisting of a prokaryotic cell and an eukaryotic cell.

28. A host cell according to claim 27 which is a prokaryotic cell selected from the group consisting of a gram negative bacterium and a gram positive bacterium.

29. A host cell according to claim 28 which is E. coli.

30. A host cell according to claim 28 which is an eukaryotic cell selected from the group consisting of a mammalian cell, an insect cell and a fungal cell.

31. A host cell according to claim 30 which is a human cell.

32. A method of producing a polypeptide that is capable of at least partially complementing the phenotype of an animal having the anx/anx genotype as described in Maltais et al., J. Hered., 1984, 75:468-472, or a similar genotype, the method comprising the steps of:
(i) providing a nucleic acid molecule according to claim 1;
(ii) introducing the nucleic acid molecule into a host cell that is capable of expressing the nucleotide sequence coding for the phenotype complementing polypeptide;
(iii) culturing the host cell under conditions permitting expression of the nucleotide sequence coding for the phenotype complementing polypeptide: and
(iv) harvesting the polypeptide.

33. A method according to claim 32 wherein the nucleic acid molecule is introduced into the host cell as the expression vector according to claim 11.

34. A method according to claim 32 wherein the host cell cultured in step (iii) is the host cell of claim 22.

35. A method of inducing, in a vertebrate animal, the production of a polypeptide that is capable of at least partially complementing the phenotype of an animal having the anx/anx genotype as described in Maltais et al., J. Hered., 1984, 75:468-472, or a similar genotype, the method comprising administering to the animal the nucleic acid molecule of claim 1.

36. A method according to claim 35 wherein the nucleic acid molecule is administered directly or by viral or non-viral means.

37. A method according to claim 35 wherein the nucleic acid molecule is administered as a vector according to claim 11.

38. An isolated polypeptide that is capable of at least partially complementing the phenotype of an animal having the anx/anx genotype as described in Maltais et al., J. Heredity, 1984, 75:468-472, or a similar genotype, said polypeptide is obtainable by the method of claim 32.

39. A method of regulating food or feed intake in a vertebrate animal, the method comprising administering a food or feed intake regulating amount of the polypeptide according to claim 38.

40. A method of regulating weight gain in a vertebrate animal, the method comprising administering to the animal a weight gain regulating amount of the polypeptide according to claim 38.

41. A method of regulating food or feed intake in a vertebrate animal, the method comprising administering to the animal an effective amount of a molecule that blocks or inhibits or enhances the biological activity of the polypeptide according to claim 38.

42. A method according to claim 41 wherein the molecule that blocks or inhibits the biological activity of the polypeptide is a molecule selected from the group consisting of an antibody capable of binding to the polypeptide, a receptor capable of binding to the polypeptide, an antagonist that prevents the polypeptide from binding to its receptor and any other molecule that affects the activity of the polypeptide.

43. A method according to claim 41 wherein the molecule that enhances the biological activity of the polypeptide is an agonist.

44. A pharmaceutical composition comprising a polypeptide that is capable of at least partially complementing the phenotype of an animal having the anx/anx genotype as described in Maltais et al., J. Hered., 1984, 75:468-472, or a similar genotype, or a pharmaceutically active part thereof, and a pharmaceutically acceptable carrier.

45. A pharmaceutical composition comprising a molecule that blocks or inhibits the biological activity of a polypeptide that is capable of at least partially complementing the phenotype of an animal having the anx/anx genotype as described in Maltais et al., J. Hered., 1984, 75:468-472, or a similar genotype, said molecule is selected from the group consisting of an antibody capable of binding to the polypeptide, a receptor capable of binding to the polypeptide, an antagonist that prevents the polypeptide from binding to its receptor and any other molecule that is capable of binding to the polypeptide and/or affecting the biological activity of the polypeptide or a pharmaceutically active part thereof, and a pharmaceutically acceptable carrier.

46. A method of isolating a molecule that is capable of interacting with a polypeptide that is capable of at least partially complementing the phenotype of an animal having the anx/anx genotype as described in Maltais et al., J. Hered., 1984, 75:468-472, or a similar genotype, the method comprising the steps of:
(i) providing the polypeptide according to claim 38,
(ii) permitting the polypeptide to react with cells or fragments or extracts thereof to form a binding pair, and
(iii) separating from the binding pair a molecule that binds to the polypeptide.

47. An antibody comprising at least one binding site that is capable of binding to the polypeptide encoded by the nucleotide sequence according to claim 1.

48. An antibody according to claim 47 which is an antibody selected from the group consisting of a polyclonal antibody and a monoclonal antibody.

49. An antibody according to claim 47 which is one selected from an animal antibody and a humanised antibody.

50. A method of detecting the presence of a polypeptide that is capable of at least partially complementing the phenotype of an animal having the anx/anx genotype as described in Maltais et al., J. Hered., 1984, 75:468-472, or a similar genotype, or a homologue of said polypeptide, the method comprising contacting an antibody capable of binding to the polypeptide with a sample suspected of containing the polypeptide or the homologue thereof to allow formation of a polypeptide/antibody complex and detecting the complex.

51. A method according to claim 50 wherein the sample is one selected from brain tissue and pancreas tissue.

52. A method according to claim 50 wherein the antibody is a labelled antibody.

53. An isolated molecule that interacts with a polypeptide that is capable of at least partially complementing the phenotype of an animal having the anx/anx genotype as described in Maltais et al., J. Hered., 1984, 75:468-472, or a similar genotype, that is provided by the method of claim 46.

54. A molecule according to claim 53 that is a receptor molecule.

55. A method of producing an antibody to the molecule according to claim 53, the method comprising administering an immunologically effective amount of the molecule to an animal and collecting from the animals serum containing antibodies against the receptor or spleen cells for the production of monoclonal antibodies against the molecule.

56. A method according to claim 55 wherein the molecule is a receptor molecule.

57. A method for detection of a molecule that is capable of binding to a polypeptide capable of at least partially complementing the phenotype of an animal having the anx/anx genotype as described in Maltais et al., J. Hered., 1984, 75:468-472, or a similar genotype, the method comprising the steps of:
(i) providing the antibody produced by the method of claim 55,
(ii) reacting the antibody with cells or fragments or extracts thereof to form a binding pair, and
(iii) detecting the presence of the binding pair.

58. A method according to claim 57 wherein the molecule is a receptor molecule.

59. A method according to claim 57 wherein the antibody is a labelled antibody.

60. A kit for detection of a polypeptide that is capable of at least partially complementing the phenotype of an animal having the anx/anx genotype as described in Maltais et al., J. Hered., 1984, 75:468-472, or a similar genotype, said kit comprising an antibody according to claim 47.

61. A kit for detection of a receptor for a polypeptide that is capable of at least partially complementing the phenotype of an animal having the anx/anx genotype as described in Maltais et al., J. Hered., 1984, 75:468-472, or a similar genotype, said kit comprising said polypeptide.

62. A kit according to claim 61 wherein the polypeptide is in a labelled form.

63. A kit for detection of antibodies to a polypeptide that is capable of at least partially complementing the phenotype of an animal having the anx/anx genotype as described in Maltais et al., J. Hered., 1984, 75:468-472, or a similar genotype, said kit comprising the polypeptide.

64. A kit according to claim 63 wherein the polypeptide is in a labelled form.

65. A method of identifying in a sample a nucleotide sequence that codes for a putative polypeptide as defined in claim 1, the method comprising the steps of.
(i) providing the coding nucleotide sequence of claim 1 or its complementary strand, or a part thereof,
(ii) contacting the sample with said sequence or part under hybridising conditions to form a binding pair; and
(iii) detecting the presence of the binding pair.

66. A method according to claim 65 wherein the coding nucleotide sequence or its complementary strand, or part thereof is labelled.

67. A method of controlling in an animal the expression of a nucleotide sequence coding for a polypeptide that is capable of at least partially complementing the phenotype of an animal having the anx/anx genotype as described in Maltais et al., J. Hered., 1984, 75:468-472, or a similar genotype, the method comprising the steps of:
(i) providing a molecule that inhibits the translation of a transcript of the coding sequence; and
(ii) administering to the animal a translation inhibiting effective amount of said molecule.

68. A method according to claim 67 wherein the translation inhibiting molecule is selected from the group consisting of antisense RNA and PNA.
